SEQUENCE LISTING (49 sequences)

SEQ ID NO: 1 - protein, 86 aa, Synthetic construct
  REGION 1..86: note = IL2RG Common Gamma Chain Intracellular domain
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 2 - protein, 286 aa, Synthetic construct
  REGION 1..286: note = IL2RB intracellular domain
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 195 aa, Synthetic construct
  REGION 1..195: note = IL7RB intracellular domain
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 4 - protein, 285 aa, Synthetic construct
  REGION 1..285: note = IL21RB intracellular domain
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 5 - protein, 107 aa, Synthetic construct
  REGION 1..107: note = FKBP domain (FKBP12)
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 6 - protein, 93 aa, Synthetic construct [sequence omitted]

SEQ ID NO: 7 - protein, 93 aa, Synthetic construct
  REGION 1..93: note = variant FRB domain (FRB mutant domain)
ILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 8 - protein, 21 aa, Synthetic construct
  REGION 1..21: note = transmembrane (TM) domain
VVISVGSMGLIISLLCVYFWL

SEQ ID NO: 9 - protein, 19 aa, Synthetic construct
  REGION 1..19: note = TM domain
VAVAGCVFLLISVLLLSGL

SEQ ID NO: 10 - protein, 25 aa, Synthetic construct
  REGION 1..25: note = TM domain
PILLTISILSFFSVALLVILACVLW

SEQ ID NO: 11 - protein, 21 aa, Synthetic construct
  REGION 1..21: note = TM domain
GWNPHLLLLLLLVIVFIPAFW

SEQ ID NO: 12 - protein, 21 aa, Synthetic construct
  REGION 1..21: note = CD8a signal sequence
MALPVTALLLPLALLLHAARP

SEQ ID NO: 13 - DNA, 1554 bp, Synthetic construct
  misc_feature 1..1554: note = nucleotide sequence encoding a CAR
  source 1..1554: mol_type = other DNA
atggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 14 - protein, 517 aa, Synthetic construct
  REGION 1..517: note = CAR amino acid sequence
MALPVTALLLPLALLLHAARPDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 1560 bp, Synthetic construct
  misc_feature 1..1560: note = nucleotide insert
  source 1..1560: mol_type = other DNA
gccaccatggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 16 - protein, 496 aa, Synthetic construct
  REGION 1..496: note = CAR amino acid sequence without the signal peptide
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 21 aa, Synthetic construct
  REGION 1..21: note = CAR amino acid sequence signal peptide
MALPVTALLLPLALLLHAARP

SEQ ID NO: 18 - RNA, 20 nt, Synthetic construct
  misc_feature 1..20: note = B2M gRNA
  source 1..20: mol_type = other RNA
ggccgagatgtctcgctccg

SEQ ID NO: 19 - RNA, 20 nt, Synthetic construct
  misc_feature 1..20: note = FKBP12 gRNA
  source 1..20: mol_type = other RNA
cacgcaggtctggccgcgct

SEQ ID NO: 20 - RNA, 20 nt, synthetic construct
  source 1..20: mol_type = other RNA
  unsure 1..20: note = FKBP12 gRNA
tgctgagccgatgcgcgcgg

SEQ ID NO: 21 - RNA, 20 nt, Synthetic construct
  misc_feature 1..20: note = FKBP12 gRNA
  source 1..20: mol_type = other RNA
ctcaccgtctcctggggaga

SEQ ID NO: 22 - DNA, 500 bp, Synthetic construct
  misc_feature 1..500: note = B2M 5' homology arm
  source 1..500: mol_type = other DNA
tacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggc

SEQ ID NO: 23 - DNA, 500 bp, Synthetic construct
  misc_feature 1..500: note = B2M 3' homology arm
  source 1..500: mol_type = other DNA
tggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctggggga

SEQ ID NO: 24 - DNA, 1189 bp, Synthetic construct
  misc_feature 1..1189: note = EF1alpha promoter
  source 1..1189: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaa

SEQ ID NO: 25 - DNA, 1188 bp, Synthetic construct
  misc_feature 1..1188: note = EF1alpha promoter
  source 1..1188: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 26 - DNA, 1205 bp, Synthetic construct
  misc_feature 1..1205: note = EF1alpha promoter
  source 1..1205: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaa

SEQ ID NO: 27 - DNA, 347 bp, Synthetic construct
  misc_feature 1..347: note = MND promoter
  source 1..347: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 28 - protein, 231 aa, Synthetic construct
  REGION 1..231: note = RACR (g)
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 29 - protein, 20 aa, Synthetic construct
  REGION 1..20: note = Signal peptide
MPLGLLWLGLALLGALHAQA

SEQ ID NO: 30 - protein, 108 aa, Synthetic construct
  REGION 1..108: note = FKBP domain (FKBP12)
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGE

SEQ ID NO: 31 - protein, 37 aa, Synthetic construct
  REGION 1..37: note = IL2RG transmembrane domain and ectodomain portion
GSNTSKENPFLFALEAVVISVGSMGLIISLLCVYFWL

SEQ ID NO: 32 - DNA, 3354 bp, Synthetic construct
  misc_feature 1..3354: note = RACR
  source 1..3354: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgccactgggactgctgtggctgggactggccctgctgggcgccctgcacgcccaggccggcgtgcaggtggagacaatcagcccaggcgacggacgcacctttcctaagcggggccagacatgcgtggtgcactacaccggcatgctggaggatggcaagaagttcgactctagcagagataggaacaagccctttaagttcatgctgggcaagcaggaagtgatcagaggctgggaggagggcgtggcccagatgtctgtgggccagagggccaagctgacaatcagcccagactacgcatatggagcaaccggacacccaggaatcatcccacctcacgccacactggtgttcgatgtggagctgctgaagctgggcgagggctctaacaccagcaaggagaatccatttctgttcgcactggaggcagtggtcatctccgtgggctctatgggcctgatcatctccctgctgtgcgtgtacttttggctggagagaacaatgcccaggatccctaccctgaagaacctggaggacctggtgaccgagtatcacggcaatttcagcgcctggtccggcgtgtctaagggactggcagagtccctgcagccagattactctgagcggctgtgcctggtgagcgagatcccacccaagggaggcgccctgggagagggacctggagcaagcccatgcaaccagcactccccatattgggcccctccatgttacaccctgaagcctgagacaggaagcggagcgacgaattttagtctactgaaacaagcgggagacgtggaggaaaaccctggacctatggcactgccagtgaccgccctgctgctgccactggccctgctgctgcacgcagcacgccccatcctgtggcacgaaatgtggcatgaaggcctggaggaggcaagcagactgtattttggcgagagaaatgtgaaaggaatgtttgaggtgctggagccactgcacgcaatgatggagaggggacctcagaccctgaaggagacatcctttaaccaggcctatggcagagacctgatggaggcccaggagtggtgcaggaagtacatgaagagcggaaatgtgaaagacctgctgcaggcctgggatctgtattatcacgtgttcagaaggatcagcaagggcaaggataccatcccttggctgggacacctgctggtgggactgtccggagcctttggcttcatcatcctggtgtacctgctgatcaactgccgcaatacaggcccatggctgaagaaggtgctgaagtgtaacaccccagacccctccaagttcttttctcagctgtcctctgagcacggcggcgatgtgcagaagtggctgagctccccttttccatctagctccttcagccctggaggactggcaccagagatctcccctctggaggtgctggagcgggacaaggtgacccagctgctgctgcagcaggataaggtgcccgagcctgcctccctgtctagcaaccacagcctgacctcctgctttacaaatcagggctatttctttttccacctgccagacgcactggagatcgaggcatgtcaggtgtacttcacatatgatccatacagcgaggaggacccagatgagggagtggccggcgcccctaccggatcctctccacagccactgcagccactgtctggcgaggacgatgcctactgtacatttcctagcagagacgatctgctgctgttctccccttctctgctgggaggaccatctccacctagcaccgcacctggaggatccggggcaggggaggagaggatgccaccatctctgcaggagagggtgccaagggactgggatcctcagccactgggacctccaaccccaggagtgcccgacctggtggatttccagccccctccagagctggtgctgagagaggcaggagaggaggtgccagacgcaggaccaagagagggcgtgagctttccatggtccaggccacctggacagggagagttcagagccctgaacgccaggctgccactgaatacagacgcctacctgtctctgcaggagctgcagggccaggatcccacccacctggtgtaaacaataaaagatctttattttcattagatctgtgtgttggttttttgtgtg

SEQ ID NO: 33 - protein, 429 aa, Synthetic construct
  REGION 1..429: note = RACR (b)
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 34 - protein, 21 aa, Synthetic construct
  REGION 1..21: note = CD8a Signal peptide
MALPVTALLLPLALLLHAARP

SEQ ID NO: 35 - protein, 29 aa, Synthetic construct
  REGION 1..29: note = IL2RB transmembrane domain and ectodomain portion
GKDTIPWLGHLLVGLSGAFGFIILVYLLI

SEQ ID NO: 36 - protein, 25 aa, Synthetic construct
  REGION 1..25: note = IL2RB transmembrane domain
IPWLGHLLVGLSGAFGFIILVYLLI

SEQ ID NO: 37 - DNA, 753 bp, Synthetic construct
  misc_feature 1..753: note = RACR (g) Nucleotides
  source 1..753: mol_type = other DNA
atgccactgggactgctgtggctgggactggccctgctgggcgccctgcacgcccaggccggcgtgcaggtggagacaatcagcccaggcgacggacgcacctttcctaagcggggccagacatgcgtggtgcactacaccggcatgctggaggatggcaagaagttcgactctagcagagataggaacaagccctttaagttcatgctgggcaagcaggaagtgatcagaggctgggaggagggcgtggcccagatgtctgtgggccagagggccaagctgacaatcagcccagactacgcatatggagcaaccggacacccaggaatcatcccacctcacgccacactggtgttcgatgtggagctgctgaagctgggcgagggctctaacaccagcaaggagaatccatttctgttcgcactggaggcagtggtcatctccgtgggctctatgggcctgatcatctccctgctgtgcgtgtacttttggctggagagaacaatgcccaggatccctaccctgaagaacctggaggacctggtgaccgagtatcacggcaatttcagcgcctggtccggcgtgtctaagggactggcagagtccctgcagccagattactctgagcggctgtgcctggtgagcgagatcccacccaagggaggcgccctgggagagggacctggagcaagcccatgcaaccagcactccccatattgggcccctccatgttacaccctgaagcctgagaca

SEQ ID NO: 38 - DNA, 1287 bp, Synthetic construct
  misc_feature 1..1287: note = RACR (b) nucleotides
  source 1..1287: mol_type = other DNA
atggcactgccagtgaccgccctgctgctgccactggccctgctgctgcacgcagcacgccccatcctgtggcacgaaatgtggcatgaaggcctggaggaggcaagcagactgtattttggcgagagaaatgtgaaaggaatgtttgaggtgctggagccactgcacgcaatgatggagaggggacctcagaccctgaaggagacatcctttaaccaggcctatggcagagacctgatggaggcccaggagtggtgcaggaagtacatgaagagcggaaatgtgaaagacctgctgcaggcctgggatctgtattatcacgtgttcagaaggatcagcaagggcaaggataccatcccttggctgggacacctgctggtgggactgtccggagcctttggcttcatcatcctggtgtacctgctgatcaactgccgcaatacaggcccatggctgaagaaggtgctgaagtgtaacaccccagacccctccaagttcttttctcagctgtcctctgagcacggcggcgatgtgcagaagtggctgagctccccttttccatctagctccttcagccctggaggactggcaccagagatctcccctctggaggtgctggagcgggacaaggtgacccagctgctgctgcagcaggataaggtgcccgagcctgcctccctgtctagcaaccacagcctgacctcctgctttacaaatcagggctatttctttttccacctgccagacgcactggagatcgaggcatgtcaggtgtacttcacatatgatccatacagcgaggaggacccagatgagggagtggccggcgcccctaccggatcctctccacagccactgcagccactgtctggcgaggacgatgcctactgtacatttcctagcagagacgatctgctgctgttctccccttctctgctgggaggaccatctccacctagcaccgcacctggaggatccggggcaggggaggagaggatgccaccatctctgcaggagagggtgccaagggactgggatcctcagccactgggacctccaaccccaggagtgcccgacctggtggatttccagccccctccagagctggtgctgagagaggcaggagaggaggtgccagacgcaggaccaagagagggcgtgagctttccatggtccaggccacctggacagggagagttcagagccctgaacgccaggctgccactgaatacagacgcctacctgtctctgcaggagctgcagggccaggatcccacccacctggtg

SEQ ID NO: 39 - DNA, 49 bp, Synthetic construct
  misc_feature 1..49: note = Synthetic polyA
  source 1..49: mol_type = other DNA
aataaaagatctttattttcattagatctgtgtgttggttttttgtgtg

SEQ ID NO: 40 - DNA, 4354 bp, Synthetic construct
  misc_feature 1..4354: note = RACR_B2M ssODN
  source 1..4354: mol_type = other DNA
tacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgccactgggactgctgtggctgggactggccctgctgggcgccctgcacgcccaggccggcgtgcaggtggagacaatcagcccaggcgacggacgcacctttcctaagcggggccagacatgcgtggtgcactacaccggcatgctggaggatggcaagaagttcgactctagcagagataggaacaagccctttaagttcatgctgggcaagcaggaagtgatcagaggctgggaggagggcgtggcccagatgtctgtgggccagagggccaagctgacaatcagcccagactacgcatatggagcaaccggacacccaggaatcatcccacctcacgccacactggtgttcgatgtggagctgctgaagctgggcgagggctctaacaccagcaaggagaatccatttctgttcgcactggaggcagtggtcatctccgtgggctctatgggcctgatcatctccctgctgtgcgtgtacttttggctggagagaacaatgcccaggatccctaccctgaagaacctggaggacctggtgaccgagtatcacggcaatttcagcgcctggtccggcgtgtctaagggactggcagagtccctgcagccagattactctgagcggctgtgcctggtgagcgagatcccacccaagggaggcgccctgggagagggacctggagcaagcccatgcaaccagcactccccatattgggcccctccatgttacaccctgaagcctgagacaggaagcggagcgacgaattttagtctactgaaacaagcgggagacgtggaggaaaaccctggacctatggcactgccagtgaccgccctgctgctgccactggccctgctgctgcacgcagcacgccccatcctgtggcacgaaatgtggcatgaaggcctggaggaggcaagcagactgtattttggcgagagaaatgtgaaaggaatgtttgaggtgctggagccactgcacgcaatgatggagaggggacctcagaccctgaaggagacatcctttaaccaggcctatggcagagacctgatggaggcccaggagtggtgcaggaagtacatgaagagcggaaatgtgaaagacctgctgcaggcctgggatctgtattatcacgtgttcagaaggatcagcaagggcaaggataccatcccttggctgggacacctgctggtgggactgtccggagcctttggcttcatcatcctggtgtacctgctgatcaactgccgcaatacaggcccatggctgaagaaggtgctgaagtgtaacaccccagacccctccaagttcttttctcagctgtcctctgagcacggcggcgatgtgcagaagtggctgagctccccttttccatctagctccttcagccctggaggactggcaccagagatctcccctctggaggtgctggagcgggacaaggtgacccagctgctgctgcagcaggataaggtgcccgagcctgcctccctgtctagcaaccacagcctgacctcctgctttacaaatcagggctatttctttttccacctgccagacgcactggagatcgaggcatgtcaggtgtacttcacatatgatccatacagcgaggaggacccagatgagggagtggccggcgcccctaccggatcctctccacagccactgcagccactgtctggcgaggacgatgcctactgtacatttcctagcagagacgatctgctgctgttctccccttctctgctgggaggaccatctccacctagcaccgcacctggaggatccggggcaggggaggagaggatgccaccatctctgcaggagagggtgccaagggactgggatcctcagccactgggacctccaaccccaggagtgcccgacctggtggatttccagccccctccagagctggtgctgagagaggcaggagaggaggtgccagacgcaggaccaagagagggcgtgagctttccatggtccaggccacctggacagggagagttcagagccctgaacgccaggctgccactgaatacagacgcctacctgtctctgcaggagctgcagggccaggatcccacccacctggtgtaaacaataaaagatctttattttcattagatctgtgtgttggttttttgtgtgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctggggga

SEQ ID NO: 41 - DNA, 66 bp, Synthetic construct
  misc_feature 1..66: note = PQR linker nucleotide
  source 1..66: mol_type = other DNA
ggaagcggagcgacgaattttagtctactgaaacaagcgggagacgtggaggaaaaccctggacct

SEQ ID NO: 42 - protein, 22 aa, Synthetic construct
  REGION 1..22: note = PQR linker
GSGATNFSLLKQAGDVEENPGP

SEQ ID NO: 43 - protein, 22 aa, Synthetic construct
  REGION 1..22: note = F2A
VKQTLNFDLLKLAGDVESNPGP

SEQ ID NO: 44 - protein, 20 aa, Synthetic construct
  REGION 1..20: note = E2A
QCTNYALLKLAGDVESNPGP

SEQ ID NO: 45 - protein, 24 aa, Synthetic construct
  REGION 1..24: note = T2A
LEGGGEGRGSLLTCGDVEENPGPR

SEQ ID NO: 46 - protein, 18 aa, Synthetic construct
  REGION 1..18: note = T2A
EGRGSLLTCGDVEENPGP

SEQ ID NO: 47 - protein, 22 aa, Synthetic construct
  REGION 1..22: note = P2A
GSGATNFSLLKQAGDVEENPGP

SEQ ID NO: 48 - protein, 19 aa, Synthetic construct
  REGION 1..19: note = P2A
ATNFSLLKQAGDVEENPGP

SEQ ID NO: 49 - protein, 106 aa, Synthetic construct
  REGION 1..106: note = FKBP domain (FKBP12)
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKL

(19) United States
(12) Patent Application Publication
(10) Pub. No.: US 2025/0367290 A1
(43) Pub. Date: Dec. 4, 2025

(54) ENGINEERED STEM CELLS AND USES THEREOF

(71) Applicant: Umoja Biopharma, Inc., Seattle, WA (US)

(72) Inventors: Samantha O'HARA, Seattle, WA (US); David T. VEREIDE, Seattle, WA (US); Ryan LARSON, Seattle, WA (US); Andrew SCHARENBERG, Seattle, WA (US); Ashley YINGST, Seattle, WA (US); Dillon JARRELL, Seattle, WA (US); Ryan KONING, Seattle, WA (US); Teisha ROWLAND, Seatlle, WA (US)

(73) Assignee: Umoja Bioharma, Inc., Seattle, WA (US)

(21) Appl. No.: 18/873,311

(22) PCT Filed: Jun. 10, 2023
(86) PCT No.: PCT/US2023/068261

§ 371 Date: Dec. 9, 2024

Related U.S. Application Data
(60) Provisional application No. 63/351,144, filed on Jun. 10, 2022.
(60) Provisional application No. 63/392,861, filed on Jul. 27, 2022.
(60) Provisional application No. 63/411,065, filed on Sep. 28, 2022.
(60) Provisional application No. 63/422,882, filed on Nov. 4, 2022.
(60) Provisional application No. 63/447,337, filed on Feb. 21, 2023.
(60) Provisional application No. 63/451,536, filed on Mar. 10, 2023.

(51) Int. Cl. A61K 40/15; A61K 40/31; A61K 40/42; A61P 35/00; C07K 14/725; C07K 16/28; C12N 5/0783

(52) U.S. Cl. CPC A61K 40/15; A61K 40/31; A61K 40/4211; A61P 35/00; C07K 14/7051; C07K 16/2803; C12N 5/0646; A61K 2239/23; C12N 2501/2302; C12N 2501/2307; C12N 2501/2315; C12N 2501/2318; C12N 2501/2321

(57) ABSTRACT

Provided are compositions and methods for a cell population comprising engineered stem cells comprising a synthetic cytokine receptor for a non-physiological ligand. The non-physiological ligand activates the synthetic cytokine receptor in the engineered stem cells to induce differentiation of the stem cells and, expansion and/or activation of resulting cytotoxic innate lymphoid cells.

### CROSS-REFERENCE TO RELATED APPLICATIONS

This application claims priority from U.S. provisional application No. 63/351,144 filed Jun. 10, 2022, entitled “Engineered Stem Cells and Uses Thereof,” U.S. provisional application No. 63/392,861 filed Jul. 27, 2022, entitled “Engineered Stem Cells and Uses Thereof.” U.S. provisional application No. 63/411,065 filed Sep. 28, 2022, entitled “Engineered Stem Cells and Uses Thereof,” U.S. provisional application No. 63/422,882 filed Nov. 4, 2022, entitled “Engineered Stem Cells and Uses Thereof.” U.S. provisional application No. 63/447,337 filed Feb. 21, 2023, entitled “Engineered Stem Cells and Uses Thereof,” and U.S. provisional application No. 63/451,536 filed Mar. 10, 2023, entitled “Engineered Stem Cells and Uses Thereof,” the contents of each of which are incorporated by reference in their entirety.

### INCORPORATION BY REFERENCE OF SEQUENCE LISTING

The present application is being filed with a Sequence Listing in electronic format. The Sequence Listing is provided as a file entitled 260132000340SeqList.xml, created on Jun. 9, 2023, which is 83,181 bytes in size. The information in electronic format of the Sequence Listing is incorporated by reference in its entirety.

### FIELD

The present disclosure provides compositions and methods related to a cell population comprising engineered stem cells comprising a synthetic cytokine receptor for a non-physiological ligand. The non-physiological ligand activates the synthetic cytokine receptor in the engineered stem cells to induce differentiation of the stem cells and, expansion and/or activation of resulting cytotoxic innate lymphoid cells.

### BACKGROUND

Cytotoxic innate lymphoid cells (CILs) are a class of immune cells that may be used in immunotherapy including cancer immunotherapy. One type of CIL is a natural killer (NK) cell, a type of cell generally identified as positive for the cell surface protein CD56 (CD56+) and other markers and as having cytotoxic activity.

CIL cells for use in immunotherapy can be obtained from primary sources such as peripheral blood or umbilical cord blood. Artificial sources for CIL cells include pluripotent stem cells, including induced pluripotent stem cells (iPSCs), which are cells derived from somatic cells (generally fibroblasts or peripheral blood mononuclear cells [PBMCs]), and human embryonic stem cells (hESCs), either induced to become capable of unlimited proliferation and of differentiation into other cell types when subjected to appropriate differentiation conditions. From iPSCs, CIL cells may be derived by sequentially differentiating the iPSCs into hematopoietic progenitor cells (HPCs), also termed hematopoietic stem cells (HSCs); the HPCs into common lymphoid progenitor cells (CLPs); and then the CLPs into CIL cells-termed iPSC-derived cytotoxic innate lymphoid cells (iPSC-CILs). Generally, iPSC-CIL cells express CD56 and have cytotoxic activity, like NK cells; but iPSC-CIL cells may differ from NK cells phenotypically and in other respects.

Methods for differentiating iPSCs into CD34+ HPCs using either embryoid embodies (EBs) or culture of single-cell iPSCs on feeder cells are known. CD34+ HPCs may then be differentiated into CLPs.

There remains a need in the art for compositions and methods related to engineered stem cells, methods for making such cells, methods for differentiating such cells into CILs, and methods of using them in immunotherapy.

### SUMMARY OF THE DISCLOSURE

The present disclosure is based, in part, on the discovery that stem cells engineered to express a synthetic cytokine receptor improve or enhance differentiation to hematopoietic progenitors and CLPs in response to the receptor's cognate non-physiological ligand. Such progenitors are subsequently differentiated into engineered CIL cells. As demonstrated herein, CRISPR is used to genetically engineer stem cells to express the synthetic cytokine receptor and in some embodiments simultaneously disrupt genes to avoid immune rejection (e.g., beta-2-microglobulin) and/or provide resistance to rapamycin.

According to the methods described herein, CIL cells may be generated in high quantities and with desirable functional characteristics from engineered stem cells. Non-limiting advantages of certain embodiments include the ability of the CIL cells described herein to be differentiated without the use of exogenous factors, such as without SCF, TPO, BMP4, FGF and/or VEGF, or to supplement exogenous factors. Further, CIL cells expressing the synthetic cytokine receptor provide the ability of the CIL cells described herein to be expanded without the use of exogenous factors, such as without IL-2, IL-7, IL-15, and/or IL-21. The CIL cells described herein, and related compositions, may be used for immunotherapy with ex vivo expansion.

Accordingly, in some aspects, the disclosure provides an engineered stem cell comprising a synthetic cytokine receptor for a non-physiological ligand, wherein the cytokine receptor comprises: a synthetic gamma chain polypeptide comprising a first dimerization domain, a first transmembrane domain, and an interleukin-2 receptor subunit gamma (IL-2RG) intracellular domain, and a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

In some embodiments, the first dimerization domain and the second dimerization domain are extracellular domains. In some embodiments, the synthetic gamma chain polypeptide comprises, in N- to C-terminal order, the first dimerization domain, the first transmembrane domain, and the interleukin-2 receptor subunit gamma (IL-2RG) intracellular domain, and the synthetic beta chain polypeptide comprises, in N- to C-terminal order, the second dimerization domain, the second transmembrane domain, and the intracellular domain.

In some embodiments, the IL-2RG intracellular domain comprises a polypeptide sequence at least 95% identical to SEQ ID NO: 1, or a polypeptide sequence as set forth in SEQ ID NO: 1. In some embodiments, the first transmembrane domain comprises the IL-2RG transmembrane domain. In some embodiments, the IL-2RG transmembrane domain comprises a polypeptide sequence at least 95% identical to SEQ ID NO: 8 or 31, or a polypeptide sequence as set forth in SEQ ID NO: 8 or 31.

In some embodiments, the beta chain intracellular domain comprises the IL-2RB intracellular domain. In some embodiments, the IL-2RB intracellular domain comprises a polypeptide sequence at least 95% identical to SEQ ID NO: 2, or a polypeptide sequence as set forth in SEQ ID NO: 2.

In some embodiments, the beta chain intracellular domain comprises the IL-7RB intracellular domain. In some embodiments, the IL-7RB intracellular domain comprises a polypeptide sequence at least 95% identical to SEQ ID NO: 3, or a polypeptide sequence as set forth in SEQ ID NO: 3.

In some embodiments the beta chain intracellular domain comprises the IL-21RB intracellular domain. In some embodiments, the IL-21RB intracellular domain comprises a polypeptide sequence at least 95% identical to SEQ ID NO: 4, or a polypeptide sequence as set forth in SEQ ID NO: 4.

In some embodiments, the second transmembrane domain comprises a transmembrane domain from the same beta chain intracellular domain. In some embodiments, the second transmembrane domain is a transmembrane domain of IL-2RB comprising a polypeptide sequence at least 95% identical to SEQ ID NO: 35 or 36, or a polypeptide sequence as set forth in SEQ ID NO: 35 or 36.

In some embodiments, the synthetic gamma chain polypeptide contains an IL-2RG TM domain comprising the sequence set forth in SEQ ID NO: 8 or 31 and a IL-2RG intracellular domain comprising the sequence set forth in SEQ ID NO:1; and the synthetic beta chain polypeptide contains an IL-2RB TM domain comprising the sequence set forth in SEQ ID NO: 35 or 36 and a IL-2RB intracellular domain comprising the sequence set forth in SEQ ID NO:2.

In some embodiments, the first dimerization domain and the second dimerization domain are heterodimerization domains selected from FK506-Binding Protein of size 12 kD (FKBP) and a FKBP12-rapamycin binding (FRB) domain. In some embodiments, the non-physiological ligand is rapamycin or a rapalog. In some embodiments, the FRB domain comprises a polypeptide sequence at least 95% identical to SEQ ID NO: 6 or SEQ ID NO: 7. In some embodiments, the FRB domain comprises the polypeptide sequence set forth in SEQ ID NO: 6 or SEQ ID NO: 7. In some embodiments, the FKBP domain comprises a polypeptide sequence at least 95% identical to SEQ ID NO: 5, SEQ ID NO:49, or SEQ ID NO: 30.

In some embodiments, the FKBP domain comprises the polypeptide sequence set forth in SEQ ID NO: 5, SEQ ID NO:49, or SEQ ID NO:30. In some embodiments, the first dimerization domain and the second dimerization domain are heterodimerization domains selected from FK506-Binding Protein of size 12 kD (FKBP) and a calcineurin domain. In some embodiments, the non-physiological ligand is FK506 or an analogue thereof. In some embodiments, the FKBP domain comprises a polypeptide sequence at least 95% identical to SEQ ID NO: 5, SEQ ID NO:49, or SEQ ID NO:30. In some embodiments, the FKBP domain comprises the polypeptide sequence set forth in SEQ ID NO: 5, SEQ ID NO:49, or SEQ ID NO: 30.

In some of any embodiments, the synthetic gamma chain polypeptide has the sequence of amino acids set forth in SEQ ID NO:28 or a sequence of amino acids that is at least 90%, at least 95%, at least 96%, at least 97%, at least 98%, at least 99%, or 100% identical to SEQ ID NO: 28, and the synthetic beta chain polypeptide has the sequence of amino acids set forth in SEQ ID NO:33 or a sequence of amino acids that is at least 90%, at least 95%, at least 96%, at least 97%, at least 98%, at least 99%, or 100% identical to SEQ ID NO: 33. In some of any embodiments, the synthetic gamma chain polypeptide has the sequence of amino acids set forth in SEQ ID O: 28 and the synthetic beta chain polypeptide has the sequence of amino acids set forth in SEQ ID NO:33.

In some embodiments, the first dimerization domain and the second dimerization domain are homodimerization domains selected from: i) FK506-Binding Protein of size 12 kD (FKBP); ii) cyclophiliA (CypA); or iii) gyrase B (CyrB); and the non-physiological ligand is, respectively: i) FK1012, AP1510, AP1903, or AP20187 or an analog thereof; ii) cyclosporin-A (CsA) or an analog thereof; or iii) coumermycin or an analog thereof.

In some of any embodiments, the stem cell is a pluripotent stem cell. In some embodiments, the stem cells are induced pluripotent stem cells (iPSCs).

In some embodiments, the stem cell is resistant to rapamycin-mediated mTOR inhibition.

In some embodiments, the stem cells express a cytosolic polypeptide that binds to the non-physiological ligand. In some embodiments, the non-physiological ligand is rapamycin or a rapalog, and the stem cells express a cytosolic FRB domain or variant thereof. In some embodiments, the cytosolic FRB domain comprises a polypeptide sequence at least 95% identical to SEQ ID NO: 6 or SEQ ID NO: 7. In some embodiments, the cytosolic FRB domain comprises a polypeptide sequence at least 98% identical to SEQ ID NO: 6 or SEQ ID NO: 7.

In some embodiments, the stem cell comprises a disrupted FKBP12 gene that reduces expression of FKBP12. In some embodiments, the stem cell comprises knock out of the FKBP12 gene.

In some embodiments, the stem cells comprise a nucleotide sequence encoding the synthetic cytokine receptor inserted into the genome of the stem cell. In some embodiments, the nucleotide sequence encoding the synthetic cytokine receptor is inserted into a non-target locus in the genome of the stem cell. In some embodiments, the nucleotide sequence encoding the synthetic cytokine receptor is inserted into an endogenous gene of the stem cell. In some embodiments, the insertion reduces expression of the endogenous gene in the locus. In some embodiments, the insertion knocks out the endogenous gene in the locus. In some embodiments, the insertion is by homology-directed repair.

In some embodiments, the endogenous gene is a housekeeping gene, a blood-lineage specific loci or an immune-related gene. In some embodiments, the endogenous gene is a housekeeping gene and the housekeeping gene is selected from eukaryotic translation elongation factor 1 alpha (EEF1A), glylceraldehyde-3-phosphate dehydrogenase (GAPDH), ubiquitin C (UBC), and actin beta (ACTB). In some embodiments, the endogenous gene is a blood-lineage specific loci and the blood-lineage specific loci is selected from protein tyrosine phosphatase receptor type C (PTPRC), IL2RG, and IL2RB. In some embodiments, the immune-related gene is selected from a beta-2-microglobulin (B2M) gene, a T cell receptor alpha constant (TRAC) gene, and a signal regulatory protein alpha (SIRPA) gene. In some embodiments, the endogenous gene is B2M. In some embodiments, the stem cell comprises a B2M knockout. In some embodiments, the cell has a disruption of a gene encoding FKBP12. In some embodiments, the disruption is a FKBP12 knockout that inactivates the gene encoding FKBP12.

In some embodiments, the stem cell comprises a B2M knockout and a FKBP12 knockout.

In some embodiments, the stem cell comprises a chimeric antigen receptor (CAR). In some embodiments, the CAR is an anti-FITC CAR.

In some of any embodiments, binding of the non-physiological ligand to the synthetic cytokine receptor activates the synthetic cytokine receptor in the stem cells to induce differentiation of the engineered stem cells in the cell population.

Also provided herein is a cell population comprising any of the provided engineered stem cells.

Accordingly, in some aspects, the disclosure provides a cell population comprising engineered stem cells comprising a synthetic cytokine receptor for a non-physiological ligand, wherein the cytokine receptor comprises: a synthetic gamma chain polypeptide comprising a first dimerization domain, a first transmembrane domain, and an interleukin-2 receptor subunit gamma (IL-2RG) intracellular domain, and a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain; wherein the non-physiological ligand activates the synthetic cytokine receptor in the stem cells to induce differentiation of the stem cells.

In some embodiments, the beta chain intracellular domain comprises the IL-2RB intracellular domain. In some embodiments, the IL-2RB intracellular domain comprises a polypeptide sequence at least 95% identical to SEQ ID NO: 2, or a polypeptide sequence as set forth in SEQ ID NO: 2.

In some embodiments, the beta chain intracellular domain comprises the IL-7RB intracellular domain. In some embodiments, the IL-7RB intracellular domain comprises a polypeptide sequence at least 95% identical to SEQ ID NO: 3, or a polypeptide sequence as set forth in SEQ ID NO: 3.

In some embodiments, the beta chain intracellular domain comprises the IL-21RB intracellular domain. In some embodiments, the IL-21RB intracellular domain comprises a polypeptide sequence at least 95% identical to SEQ ID NO: 4, or a polypeptide sequence as set forth in SEQ ID NO: 4.

In some or any of the foregoing embodiments, the first dimerization domain and the second dimerization domain are extracellular domains; the synthetic gamma chain polypeptide comprises, in N- to C-terminal order, the first dimerization domain, the first transmembrane domain, and the interleukin-2 receptor subunit gamma (IL-2RG) intracellular domain, and the synthetic beta chain polypeptide comprises, in N- to C-terminal order, the second dimerization domain, the second transmembrane domain, and the intracellular domain.

In some or any of the foregoing embodiments, the first dimerization domain and the second dimerization domain are heterodimerization domains selected from FK506-Binding Protein of size 12 kD (FKBP) and a FKBP12-rapamycin binding (FRB) domain; and/or wherein the non-physiological ligand is rapamycin or a rapalog. In some embodiments, the FKBP domain comprises a polypeptide sequence at least 95% identical to SEQ ID NO: 5. In some embodiments, the FKBP domain comprises the polypeptide sequence set forth in SEQ ID NO: 5. In some embodiments, the FKBP domain comprises a polypeptide sequence at least 95% identical to SEQ ID NO: 49. In some embodiments, the FKBP domain comprises the polypeptide sequence set forth in SEQ ID NO: 49. In some embodiments, the FRB domain comprises a polypeptide sequence at least 95% identical to SEQ ID NO: 6 or SEQ ID NO: 7. In some embodiments, the FRB domain comprises the polypeptide sequence set forth in SEQ ID NO: 6 or SEQ ID NO: 7.

In some or any of the foregoing embodiments, the first dimerization domain and the second dimerization domain are heterodimerization domains selected from FK506-Binding Protein of size 12 kD (FKBP) and a calcineurin domain; and/or wherein the non-physiological ligand is FK506 or an analogue thereof. In some embodiments, the FKBP domain comprises a polypeptide sequence at least 95% identical to SEQ ID NO: 5. In some embodiments, the FKBP domain comprises the polypeptide sequence set forth in SEQ ID NO: 5. In some embodiments, the FKBP domain comprises a polypeptide sequence at least 95% identical to SEQ ID NO: 49. In some embodiments, the FKBP domain comprises the polypeptide sequence set forth in SEQ ID NO: 49.

In some or any of the foregoing embodiments, the first dimerization domain and the second dimerization domain are homodimerization domains selected from:
i) FK506-Binding Protein of size 12 kD (FKBP);
ii) cyclophiliA (CypA); or
iii) gyrase B (CyrB);
   and the non-physiological ligand is, respectively:
i) FK1012, AP1510, AP1903, or AP20187 or an analog thereof;
ii) cyclosporin-A (CsA) or an analog thereof; or
iii) coumermycin or an analog thereof.

In some or any of the foregoing embodiments, the stem cells express a cytosolic polypeptide that binds to the non-physiological ligand. In some embodiments, the non-physiological ligand is rapamycin or a rapalog, and the stem cells express a cytosolic FRB domain or variant thereof. In some embodiments, the cytosolic FRB domain comprises a polypeptide sequence at least 95% identical to SEQ ID NO: 6 or SEQ ID NO: 7. In some embodiments, the cytosolic FRB domain comprises a polypeptide sequence at least 98% identical to SEQ ID NO: 6 or SEQ ID NO: 7.

In some or any of the foregoing embodiments, the stem cells are induced pluripotent stem cells (iPSCs).

In some or any of the foregoing embodiments, the stem cells comprise a nucleotide sequence encoding the synthetic cytokine receptor. In some embodiments, the nucleotide sequence is inserted into an endogenous gene of the stem cells. In some embodiments, the endogenous gene is a housekeeping gene or a blood-lineage specific locus. In some embodiments, the housekeeping gene is selected from eukaryotic translation elongation factor 1 alpha (EEF1A), glylceraldehyde-3-phosphate dehydrogenase (GAPDH), ubiquitin C (UBC), and actin beta (ACTB). In some embodiments, the blood-lineage specific loci are selected from protein tyrosine phosphatase receptor type C (PTPRC), IL2RG, and IL2RB. In some embodiments, the nucleotide sequence is inserted into a disrupted gene of the stem cells. In some embodiments, the disrupted gene is selected from a disrupted beta-2-microglobulin (B2M) gene, a disrupted T cell receptor alpha constant (TRAC) gene, and a disrupted signal regulatory protein alpha (SIRPA) gene.

In some or any of the foregoing embodiments, the stem cells comprise a disrupted B2M gene. In some embodiments, the stem cells comprise reduced expression of B2M. In some embodiments, the stem cells are knocked out for B2M.

In some or any of the foregoing embodiments, the stem cells are rapamycin resistant. In some embodiments, the rapamycin resistant stem cells comprise a disrupted FKBP12 gene. In some embodiments, the stem cells comprise reduced expression of FKBP12. In some embodiments, the stems cells are knocked out for FKBP12.

In some or any of the foregoing embodiments, the non-physiological ligand activates the synthetic cytokine receptor in the stem cells to induce differentiation of the stem cells into hematopoietic progenitors. In some embodiments, the non-physiological ligand activates the synthetic cytokine receptor in the stem cells to induce differentiation of the stem cells into common lymphoid progenitors (CLPs) or common myeloid progenitors (CMPs).

In some or any of the foregoing embodiments, the stem cells comprise a chimeric antigen receptor (CAR).

In some aspects, the disclosure provides a method for generating cytotoxic innate lymphoid (iCIL) cells, comprising contacting a cell population of any one of the provided embodiments with the non-physiological ligand for a first period of time sufficient to generate CLPs, and contacting the CLPs with a differentiation media for a second period of time sufficient to generate iCILs.

In some embodiments, the differentiation media comprises stem cell factor (SCF), FLT3L, IL-7, IL-12, IL-15, SR-1 and UM729. In some embodiments, the differentiation media comprises the non-physiological ligand.

In some embodiments, the first period of time is 1-15 days, and the second period of time is 1-15 days. In some embodiments, the method comprises contacting the iCILs with a pre-activation media comprising IL-7, IL-12, IL-15, IL-18 and IL-21 for a third period of time sufficient to generate mature iCILs. In some embodiments the pre-activation media comprises the non-physiological ligand. In some embodiments the third period of time is 1-10 days.

In some or any of the foregoing embodiments, mature iCILs express NKp46, NKG2D, LFA1, DNAM1, CD16 and CD56.

In some aspects, the disclosure provides a method of genetically engineering stem cells to express a synthetic cytokine receptor, comprising: contacting a population of stem cells with (i) a guide RNA (gRNA) targeting a target site in an endogenous gene, (ii) an RNA-guided endonuclease, and (iii) a recombinant vector comprising a nucleotide sequence encoding a synthetic cytokine receptor for a non-physiological ligand,
thereby inserting the nucleotide sequence into the endogenous gene;
wherein the cytokine receptor comprises:
a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

In some or embodiments, the nucleotide sequence is inserted via homology directed repair (HDR). In some embodiments, the recombinant vector contains 5′ and 3′ homology arms flanking the nucleotide sequence encoding the synthetic cytokine receptor, in which the 3′ homology arm is homologous with a region upstream of the gRNA target site and the 5′ homology arm is homologous with a region downstream of the gRNA target site. In some or embodiments, the method comprises contacting the cells with a vector comprising a nucleic acid comprising from 5′ to 3′ (a) a nucleotide sequence homologous with a region located upstream of the target site, (b) the nucleotide sequence encoding a synthetic cytokine receptor for a non-physiological ligand, and (c) a nucleotide sequence homologous with a region located downstream, wherein a double-strand break occurs at the target site in the endogenous gene, and the nucleic acid is exchanged with a homologous nucleotide sequence of the endogenous gene.

In some or embodiments, the nucleotide sequence is inserted via non-homologous end joining (NHEJ).

In some or any of the foregoing embodiments, the RNA-guided endonuclease is selected from a Cas endonuclease, a Mad endonuclease, and a Cpf1 endonuclease. In some embodiments, the RNA-guided endonuclease is Cas9. In some embodiments, the RNA-guided endonuclease is Mad7.

In some or any of the foregoing embodiments, the method comprises disrupting a target gene and inserting the nucleotide sequence encoding the synthetic cytokine receptor into the disrupted target gene, wherein disrupting the target gene comprises contacting the population of stem cells with (i) a gRNA targeting a target site in a target gene, and (ii) an RNA-guided endonuclease. In some embodiments, the endogenous target gene is selected from B2M, TRAC and SIRPA.

In some of any embodiments, the endogenous gene is B2M. In some embodiments, the gRNA comprises the sequence set forth in SEQ ID NO:18.

In some of any embodiments, the nucleotide sequence homologous with a region located upstream of the target site comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 22; and the nucleotide sequence homologous with a region located downstream comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 23.

In some of any embodiments, the nucleotide sequence encoding the synthetic cytokine receptor comprises a first nucleic acid sequence encoding a gamma chain that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 37, and a second nucleic acid sequence encoding a beta chain that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 38. In some embodiments, the nucleotide sequence encoding the synthetic cytokine receptor comprises a first nucleic acid set for thin SEQ ID NO: 37 and a second nucleic acid set forth in SEQ ID NO:38.

In some embodiments, the first nucleic acid sequence and second nucleic acid sequence are separated by a cleavable linker or an IRES. In some embodiments, the cleavable linker is a protein quantitation reporter linker (PQR). In some embodiment, the PQR linker has the sequence set forth in SEQ ID NO:42.

In some of any embodiments, the nucleotide sequence encoding a synthetic cytokine receptor for a non-physiological ligand is under the operable control of a heterologous promoter. In some embodiments, the heterologous promoter is the EF1α promoter or the MND promoter. In some embodiments, the promoter is a dual promoter in which the synthetic cytokine receptor is under the operable control of two promoters. In some embodiments, the dual promoter is a dual EF1α promoter.

In some of any embodiments, the nucleotide sequence encoding the synthetic cytokine receptor comprises a polyadenylation sequence.

In some embodiments, the recombinant vector comprises the sequence set forth in SEQ ID NO:40 or a sequence that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 40. In some embodiments, the recombinant vector has the sequence set for in SEQ ID NO:40.

In some or any of the foregoing embodiments, the method comprises engineering the population of stem cells to be resistant to rapamycin. In some embodiments, the resistance to rapamycin is rapamycin mediated mTOR inhibition. In some embodiments, the population of stem cells to be resistant to rapamycin comprises disrupting a FKBP12 gene in the stem cell. In some embodiments, the population of stem cells to be resistant to rapamycin has reduced expression of FKBP12. In some embodiments, the population of stem cells to be resistant to rapamycin comprises knocking out a FKBP12 gene.

In some embodiments, the stem cell is engineered with a CRISPR-Cas and gRNA targeting the FKBP12 gene for disrupting FKBP12 in the cell. In some of any embodiments, the method comprises further contacting the population of stem cells with a guide RNA (gRNA) targeting a target site in the FKBP12 gene. In some embodiments, the RNA-guided endonuclease is selected from a Cas endonuclease, a Mad endonuclease, and a Cpf1 endonuclease. In some embodiments, the RNA-guided endonuclease is Cas9. In some embodiments, the RNA-guided endonuclease is Mad7. In some embodiments, the further contacting is carried out simultaneously with the contacting in (i) with a guide RNA (gRNA) targeting a target site in an endogenous gene, optionally in combination with the same RNA-guided endonuclease.

In some embodiments, the gRNA comprises one or more gRNA selected from a gRNA comprising the sequence set forth in SEQ ID NO: 19, SEQ ID NO:20 or SEQ ID NO: 21. In some embodiments, the one or more gRNA is a pool of gRNA comprising 2 or 3 gRNA.

In some of any embodiments, the method further comprises introducing into the population of stem cells a chimeric antigen receptor (CAR). In some embodiments the CAR is an anti-FITC CAR.

In some of any embodiments, the stem cells are pluripotent stem cells.

In some or any of the foregoing embodiments, the stem cells are iPSCs.

In some of any embodiments, provided herein is a method for generating hematopoietic progenitor (HP) cells, the method comprising: a) culturing a cell population comprising any of the engineered iPSCs provided herein under conditions to form an aggregate; b) culturing the cells produced in a) under conditions to induce mesoderm formation in a plurality of the cells, wherein the initiation of the culturing in b) is day 0; and c) culturing the cells produced in b) under conditions to differentiate cells into a population of hematopoietic progenitors (HP), wherein at least a portion of one or more of steps a)-c) are carried out in the presence of a non-physiological ligand of the synthetic cytokine receptor.

In some embodiments, provided herein is a method for generating cytotoxic innate lymphoid (iCIL) cells, the method comprising culturing a cell population comprising engineered iPSCs of any of the provided embodiments under conditions to differentiate the iPSCs to cytotoxic innate lymphoid (iCILs), wherein a non-physiological ligand of the synthetic cytokine receptor is added during at least a portion of the culturing. In some embodiments, the culturing comprises: a) culturing the cell population comprising engineered iPSCs under conditions to form an aggregate; b) culturing the cells produced in a) under conditions to induce mesoderm formation in a plurality of the cells, wherein the initiation of the culturing in b) is on day 0; c) culturing the cells produced in b) under conditions to differentiate cells into a population of hematopoietic progenitors (HP); and d) culturing the cells produced in c) under conditions to generate iCIL cells, wherein at least a portion of one or more of steps a)-d) are carried out in the presence of the non-physiological ligand of the synthetic cytokine receptor.

Accordingly, in some embodiments provided herein is a method for generating cytotoxic innate lymphoid (iCIL) cells, the method comprising: a) culturing a cell population comprising engineered iPSCs of any of the provided embodiments under conditions to form an aggregate; b) culturing the cells produced in a) under conditions to induce mesoderm formation in a plurality of the cells, wherein the initiation of the culturing in b) is day 0; c) culturing the cells produced in b) under conditions to differentiate cells into a population of hematopoictic progenitors (HP); and d) culturing the cells produced in c) under conditions to generate iCIL cells, wherein at least a portion of one or more of steps a)-d) are carried out in the presence of a non-physiological ligand of the synthetic cytokine receptor.

In some embodiments, the culturing is carried out in a vessel treated to promote cell adhesion and growth. In some embodiments, the vessel is a Matrigel. In some embodiments, the culturing is carried out in in a non-adherent culture vessel. In some embodiments, the non-adherent culture vessel is Aggrewell™ plate. In some embodiments of the methods, the aggregate in a) is an Embryoid body (EB).

In some embodiments, the culturing is carried out in suspension. In some embodiments, the culturing is carried out in culture vessel that is not treated to promote cell adhesion and proliferation. In some of any such embodiments, the culturing in step a) comprises: (i) performing a first incubation comprising culturing the cell population of engineered stem cells under conditions to form a first aggregate; (ii) contacting the aggregate with a dissociating agent to form a population of dissociated cells; and (iii) performing a second incubation comprising culturing the population of dissociated cells under conditions to form the second aggregate. In some embodiments, the incubations are carried out in suspension.

In some of any embodiments of the methods, the culturing in b) is in a media comprising one or more of BMP4, FGF2, VEGF and a Rock Inhibitor In some embodiments, the Rock Inhibitor is Y27632. In some embodiments, the culturing in b) is in a media comprising BMP4, FGF2, VEGF and Y27632. In some embodiments, the culturing in b) is in a media comprising BMP4, FGF2 and VEGF. In some of any embodiments, the culturing in b) is in a media comprising the non-physiological ligand. In some embodiments, the culturing in b) is in a media comprising the non-physiological ligand without any additional growth factors. In some embodiments, the culturing in b) is for 2 to 4 days. In some embodiments, the culturing in b) is for at or about 3 days

In some of any embodiments, the culturing in c) is in a media comprising one or more of BMP4, FGF2, VEGF, TPO, SCF, and LDL. In some of any embodiments, the culturing in c) is in a media comprising one or more of BMP4, FGF2, VEGF and LDL. In some of any embodiments, the culturing in c) is in a media comprising BMP4 and FGF2. In some of any embodiments, the culturing in c) with BMP4 and FGF2 is for days 3 to 15. In some of any embodiments, the culturing in c) comprises a PI3K inhibitor. In some of any embodiments, the PI3K inhibitor is LY2940002. In some of any embodiments, the PI3K inhibitor is added during a portion of the culturing in c). In some of any embodiments, the PI3K inhibitor is added from about day 6 to day 15.

In some of any embodiments, the culturing in c) is in a media without SCF and TPO. In some embodiments, the culturing in c) is in a media comprising the non-physiological ligand. In some embodiments, the culturing in c) is in a media comprising the non-physiological ligand without any additional growth factors, cytokines or both.

In some embodiments, the culturing in c) is on days 3 to 15 days. In some embodiments, during at least a portion of the culturing in c) the media comprises an aryl hydrocarbon receptor (AHR) antagonist (e.g. StemRegenin-1), a pyrimido-[4,5-b]-indole derivative (e.g. UM729) or both. In some embodiments, the portion of the culturing is on or about days 9-15. In some embodiments, the AHR antagonist is StemRegenin 1 (SR1). In some embodiments, the pyrimido-[4,5-b]-indole derivative is UM729. In some embodiments, SR1 and UM729 are added to the culturing in c) beginning at a day from day 6 to day 9. In some embodiments, SR1 and UM729 are added to the culturing in c) beginning at about day 6.

In some embodiments, the culturing in d) is in a media comprising one or more of FLT3L, IL-7, IL-12, IL-15, SR-1 and UM729. In some embodiments, the culturing in d) is in a media comprising the non-physiological ligand. In some embodiments, the culturing in d) is in a media comprising the non-physiological ligand without any additional growth factors, cytokines or both. In some embodiments, the culturing in d) is for a time between days 15 and 40. In some embodiments, the culturing in d) is for days 15 and 30.

In some aspects, provided herein is a method for generating cytotoxic innate lymphoid (iCIL) cells, the method comprising: a) culturing a cell population comprising engineered iPSCs of any of claims 1-48 under conditions to form an aggregate; b) culturing the cells produced in a) in a media comprising one or more selected from the group of BMP4, VEGF. FGF2, and ROCKi to induce mesoderm formation in a plurality of the cells, wherein the initiation of the culturing in b) is day 0; c) culturing the cells produced in b) in a media comprising BMP4, FGF2, and LY2940002 to differentiate cells into a population of hematopoictic progenitors (HP), wherein the initiation of the culturing in c) is day 3; and d) culturing the cells produced in d) in a media comprising SCF and IL-15 to generate iCIL cells, wherein the initiation of the culturing in b) is day 15, wherein at least a portion of one or more of steps a)-d) are carried out in the presence of a non-physiological ligand of the synthetic cytokine receptor. In some embodiments, SR1 and UM729 are added to the culturing in c) beginning at a day from day 6 to day 9. In some embodiments, SR1 and UM729 are added to the culturing in c) beginning at about day 6.

Also provided is a method for generating cytotoxic innate lymphoid (iCIL) cells, comprising contacting a cell population comprising an engineered stem cell of any one of the provided embodiments with the non-physiological ligand for a first period of time sufficient to generate CLPs, and contacting the CLPs with a differentiation media for a second period of time sufficient to generate iCILs. In some embodiments, the differentiation media comprises stem cell factor (SCF), FLT3L, IL-7, IL-12, IL-15, SR-1 and UM729. In some embodiments, the differentiation media comprises the non-physiological ligand. In some embodiments, the first period of time is 1-15 days, and wherein the second period of time is 1-15 days. In some embodiments, the methods comprise contacting the iCILs with a pre-activation media comprising IL-7, IL-12, IL-15, IL-18 and IL-21 for a third period of time sufficient to generate mature iCILs. In some embodiments, the pre-activation media comprises the non-physiological ligand. In some embodiments the third period of time is 1-10 days. In some embodiments, mature iCILs express NKp46, NKG2D, LFA1, DNAM1, CD16 and CD56.

In some of any embodiments, the non-physiological ligand is rapamycin or a rapamycin analog. In some embodiments, the rapamycin analog is rapalog. In some embodiments, the non-physiological ligand is added to the media at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM and 150 nM and 200 nM. In some embodiments, the non-physiological ligand is added to the media at a concentration of at or about 10 nM. In some embodiments, the non-physiological ligand is added to the media at a concentration of at or about 100 nM. In some embodiments, the non-physiological ligand is added to the media at a concentration from 2.5 nM to 10 nM. In some embodiments, the non-physiological ligand is added to the media at a concentration from 3 nM to 7 nM. In some embodiments, the non-physiological ligand is added to the media at a concentration is at or about 3.1 nM.

In some of any embodiments, provided herein is a hematopoietic progenitor (HP) cell produced by any of the methods provided herein. In some embodiments, the HP cells comprise lower expression of HLF, HOXA9, and/or CD133 compared to a CD34+ cord blood cell. In some embodiments, the expression of HLF, HOXA9, and/or CD133 in HP cells is 8-fold, 7-fold, 6-fold, 5-fold, 4-fold, 3-fold, 2-fold, or 1-fold lower compared to a CD34+ cord blood cell. In some embodiments, the CD34+ cord blood cell comprises a hematopoietic stem cell (HSC).

In some of any embodiments, provided herein is a hematopoietic progenitor (HP) cell that has been differentiated from a pluripotent stem cell according to any of the methods provided herein, wherein the HP comprises a synthetic cytokine receptor.

In some of any embodiments, provided herein is a population of hematopoietic progenitor (HP) cells produced by any of the methods provided herein. In some of any embodiments, the population of HP cells comprise lower expression of HLF, HOXA9, and/or CD133 compared to a population of CD34+ cord blood cells. In some of any embodiments, the expression of HLF, HOXA9, and/or CD133 in HP cells is 8-fold, 7-fold, 6-fold, 5-fold, 4-fold, 3-fold, 2-fold, or 1-fold lower compared to a population of CD34+ cord blood cells. In some of any embodiments, the population of CD34+ cord blood cell comprises a hematopoietic stem cell (HSC).

Also provided herein is a cytotoxic innate lymphoid (iCIL) cell produced by any of the provided methods.

Also provided herein is a cytotoxic innate lymphoid (iCIL) that has been differentiated from any of the provided engineered stem cells, wherein the iCIL comprises a synthetic cytokine receptor.

Also provided is a population of cytotoxic innate lymphoid (iCIL) cells produced by any of the provided methods.

Also provided is a pharmaceutical composition comprising the iCIL or population of iCILs of any of the provided embodiments.

In some aspects, the disclosure provides a cell population produced by a method described herein.

In some aspects, the disclosure provides a pharmaceutical composition comprising a cell population described herein.

Also provided herein is a method of expanding a cytotoxic innate lymphoid cell (iCIL), the method comprising contacting an iCIL or population of iCILs as provided herein or a pharmaceutical composition comprising the same with the non-physiological ligand of the synthetic cytokine receptor. Also provided herein, is a method of killing or inhibiting the proliferation of cancer cells, the method comprising contacting cancer cells with an iCIL or population of iCILs as provided herein or a pharmaceutical composition comprising the same with the non-physiological ligand of the synthetic cytokine receptor. In some embodiments, the synthetic cytokine receptor has a first dimerization domain and a second dimerization domain that are heterodimerization domains selected from FK506-Binding Protein of size 12 KD (FKBP) and a FKBP12-rapamycin binding (FRB) domain. In some embodiments, the non-physiological ligand is rapamycin or or a rapamycin analog. In some embodiments, the rapamycin analog is rapalog.

In some embodiments, such methods are performed in vitro or ex vivo. In some of any embodiments, the method is performed ex vivo in a subject and the non-physiological ligand is contacted with stem cells (e.g., iPSCs) from the subject.

In some embodiments, the non-physiological ligand is contacted at a concentration of between 2.5 nM and 200 nM, 2.5 nM and 150 nM, 2.5 nM and 100 nM, 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM and 150 nM and 200 nM. In some embodiments, the non-physiological ligand is contacted at a concentration of at or about 10 nM. In some embodiments, the non-physiological ligand is contacted at a concentration of at or about 100 nM.

In some of any embodiments, the method is performed in vivo in a subject and the non-physiological ligand is administered to the subject.

In some aspects, the disclosure provides a method of treating a cancer in a subject, comprising administering to the subject an effective amount of a cell population or pharmaceutical composition described herein. In some embodiments, the cell population is a population of iCILs as provided herein. In some embodiments, the method comprises administering to the subject the non-physiological ligand in an amount effective to induce expansion of the iCILs in the subject.

In some of any embodiments, the subject has not been administered a lymphodepleting therapy prior to the administering the iCIL, population of iCILs or the pharmaceutical composition containing such cells.

In some of any embodiments, the iCIL express a CAR targeting cancer cells in the subject. In some of any embodiments, the CAR is an anti-FITC CAR and the subject has been administered a FITC-ligand to tag a cancer cell in the subject, wherein the ligand specifically binds a molecule expressed on a tumor. In some of any embodiments, the FITC-ligand is FITC-folate.

In some of any embodiments, the method comprises administering to the subject the non-physiological ligand of the synthetic cytokine receptor.

In some of any embodiments, the non-physiological ligand is rapamycin or a rapamycin analog. In some of any embodiments, the rapamycin analog is rapalog.

In some of any embodiments, the non-physiological ligand is administered at a dose of 1 mg to 100 mg. In some embodiments, the non-physiological ligand is administered at a dose 10-100 mg. In some of any embodiments, the non-physiological ligand is administered at a dose of 10 mg, 20 mg, 25 mg, 30 mg, 40 mg, 50 mg or any value between any of the foregoing.

In some of any embodiments, multiple doses of the non-physiological ligand are administered to the subject. In some of any embodiments, the multiple doses are administered intermittently or at regular intervals after administration of the iCIL or population or composition thereof to the subject. In some embodiments, the doses are administered for a predetermined period of time. In some of any embodiments, 2 to 8 doses of the non-physiological ligand are administered to the subject. In some of any embodiments, a single dose of the non-physiological ligand is administered to the subject.

In some of any embodiments, the iCIL population or composition thereof is administered at a dose that is from at or about from at or about 1×10⁸ iCIL cells to at or about 100×10⁹ iCIL cells. In some of any embodiments, the iCIL population or composition thereof is administered at a dose that is greater than at or about 5×10⁹ iCIL cells. In some embodiments, the dose is from at or about from at or about 5×10⁹ iCIL cells to at or about 100×10⁹ iCIL cells.

In some aspects, the disclosure provides a kit comprising a cell population described herein and instructions for administering the cell population to a subject in need thereof. In some embodiments, the kit comprises a container comprising the non-physiological ligand and instructions for administering the non-physiological ligand to the subject after administration of the cell population. In some embodiments, the subject has a cancer.

In some aspects, provided herein is a population of induced cytotoxic innate lymphoid (iCIL) cells, wherein the iCILs are mature iCILs expressing CD56 and LFA1, and wherein: at least 25% of the iCILs express a cytotoxicity receptor; no more than 75% of the iCILs express a dysfunction receptor; and/or at least 25% of the iCILs are proliferative. In some embodiments, at least 25% of the iCILs express a cytotoxicity receptor. In some embodiments, the cytotoxicity receptor is one or more of NKp30, NKp46, and NKG2D. In some embodiments, at least 25%, at least 30%, at least 35%, at least 40%, at least 45%, at least 50%, at least 55%, or at least 60% of the iCILs express NKp30+. In some embodiments, at least 55%, at least 60%, at least 65%, at least 70%, at least 75%, or at least 80% of the iCILs express NKp46. In some embodiments, at least 50%, at least 55%, at least 60%, at least 65%, at least 70%, at least 75%, at least 80%, at least 85%, or at least 90% of the iCILs express NKG2D. In some embodiments, no more than 75% of the iCILs express a dysfunction receptor. In some embodiments, the dysfunction receptor is one or more of KLRG1, CD73, and CD38. In some embodiments, no more than 45%, no more than 40%, no more than 35%, no more than 30%, no more than 25%, no more than 20%, no more than 15%, no more than 10%, or no more than 5% of the iCILs express KLRG1. In some embodiments, no more than 10%, no more than 9%, no more than 8%, no more than 7%, no more than 6%, no more than 5%, no more than 4%, no more than 3%, no more than 2%, or no more than 1% of the iCILs express CD73. In some embodiments, no more than 75%, no more than 65%, no more than 55%, no more than 45%, no more than 35%, no more than 25%, no more than 15%, or no more than 5% of the iCILs express CD38.

In some embodiments, at least 25%, at least 30%, at least 35%, at least 40%, at least 45%, at least 50%, at least 55%, or at least 60% of the iCILs are proliferative. In some embodiments, the iCILs that are proliferative are CD56bright CD57−.

In some embodiments, the iCILs further comprise a synthetic cytokine receptor for a non-physiological ligand, wherein the cytokine receptor comprises: a synthetic gamma chain polypeptide comprising a first dimerization domain, a first transmembrane domain, and an interleukin-2 receptor subunit gamma (IL-2RG) intracellular domain, and a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain. In some embodiments, the first dimerization domain and the second dimerization domain are extracellular domains.

In some embodiments, the synthetic gamma chain polypeptide comprises, in N-to C-terminal order, the first dimerization domain, the first transmembrane domain, and the interleukin-2 receptor subunit gamma (IL-2RG) intracellular domain, and the synthetic beta chain polypeptide comprises, in N- to C-terminal order, the second dimerization domain, the second transmembrane domain, and the intracellular domain.

In some embodiments, wherein the IL-2RG intracellular domain comprises a polypeptide sequence at least 95% identical to SEQ ID NO: 1, or a polypeptide sequence as set forth in SEQ ID NO: 1. In some embodiments, the first transmembrane domain comprises the IL-2RG transmembrane domain. In some embodiments, the IL-2RG transmembrane domain comprises a polypeptide sequence at least 95% identical to SEQ ID NO: 8 or 31, or a polypeptide sequence as set forth in SEQ ID NO: 8 or 31. In some embodiments, the beta chain intracellular domain comprises the IL-2RB intracellular domain. In some embodiments, IL-2RB intracellular domain comprises a polypeptide sequence at least 95% identical to SEQ ID NO: 2, or a polypeptide sequence as set forth in SEQ ID NO: 2. In some embodiments, the beta chain intracellular domain comprises the IL-7RB intracellular domain. In some embodiments, the IL-7RB intracellular domain comprises a polypeptide sequence at least 95% identical to SEQ ID NO: 3, or a polypeptide sequence as set forth in SEQ ID NO: 3. In some embodiments, the beta chain intracellular domain comprises the IL-21RB intracellular domain. In some embodiments, the IL-21RB intracellular domain comprises a polypeptide sequence at least 95% identical to SEQ ID NO: 4, or a polypeptide sequence as set forth in SEQ ID NO: 4. In some embodiments, the second transmembrane domain comprises a transmembrane domain from the same beta chain intracellular domain. In some embodiments, wherein the second transmembrane domain is a transmembrane domain of IL-2RB comprising a polypeptide sequence at least 95% identical to SEQ ID NO: 35 or 36, or a polypeptide sequence as set forth in SEQ ID NO: 35 or 36. In some embodiments, the synthetic gamma chain polypeptide contains an IL-2RG TM domain comprising the sequence set forth in SEQ ID NO: 8 or 31 and a IL-2RG intracellular domain comprising the sequence set forth in SEQ ID NO:1; and
the synthetic beta chain polypeptide contains an IL-2RB TM domain comprising the sequence set forth in SEQ ID NO: 35 or 36 and a IL-2RB intracellular domain comprising the sequence set forth in SEQ ID NO:2.

In some embodiments, wherein the first dimerization domain and the second dimerization domain are heterodimerization domains selected from FK506-Binding Protein of size 12 kD (FKBP) and a FKBP12-rapamycin binding (FRB) domain; and/or wherein the non-physiological ligand is rapamycin or a rapalog. In some embodiments, the FRB domain comprises a polypeptide sequence at least 95% identical to SEQ ID NO: 6 or SEQ ID NO: 7. In some embodiments, the FRB domain comprises the polypeptide sequence set forth in SEQ ID NO: 6 or SEQ ID NO: 7. In some embodiments, first dimerization domain and the second dimerization domain are heterodimerization domains selected from FK506-Binding Protein of size 12 kD (FKBP) and a calcineurin domain; and/or wherein the non-physiological ligand is FK506 or an analogue thereof.

In some embodiments, the FKBP domain comprises a polypeptide sequence at least 95% identical to SEQ ID NO: 5 or SEQ ID NO:30. In some embodiments, the FKBP domain comprises the polypeptide sequence set forth in SEQ ID NO: 5 or SEQ ID NO:30. In some embodiments, the synthetic gamma chain polypeptide has the sequence of amino acids set forth in SEQ ID NO:28 or a sequence of amino acids that is at least 90%, at least 95%, at least 96%, at least 97%, at least 98%, at least 99%, or 100% identical to SEQ ID NO: 28, and the synthetic beta chain polypeptide has the sequence of amino acids set forth in SEQ ID NO: 33 or a sequence of amino acids that is at least 90%, at least 95%, at least 96%, at least 97%, at least 98%, at least 99%, or 100% identical to SEQ ID NO: 33. In some embodiments, the synthetic gamma chain polypeptide has the sequence of amino acids set forth in SEQ ID O: 28 and the synthetic beta chain polypeptide has the sequence of amino acids set forth in SEQ ID NO:33.

In some embodiments, the first dimerization domain and the second dimerization domain are homodimerization domains selected from: i) FK506-Binding Protein of size 12 kD (FKBP); ii) cyclophiliA (CypA); or iii) gyrase B (CyrB); and the non-physiological ligand is, respectively: i) FK1012, AP1510, AP1903, or AP20187 or an analog thereof; ii) cyclosporin-A (CsA) or an analog thereof; or iii) coumermycin or an analog thereof. In some embodiments, the population comprises from about 1×10⁶ to about 1×10¹² iCILs, from about 1×10⁶ to about 1×10¹⁰ iCILs, from about 1×10⁶ to about 1×10⁸ iCILs, from about 1×10⁸ to about 1×10¹² iCILs, from about 1×10⁸ to about 1×10¹⁰ iCILs, or from about 1×10¹⁰ to about 1×10¹² iCILs. In some embodiments, the volume of the population is from about 1 mL to about 100 mL, from about 1 mL to about 80 mL, from about 1 mL to about 60 mL, from about 1 mL to about 40 mL, from about 1 mL to about 20 mL, from about 1 mL to about 10 mL, from about 10 mL to about 100 mL, from about 10 mL to about 80 mL, from about 10 mL to about 60 mL, from about 10 mL to about 40 mL, from about 10 mL to about 20 mL, from about 20 mL to about 100 mL, from about 20 mL to about 80 mL, from about 20 mL to about 60 mL, from about 20 mL to about 40 mL, from about 40 mL to about 100 mL, from about 40 mL to about 80 mL, from about 40 mL to about 60 mL, from about 60 mL to about 100 mL, from about 60 mL to about 80 mL, or from about 80 mL to about 100 mL.

In some aspects, provided herein is a pharmaceutical composition comprising any population of iCILs of any one of embodiments provided herein. In some embodiments, the pharmaceutical composition further comprises a cryoprotectant. In some aspects, provided herein is a cryopreserved composition comprising any population of iCILs of any one of embodiments provided herein. In some embodiments, the composition comprises from about 1×10⁶ to about 1×10¹² iCILs, from about 1×10⁶ to about 1×10¹⁰ iCILs, from about 1×10⁶ to about 1×10⁸ iCILs, from about 1×10⁸ to about 1×10¹² iCILs, from about 1×10⁸ to about 1×10¹⁰ iCILs, or from about 1×10¹⁰ to about 1×10¹² iCILs. In some embodiments, the volume of the composition is from about 1 mL to about 100 mL, from about 1 mL to about 80 mL, from about 1 mL to about 60 mL, from about 1 mL to about 40 mL, from about 1 mL to about 20 mL, from about 1 mL to about 10 mL, from about 10 mL to about 100 mL, from about 10 mL to about 80 mL, from about 10 mL to about 60 mL, from about 10 mL to about 40 mL, from about 10 mL to about 20 mL, from about 20 mL to about 100 mL, from about 20 mL to about 80 mL, from about 20 mL to about 60 mL, from about 20 mL to about 40 mL, from about 40 mL to about 100 mL, from about 40 mL to about 80 mL, from about 40 mL to about 60 mL, from about 60 mL to about 100 mL, from about 60 mL to about 80 mL, or from about 80 mL to about 100 mL.

In some aspects, provided herein is a method of killing or inhibiting the proliferation of target cells, comprising contacting target cells with the population of iCILs of any one of embodiments provided herein or the composition of any one of embodiments provided herein.

In some embodiments, the target cells are cancer cells. In some embodiments, the iCILs further comprise the synthetic cytokine receptor for the non-physiological ligand, and the method comprises contacting the target cells with the non-physiological ligand of the synthetic cytokine receptor. In some embodiments, the method is performed in vitro or ex vivo. In some embodiments, the non-physiological ligand is rapamycin or a rapamycin analog. In some embodiments, the rapamycin analog is rapalog. In some embodiments, the non-physiological ligand is contacted at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 M and 200 nM, 50 nM and 150 nM, 50 nM and 100 nM, 100 nM and 200 nM, 100 nM and 150 nM, or 150 nM and 200 nM. In some embodiments, the non-physiological ligand is contacted at a concentration of at or about 10 nM. In some embodiments, the non-physiological ligand is contacted at a concentration of at or about 100 nM. In some embodiments, the non-physiological ligand is added to the media at a concentration from 2.5 nM to 10 nM. In some embodiments, the non-physiological ligand is added to the media at a concentration from 3 nM to 7 nM. In some embodiments, the method is performed in vivo in a subject, and the population of iCILs or composition thereof is administered to the subject. In some embodiments, the iCILs further comprise the synthetic cytokine receptor for the non-physiological ligand, and the method comprises administering the non-physiological ligand to the subject.

In some aspects, provided herein is a method of inducing natural killer (NK) cell-mediated cell killing in a subject, comprising administering to the subject any effective amount of the population of iCILs of any one of embodiments provided herein or any composition of any one of embodiments provided herein. In some aspects, provided herein is a method of treating a cancer in a subject, comprising administering to the subject an effective amount of any population of iCILs of any one of embodiments provided herein or any composition of any one of embodiments provided herein. In some embodiments, the subject has not been administered a lymphodepleting therapy prior to the administering of the population of iCILs or composition thereof.

In some embodiments, the iCILs express a CAR targeting cancer cells in the subject. In some embodiments, the CAR is an anti-FITC CAR, and the subject has been administered a FITC-ligand to tag a cancer cell in the subject, wherein the ligand specifically binds a molecule expressed on a tumor. In some embodiments, the FITC-ligand is FITC-folate. In some embodiments, the iCILs further comprise the synthetic cytokine receptor for the non-physiological ligand, and the method comprises administering to the subject the non-physiological ligand of the synthetic cytokine receptor. In some embodiments, the non-physiological ligand is rapamycin or a rapamycin analog. In some embodiments, the rapamycin analog is rapalog. In some embodiments, the non-physiological ligand is administered at a dose of 1 mg to 100 mg, optionally between 10-100 mg, optionally at or about 10 mg. 20 mg, 25 mg, 30 mg, 40 mg, 50 mg or any value between any of the foregoing. In some embodiments, multiple doses of the non-physiological ligand are administered to the subject. In some embodiments, the multiple doses are administered intermittently or at regular intervals after the administration of the population of iCILs or composition thereof to the subject, optionally for a predetermined period of time. In some embodiments, 2 to 8 doses of the non-physiological ligand are administered to the subject. In some embodiments, a single dose of the non-physiological ligand is administered to the subject.

In some embodiments, the population of iCILs or composition thereof is administered at a dose that is from at or about 1×10⁸ iCIL cells to at or about 100×10⁹ iCIL cells. In some embodiments, the population of iCILs or composition thereof is administered at a dose that is greater than at or about 5×10⁹ iCIL cells, optionally wherein the dose is from at or about 5×10⁹ iCIL cells to at or about 100×10⁹ iCIL cells. In some embodiments, multiple doses of the iCIL cells are administered to the subject. In some embodiments, the multiple doses of iCIL cells are administered intermittently or at regular intervals, optionally for a predetermined period of time.

In some embodiments, 2 to 8 doses of the iCIL cells are administered to the subject. In some embodiments, a single dose of the iCIL cells is administered to the subject.

In some aspects, provided herein is a kit comprising any population of iCILs of any one of embodiments provided herein or any composition of any one of embodiments provided herein and instructions for administering the population of iCILs or composition thereof to a subject in need thereof. In some embodiments, the iCILs further comprise the synthetic cytokine receptor for the non-physiological ligand, and the kit further comprises a container comprising the non-physiological ligand and instructions for administering the non-physiological ligand to the subject after administration of the population of iCILs or composition thereof.

In some embodiments, the subject has a cancer.

### BRIEF DESCRIPTION OF FIGURES

FIG. 1A is a diagram of an embodiment, showing expansion of an engineered cytotoxic innate lymphoid cell.

FIG. 1B is a diagram of an embodiment, showing differentiation of an engineered stem or progenitor cell.

FIG. 2A depicts RACR-Expansion of CD19 CAR Blood-Derived NK (bdNK) cells. The top panel shows a timeline of the experiment. Percent CAR expression over time for each group is shown in the left panel and total CAR+ NK cells over time are shown in the right panel.

FIG. 2B depicts CAR expression analysis by flow cytometry. CAR expression is shown on the top panels for bdNK cells expanded in the presence of either IL-2 (1000 U/ml) or AP21967 (20 nM). CD56 and CD16 expression of the CAR+ cells is shown on the bottom panels.

FIG. 2C depicts RACR-expanded blood-derived NK cells' ability to recognize and target tumor cells. The top panel shows a timeline of the experiment. A flow cytometry plot depicting CAR expression is shown on the upper right panel at Day 31 after transduction. 1×10⁵ NK cells were incubated with 1×10⁵ K562 cells, Nalm6 cells, or Nalm6 CD19 knock out cells in the presence of brefeldin A, monensin, and anti-CD107 for 5 hours in 100 μl RPMI media in a 96 well plate. Shown are levels of CD107a (left panel) and IFNγ (middle panel) expression on AP21967-expanded CAR+ cells (AP-20) or untransduced controls. % Dead (right panel) was calculated by comparing the total number of viable, cell trace violet+ cells in each well to a non-effector control well. NK effector cells were added at varying Effector cell:Target cell (E:T) ratios.

FIG. 3A is a diagram of the iPSC-derived CIL cell generation process.

FIG. 3B is a graph depicting % CD34+ cells before and after cell selection and analysis at Day 12 of the differentiation process.

FIG. 3C depicts CD34+ selection by flow cytometry analysis at Day 12 of the differentiation process.

FIG. 3D depicts the percentage of CD45+ cells in leukocytes and the percentage of leukocytes in progenitor cells and CIL cells. The percentage of CD45+ cells are plotted as % Leukocytes, CD7+ cells are plotted as progenitor cells, and CD56+ cells are plotted as CIL cells.

FIG. 3E depicts flow cytometry analysis of differentiated leukocytes (CD45+), progenitors (CD45+/CD5−/CD7+) and CIL cells (CD45+/CD5−/CD7+/CD56+).

FIG. 3F depicts the % of leukocytes or CIL cells at Day 40 (left panel) and a flow cytometry analysis of differentiated cells at Day 40 (right panel).

FIG. 3G depicts the percentage of iCIL cells which were harvested and immunophenotyped by flow cytometry for detection of the markers: CD16, IL7R, KIR, NKp30, and NKp46 (left panel) and cytotoxicity as measured by % 4HLysis of K562 cells for various Effector cell:Target cell (E:T) ratios.

FIG. 4A depicts the timeline of the experiment.

FIG. 4B depicts representative flow cytometry analysis of CD56+ cells and TagCAR (anti-fluorescein isothiocyanate (FITC) chimeric antigen receptor (CAR)) enrichment over the course of the experiment. The x-axis is TagCAR detection by FITC-AF647 and the y-axis is side scatter. Cells analyzed following ‘Mock’ transduction, at Day 32, Day 35, Day 38, and Day 42 following transduction.

FIG. 4C depicts RACR-enrichment of TagCAR cells over time (left panel), total cell counts (middle panel) and percent of cells expressing CD45 and CD56 (right panel) over time for the BXS cell line.

FIG. 4D depicts RACR-enrichment of TagCAR cells over time (left panel), total cell counts (middle panel) and percent of cells expressing CD45 and CD56 (right panel) over time for the NHS cell line.

FIG. 5A is a panel of histograms depicting different activation markers of cytokine-differentiated Mock CIL cells (BXS line) and RACR-iCIL cells (BXS and NH5 lines). Cells were gated on CD45+ CD7+ CD5− and then plotted for activation markers CD56, CD16, NKp30, NKp40 and NKG2D.

FIG. 5B is a graph of a cytotoxicity assay depicting killing of MDA-mCherry tumor cell line by cytokine differentiated Mock CIL cells (BXS cell line) and RACR-iCIL cells (BXS and NH5 cell lines).

FIG. 6A is a diagram depicting the timeline of a differentiation and expansion experiment, results of which are shown in FIG. 6B-6E.

FIG. 6B depicts % VT103 iCIL cells over time (left panel) and total number of VT103 iCIL cells over time (right panel) for cells treated with expansion media containing IL-2, IL-15, IL-21, IL-18, IL-7 (Cytokine Mix) or cells treated with A/C Heterodimerizer AP21967 (AP 100 nM).

FIG. 6C is panel of flow cytometry plots depicting cells stained for RACR-FRB positive cells detected through mCherry.

FIG. 6D is a panel of graphs depicting % FITC-CAR iCIL cells over time (top panel) and total number of FLCAR iCIL cells over time (bottom panel) for cells transduced with viral constructs containing TagCAR-RACR-FRB (206) and FRB-RACR-TagCAR (205).

FIG. 6E is a panel of flow cytometry plots depicting cells stained for FLCAR positive cells.

FIG. 7A is a panel of flow cytometry plots depicting cells stained for CD56 and Tag-CAR.

FIG. 7B is a panel of histograms depicting different markers of activation and cytotoxicity including NKp30, NKp46, NKG2D, NKG2A, and CD57 in TagCAR+ RACR-expanded iCIL cells, Mock IL2-expanded CIL cells, and unstained cells.

FIG. 7C is a panel of histograms depicting CD107a secretion in response to antigen in Mock IL2-expanded CIL cells and TagCAR RACR-expanded iCIL cells.

FIG. 8 is a diagram of an illustrative CAR of the disclosure where the fusion protein is encoded by a lentivirus expression vector and where “SP” is a signal peptide, the CAR is an anti-FITC CAR, a CD8a hinge is present, a transmembrane domain is present (“TM”), the co-stimulation domain is 4-1BB, and the activation signaling domain is CD32.

FIG. 9 is a diagram depicting differentiation factors involved in a transition from iPSC to CIL cells alongside a timeline. Differentiation factors involved in each phase of differentiation are depicted in the outlined boxes; in bold are differentiation factors illustrative of embodiments of the disclosure. In some embodiments, beginning at week 4, cells are differentiated in media comprising IL7, IL15, SCF, FLT3L, and UM729, optionally with or without rapalog. Beginning week 6, these cells are expanded in media comprising IL7, IL15, SCF, FLT3L, UM729, and CD2/NKp46 beads, optionally with or without rapalog. In some embodiments, beginning at week 4, cells are differentiated in media comprising SCF, FLT3L, and UM729, optionally with or without rapalog. Beginning week 6, these cells are expanded in media comprising SCF, FLT3L, UM729, and CD2/NKp46 beads, optionally with or without rapalog. In some embodiments, beginning at week 4, cells are differentiated in media comprising IL7, IL15, and UM729, optionally with or without rapalog. Beginning week 6, these cells are expanded in media comprising IL7, IL15, UM729, and CD2/NKp46 beads, optionally with or without rapalog. In some embodiments, beginning at week 4, cells are differentiated in media comprising UM729, optionally with or without rapalog. Beginning week 6, these cells are expanded in media comprising UM729 and CD2/NKp46 beads, optionally with or without rapalog.

FIG. 10 is a graph depicting % of RACR CD19-CAR positive cells as a function of Culture day. Blood-derived NK cells were transduced with CD19-CAR-RACR (IL-2RG/IL-2RB; RACR2), CD19-CAR-RACR (IL-2RG/IL-7RB; RACR7), or CD19-CAR-RACR (IL-2RG/IL-21RB; RACR21) containing virus. Cells were then expanded in either 100 IU/mL human IL-2 or 100 nM AP219667 in complete media with membrane bound IL-21 (mbIL-21) 41BBL and K562 feeder cells added weekly. RACR (IL-2RG/IL-2RB) “RACR2” supported the highest expansion in AP219667 with both RACR (IL-2RG/IL-7RB) “RACR7” and RACR (IL-2RG/IL-21RB) “RACR21” supported RACR expansion to a lesser extent.

FIG. 11 depicts RACR-enrichment of CAR expressing cells over time (left panel), fold change in progenitor cells (0-4 weeks) and fold change in RACR-iCIL cells (4-9 weeks) (middle panel), and cytotoxicity as measured by % Killing of K562 cells by iCIL and RACR-iCIL cells for various Effector cell:Target cell (E:T) ratios (right panel). The top of the figure depicts a diagram of hematopoietic lineage differentiation and corresponding cell culture.

FIG. 12A is a graph depicting the % of cells comprising markers CD45+ CD5−, CD45+ CD5− CD7+, or CD45+ CD5− CD7+ CD56+ for cells thawed on Day 26 or cells analyzed on Day 29 post transduction.

FIG. 12B is a graph depicting the % of CAR+ cells comprising markers CD45+ CD5−, CD45+ CD5− CD7+, or CD45+ CD5− CD7+ CD56+ for cells thawed on Day 26 and mock transduced or cells analyzed on Day 29 post viral vector transduction.

FIG. 13A is a graph depicting the % of CAR+ iCIL cells following analysis of cells placed in media comprising IL7, IL15, SCF, FLT3L, and UM729 (Full Mix), IL7, IL15, and UM729 (IL7/IL15), or UM729 (None). Each media condition was treated with or without rapalog.

FIG. 13B is a graph depicting the Fold Change (FC) in % CAR+ iCIL cells following analysis of cells placed in media comprising IL7, IL15, SCF, FLT3L, and UM729 (Full Mix), IL7, IL15, and UM729 (IL7/IL15), or UM729 (None). Each media condition was treated with or without rapalog.

FIG. 13C is a graph depicting the total number of CAR+ iCIL cells following analysis of cells placed in media comprising IL7. IL15, SCF, FLT3L, and UM729 (Full Mix), IL7, IL15, and UM729 (IL7/IL15), or UM729 (None). Each media condition was treated with or without rapalog.

FIG. 13D is a graph depicting the Fold Change (FC) in total number of CAR+ iCIL cells following analysis of cells placed in media comprising IL7, IL15, SCF, FLT3L. and UM729 (Full Mix), IL7, IL15, and UM729 (IL7/IL15), or UM729 (None). Each media condition was treated with or without rapalog.

FIG. 14A is a panel of histograms depicting different markers including CD45, CD7, and CD56 in TagCAR+ RACR-iCIL cells, placed in media comprising IL7, IL15, SCF, FLT3L, and UM729 (Full Mix), SCF, FLT3L, and UM729 (SCF/FLT3L), IL7, IL15, and UM729 (IL7/IL15), UM729 (None), or unstained cells. Each media condition was treated with or without rapalog.

FIG. 14B is a histogram depicting the CD56 marker in progenitor cells on Day 26 (CD56−) and cells placed in media comprising IL7, IL15, SCF, FLT3L, and UM729 (Full Mix) analyzed at Day 40 (CD56+).

FIG. 15A shows a schematic for CRISPR-Cas mediated site-specific knock-in of constructs encoding RACR.

FIG. 15B is a graph that shows RACR mRNA detection after knock-in at various promoters including EEF1A1 (locus one), ACTB (locus 2), and B2M-EF1a.

FIG. 15C shows a panel of histograms that depicts RACR protein detection after knock-in at various promoters.

FIG. 15D is a graph that shows RACR protein detection after knock-in at various promoters including locus one (EEF1A1) and locus 2 (ACTB).

FIG. 15E shows a graph depicting CD8 T cell mis-match response after B2M gene knock out.

FIG. 16A shows a schematic depicting the role of FKBP12 in the inhibition of proliferation by rapamycin via mTOR.

FIG. 16B is a graph showing protection from rapamycin-mediated inhibition of iPSC proliferation in polyclonal FKBP12 knock-out (KO) lines (left panel) and phase-contrast images of morphology in wild type and FKBP12 KO cells (right panel).

FIG. 16C is a graph showing confluency of wildtype iPSCs after four days of treatment with varying doses of rapamycin.

FIG. 16D is a graph showing confluency of FKBP12 KO iPSCs growth during four days of treatment with varying doses of rapamycin.

FIG. 16E is a graph showing ratio of hematopoietic progenitors (HPs) to iPSCs of clonal FKBP12 KO iPSCs compared to control iPSCs.

FIG. 16F is a graph showing confluency of FKBP12 KO iPSCs growth during four days of treatment with 25 nM of rapamycin.

FIG. 17A is a graph showing hematopoietic progenitor cell yield from iPSCs genetically engineered to express RACR relative to non-modified iPSCs after treatment with various differentiation factors.

FIG. 17B is a panel of flow cytometry plots depicting triple positive HP cells derived from RACR-engineered iPSCs after treatment with and without SR-1.

FIG. 17C depicts HP yield in RACR-engineered FKBP12 KO iPSCs in the presence (“on”) or absence (“off”) of rapamycin and HP yield in iPSCs by conventional processes (protocol 1 and 2).

FIG. 17D shows flow cytometry plots depicting phenotypic analysis of HP cells markers CD43, CD45, CD34 and CD38. Unstained controls are also shown.

FIG. 18A is a graph that shows the percent of CD45+ CD5−CD56+LFA1+ in WT iNK cells and RACR-iCIL cells after 37 days in culture, with or without culture in a pre-Activation medium.

FIG. 18B shows immunophenotyping that compares iPSC-derived NK cells to RACR-iCILs.

FIG. 18C shows a line graph depicting the fold increase of RACR-iCILs stimulated with rapamycin and cytokines one to two months post harvest.

FIG. 18D depicts flow plots showing the purity of cells and upregulation of LFA-1+/CD56+ cells and NKp46+/NKGD+ cells one and two months post harvest.

FIG. 18E shows killing curves of RACR-iCILs targeting MDA-MBA-231 cells one or two days post harvest.

FIG. 19A shows a graph of the number of tumor cells after incubation with either untreated iCILs, cytokine stimulated iCILs, or RACR-Stimulated iCILs.

FIG. 19B shows a graph of D47 RACR-iCILs proliferation in different cytokine treatments or with a rapalog.

FIG. 19C shows a graph depicting cytotoxicity as measured by % 4HLysis of MDA tumor cells for various Effector cell:Target cell (E:T) ratios.

FIG. 19D shows a graph depicting tumor cell growth after incubating breast adenocarcinoma cells with unstimulated iCILs (“RACR-iCILs+RACR-OFF”), cytokine-stimulated iCILs (“RACR-iCILs+IL2/IL15”), or RACR-stimulated iCILs (“RACR-iCILs+RACR-ON”). Breast adenocarcinoma cells that have not been incubated with iCILs are shown in the bold, black line. Tumor cells were reintroduced (a.k.a., tumor rechallenge) as indicated by arrow.

FIG. 20A shows a graph depicting tumor cell growth after incubating ovarian carcinoma cells with unstimulated NK cells, cytokine-stimulated NK cells or RACR-stimulated NK cells. Ovarian carcinoma cells that have not been incubated with NK cells are shown in the bold, black line. Tumor cells were reintroduced (i.e., tumor cell re-challenge) after 50 hours.

FIG. 20B shows a graph depicting tumor cell growth after incubating bladder carcinoma cells with unstimulated NK cells, cytokine-stimulated NK cells or RACR-stimulated NK cells. Bladder carcinoma cells that have not been incubated with NK cells are shown in the bold, black line. Tumor cells were reintroduced (i.e. tumor cell re-challenge) at 50 and 100 hours.

FIG. 20C shows a graph depicting tumor cell growth after incubating breast adenocarcinoma cells with unstimulated NK cells, cytokine-stimulated NK cells or RACR-stimulated NK cells. Breast adenocarcinoma cells that have not been incubated with NK cells are shown in the bold, black line. Tumor cells were reintroduced (a.k.a., tumor cell re-challenge) after 40 hours.

FIG. 20D shows a graph depicting RACR-NK cell growth after incubating without rapamycin or cytokines (unstimulated NK), with rapamycin (RACR-stimulated NK) or with cytokines (cytokine-stimulated).

FIG. 21A shows a graph depicting tumor cell killing in tumor cells incubated with RACR engineered iCILs and no CAR antigen, RACR engineered iCILS and medium CAR antigen expression, or RACR engineered iCILs and high CAR antigen expression. The CAR antigen is specific to FITC conjugated to folate, wherein the folate is bound to folate receptor on the tumor cells.

FIG. 21B shows a graph depicting the function of iPSC-derived NK cells and CAR and RACR engineered iPSC-derived CILs. The CAR-RACR-iCIL cells secrete CD107a in response to an antigen recognized by the cells.

FIG. 21C shows a graph depicting RACR-CAR-NK cell growth after incubating without rapamycin or cytokines (unstimulated NK), with rapamycin (RACR-stimulated NK) or with cytokines (cytokine-stimulated).

FIG. 22A shows a timeline of an in vivo mouse model of breast cancer. Five days before the experiment began, mice were injected with MDA-231 mCherry/luciferase cells. One day before the experiment began, mice were injected with FITC-Folate subcutaneously for two weeks. On the first day of the experiment (DO), mice were injected with RACR-TagCAR-NK-92 cells intraperitoneally and subsequently treated with rapamycin three times per week or IL2/IL15 three times per week.

FIG. 22B shows a graph depicting quantification of luminescence correlating to tumor growth in mice receiving various treatments over three weeks.

FIG. 22C shows a raw luminescence images of tumor growth in mice receiving various treatments over three weeks.

FIG. 22D depicts RACR-NK in the blood of mice in FIG. 22C.

FIG. 22E depicts RACR-NK detection in tissues of mice in FIG. 22C.

FIG. 23 depicts the ratio of hematopoietic progenitor (HP) cells to iPSC in 3D suspension cultures at day 14 produced following differentiation of unedited iPSC (no RACR) or RACR edited iPSC cells treated in the presence (+Rapa) or absence (−Rapa) of rapamycin.

FIG. 24A depicts fold expansion of iCIL from RACR engineered iPSC compared to unmodified iPSC generated with standard commercially available cell culture protocols in the presence (“on”) or absence (“off”) of rapamycin.

FIG. 24B depicts phenotypic analysis of iCIL markers CD56, LFA1, NKG2D, NKp46, NKp30 and DNAMI in iCIL generated from RACR engineered iPSC.

FIG. 24C depicts fold expansion of iCIL from RACR engineered iPSC with FKBP12 KO compared to unmodified iPSC generated with standard commercially available cell culture protocols in the presence (“on”) or absence (“off”) of rapamycin.

FIG. 24D depicts fold expansion of iCIL from RACR-engineered FKBP12 KO iPSCs compared to CILs generated with standard commercially available cell culture protocols in the presence (“on”) or absence (“off”) of rapamycin.

FIG. 24E depicts phenotypic analysis of iCIL markers CD45, CD56, LFA1 and FSC-A on Day 40 of differentiation.

FIG. 25 depicts tumor cell killing in vitro in co-culture of RACR iCILs with MDA-MB-231 breast adenocarcinoma cells. Breast cancer cells were cultured alone and left untreated, treated with cytokines or rapalog (left legend), or co-cultured with RACR iCILs and left untreated, treated with cytokines or rapalog (right legend).

FIG. 26A depicts a timeline of an in vivo mouse model of breast cancer. Five days before the experiment began, mice were injected with MDA-231 mCherry/luciferase cells. One day before the experiment began, mice were imaged and blood was drawn once a week for the duration of the experiment. On the first day of the experiment (DO), mice were injected with RACR-iCIL cells intraperitoneally and subsequently treated with rapamycin three times per week or IL2/IL15 three times per week.

FIG. 26B depicts raw luminescence images of tumor growth in mice receiving various treatments over three weeks.

FIG. 27A depicts a timeline of an in vivo mouse model of breast cancer. Five days before the experiment began, mice were injected with MDA-231 mCherry/luciferase cells. One day before the experiment began, mice were imaged once a week for the duration of the experiment. On the first day of the experiment (DO), mice were injected with RACR-iCIL cells and subsequently treated with rapamycin three times per week or IL2/IL15 three times per week. On day 27 of the experiment, mice were injected again with RACR-iCIL cells.

FIG. 27B depicts raw luminescence images of tumor growth in mice receiving various treatments over six weeks.

FIG. 27C depicts the absolute quantification of the raw luminescence depicted in FIG. 27B.

FIG. 27D depicts the percentage of weight loss in the mice depicted in FIG. 27B.

FIG. 27E shows a survival plot of mice described in FIG. 27B.

FIG. 28A shows a heat map of differentially expressed hematopoietic stem cell (HSC) genes in hematopoietic progenitor (HP) cells derived from RACR-engineered FKBP12 KO iPSC.

FIG. 28B shows flow cytometry plots depicting EPCR+/CD90+ cells at day 9 of differentiation.

FIGS. 28C-28E depict myeloid and crythroid potency of HP cells derived from RACR-engineered HSC at Day 9 (FIG. 28C), Day 12 (FIG. 28D) and Day 15 (FIG. 28E) of differentiation.

FIG. 29A shows principal component analysis of RNA in iCILs derived from RACR-engineered iPSC relative to: hematopoietic progenitors (HP) derived from RACR-engineered iPSCs; peripheral blood derived NK (bdNK) cells; cord blood derived NK (cbNK) cells; iNK cells; cord blood CD34+ HSC (cbCD34+); and number of feeder cell stimulations (e.g., feed 1 or feed 2).

FIG. 29B shows a heat map of differentially expressed genes in iCIL cells derived from RACR-engineered iPSC cells compared to NK cells including NK cells derived from iPSCs (iNKs), blood derived NK cells (bdNKs), and cord blood derived NK cells (cbNKs).

FIG. 29C shows the gene expression of Fc receptors and genes related to the CD3 complex in iCIL cells derived from RACR-engineered iPSC cells compared to HP cells and NK cells including blood derived NK cells (bdNKs). The groups are as follows, from left to right, for each condition depicted on the x-axis: hematopoietic progenitor (HP), iCIL, blood derived NK cell (BD NK).

FIG. 29D shows the gene expression of cytokine receptors in iCIL cells derived from RACR-engineered iPSC cells compared to HP cells and NK cells including blood derived NK cells (bdNKs). The groups are as follows, from left to right, for each condition depicted on the x-axis: hematopoietic progenitor (HP), iCIL, blood derived NK cell (BD NK).

FIG. 29E shows the gene expression of KIR receptors in iCIL cells derived from RACR-engineered iPSC cells compared to HP cells and NK cells including blood derived NK cells (bdNKs). The groups are as follows, from left to right, for each condition depicted on the x-axis: hematopoietic progenitor (HP), iCIL, blood derived NK cell (BD NK).

FIG. 29E shows gene expression in iCIL cells derived from RACR-engineered iPSC cells compared to HP cells and NK cells including blood derived NK cells (bdNKs). The groups are as follows, from left to right, for each condition depicted on the x-axis: hematopoietic progenitor (HP), iCIL, blood derived NK cell (BD NK).

FIG. 30A shows the percentage of cells expressing cytotoxicity receptors. Specifically depicted are the percentage of bdNK cells, iCIL cells, and iNK cells+feeder cells that are NKp30+/CD56+/LFA1+, NKp46+/CD56+/LFA1+, and NKG2D+/CD56+/LFA1+.

FIG. 30B shows the percentage of cells expressing dysfunction receptors. Specifically depicted are the percentage of bdNK cells, iCIL cells, and iNK cells+feeder cells that are KLRG1+/CD56+/LFA1+, CD73+/CD56+/LFA1+ and CD38+/CD56+/LFA1+.

FIG. 30C shows the percentage of CD56+/LFA1+ bdNK cells, iCIL cells and iNK+feeder cells that are highly proliferative (CD56bright CD57−), transitional (CD56dim CD57−), and senescent (CD57+ CD56dim).

FIG. 30D shows a panel of histograms depicting different markers including cytotoxicity receptors (NKG2D, NKp30, NKG2C, NKp46), phenotype markers (NKG2A, CD161, CD96), and dysfunction markers (KLGR1, CD73, CD57) in iNK cells+feeder cells, iCILs, and bdNK cells.

FIG. 31A shows a graph depicting an in vitro serial killing assay of MDA-MB-231 breast cancer cells by iCIL, bdNK and iNK+feeder cells across 200 hours. Serial killing is determined by the percent of cells remaining. Arrows indicate periods where the breast cancer cells were reintroduced.

FIG. 31B shows a graph depicting effector cell (iCIL, bdNK, feeder-iNK) growth across 200 hours. Arrows indicate periods where the breast cancer cells were reintroduced.

FIG. 32 is a timeline depicting differentiation and expansion in a process starting from RACR-engineered iPSC, differentiation to iCIL and expansion of iCIL cells.

FIG. 33 is a diagram depicting differentiation factors involved in a transition from iPSC to CIL cells in an exemplary process (“version 5”) compared to the total control. Differentiation factors involved in each phase of differentiation are depicted in the boxes and correspond to the timeline depicted on the y-axis.

FIG. 34 is a diagram depicting hematopoietic progenitor (HP) and iCIL formation from RACR-engineered iPSC cells over time.

FIG. 35A shows a graph depicting the number of hematopoietic progenitor (HP) cells per iPSC cell in response to cell confluency.

FIG. 35B shows a graph depicting the number of iCIL cells per iPSC cell in response to cell confluency at the start of differentiation.

FIG. 35C shows a graph depicting the fold expansion of hematopoietic progenitor (HP) cells in wells seeded with iPSC cells of various densities.

FIG. 36A shows a graph depicting the fold expansion of hematopoietic progenitor (HP) cells in various media.

FIG. 36B shows a graph depicting the fold expansion of hematopoietic progenitor (HP) cells with the addition of UM729 and SR1 on Day 6, as compared to Day 9.

FIG. 37A shows a graph depicting the fold expansion of hematopoietic progenitor (HP) cells with the removal of rapamycin at various stages.

FIG. 37B shows a graph depicting the fold expansion of hematopoietic progenitor (HP) cells at different concentrations of rapamycin in the culture.

FIG. 38A shows a graph depicting the fold expansion of iCIL cells at Day 33 with the removal of various differentiation factors in the culture during the differentiation phase.

FIG. 38B shows a graph depicting the fold expansion of hematopoietic progenitor (HP) cells at Day 15 with the removal of various differentiation factors in the culture during the differentiation phase.

FIG. 39A shows a graph depicting the fold expansion of iCIL cells at Day 40 when differentiated and expanded in a bioreactor at Day 15.

FIG. 39B shows a graph depicting the fold expansion of iCIL cells at various seeding densities in the bioreactor.

FIG. 39C shows a graph depicting the fold expansion of iCIL cells at Day 35 when differentiated and expanded in a vertical wheel bioreactor compared to a GRex bioreactor.

FIG. 39D shows a graph depicting the fold expansion of iCIL cells at Day 35 when differentiated and expanded in a stirred-tank bioreactor compared to a vertical wheel bioreactor.

FIG. 40 shows a graph depicting the fold expansion of iCIL cells using different medias during the iCIL differentiation phase.

FIG. 41 shows a graph depicting the fold expansion of iCIL cells using different concentrations of rapamycin during the iCIL differentiation phase.

FIG. 42A shows a graph depicting the fold expansion of iCIL cells with the removal of IL7 and FLT3L from the media in the iCIL differentiation phase. The groups are as follows, from left to right, for each condition depicted on the x-axis: Clone 3.1, Clone 56.1.

FIG. 42B shows a graph depicting the cytotoxicity of iCIL cells against tumor cells with the removal of IL7 and FLT3L from the media in the iCIL differentiation phase.

FIG. 43 shows a graph depicting the fold expansion of iCIL cells with the addition of SCF and IL-15 in the media during the iCIL differentiation phase from Days 21-28.

FIG. 44 shows a graph depicting the fold expansion of iCIL cells with the addition of SCF and IL-15 in the media during the iCIL differentiation phase from Days 24-28.

FIG. 45 shows a graph depicting the fold expansion of iCIL cells with the removal of various differentiation and expansion factors from the media during the HP differentiation and the iCIL differentiation phases.

FIG. 46 shows graphs depicting percentage of tumor remaining after in vitro in co-culture of RACR iCILs with MDA-MB-231 breast adenocarcinoma cells.

FIG. 47A shows the expression of LFA-1 and CD19 in RACR-expanded iCIL cells by flow cytometry.

FIG. 47B shows the fold expansion of iCIL cells following expansion in a bioreactor utilizing the exemplary version 5 protocol depicted in FIGS. 32 and 33.

FIG. 48A shows graphs depicting percentage of tumor remaining after in vitro in co-culture of RACR iCILs with MDA-MB-231 breast adenocarcinoma cells with and without cytokines and rapamycin.

FIG. 48B shows graphs depicting percentage of tumor remaining after in vitro in co-culture of RACR iCILs with SW620 colorectal cancer cells with and without cytokines and rapamycin.

FIG. 49A shows graphs depicting percentage of tumor remaining after in vitro in co-culture of RACR iCILs with Burkitt's lymphoma tumor cells with and without cytokines, rapamycin and/or rituximab.

FIG. 49B shows graphs depicting percentage of tumor remaining after in vitro in co-culture of RACR iCILs with non-Hodgkin's lymphoma tumor cells with and without cytokines, rapamycin and/or rituximab.

FIG. 50 shows a graph depicting percentage of tumor remaining after in vitro in co-culture of RACR iCILs with ten tumor cell types with and without cytokines.

FIG. 51 shows a graph depicting percentage of tumor remaining after in vitro in co-culture of RACR iCILs with triple-negative breast adenocarcinoma cells in a serial killing assay (left panel). FIG. 51, right panel shows raw luminescence images of tumor growth in co-culture with RACR iCILs.

### DETAILED DESCRIPTION OF THE INVENTION

Induced pluripotent stem cells (iPSCs) are a renewable, modifiable, and scalable source of material for cell therapy manufacturing. However, major challenges exist in current iPSC-based approaches to cell therapy. Specifically, current approaches for differentiating iPSCs into therapeutic immune cell types require exogenous growth factors, and in some cases, the presence of feeder cells.

iPSCs can be made by reprogramming adult cells into a cellular state akin to embryonic stem cells. iPSCs are thought to be capable of differentiating into all cell types found in the human body and possess an unlimited expansion capacity, meaning they can reproduce and proliferate indefinitely, generating a nearly endless supply of starting material. Additionally, iPSCs are amenable to precision multiplex genome editing, allowing safe introduction of multiple genetic modifications. Because of these properties, iPSCs provide a consistent starting material, originating from a single cell (clone), which enables consistent genome integrity in process intermediates and the final cell product.

Current approaches to cell therapy manufacturing include cither autologous or allogeneic cellular starting material from which a disease-targeted therapeutic cell product is engineered. In some embodiments, an allogeneic or “off-the-shelf” cell therapy has the potential to transform cell therapy from personalized medicine into a routine treatment. However, current “off-the-shelf” cell therapies are struggling to show the same engraftment and persistence of cells in vivo that has been reached by approved autologous cell therapy products. In some aspects, this is due to the foreign nature of allogeneic or even engineered elements of autologous cells, which can be recognized and rejected by the host immune system. To date, cell therapies achieve cell engraftment by treating patients with highly toxic chemotherapy regimens, termed lymphodepletion (LD), given prior to administration of the cell therapy product. LD essentially removes the host immune system and provides many benefits to the cell therapy product, firstly providing free “homeostatic cytokines” for an ex vivo cell therapy product as well as reducing anti-graft responses against the foreign graft by the host immune system. However, LD is a transient solution, and the host immune system rapidly reconstitutes. Thus, to sustain allogeneic cell exposure, multiple rounds of LD and cell infusion are required. Additionally, exogenous cytokines such as IL-2 are administered, and these cytokine treatments have low exposure times with high toxicities associated with their use. Finding better ways to increase cell persistence is key to achieving durable tumor remission and has proven to be a challenge in the allogeneic cell therapy space.

Allogeneic cells can be further broken down into donor- or iPSC-derived cells. Donor-derived cells are generally sourced from the circulation or cord blood of a healthy donor and the therapeutic cell type (e.g., natural killer or NK cells) is selected, subsequently harvested, and expanded in a complex cell culture process that generally includes multiple cytokines, growth factors, gene engineering, and feeder cells to generate many doses. Alternatively, iPSC-derived cells, which also require multiple complex cell culture conditions, must have these conditions implemented in a stepwise fashion to drive cells through the necessary progenitor stages to ultimately obtain the intended final cell product (e.g., immune effector cell). For example, effective methods for CIL cell expansion for clinical-scale purposes are known to require exogenous cytokines, including IL-2, IL-15, and/or IL-7, as well as antigen molecules, co-stimulatory molecules, and/or cell adhesion molecules. CIL cells are cytotoxic lymphocytes characterized by their ability to discriminate between self and non-self by monitoring the expression of MHC class I molecules, release of cytokines, and directly kill non-self or infected target cells. It is known in the art that CIL cells do not represent a uniform population. Rather, there are many distinguishable subsets of CIL cells. In many studies, these exogenous factors are supplemented during ex vivo expansion of CIL cells and/or after infusion of the CIL cells into a subject. Effectively expanding CIL cells that rely on a large quantity of diverse exogenous factors using currently known methods often requires complex and expensive manufacturing processes. A further difficulty in the field involves modulating the activity of endogenous CIL cells in vivo, especially given that patients with cancer exhibit significantly reduced NK cell activity as compared with healthy patients.

Furthermore, the gold standard for cell therapy is autologous chimeric antigen receptor (CAR) T cell therapy. Decades of CAR T cell therapy efforts, starting with “first generation” CAR T cell therapies in the 1990s and leading to the first CAR T cell therapies receiving FDA approval in 2017, have resulted in successes in treating B cell malignancies, with long-term remission achieved in 30-40% of certain patient populations. Importantly, CAR T cell efficacy requires lymphodepleting chemotherapy to eliminate sinks for survival factors such as IL-15. While CAR T cell therapies have revolutionized the treatment of malignancies (e.g., hematologic), major limitations hinder its widespread application. The allogeneic CAR T therapy field has shown promising early clinical results; however, the durable response profile has been generally poor in comparison to autologous CAR T cell therapies, despite the use of ever increasing intensity LD regimens. This is likely due to limitations of the drug product cell type, manufacturing processes, as well as anti-allograft responses against the therapeutic cells. Thus, despite the promising clinical efficacy of CAR T cells in hematologic malignancies, significant challenges remain, including patient access, complex manufacturing, and high cost. The provided engineered CIL cells and methods related to the same provide for “off-the-shelf” cancer therapies to overcome these challenges.

iPSCs can also be modified via CRISPR to express a CAR to overcome challenges associated with targeting, for example, the heterogeneous solid tumor microenvironment.

Overall, iPSC-based cell therapy is generally inefficient in generating the necessary intermediate progenitor cells, resulting in a low initial yield of the therapeutic cell type (e.g., cytotoxic innate lymphocyte (CIL) cells), which then requires feeder cell-driven expansion. This feeder cell-driven expansion can dramatically reduce the proliferative capacity of the final cell therapy product. Thus, in order to achieve the necessary engraftment to have any therapeutic effect, high cell numbers (˜1 billion cells) and repeat dosing are required in addition to repeated cycles of lymphodepleting chemotherapy.

Current approaches for differentiating iPSCs into therapeutic immune effector cells, such as natural killer (NK) cells, require complex growth factors and feeder cells to achieve sufficient yields. Provided herein is a Synthetic Receptor Enabled Differentiation (ShRED), a directed differentiation and expansion process controlled by the Rapamycin-Activated Cytokine Receptor (RACR). RACR is activated via the addition of its synthetic ligand rapamycin, which induces a JAK/STAT signal that drives differentiation and expansion of cells into hematopoietic progenitors (HPs) and then into immune effector cells, termed RACR-induced Cytotoxic Innate Lymphocytes (RACR-iCILs). Furthermore, because rapamycin is a safe, effective, and approved therapeutic for immune suppression, RACR can also be engaged in vivo through rapamycin dosing to increase the persistence of RACR-iCILs, while simultaneously protecting these cells from allogeneic rejection

Provided herein is a platform for producing immune effector cells in the absence of exogenous cytokines and feeder cells by genetically modifying iPSCs and iPSC-derived progenitor cells to express a synthetic cytokine receptor. A synthetic small molecule ligand (e.g., rapamycin) activates the receptor to drive the differentiation and expansion of immune effector cells. The compositions and methods provided herein comprise CIL cells engineered to express a synthetic cytokine receptor. Non-limiting advantages of the engineered CIL cells include superior and controllable expansion when administered to a subject, similar cytotoxic activity as compared to native CIL cells, improved iPSC-derived cell manufacturing and enhanced anti-tumor activity.

Improved Cell Manufacturing. In provided aspects, the RACR engineering platform provided herein improves iPSC-derived cell manufacturing by controlling cell production. Through rapamycin dosing and activation of RACR, a more reproducible differentiation process and homogeneous cell product results. The RACR engineering platform also reduces manufacturing costs as RACR activation eliminates the need to add expensive growth factors, cytokines and other raw materials. In certain embodiments, the methods disclosed herein may further enhance expansion through the ability of the CIL cells described herein to be expanded without or with fewer exogenous factors, such as without IL-2, IL-15, and/or IL-7. In some embodiments, the methods disclosed herein may further enhance differentiation and/or expansion through the ability of the iCIL cells described herein to be generated with the removal of one or more exogenous factors as compared to a conventional process. For example, in some cases at least 7 fewer exogenous factors are necessary for the described processes. The RACR engineering platform increases yields of highly pure intermediate and final cell products. The RACR engineering platform provided herein generated highly pure hematopoietic progenitors (HPs), an intermediated progenitor population, and resultant CILs that are highly pure and phenotypically mature. The RACR engineering platform increases patient-compatibility of the cells as the manufacturing process is completely feeder cell and xenogeneic cell free. The RACR engineering platform is also compatible with cells in suspension, promoting scalability of cell production.

In other provided aspects, the RACR engineering platform removes the need for additional physical processing of differentiated progenitor cells. In conventional processes of differentiating progenitor cells, residual cell aggregates must be removed prior to blood cell differentiation. Physical processing includes enzymatic digestion (e.g., collagenase or TrypLE™ enzymes) and filtration (e.g., cells are strained to remove undesired cell aggregates). In contrast, the RACR engineering platform results in embryoid bodies that completely dissociate into pure HPs with no cell filtration required.

Enhanced Anti-Tumor Activity. In provided aspects, the RACR engineering platform provided herein improves anti-tumor activity of iPSC-derived cells by increasing cell engraftment, persistence and effector function. The RACR engineering platform provided herein also improves anti-tumor activity of iPSC-derived cells by inhibiting host immune response via rapamycin dosing, which further enables engraftment of the cells (e.g., CILs). The RACR engineering platform provided herein also improves anti-tumor activity of iPSC-derived cells by removing the need for toxic LD due by activating the RACR system to selectively support RACR cell expansion and survival.

In additional provided aspects, the results demonstrate the surprising finding that RACR-engineered iPSC-derived CIL (iCIL) cells express low levels of CD38, which is the target of certain therapeutic antibodies such as daratumumab. A problem with many iPSC derived cell therapies against certain cancer or tumor target antigens such as CD38, is that the iPSC derived cells may express CD38. In fact, others have reported that NK cell compositions comprise a large population of cells expressing a high percentage (e.g., >90%) of CD38+ NK cells. Expression of CD38 on iPSC derived cell therapies, like NK cell therapies, can be a problem because when anti-CD38 targeted antibodies (e.g. daratumumab) binds CD38, fratricide occurs whereby ADCC leads to elimination of the tumor and the cell therapy. In contrast, the findings herein demonstrate that the percentage of CD38+ cells is markedly lower on iCIL cells compared to conventional bdNK cells or iNK cells differentiated with feeder cells. These results support utility of a combination therapy comprising the provided iCIL cells and anti-CD38 antibodies. This combination therapy would confer enhanced anti-tumor activity without the iCIL cells suffering fratricide-related depletion.

In some aspects, among advantages of the RACR system on CILs, including the engineered synthetic cytokine receptor and activation thereof with rapamycin or rapalog, includes: the ability to engineer unlimited starting material that is highly efficient at generating immediate progenitors and that is characterized by minimized expansion requirements on the final cell type; the ability to efficiently edit cells; no requirement for feeder cells, thereby minimizing complex, raw materials; no requirement for lymphodepletion in subjects receiving RACR-engineered cells; low to no cytokine release syndrome (CRS) or Immune effector cell-associated neurotoxicity syndrome (ICAN); and promotion of engraftment, expansion, and persistence with administration of rapamycin or rapalogs. In some embodiments the RACR-iCILs provide a source of cells for allogenic cell therapy, which, in some aspects, can be achieved while minimizing or eliminating hypoimmune engineering requirements.

Provided here are synthetic cytokine receptors that can be applied to support the derivation of cytotoxic innate lymphoid (CIL) cells. CIL cells may be derived from stem or progenitor cells, and such cells are termed herein “induced cytotoxic innate lymphoid” (iCIL) cells. iCIL cells share distinguishing cell surface markers and functional attributes as described herein. As used herein, the terms “induced cytotoxic innate lymphoid cell” or “iCIL” refers to a CIL made by inducing differentiation of progenitor cells. As disclosed herein, iCIL may be made and/or expanded by expressing a synthetic cytokine receptor in a stem or progenitor cell and acting the synthetic cytokine receptor by the non-physiological ligand. Such a process may involve differentiation of a progenitor cell engineered to express a synthetic cytokine receptor by activation of the synthetic cytokine receptor. The process may also or alternatively involve expansion of the progenitor cell or the CIL by activation of the synthetic cytokine receptor.

In some embodiments, the present disclosure provides stem cells (e.g., iPSCs) and CIL cells engineered to express a rapamycin activated cytokine receptor (RACR), a synthetic cytokine receptor activated by the small molecule rapamycin or rapalogs. CIL cells comprising a RACR and activated with rapamycin or a rapalog are termed herein “RACR-iCIL” cells. Stem cells comprising a RACR and activated with rapamycin or a rapalog are termed herein “RACR-SCs”. RACR is demonstrated to support differentiation and/or expansion of RACR-SCs and RACR-iCIL cells in a feeder-free manufacturing process. RACR-iCIL cells express multiple innate tumor targeting receptors and when engineered to express a chimeric antigen receptor (CAR), are able to exert CAR-directed cytolytic activity. Accordingly, RACR-iCIL cells provide an “off-the-shelf” allogeneic cell therapy.

The disclosure relates, in part, to the surprising discovery that stem cells engineered to express a synthetic cytokine receptor differentiate to hematopoietic progenitors, CLPs or CMPs in response to the receptor's cognate non-physiological ligand. CIL cells differentiated from the engineered stem cells retain the synthetic cytokine receptor and expand in response to the receptor's cognate non-physiological ligand. The engineered CIL cells may be generated in high quantities and with functional activity equal to or greater than CIL cells from other sources.

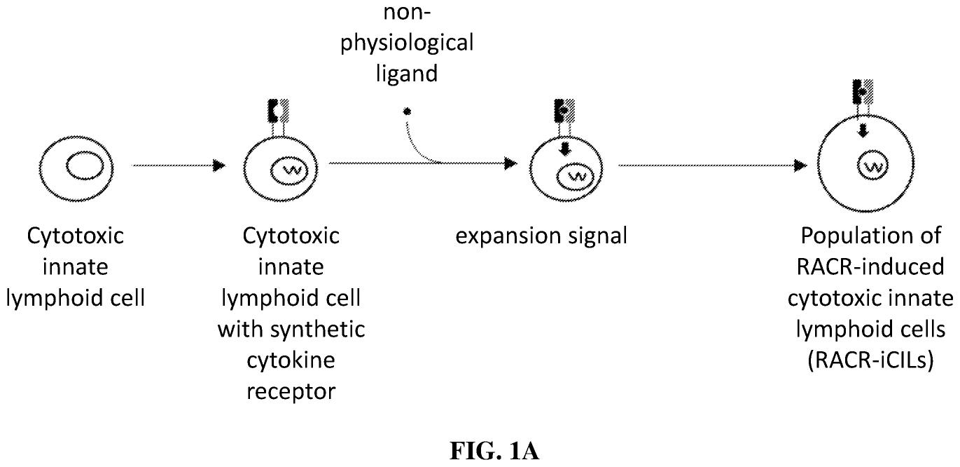

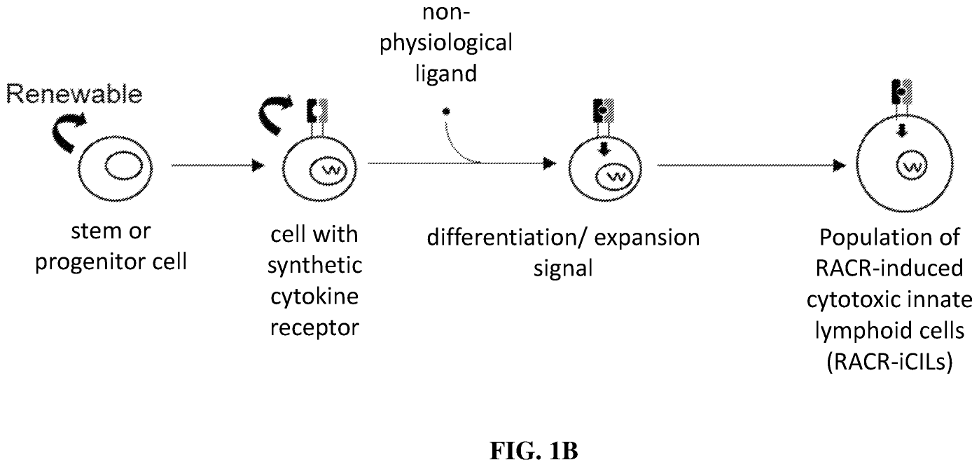

As shown in the diagram of FIG. 1A, an isolated, CIL cell may be transduced with a vector comprising at least one polynucleotide encoding a synthetic cytokine receptor. Upon contacting the transduced CIL cell with a non-physiological ligand, the extracellular domains of the cytokine receptor dimerize through mutual binding of the non-physiological ligand. This dimerization generates an expansion signal within the CIL cell which produces a population of phenotypically enriched and functionally active, engineered, CIL cells.

As shown in the diagram of FIG. 1B, a stem or progenitor cell may be transduced with a vector comprising at least one polynucleotide encoding a synthetic cytokine receptor. Upon contacting the transduced cell with a non-physiological ligand, the extracellular domains of the cytokine receptor dimerize. The dimerization generates a differentiation signal within the stem or progenitor cell which induces sequential differentiation to becoming CIL cells.

The CIL cells may be derived from iPSCs, common lymphoid progenitor cells (CLPs), or other stem or progenitor cells. Further provided herein are stem or progenitor cells engineered to express synthetic cytokine receptors, and methods of differentiating engineered stem or progenitor cells into CIL cells by contacting the stem or progenitor cells with the cognate non-physiological ligand for the cytokine receptor.

In certain embodiments, provided herein are ex vivo generated CIL cells. The engineered cells described herein, and related compositions, may be used for immunotherapy with ligand-controlled ex vivo expansion. Further provided herein are methods of expanding CIL cells by contacting the cells with the cognate non-physiological ligand for the synthetic cytokine receptor. Moreover, the engineered CIL cells disclosed herein may be further engineered to express a chimeric antigen receptor (CAR), enabling targeting of the engineered CIL cells to cells expressing or labelled with the antigen recognized by the CAR.

In some embodiments, the provided engineered CIL cells and methods provided for an improved immunotherapy compared to existing strategies. While chimeric antigen receptor (CAR) T cell therapies have revolutionized the treatment of hematologic malignancies, major limitations hinder their widespread application.

In some embodiments, engagement of the synthetic cytokine receptor not only is able to promote differentiation but also is able to increase cell growth and promote expansion through engagement of the synthetic cytokine receptor on provided engineered iCIL cells. In some aspects, the provided engineered iCIL cells and related methods can be used to increase cell growth and expansion in vivo of the engineered cell therapy through rapamycin dosing of patients after the cell therapy product. In some embodiments, rapamycin simultaneously expands and protects the cells. Expansion is achieved through the JAK/STAT signal activation and protection is achieved through rapamycin suppression of host anti-graft responses. In some embodiments, the need for lymphodepletion as well as exogenous cytokine dosing is not necessary. In some embodiments, provided methods of administration and treatment with the engineered iCIL cells can be carried out without lymphodepletion (e.g. without the need to administer a lymphodepleting therapy such as cyclophosphamide and/or fludarabine). In some embodiments, provided methods of administration and treatment with the engineered iCIL cells can be carried out without exogenous cytokine administration (e.g. without the need to administer IL-2 and/or IL-15).

In some embodiments, the provided methods also can include administering the non-physiological ligand of the synthetic cytokine receptor (e.g. rapamycin or rapalog) to the subject to expand or reinvigorate the engineered iCILs in the subject. Thus, in some embodiments, it is not necessary to further re-dose the subject with iCILs, since it is possible to expand the cells in vivo with the non-physiological ligand. However, re-dosing of iCILs also is possible due to the hypoimmune engineering as described herein making allogeneic cell therapy possible. Furthermore, since the provided methods can be carried out without lymphodepletion this further provides advantages to promote expansion of the transferred cells as well as promote a host anti-tumor response. This is because without lymphodepletion the host immune system remains and is not heavily depleted. The immune response generated by the iCIL cells (e.g. release of cytokines and other pro-inflammatory factors) therefore could stimulate the existing immune system of the host against the tumor. Moreover, the exemplary non-physiological ligand rapamycin not only promotes expansion of the transferred cells via engagement of the synthetic cytokine receptor, but transient mTOR suppression like achieved via rapamycin can reinvigorate T cells as well as promote apoptosis of suppressive macrophages. Also, while the non-physiological ligand rapamycin or an analog can suppress an anti-graft response by the host, this is expected to be only transient and such that a more normal host anti-tumor response would resume once administration of the non-physiological ligand is discontinued.

In some embodiments, engineered cells herein are further modified to be resistant to the effects of rapamycin on inhibiting or reducing cell growth and expansion. In some embodiments, the cells can be made “rapamycin resistant” by providing free cytosolic FRB to the cell in order to complex with rapamycin and thereby eliminate or reduce rapamycin-mediated growth inhibition of a source cell or iCIL. In other embodiments, the cells can be made “rapamycin resistant” by disrupting, such as inactivating or knocking out, FKBP12 in the engineered cell. It is found herein that, in some cases, overexpression of FRB may not result in free-FRB that is able to completely quench rapamycin. Thus, in cases, editing endogenous genes in the cell, such as by FKBP12 knockout, can provide for full rapamycin resistance of cells.

Accordingly, provided embodiments employing a synthetic cytokine receptor system, such as a rapamycin activated cytokine receptor (RACR) that can be engaged by rapamycin or an analog, e.g. rapalog, both protects and expands cells in a single technology. In addition, the additional inclusion of genetic disruption, such as knockout of certain immune genes such as beta-2-microgloublin (B2M), also can produce “stealth” cells that have additional advantages for allogeneic cell therapy.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described. Those skilled in the art will recognize that several embodiments are possible within the scope and spirit of the present disclosure. The following description illustrates the disclosure and, of course, should not be construed in any way as limiting the scope of the inventions described herein.

### I. DEFINITIONS

All publications, patent applications, patents and other references mentioned herein are incorporated by reference in their entirety.

Unless the context indicates otherwise, the various features described herein can be used in any combination with any feature or combination of features set forth herein, and each feature can be excluded or omitted from the combination.

As used herein, the singular forms “a”, “an”, and “the” are include the plural forms as well, unless the context indicates otherwise. The conjugation “and/or” denotes all possible combinations of one or more of listed items.

“Subject” as used herein refers to the recipient of an engineered CIL cell or other agent. The term includes mammal, such as primate, mouse, rat, dog, cat, cow, horse, goat, camel, sheep or a pig, preferably a human.

“Treat,” “treating” or “treatment” as used herein refers to any type of action or administration that imparts a benefit to a subject that has a disease or disorder, including improvement in the condition of the patient (i.e., improvement, reduction, or amelioration of one or more symptoms, and partial or complete response to treatment).

The term “effective amount” refers to an amount effective to generate a desired biochemical, cellular, or physiological response. The term “therapeutically effective amount” refer to the amount, dosage, or dosage regime of a therapy effective to cause a desire treatment effect.

“Polynucleotide” as used herein refers to a biopolymer composed of two or more nucleotide monomers covalently bonded through ester linkages between the phosphoryl group of one nucleotide and the hydroxyl group of the sugar component of the next nucleotide in a chain. DNA and RNA are non-limiting examples of polynucleotides.

“Polypeptide” as used herein refers to a polymer consisting of amino acid residues chained together by peptide bonds, forming part of (or the whole of) a protein.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described herein to reflect the codon usage of any particular host organism in which the polypeptides are to be expressed.

Nucleic acids may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The term “variant” means a polynucleotide or polypeptide having at least one substitution, insertion, or deletion in its sequence compared to a reference polynucleotide or polypeptide. A “functional variant” is a variant that retains one or functions of the reference polynucleotide or polypeptide.

As used herein the term “sequence identity”, or “identity” in relation to polynucleotides or polypeptide sequences, refers to the extent to which two optimally aligned polynucleotides or polypeptide sequences match at each position in the alignment across the full length of the reference sequence. The “percent identity” is the number of matched positions in the optimal alignment, divided by length of the reference sequence plus the sum of the lengths of any gaps in the reference sequence in the alignment. The optimal alignment is the alignment that results in the maximum percent identity. Alignment of sequences to determine percent identity can be accomplished by a number of well-known methods, including for example by using mathematical algorithms, such as, for example, those in the BLAST suite or Clustal Omega sequence analysis programs. Unless noted otherwise, the term “sequence identity” in the claims refers to sequence identity as calculated by BLAST version 2.12.0 using default parameters. And, unless noted otherwise, the alignment is an alignment of all or a portion of the polynucleotide or polypeptide sequences of interest across the full length of the reference sequence.

As used herein, “small molecule” refers to a low molecular weight (<1000 Daltons), organic compound. Small molecules may bind specific biological macromolecules and can have a variety of biological functions or applications including, but not limited to, serving as cell signaling molecules, drugs, secondary metabolites, or various other modes of action.

The term “analog” in relation to a small molecule refers to a compound having a structure and/or function similar to that of another compound but differing from it in respect to a certain component. The analog may differ in one or more atoms, functional groups, or substructures, which are replaced with other atoms, groups, or substructures. Despite a high structural and/or functional similarity, analogs can have different physical, chemical, physiochemical, biochemical, or pharmacological properties.

The term “rapalog” is an art-recognized group of analogs of rapamycin analog that share structural and functional similarity to rapamycin. Certain rapalogs are known to share some but not all functional attributes of rapamycin. For example, some rapalogs are suitable for uses as a non-physiological ligand because they promote dimerization but have substantially no immunosuppressive activity (e.g., AP21967, AP23102, or iRAP).

An illustrative rapalog of the disclosure is AP21967

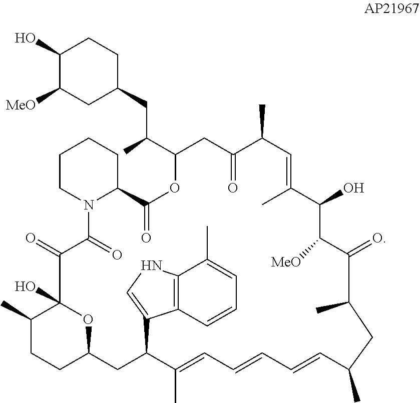 [SMILES: CO[C@@H]1C[C@H](C[C@H](C)C2CC(=O)[C@@H](C)/C=C(\C)[C@@H](O)[C@@H](OC)C(=O)[C@H](C)C[C@@H](C)/C=C/C=C/C=C(\C)[C@H](c3c[nH]c4c(C)cccc34)C[C@H]3CC[C@@H](C)[C@@](O)(O3)C(=O)C(=O)N3CCCC[C@H]3C(=O)O2)CC[C@@H]1O]

An illustrative rapalog of the disclosure is AP23102

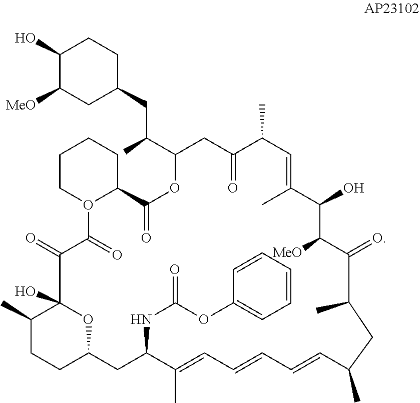

An illustrative rapalog of the disclosure is iRAP

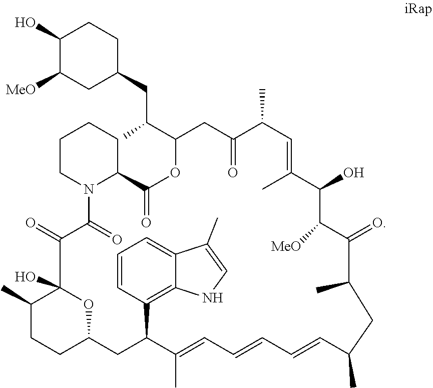 [SMILES: CO[C@@H]1C[C@H](C[C@@H](C)C2CC(=O)[C@H](C)/C=C(\C)[C@@H](O)[C@@H](OC)C(=O)[C@H](C)C[C@@H](C)/C=C/C=C/C=C(\C)[C@H](c3cccc4c(C)c[nH]c34)C[C@@H]3CC[C@@H](C)[C@@](O)(O3)C(=O)C(=O)N3CCCC[C@H]3C(=O)O2)CC[C@@H]1O]

The term “cell population” refers to mixture of cells suspended in solution, attached to a substrate, or stored in a container. The characteristics of a cell population as a whole can be studied with bulk measurements of sample volumes having a plurality of cells. Flow cytometry methods may be employed to reduce problems with background fluorescence which are encountered in bulk cell population measurements.

As used herein, the term “Cytotoxic Innate Lymphoid cell” or “CIL cell” is used to refer to a class of cytotoxic lymphocytes that constitute a major component of the innate immune system. In humans, cytotoxic innate lymphoid cells usually express the surface markers CD16 (FCyRIII) and CD56, and may express CD127. They may express one or more of CD45, CD94, CD122, KIR, NKG2A, NKG2D, NKp30, NKp44, NKP46, NKp80. Cytotoxic innate lymphoid cells generally do not express CD3, or express lower levels of CD3 than CD3+ T cells. CIL cells are cytotoxic and comprise small granules in their cytoplasm that contain special proteins such as perforin and proteases known as granzymes. CIL cells provide rapid responses to virally infected cells and respond to transformed cells. Upon release in close proximity to a cell slated for killing, perforin forms pores in the cell membrane of the target cell through which the granzymes and associated molecules can enter, inducing apoptosis. CIL cells may act as effectors of lymphocyte cell populations in anti-tumor and anti-infection immunity. In some embodiments, the CIL cell is a NK cell. In some embodiments, the CIL cell is a blood derived NK cell (bdNK), iPSC derived NK cell (iPSC-NK), or other cytotoxic innate lymphoid cell. For clarity, the term “CIL” cells excludes adaptive immune cells and their progenitors and excludes common lymphoid progenitor cells (CLPs). However, CILs may be derived from CLPs. In some embodiments, the CIL is an induced cytotoxic innate lymphoid cell (iCIL) in which iPSCs are induced to differentiate to cytotoxic innate lymphoid cells using methods described herein. In some aspects, iCILs may exhibit one or more phenotypic or functional features that are unique compared to conventional NK cells, such as bdNK cells or iPSC-NK cells.

As used herein, the term “engineered” refers to a cell that has been stably transduced with a heterologous polynucleotide or subjected to gene editing to introduce, delete, or modify polynucleotides in the cell, or cells transiently transduced with a polynucleotide in a manner that causes a stable phenotypic change in the cell.

As used herein, the term “stem cell” is used to describe a cell with an undifferentiated phenotype, capable, for example, of differentiating into hematopoietic progenitors, common lymphoid progenitors, cytotoxic innate lymphoid cells, and/or NK cells.

As used herein, the term “pluripotent” means the stem cell is capable of forming substantially all of the differentiated cell types of an organism, at least in culture. For example, embryonic stem cells are a type of pluripotent stem cells that are able to form cells from each of the three germs layers, the ectoderm, the mesoderm, and the endoderm.

As used herein, the terms “induced pluripotent stem cell” and “iPSC” are used to refer to cells, derived from somatic cells, that have been reprogrammed back to a pluripotent state and are capable of proliferation, selectable differentiation, and maturation. iPSCs are stem cells produced from differentiated adult, neonatal, or fetal cells that have been induced or changed, i.e., reprogrammed, into cells capable of differentiating into tissues of all three germ or dermal layers: mesoderm, endoderm, and ectoderm. The iPSCs produced do not refer to cells as they are found in nature.

As used herein, the term “hematopoietic stem cell” refers to stem cells capable of giving rise to both mature myeloid and lymphoid cell types including natural killer cells, T cells, and B cells. Hematopoietic stem cells are typically characterized as CD34+.

The term “progenitor” refers to a cell partially differentiated into a desired cell type. Progenitor cells retain a degree of pluripotency and may differentiate to multiple cell types.

As used herein, the term “hematopoietic progenitor cell” refers to cells of an intermediate cell type capable of differentiating down blood cell lineages, wherein the hematopoietic progenitor cell may differentiate into either common myeloid progenitor cells or common lymphoid progenitor cells. Hematopoietic progenitor cells are typically characterized as CD34+ and CD45+. CD38 is also considered a marker for hematopoietic progenitor cells. CD45 is considered a hematopoietic lineage marker.

As used herein, the terms “lymphoid progenitor cell” or “lymphoblast” or “common lymphoid progenitor” refer to cells that are precursors to lymphoid cells, e.g., CIL and NK cells. Lymphoid progenitor cells are the first stage of differentiation of hematopoietic stem cells that follow the lymphoid lineage of differentiation. As used herein, the term “lymphoid progenitor” refers to cells capable of hematopoietic transition to hematopoietic cell-types. Lymphoid progenitor cells may be characterized by being CD45+ CD7+ CD5+/lo CD3− CD56−. Lymphoid progenitor cells may be characterized by being CD45+ CD5+/lo CD7+.

As used herein, “differentiate” or “differentiated” are used to refer to the process and conditions by which undifferentiated, or immature (e.g., unspecialized), cells acquire characteristics becoming mature (specialized) cells thereby acquiring particular form and function. Stem cells (unspecialized) are often exposed to varying conditions (e.g., growth factors and morphogenic factors) to induce specified lineage commitment, or differentiation, of said stem cells.

As used herein, “expand” or “expansion” refer to an increase in the number and/or purity of a cell type within a cell population through mitotic division of cells having limited proliferative capacity, e.g., CIL cells.

As used herein, “activity”, “activate”, or “activation” refer to stimulation of activating receptors on a cytotoxic innate lymphoid cell leading to cell division, cytokine secretion (e.g., IFNγ and/or TNFα), and/or release of cytolytic granules to regulate or assist in an immune response.

### II. SOURCE CELLS AND METHODS OF DIFFERENTIATING SAME TO CYTOKINE INNATE LYMPHOID (CIL) CELL

Provided herein are stem or progenitor cells containing a synthetic cytokine receptor. In some embodiments, the synthetic cytokine receptor is any as described in Section II. B. In some embodiments, the synthetic cytokine receptor contain a common gamma chain intracellular signaling domains (e.g. interleukin-2 receptor subunit gamma, IL-2RG) and a intracellular domain from interleukin-2 receptor subunit beta (IL-2RB), interleukin-7 receptor subunit beta (IL-7RB) or interleukin-21 receptor subunit beta (IL-21RB). In some embodiments, the synthetic cytokine receptor also contains an extracellular domain that is able to be bound by a non-physiological ligand (e.g. rapamycin or an analog). In this way, binding of the non-physiological ligand to the extracellular domain of the synthetic cytokine receptor activates cytokine receptor-mediated signaling to include JAK/STAT signaling, which is an important pathway for differentiation of stem cells, such as iPSCs or other pluripotent stem cells, to downstream cell linears, such as CILs. Thus, in the presence of a non-physiological ligand (e.g. rapamycin) the synthetic cytokine receptor can be engaged during cell differentiation removing the need for endogenous receptors or exogenous growth factors. In some embodiments, this increases the control and decreases the variability of JAK/STAT signaling during cell differentiation to thereby permit efficient generation of induced CILs (iCILs).

As described above, provided herein are stem or progenitor cells that may be differentiated into lymphoid cells using a synthetic cytokine receptor complex activated by a non-physiological ligand, and differentiated cells produced from those stem or progenitor cells for use in medical treatment. The differentiated cells may be, but are not limited to, iCIL cells. As a non-limiting illustration of the compositions and methods described herein, cytotoxic innate lymphoid cells may be produced from pluripotent stem cells, such as induced pluripotent stem cells, engineered to express synthetic cytokine receptor able to be activated by a non-physiological ligand (e.g. rapamycin) as described to induce differentiation, in addition to or instead of an exogenous cytokine. In some embodiments, the synthetic cytokine receptor is a rapamycin activated cytokine receptor (RACR) using rapamycin or a rapalog to induce differentiation, in addition to or instead of an exogenous cytokine. Advantages of embodiments may include the ability to generate from a plentiful cell source (e.g., induced pluripotent stem cells) effector cells expressing synthetic cytokine receptor complex activated by a non-physiological ligand, so that proliferation of the effector cells in patients may be controlled by administering or ceasing administration of the non-physiological ligand. Other advantages of embodiments include, but are not limited to, the ability to generate effector cells from source cells in media substantially free of cytokines conventionally used in the art for CIL cell differentiation, such as IL-2, IL-7, and/or IL-15.

### A. Source Cells

CIL cells may be generated from multiple sources, illustrative examples include: iPSCs, PBMCs, or UCBs. In some embodiments, the CIL source cells are autologous cells. In some embodiments, the CIL source cells are allogeneic cells. In some embodiments, the CIL source cells are heterologous cells. For example, when the subject being treated using the compositions of the present disclosure has received high-dose chemotherapy or radiation treatment to destroy the subject's immune system, allogenic cells may be used.

As used herein, the term “peripheral blood cell” is used to refer to cells that originate from circulating blood and comprise hematopoietic stem cells that are capable of proliferation, selectable differentiation, and maturation. As such, peripheral blood NK cells may alternatively be referred to as differentiated blood-derived NK cells (bdNK).

In some embodiments, the lymphocytes used to generate engineered stem cells or CIL cells may be obtained from a donor or a subject (for autologous therapy) by various means well-known in the art. For example, lymphocytes can be obtained by collecting peripheral blood from the patient and subjecting the blood to Ficoll density gradient centrifugation and/or leukapheresis, and then using an isolation kit to isolate a population of lymphocytes from the peripheral blood. In one illustrative embodiment, the population of lymphocytes need not be pure of the selected cell type and may contain other cell types such as T cells, monocytes, macrophages, natural killer cells, and B cells. In some embodiments, the cell population being collected can comprise at least about 90% of the selected cell type, at least about 91%, 92%, 93%, 94%, 95%, 96%, 97%, 98%, 99%, or 100% of the selected cell type.

In particular embodiments, the CIL cells are generated from a stem cell source.

In some embodiments, the source cell includes hematopoietic stem cells, characterized as being CD34+ and/or CD45+; common lymphoid progenitor cells, characterized as being CD45+ CD7+ CD56−; CIL progenitor cells characterized as being CD45+ CD5− CD7+; and/or CIL cells, characterized as being CD45+ CD56+ CD3− and optionally CD5− and/or CD7+.

In some embodiments, the stem cells are pluripotent stem cells. Various sources of pluripotent stem cells can be used in the method, including embryonic stem (ES) cells and induced pluripotent stem cells (iPSCs). Various sources of pluripotent stem cells can be used in the method, including embryonic stem (ES) cells and induced pluripotent stem cells (iPSCs). In some embodiments, pluripotent stem cells are induced pluripotent stem cells (iPSCs), artificially derived from a non-pluripotent cell. In some aspects, a non-pluripotent cell is a cell of lesser potency to self-renew and differentiate than a pluripotent stem cell. iPSCs may be generated by a process known as reprogramming, wherein non-pluripotent cells are effectively “dedifferentiated” to an embryonic stem cell-like state by engineering them to express genes such as OCT4, SOX2, and KLF4. Takahashi and Yamanaka Cell (2006) 126:663-76.

In some embodiments, source cells may be human embryonic stem cell (hESC) or induced pluripotent stem cell (iPSC). In immunotherapy, source cells be allogeneic or autologous, meaning from a donor or from the subject, respectively.

In some embodiments, CIL cells may be generated from induced pluripotent stem cells (iPSCs). iPSCs are a type of pluripotent stem cell derived from adult somatic cells that have been genetically reprogrammed to an embryonic stem cell-like state through the forced expression of genes and factors important for maintaining the defining properties of embryonic stem cells. iPSCs may be generated from tissues with somatic cells, including, but not limited to, the skin, dental tissue, peripheral blood, and urine. To generate iPSCs, somatic cells may be reprogrammed through methods including, but not limited to, the transient expression of reprogramming factors, virus-free methods, adenoviruses, plasmids, minicircle vectors, episomal vectors, Sendai viruses, synthetic mRNAs, self-replicating RNAs, retroviruses, lentiviruses, PhiC31 integrases, excisable transposons, CRISPR-based gene editing, or recombinant proteins.

While the cell therapy industry has demonstrated the transformative potential of using gene-engineered, patient-derived cells for treating specific disease indications, many challenges remain with using patient-derived materials, including limited expansion capacity and scalability, manufacturing complexity, high cost, variability from patient to patient, and patient access. In contrast, iPSCs are pluripotent stem cells, a type of cell theoretically capable of differentiating into any other cell type-including natural killer (NK) cells that are applicable to the treatment of cancer. Using iPSCs, it is possible to provide a scalable and simplified manufacturing of a target cell-fighting (e.g. cancer fighting) cell therapy like NK cells, thus reducing costs and improving patient access to the cell therapies. iPSCs possess an unlimited expansion capacity, meaning they can reproduce and proliferate indefinitely, potentially generating a nearly endless supply of differentiated immune cells for therapy, such as for cancer therapies. iPSCs are also amenable to precision multiplex genome editing, allowing introduction of multiple genetic modifications to enhance their disease targeting capabilities and safety of the immune cells they eventually become. iPSCs can similarly be engineered with the goal of protecting them against allogeneic rejection by the patient's own immune system, improving both their initial expansion and duration of engraftment. Furthermore, while either patient-derived or donor-derived blood materials are inconsistent, iPSCs provide a consistent starting material originating from a single cellular clone, which can permit genomic consistency and integrity in the final cellular product.

In some embodiments, the PSCs (e.g. iPSCs) are autologous to the subject to be treated, i.e. the PSCs are derived from the same subject to whom the differentiated cells are administered. In some embodiments, non-pluripotent cells (e.g., fibroblasts) derived from patients to be treated are reprogrammed to become iPSCs before differentiation into CILs as described herein. In some embodiments, fibroblasts may be reprogrammed to iPSCs by transforming fibroblasts with genes (OCT4, SOX2, NANOG, LIN28, and KLF4) cloned into a plasmid (for example, see, Yu, et al., Science DOI: 10.1126/science.1172482). In some embodiments, non-pluripotent fibroblasts derived from patients are reprogrammed to become iPSCs before differentiation into CILs, such as by use of the non-integrating Sendai virus to reprogram the cells (e.g., use of CTS™ CytoTune™-iPS 2.1 Sendai Reprogramming Kit). In some embodiments, the resulting differentiated cells are then administered to the patient from whom they are derived in an autologous cell therapy.

In some embodiments, the PSCs (e.g., iPSCs) are allogeneic to the subject to be treated, i.e. the PSCs are derived from a different individual than the subject to whom the differentiated cells will be administered. In some embodiments, non-pluripotent cells (e.g., fibroblasts) derived from another individual (e.g. an individual not having a disease or condition to be treated, such as a healthy subject) are reprogrammed to become iPSCs before differentiation into CILs. In some embodiments, reprogramming is accomplished, at least in part, by use of the non-integrating Sendai virus to reprogram the cells (e.g., use of CTS™ CytoTunc™-iPS 2.1 Sendai Reprogramming Kit). In some embodiments, the resulting differentiated cells are then administered to an individual who is not the same individual from whom the differentiated cells are derived (e.g. allogeneic cell therapy or allogeneic cell transplantation). In such embodiments, the PSCs described herein (e.g. allogeneic cells) may be genetically engineered to be hypoimmunogenic. Methods for reducing the immunogenicity are known, and include ablating polymorphic HLA-A/-B/-C and HLA class II molecule expression. Exemplary methods for reducing one or more HLA molecules include disrupting the beta-2-microglobulin (B2M) gene, such as described herein.

In some embodiments, iPSCs are genetically edited using a lentivirus. In some embodiments, iPSCs are genetically edited using CRISPR.

In some embodiments, HSCs are genetically edited using a lentivirus. In some embodiments, HSCs are genetically edited using CRISPR. In some embodiments, blood progenitor cells are genetically edited using a lentivirus. In some embodiments, blood progenitor cells are genetically edited using CRISPR. In some embodiments, common lymphoid progenitor cells are genetically edited using a lentivirus. In some embodiments, common lymphoid progenitor cells are genetically edited using CRISPR. In some embodiments, common lymphoid progenitor (CLP) cells are genetically edited using a lentivirus. In some embodiments, common lymphoid progenitor (CLP) cells are genetically edited using CRISPR. Exemplary methods for gene editing are described in Section III.

In some embodiments, stem cells may be engineered to express a synthetic cytokine receptor.

In some embodiments, iPSCs may be engineered to express a synthetic cytokine receptor.

In some embodiments, hematopoietic stem cells may be engineered to express a synthetic cytokine receptor. In some embodiments, blood progenitor cells (leukocytes) cells may be engineered to express a synthetic cytokine receptor. In some embodiments, common lymphoid progenitor cells may be engineered to express a synthetic cytokine receptor. In some embodiments, CIL cells may be engineered to express a synthetic cytokine receptor.

The methods for producing CIL cells may comprise an ex vivo culturing process, wherein the CIL cells are differentiated from a non-terminally differentiated cell. In some embodiments, the non-terminally differentiated cell is a stem cell. In some embodiments, the non-terminally differentiated cell is an iPSC cell. In some embodiments, the non-terminally differentiated cell is a progenitor cell. In embodiments, the non-terminally differentiated cells (e.g. stem cells, such as iPSC) expresses a synthetic cytokine receptor. In an aspect, the disclosure provides a method of producing CIL cells comprising providing stem or progenitor cells and differentiating the cells into CIL cells by controlled activation of the synthetic cytokine receptor, or without activation of the synthetic cytokine receptor. In some embodiments, the differentiation is carried out by activation of the synthetic cytokine receptor without any additional cytokines (e.g. without one or more of IL-2, IL-15, and IL-7). In some embodiments, the differentiation is carried out by activation of the synthetic cytokine receptor with one or more additional cytokines. In some embodiments, differentiation also may be carried out with cytokines without activation of the synthetic cytokine receptor.

In any of the provided embodiments, the synthetic cytokine receptor may be any as described herein that is able to be activated by a non-physiological ligand (e.g. rapamycin). In some embodiments, the synthetic cytokine receptor is a rapamycin activated cytokine receptor (RACR) that is able to be activated by rapamycin or a rapalog. In provided embodiments, activation of the synthetic cytokine receptor induces differentiation, in addition to or instead of an exogenous cytokine.

In some embodiments, the non-physiological ligand may induce differentiation, in addition to or instead of an exogenous cytokine. In some embodiments, the non-physiological ligand may induce differentiation during one or more of mesoderm formation, hematopoietic specification, lymphoid progenitor differentiation, CIL cell differentiation.

In some embodiments, the non-physiological ligand may be contacted with cells in a differentiation phase requiring an IL-7 and/or IL-15 signal.

In some embodiments, the non-physiological ligand may be contacted with cells in an expansion phase requiring an IL-2 signal.

In some embodiments, engineering cells to express a synthetic cytokine receptor and activating the receptor with a non-physiological ligand allows for the generation of CIL cells that may be differentiated and/or expanded without the use of exogenous factors.

In some embodiments, engineering iPSCs to express a RACR and activating the receptor with rapamycin or a rapalog allows for the generation of CIL cells that are differentiated without the use of exogenous factors, such as without IL-15 and/or IL-7.

In some embodiments, engineering iPSCs to express a RACR and activating the receptor with rapamycin or a rapalog allows for the generation of CIL cells that are expanded without the use of exogenous factors, such as without IL-2 and/or IL-7.

Because it is well-known in the relevant art that differentiation and/or expansion of CIL cells depends on the presence of various exogenous stimuli, the differentiation and/or expansion of the engineered CIL cells without one, two, or all of IL-2, IL-15, and IL-7, as described herein, is surprising and unexpected.

In some embodiments, engineered common lymphoid progenitors are differentiated into CIL cells without IL-15 in the differentiation medium.

In some embodiments, engineered common lymphoid progenitors are differentiated into CIL cells without IL-7 in the differentiation medium.

In some embodiments, engineered common lymphoid progenitors are differentiated into CIL cells without IL-15 or IL-7 in the differentiation medium.

In some embodiments, common lymphoid progenitors are engineered to express a synthetic cytokine receptor, such as RACR, and differentiated into CIL cells with a rapalog in the differentiation medium and without IL-15 or IL-7 in the differentiation medium.

### B. Synthetic Cytokine Receptor Complex

The synthetic cytokine receptors of the present disclosure comprise a synthetic gamma chain and a synthetic beta chain, each comprising a dimerization domain. The dimerization domains controllable dimerize in the present of a non-physiological ligand, thereby activating signaling the synthetic cytokine receptor.

The synthetic gamma chain polypeptide comprises a first dimerization domain, a first transmembrane domain, and an interleukin-2 receptor subunit gamma (IL-2RG) intracellular domain. The dimerization domain may be extracellular (N-terminal to the transmembrane domain) or intracellular (C-terminal to the transmembrane domain and N- or C-terminal to the IL-2G intracellular domain.

The synthetic beta chain polypeptide comprises a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, or an interleukin-21 receptor subunit beta (IL-21RB) intracellular domain. The dimerization domain may be extracellular (N-terminal to the transmembrane domain) or intracellular (C-terminal to the transmembrane domain and N- or C-terminal to the IL-2RB or IL-7RB intracellular domain).

In some embodiments, the synthetic gamma chain polypeptide is encoded by a nucleic acid sequence that encodes a signal peptide. In some embodiments, the synthetic beta chain polypeptide is encoded by a nucleic acid sequence that encodes a signal peptide. A skilled artisan is readily familiar with signal peptides that can provide a signal to transport a nascent protein in the cells. Any of a variety of signal peptides can be employed.

In some embodiments, the signal peptide is a CD8a signal sequence shown as SEQ ID NO: 12: MALPVTALLLPLALLLHAARP.

In some embodiments, the signal peptide is a signal sequence shown as SEQ ID NO: 29: MPLGLLWLGLALLGALHAQA

In some embodiments, the non-physiological ligand activates the synthetic cytokine receptor in the cytotoxic innate lymphoid cells to induce expansion and/or activation of the engineered cytotoxic innate lymphoid cells. In some embodiments, the non-physiological ligand is rapamycin or a rapalog, such synthetic cytokine receptor termed a rapamycin-activated cytokine receptor (RACR).

In some embodiments, the non-physiological ligand activates the synthetic cytokine receptor in the CIL cells to induce expansion of the CIL cells. In some embodiments, the activation of the synthetic cytokine receptor results in at least about 10-fold, at least about 50-fold, at least about 100-fold, at least about 200-fold, at least about 300-fold, at least about 400-fold, at least about 500-fold, at least about 1000-fold, at least about 1500-fold, at least about 2000-fold, at least about 2500-fold, at least about 3000-fold, at least about 3500-fold, or at least about 4000-fold increased number of CIL cells compared to uninduced cells. In some embodiments, the activation of the synthetic cytokine receptor results in at least about 5000-fold, at least about 6000-fold, at least about 7000-fold, at least about 8000-fold, at least about 9000-fold, at least about 10,000-fold, at least about 50,000-fold, at least about 100,000-fold, at least about 250,000-fold, at least about 500,000-fold, at least about 750,000-fold, or at least about 1,000,000-fold increased number of CIL cells compared to uninduced cells.

In some embodiments, the CIL cells increase by about 10-fold to about 100-fold, about 50-fold to about 200-fold, about 100-fold to about 300-fold, about 200-fold to about 400-fold, about 300-fold to about 500-fold, about 400-fold to about 1000-fold, about 500-fold to about 1500-fold, about 1000-fold to about 2000-fold, about 1500-fold to about 2500-fold, about 2000-fold to about 3000-fold, about 2500-fold to about 3500-fold, about 3000-fold to about 4000-fold, or any value in between these ranges. In some embodiments, the CIL cells increase by about 4000-fold to about 6000-fold, about 5000-fold to about 7000-fold, about 6000-fold to about 8000-fold, about 7000-fold to about 9000-fold, about 8000-fold to about 10000-fold, about 9000-fold to about 50,000-fold, about 10000-fold to about 100,000-fold, about 50,000-fold to about 250,000-fold, about 100,000-fold to about 500,000-fold, about 250,000-fold to about 750,000-fold, about 500,000-fold to about 1,000,000-fold, or any value in between these ranges.

In some embodiments, the non-physiological ligand activates the synthetic cytokine receptor in the stem cells to induce differentiation. In some embodiments, the non-physiological ligand is rapamycin or a rapalog, such synthetic cytokine receptor termed a rapamycin-activated cytokine receptor (RACR).

In some embodiments, the non-physiological ligand activates the synthetic cytokine receptor in the stem cells to induce expansion of the hematopoietic progenitors or CLPs differentiated from the stem cells. In some embodiments, the activation of the synthetic cytokine receptor results in at least about 10-fold, at least about 50-fold, at least about 100-fold, at least about 200-fold, at least about 300-fold, at least about 400-fold, at least about 500-fold, at least about 1000-fold, at least about 1500-fold, at least about 2000-fold, at least about 2500-fold, at least about 3000-fold, at least about 3500-fold, or at least about 4000-fold increased number of hematopoietic progenitors or CLPs compared to non-engineered cells.

In some embodiments, the hematopoietic progenitors or CLPs increase by about 10-fold to about 100-fold, about 50-fold to about 200-fold, about 100-fold to about 300-fold, about 200-fold to about 400-fold, about 300-fold to about 500-fold, about 400-fold to about 1000-fold, about 500-fold to about 1500-fold, about 1000-fold to about 2000-fold, about 1500-fold to about 2500-fold, about 2000-fold to about 3000-fold, about 2500-fold to about 3500-fold, about 3000-fold to about 4000-fold, or any value in between these ranges.

### 1. Intracellular Domain

In some embodiments, the intracellular signaling domain of the first transmembrane receptor protein comprises an interleukin-2 receptor subunit gamma (IL2Rg) domain. In some embodiments, the IL2Rg domain comprises the sequence set forth in SEQ ID NO: 1. In some embodiments, the IL2Rg Common Gamma Chain Intracellular domain has at least 80% amino acid identity, at least 85% amino acid identity, at least 90% amino acid identity, at least 95% amino acid identity, or 100% amino acid identity to SEQ ID NO: 1.

The sequence of a IL2RG Common Gamma Chain Intracellular domain is set forth in SEQ ID NO: 1:

ERTMPRIPTLKNLEDLVTEYHGNFSAWSGVSKGLAESLQPDYSERLCLVS
EIPPKGGALGEGPGASPCNQHSPYWAPPCYTLKPET.

In some embodiments, the synthetic cytokine receptor comprises a first transmembrane receptor protein comprising an IL-2RG intracellular domain, a first dimerization domain, a second transmembrane receptor protein comprising an IL-2RB intracellular domain, and a second dimerization domain.

In some embodiments, the synthetic beta chain comprises an interleukin-2 receptor subunit beta (IL2RB) intracellular domain. IL2RB is also known as IL15RB or CD122. Thus, when referred to herein, IL2RB can also mean IL15RB. That is, the terms are used interchangeably in the present disclosure. In some embodiments, the IL2RB intracellular domain comprises the sequence set forth in SEQ ID NO: 2. In some embodiments, the IL2RB intracellular domain has at least 80% amino acid identity, at least 85% amino acid identity, at least 90% amino acid identity, at least 95% amino acid identity, or 100% amino acid identity to SEQ ID NO: 2.

The sequence of a IL2RB intracellular domain is set forth in SEQ ID NO: 2:

NCRNTGPWLKKVLKCNTPDPSKFFSQLSSEHGGDVQKWLSSPFPSSSFSP
GGLAPEISPLEVLERDKVTQLLLQQDKVPEPASLSSNHSLTSCFTNQGYF
FFHLPDALEIEACQVYFTYDPYSEEDPDEGVAGAPTGSSPQPLQPLSGED
DAYCTFPSRDDLLLFSPSLLGGPSPPSTAPGGSGAGEERMPPSLQERVPR
DWDPQPLGPPTPGVPDLVDFQPPPELVLREAGEEVPDAGPREGVSFPWSR
PPGQGEFRALNARLPLNTDAYLSLQELQGQDPTHLV

In some embodiments, the synthetic cytokine receptor comprises a first transmembrane receptor protein comprising an IL-2RG intracellular domain, a first dimerization domain, a second transmembrane receptor protein comprising an IL-7RB intracellular domain, and a second dimerization domain.

In some embodiments, the synthetic beta chain comprises an interleukin-7 receptor subunit beta (IL7RB) intracellular domain. In some embodiments, the IL7RB intracellular domain comprises the sequence set forth in SEQ ID NO: 3. In some embodiments, the IL7RB intracellular domain has at least 80% amino acid identity, at least 85% amino acid identity, at least 90% amino acid identity, at least 95% amino acid identity, or 100% amino acid identity to SEQ ID NO: 3.

The sequence of a IL7RB intracellular domain is set forth in SEQ ID NO: 3:

KKRIKPIVWPSLPDHKKTLEHLCKKPRKNLNVSFNPESFLDCQIHRVDDI
QARDEVEGFLQDTFPQQLEESEKQRLGGDVQSPNCPSEDVVITPESFGRD
SSLTCLAGNVSACDAPILSSSRSLDCRESGKNGPHVYQDLLLSLGTTNST
LPPPFSLQSGILTLNPVAQGQPILTSLGSNQEEAYVTMSSFYQNQ

In some embodiments, the synthetic cytokine receptor comprises a first transmembrane receptor protein comprising an IL-2RG intracellular domain, a first dimerization domain, a second transmembrane receptor protein comprising an IL-21RB intracellular domain, and a second dimerization domain.

In some embodiments, the synthetic beta chain comprises an interleukin-21 receptor subunit beta (IL21RB) intracellular domain. In some embodiments, the IL21RB intracellular domain comprises the sequence set forth in SEQ ID NO: 4. In some embodiments, the IL21RB intracellular domain has at least 80% amino acid identity, at least 85% amino acid identity, at least 90% amino acid identity, at least 95% amino acid identity, or 100% amino acid identity to SEQ ID NO: 4.

The sequence of a IL21RB intracellular domain is set forth in SEQ ID NO: 4:

SLKTHPLWRLWKKIWAVPSPERFFMPLYKGCSGDFKKWVGAPFTGSSLEL
GPWSPEVPSTLEVYSCHPPRSPAKRLQLTELQEPAELVESDGVPKPSFWP
TAQNSGGSAYSEERDRPYGLVSIDTVTVLDAEGPCTWPCSCEDDGYPALD
LDAGLEPSPGLEDPLLDAGTTVLSCGCVSAGSPGLGGPLGSLLDRLKPPL
ADGEDWAGGLPWGGRSPGGVSESEAGSPLAGLDMDTFDSGFVGSDCSSPV
ECDFTSPGDEGPPRSYLRQWVVIPPPLSSPGPQAS

### 2. Dimerization Domain

The dimerization domains may be heterodimerization domains, including but not limited to FK506-Binding Protein of size 12 kD (FKBP) and a FKBP12-rapamycin binding (FRB) domain, which are known in the art to dimerize in the presence of rapamycin or a rapalog. In some embodiments, the FRB domain may comprise a polypeptide sequence at least 80%, at least 90%, at least 95%, at least 96%, at least 97%, at least 98%, at least 99%, or 100% identical to SEQ ID NO: 6 or SEQ ID NO:7. In some embodiments, The FKBP domain may comprise a polypeptide sequence at least 80%, at least 90%, at least 95%, at least 96%, at least 97%, at least 98%, at least 99%, or 100% identical to SEQ ID NO: 5. In some embodiments, The FKBP domain may comprise a polypeptide sequence at least 80%, at least 90%, at least 95%, at least 96%, at least 97%, at least 98%, at least 99%, or 100% identical to SEQ ID NO: 49. In some embodiments, The FKBP domain may comprise a polypeptide sequence at least 80%, at least 90%, at least 95%, at least 96%, at least 97%, at least 98%, at least 99%, or 100% identical to SEQ ID NO: 30.

In some embodiments, the sequence of an illustrative FKBP domain is set forth in SEQ ID NO: 5:

GVQVETISPGDGRTFPKRGQTCVVHYTGMLEDGKKFDSSRDRNKPFKFML
GKQEVIRGWEEGVAQMSVGQRAKLTISPDYAYGATGHPGIIPPHATLVFD
VELLKLE

In some embodiments, the sequence of an illustrative FKBP domain is set forth in SEQ ID NO: 49:

GVQVETISPGDGRTFPKRGQTCVVHYTGMLEDGKKFDSSRDRNKPFKFM
LGKQEVIRGWEEGVAQMSVGQRAKLTISPDYAYGATGHPGIIPPHATLV
FDVELLKL

In some embodiments, the sequence of an illustrative FKBP domain is set forth in SEQ ID NO: 30:

GVQVETISPGDGRTFPKRGQTCVVHYTGMLEDGKKFDSSRDRNKPFKFM
LGKQEVIRGWEEGVAQMSVGQRAKLTISPDYAYGATGHPGIIPPHATLV
FDVELLKLGE

In some embodiments, the sequence of an illustrative FRB domain is set forth in SEQ ID NO: 6:

ILWHEMWHEGLEEASRLYFGERNVKGMFEVLEPLHAMMERGPQTLKETS
FNQAYGRDLMEAQEWCRKYMKSGNVKDLTQAWDLYYHVFRRISK

In some embodiments, the sequence of variant FRB domain (FRB mutant domain) is set forth in SEQ ID NO: 7:

ILWHEMWHEGLEEASRLYFGERNVKGMFEVLEPLHAMMERGPQTLKETS
FNQAYGRDLMEAQEWCRKYMKSGNVKDLLQAWDLYYHVFRRISK

In some embodiments, the first dimerization domain is set forth in SEQ ID NO:5 and the second dimerization domain is set forth in SEQ ID NO:6.

In some embodiments, the first dimerization domain is set forth in SEQ ID NO:49 and the second dimerization domain is set forth in SEQ ID NO:6.

In some embodiments, the first dimerization domain is set forth in SEQ ID NO:30 and the second dimerization domain is set forth in SEQ ID NO:6.

In some embodiments, the first dimerization domain is set forth in SEQ ID NO:5 and the second dimerization domain is set forth in SEQ ID NO:7.

In some embodiments, the first dimerization domain is set forth in SEQ ID NO:49 and the second dimerization domain is set forth in SEQ ID NO:7.

In some embodiments, the first dimerization domain is set forth in SEQ ID NO:30 and the second dimerization domain is set forth in SEQ ID NO:7.

Alternatively, the first dimerization domain and the second dimerization domain may be a FK506-Binding Protein of size 12 kD (FKBP) and a calcineurin domain, which are known in the art to dimerize in the presence of FK506 or an analogue thereof.

In some embodiments the dimerization domains are homodimerization domains selected from:
i) FK506-Binding Protein of size 12 kD (FKBP);
ii) ii) cyclophiliA (CypA); or
iii) iii) gyrase B (CyrB);
with the corresponding non-physiological ligands being, respectively
i) FK1012, AP1510, AP1903, or AP20187;
ii) ii) cyclosporin-A (CsA); or
iii) iii) coumermycin or analogs thereof.

In some embodiments, the first and second dimerization domains of the transmembrane receptor proteins are a FKBP domain and a cyclophilin domain.

In some embodiments, the first and second dimerization domains of the transmembrane receptor proteins are a FKBP domain and a bacterial dihydrofolate reductase (DHFR) domain.

In some embodiments, the first and second dimerization domains of the transmembrane receptor proteins are a calcineurin domain and a cyclophilin domain.

In some embodiments, the first and second dimerization domains of the transmembrane receptor proteins are PYR1-like 1 (PYL1) and abscisic acid insensitive 1 (ABI1).

### 3. Transmembrane Domains

The transmembrane domain is the sequence of the synthetic cytokine receptor that spans the membrane. The transmembrane domain may comprise a hydrophobic alpha helix. In some embodiments, the transmembrane domain is derived from a human protein.

The sequence of a transmembrane (TM) domain is shown as SEQ ID NO: 8:

VVISVGSMGLIISLLCVYFWL

The sequence of a TM domain is shown as SEQ ID NO: 9:

VAVAGCVFLLISVLLLSGL

The sequence of TM domain is shown as SEQ ID NO: 10:

PILLTISILSFFSVALLVILACVLW

The sequence of a TM domain is shown as SEQ ID NO: 11:

GWNPHLLLLLLLVIVFIPAFW

The sequence of a TM domain is shown as SEQ ID NO: 36:

IPWLGHLLVGLSGAFGFIILVYLLI.

In some embodiments, the TM domain and the intracellular signaling domain are from the same cytokine receptor. In some embodiments, the synthetic gamma chain polypeptide contains an IL-2RG TM domain and a IL-2RG intracellular domain. In some embodiments, the synthetic beta chain polypeptide contains an IL-2RB TM domain and a IL-2RB intracellular domain. In some embodiments, the synthetic beta chain polypeptide contains an IL-7RB TM domain and a IL-7RB intracellular domain. In some embodiments, the synthetic beta chain polypeptide contains an IL-21RB TM domain and a IL-21RB intracellular domain.

In some embodiments, one or more additional contiguous amino acids of the ectodomain directly adjacent to the TM domain of the cytokine receptor also can be included as part of the polypeptide sequence of a chain of the synthetic cytokine receptor. In some embodiments, 1-20 contiguous amino acids of the ectodomain adjacent to the TM domain of the cytokine receptor is included as part of the polypeptide sequence of a chain of the synthetic cytokine receptor. The portion of the ectodomain may be a contiguous sequence of 1, 2, 3, 4, 5, 6, 7, 8, 9, 10, 11, 12, 13, 14, 15, 16, 17, 18, 19 or 20 amino acids directly adjacent (e.g. N-terminal to) the TM sequence.

In some embodiments, the synthetic gamma chain polypeptide contains an IL-2RG TM domain comprising the sequence set forth in SEQ ID NO: 8 and a IL-2RG intracellular domain comprising the sequence set forth in SEQ ID NO: 1. In some embodiments, the synthetic gamma chain polypeptide contains an IL-2RG TM domain comprising the sequence set forth in SEQ ID NO: 31 and a IL-2RG intracellular domain comprising the sequence set forth in SEQ ID NO:1.

In some embodiments, the synthetic beta chain polypeptide contains an IL-2RB TM domain comprising the sequence set forth in SEQ ID NO: 36 and a IL-2RB intracellular domain comprising the sequence set forth in SEQ ID NO:2. In some embodiments, the synthetic beta chain polypeptide contains an IL-2RB TM domain comprising the sequence set forth in SEQ ID NO: 35 and a IL-2RB intracellular domain comprising the sequence set forth in SEQ ID NO:2.

In some embodiments, the synthetic cytokine receptor is composed of a synthetic gamma chain polypeptide that contains a FKBP12 dimerization domain and an IL-2RG intracellular domain, and a synthetic beta chain polypeptide that contains a FRB dimerization domain and an IL-2RB intracellular domain. In some embodiments, the synthetic gamma chain polypeptide has the sequence of amino acids set forth in SEQ ID NO:28 or a sequence of amino acids that is at least 90%, at least 95%, at least 96%, at least 97%, at least 98%, at least 99%, or 100% identical to SEQ ID NO: 28. In some embodiments, the synthetic beta chain polypeptide has the sequence of amino acids set forth in SEQ ID NO:33 or a sequence of amino acids that is at least 90%, at least 95%, at least 96%, at least 97%, at least 98%, at least 99%, or 100% identical to SEQ ID NO: 33.

In some embodiments, the synthetic cytokine receptor is composed of a synthetic gamma chain polypeptide set forth in SEQ ID NO:28 and a synthetic beta chain polypeptide set forth in SEQ ID NO:33.

In some embodiments, the synthetic cytokine receptor is able to be bound by the non-physiological ligand rapamycin or a rapamycin analog. In some embodiments, the synthetic cytokine receptor is responsive to the non-physiological ligand rapamycin or a rapamycin analog, in which binding of the non-physiological ligand to the dimerization domains of the synthetic cytokine receptor induces cytokine receptor-mediated signaling in the cell, such as via the JAK/STAT pathway.

### 4. Non-Physiological Ligand

In various embodiments of the compositions and methods of the disclosure, the system comprises a non-physiological ligand. Illustrative small molecules useful as ligands include, without limitation: rapamycin, fluorescein, fluorescein isothiocyanate (FITC), 4-[(6-methylpyrazin-2-yl) oxy] benzoic acid (aMPOB), folate, rhodamine, acetazolamide, and a CA9 ligand.

In some embodiments, the synthetic cytokine receptor is activated by a ligand. In some embodiments, the ligand is a non-physiological ligand.

In some embodiments, the non-physiological ligand is a rapalog.

In some embodiments, the non-physiological ligand is rapamycin.

In some embodiments, the non-physiological ligand is AP21967.

In some embodiments, the non-physiological ligand is FK506.

In some embodiments, the non-physiological ligand is FK1012. In some embodiments, the non-physiological ligand is AP1510. In some embodiments, the non-physiological ligand is AP1903. In some embodiments, the non-physiological ligand is AP20187. In some embodiments, the non-physiological ligand is cyclosporin-A (CsA). In some embodiments, the non-physiological ligand is coumermycin.

In some embodiments, the synthetic cytokine receptor complex activated by folate, fluorescein, aMPOB, acetazolamide, a CA9 ligand, tacrolimus, rapamycin, a rapalog (a rapamycin analog), CD28 ligand, poly (his) tag, Strep-tag, FLAG-tag, VS-tag, Myc-tag, HA-tag, NE-tag, biotin, digoxigenin, dinitrophenol, or a derivative thereof.

In some embodiments, the non-physiological ligand may be an inorganic or organic compound that is less than 1000 Daltons.

In some embodiments, the ligand may be rapamycin or a rapamycin analog (rapalog). In some embodiments, the rapalog comprises variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring.

Thus, in some embodiments, the rapalog is everolimus, novolimus, pimecrolimus, ridaforolimus, tacrolimus, temsirolimus, umirolimus, zotarolimus, Temsirolimus (CCI-779), C20-methallylrapamycin, C16-(S)-3-methylindolerapamycin, C16-(S)-3-methylindolerapamycin (C16-iRap), AP21967 (A/C Heterodimerizer, Takara Bio®), sodium mycophenolic acid, benidipine hydrochloride, rapamine, AP23573 (Ridaforolimus), AP1903 (Rimiducid), or metabolites, derivatives, and/or combinations thereof.

In some embodiments, the ligand comprises FK1012 (a semisynthetic dimer of FK506), tacrolimus (FK506), FKCsA (a composite of FK506 and cyclosporine), rapamycin, coumermycin, gibberellin, HaXS dimerizer (chemical dimerizers of HaloTag and SNAP-tag), TmP-HTag (trimethoprim haloenzyme protein dimerizer), or ABT-737 or functional derivatives thereof.

In some embodiments, the non-physiological ligand is present or provided in an amount from 0 nM to 1000 nM such as, e.g., 0.05 nM, 0.1 nM, 0.5 nM, 1.0 nM, 5.0 nM, 10.0 nM, 15.0 nM, 20.0 nM, 25.0 nM, 30.0 nM, 35.0 nM, 40.0 nM, 45.0 nM, 50.0 nM, 55.0 nM, 60.0 nM, 65.0 nM, 70.0 nM, 75.0 nM, 80.0 nM, 90.0 nM, 95.0 nM, 100 nM, 200 nM, 300 nM, 400 nM, 500 nM, 600 nM, 700 nM, 800 nM, 900 nM, or 1000 nM, or an amount that is within a range defined by any two of the aforementioned amounts.

In some embodiments, the non-physiological ligand is AP21967 and is present or provided at 10 nM. In some embodiments, the non-physiological ligand is AP21967 and is present or provided at 20 nM. In some embodiments, the non-physiological ligand is AP21967 and is present or provided at 50 nM. In some embodiments, the non-physiological ligand is AP21967 and is present or provided at 100 nM.

In some embodiments, the non-physiological ligand is rapamycin and is present or provided at 1 nM. In some embodiments, the non-physiological ligand is rapamycin and is present or provided at 10 nM. In some embodiments, the non-physiological ligand is rapamycin and is present or provided at 20 nM. In some embodiments, the non-physiological ligand is rapamycin and is present or provided at 50 nM.

In some embodiments, the non-physiological ligand is a rapalog and is present or provided at 1 nM. In some embodiments, the non-physiological ligand is a rapalog and is present or provided at 10 nM. In some embodiments, the non-physiological ligand is a rapalog and is present or provided at 20 nM. In some embodiments, the non-physiological ligand is a rapalog and is present or provided at 50 nM. In some embodiments, the non-physiological ligand is a rapalog and is present or provided at 100 nM.

In some embodiments, the non-physiological ligand is present or provided at 1 nM.

In some embodiments, the non-physiological ligand is present or provided at 10 nM.

In some embodiments, the non-physiological ligand is present or provided at 100 nM.

In some embodiments, the non-physiological ligand is present or provided at 1000 nM.

### C. Cytosolic FRB

In some embodiments, the engineered cells, such as stem cells or iCIL cells, can be contacted with free cytosolic FRB. As described in more detail elsewhere herein, rapamycin normally binds to FBP12, and the FKBP12-rapamycin complex then binds to the FRB subunit of mTOR and blocks mTOR signaling. Thus, contacting a cell with rapamycin could, in some cases, inhibit or reduce cell growth and expansion. In some embodiments, the cells can be made “rapamycin resistance” by providing free cytosolic FRB to the cell in order to complex with rapamycin and thereby eliminate or reduce rapamycin-mediated growth inhibition of a source cell or iCIL.

In some embodiments, soluble FRB can be microinjected into a stem cell or NK cell to eliminate or reduce rapamycin-mediated growth inhibition. In some embodiments, a stem cell or NK cell can be transduced with a vector containing soluble FRB to eliminate or reduce rapamycin-mediated growth inhibition. In some embodiments, soluble FRB can be added to cell culture media to eliminate or reduce rapamycin mediated growth inhibition.

In an embodiment where soluble FRB is microinjected into a stem cell or NK cell, the soluble FRB is injected at a concentration of 4 mg/mL, 4.5 mg/mL, 5 mg/mL, 5.5 mg/mL, or 6 mg/mL. In an embodiment where soluble FRB is microinjected into a stem cell or NK cell, the soluble FRB is injected at a concentration of 1 μM.

In some embodiments, a nucleic acid molecule encoding FRB, such as by introduction of a vector construct encoding FRB, is introduced into the cell. In some embodiments, the construct is designed for insertion of the nucleic acid encoding FRB into an endogenous locus in the cell. Methods of gene insertion or knock-in are known, including any of the methods described in Section III. In some embodiments, insertion of an FRB-encoding construct is by homology directed repair, such as by using a CRISPR-Cas system. In some embodiments, the engineered cell that expresses FRB at an endogenous loci is able to express free cytosolic FRB in the cell.

The FRB domain is an approximately 100 amino acid domain derived from the mTOR protein kinase. It may be expressed in the cytosol as a freely diffusible soluble protein. Advantageously, the FRB domain reduces the inhibitory effects of rapamycin on mTOR in the engineered cells and promote consistent activation of engineered cells giving the cells a proliferative advantage over native cells.

In some embodiments, synthetic cytokine receptor complex comprises a cytosolic polypeptide that binds to the ligand or a complex comprising the ligand.

In some embodiments, the cytosolic polypeptide comprises an FRB domain. In some embodiments, the cytosolic polypeptide comprises an FRB domain and the ligand is rapamycin. The cytosolic FRB domain may comprise a polypeptide sequence at least 80%, at least 90%, at least 95%, at least 96%, at least 97%, at least 98%, at least 99%, or 100% identical to SEQ ID NO: 6 or SEQ ID NO: 7. FRB domain may be a naked FRB domain consisting essentially of a polypeptide having a polypeptide sequence at least 80%, at least 90%, at least 95%, at least 96%, at least 97%, at least 98%, at least 99%, or 100% identical to SEQ ID NO: 6 or SEQ ID NO: 7.

In some embodiments, the cells are contacted with an FRB domain protein that has the sequence set forth in SEQ ID NO:6.

In some embodiments, the cells are contacted with an FRB domain protein that has the sequence set forth in SEQ ID NO:7.

Advantageously, the cytosolic FRB confers resistance to the immunosuppressive effect of the non-physiological ligand (e.g., rapamycin or rapalog).

### D. Engineered Stem Cells

In some embodiments, the disclosure provides engineered stem cells transiently or stably expressing a synthetic cytokine receptor complex. In some embodiments, the disclosure provides engineered stem cells stably expressing a synthetic cytokine receptor complex.

In some embodiments, the engineered stem cells comprise a genome comprising a nucleotide sequence encoding a synthetic cytokine receptor complex, such as described in Section II.B. In some embodiments, the genome further comprises a disrupted B2M, TRAC, and/or SIRPA locus. In some embodiments, the genome further comprises a disrupted FKBP12 locus. In some embodiments, a locus of a gene is disrupted by gene editing technologies, such as CRISPR-Cas systems. In some embodiments, a disrupted locus inactivates the gene in the cell. In some embodiments, a disrupted locus involves knockout of the gene in the cell. In some embodiments, the disrupted locus comprises an indel in the endogenous gene or a deletion of a contiguous stretch of genomic DNA of the endogenous ene. In some embodiments, the indel is a frameshift mutation or a deletion of a contiguous stretch of genomic DNA of the gene. In some embodiments, the indel is in both alleles of the gene (indellindel). Methods of gene editing and engineering are known, including methods described in Section III. Any of such methods can be used to generate engineered stem cells that further comprise a synthetic cytokine receptor complex as described herein.

In some embodiments, the engineered stem cells comprise a genome comprising (i) a nucleotide sequence encoding a synthetic cytokine receptor complex, such as described in Section II.B, and (ii) a disrupted B2M locus. In some embodiments, the engineered stem cells comprise a genome comprising (i) a nucleotide sequence encoding a synthetic cytokine receptor complex, such as described in Section II.B, (ii) a disrupted B2M locus, and (iii) a disrupted FKBP12 locus. In some embodiments, the engineered stem cells comprise a genome comprising (i) a nucleotide sequence encoding a synthetic cytokine receptor complex, (ii) a disrupted TRAC locus, and (iii) a disrupted FKBP12 locus. In some embodiments, the engineered stem cells comprise a genome comprising (i) a nucleotide sequence encoding a synthetic cytokine receptor complex, (ii) a disrupted SIRPA locus, and (iii) a disrupted FKBP12 locus. In some embodiments, the engineered stem cells comprise a genome comprising (i) a nucleotide sequence encoding a synthetic cytokine receptor complex, (ii) a disrupted B2M locus, (iii) a disrupted TRAC locus, and (iv) a disrupted FKBP12 locus. In some embodiments, the engineered stem cells comprise a genome comprising (i) a nucleotide sequence encoding a synthetic cytokine receptor complex, (ii) a disrupted B2M locus, (iii) a disrupted SIRPA locus, and (iv) a disrupted FKBP12 locus. In some embodiments, the engineered stem cells comprise a genome comprising (i) a nucleotide sequence encoding a synthetic cytokine receptor complex, (ii) a disrupted SIRPA locus, (iii) a disrupted TRAC locus, and (iv) a disrupted FKBP12 locus.

In some cases, the engineered stem cells further comprise a polynucleotide encoding a chimeric antigen receptor (CAR), thereby generating engineered stem cells expressing the CAR. Exemplary CARs and methods for engineering cells with a CAR are described in Sections III and IV.

In some cases, the engineered stem cells further comprise a polynucleotide encoding FRB, thereby generating engineered stem cells expressing cytosolic FRB. The FRB can have a sequence as described in Section II.C. Methods of engineering cells, such as with an exogenous FRB, are known, including any as described in Section III.

In some embodiments, engineered stem cells are iPSCs. In some embodiments, the engineered iPSCs are sequentially differentiated into hematopoietic progenitor cells (HPCs); the HPCs into common lymphoid progenitor cells (CLPs); and then the CLPs into CIL cells-termed “iCIL” cells. In a variation, CIL cells may be derived from HPCs by sequentially differentiating the HPCs into CLPs; and then the CLPs into iCIL cells. In a further variation, CIL cells may be derived by differentiating CLPs into iCIL cells.

### E. Differentiation of Cells

Provided herein are methods of differentiating pluripotent stem cells, such as iPSCs, engineered with a synthetic cytokine receptor into a CIL cell. In some embodiments, the iPSC differentiation is by a pathway that includes differentiation into hematopoietic progenitors (HP) and common lymphoid progenitors (CLP). In some embodiments, the differentiation is directed, at least in part, by the signaling induced from the synthetic cytokine receptor. In embodiments of the provided methods, engagement of the synthetic cytokine receptor (e.g. RACR) by its cognate non-physiological ligand (e.g. rapamycin) is able to deliver a cytokine signal into the cell inducing the JAK/STAT pathway and driving differentiation. In some aspects, the requirement for further growth factors or cytokines to drive differentiation at one or more different steps of the process is reduced or eliminated, thereby providing for a directed and consistent differentiation.

In some embodiments, directed differentiation of stem cells, such as pluripotent stem cells (e.g. iPSC) to hematopoietic progenitors (HP) and common lymphoid progenitors (CLP) can be achieved by engaging the synthetic cytokine receptor with the non-physiological ligand. In some embodiments, engagement of the synthetic cytokine receptor (e.g. RACR) as provided with a non-physiological ligand (e.g. rapamycin or an analog) drives differentiation through inducing STAT5 signaling via the JAK/STAT pathway, thereby eliminating or reducing requirements for other growth factors or cytokines that may provide a similar STAT5 signaling. Observations herein demonstrate that engagement of the synthetic cytokine receptor (e.g. RACR) engineered on iPSC with a non-physiological ligand (e.g. rapamycin) is sufficient to drive iPSC to the CLPs. This result was surprising since conventional approaches for cell differentiation incorporate complex milieu of media additives to achieve differentiation to CLP or HP.

Although iPSC-derived cell therapies are promising, the current processes of deriving an immune cell from an iPSC is complex, variable, and costly. Current protocols for deriving downstream cell types from iPSCs use on sequential steps of “coaxing” of iPSCs down a differentiation pathway by feeding in external factors to engage endogenous receptors. In some embodiments, this process can require long culture times, expensive protein material, and can be highly variable due to dependency on constantly changing expression patterns of endogenous genes and receptors. The provided embodiments in which iPSCs are engineered with a synthetic cytokine receptor promotes differentiation through highly controlled synthetic receptors, which has the potential to reduce the variability of cell differentiation as well as decrease the cost of manufacturing of these cells by replacing expensive growth factors and cytokines with small molecule engagers (e.g. rapamycin).

In some embodiments, the synthetic cytokine receptor (e.g. RACR) that is engineered into stem cells such as iPSCs provides an opportunity to derive cells through a cytokine receptor signal that mimics normal signaling during cell differentiation. In particular, the synthetic cytokine receptor induces JAK/STAT signaling, a downstream signaling pathway that is essential in blood development. Current protocols use multiple growth factors to induce hematopoiesis of iPSCs, such as thrombopoietin (TPO), stem cell factor (SCF), bone morphogenic protein (BMP4), and fibroblast activated protein (FGF2)-all these growth factors induce JAK/STAT signaling in combination with other signaling pathways that help to drive blood development. Thus, replacing or supplementing these endogenous receptor pathways with a non-physiological stimulus of the synthetic cytokine receptor, such as via rapamycin or an analog, to drive JAK/STAT signals has the potential to improve the process of blood development and reduce the need for multiple exogenous protein signals. After blood cells emerge during differentiation, blood progenitors can be further differentiated into lymphoid progenitors, including CLP. In some aspects, the common gamma chain cytokines such as IL-7, IL-15, IL-2, and IL-21 are typically used in the process of driving CD34+ (HP) progenitors to differentiated immune cells. However, since the synthetic cytokine receptors are designed from common gamma chain cytokine signaling, the provided embodiments and methods also can be carried out with reduced or no additional cytokine support and thus can replace the need for these cytokine mixtures during cell differentiation, reducing the cost, variability, and complexity of cell generation.

Moreover, generation of HP and CLP from iPSC using conventional approaches is generally inefficient and is a current bottleneck in the manufacture of effector immune cell therapies, in some cases achieving only ˜1× yield of HP from iPSC (1 HP for every 1 iPSC). In some embodiments, the provided methods can result in increased yields greater than at or about 10-fold (10×), 20-fold, 40-fold, 50-fold, 60-fold, 70-fold, 80-fold, 90-fold, 100-fold, 150-fold, 200-fold, 250-fold, 300-fold of HP from iPSC. In some embodiments, the provided methods can result in increased yields greater than at or about 350-fold, 400-fold, 450-fold, 500-fold, 550-fold, 600-fold, 650-fold, 1,000-fold, 1,500-fold, 2,000-fold, 2,500-fold, 3,000-fold, 3,500-fold of HP from iPSC. In some embodiments, the provided methods can result in increased yields greater than at or about 630-fold of HP from iPSC. In some embodiments, the provided methods can result in increased yields greater than at or about 3,000-fold of HP from iPSC. This result represents a substantial improvement over other approaches to differentiation iPSC to HP. Furthermore, generation of iCIL from HP using the provided embodiments result in iCIL that are highly potent as demonstrated by killing of tumor target cells.

In some embodiments, the provided methods can result in increased yields greater than at or about 1×10⁶-fold (1,000,000×), 1.5×10⁶-fold, 2×10⁶-fold, 2.5×10⁶-fold, 3×106-fold, 3.5×10⁶-fold, 4×10⁶-fold, 4.5×10⁶-fold, 5×10⁶-fold, 5.5×10⁶-fold, 6×10⁶-fold, 6.5×10⁶-fold, 7×10⁶-fold, 7.5×10⁶-fold, 8×10⁶-fold, 8.5×10⁶-fold, 9×10⁶-fold, 9.5×10⁶-fold, 10×10⁶-fold of iCIL from iPSC. In some embodiments, the provided methods can result in increased yields greater than at or about 9×10⁶-fold of iCIL from iPSC.

In some embodiments, the cells selected to undergo differentiation are pluripotent stem cells (PSCs), e.g. e.g., iPSCs, that have been engineered with a synthetic cytokine receptor, e.g., as described in Section II.B. In some embodiments, the cells selected to undergo differentiation are pluripotent stem cells (PSCs), e.g., iPSCs, that are further disrupted in a gene encoding B2M such as to reduce expression or knockout the gene encoding B2M. In some embodiments, the engineered synthetic cytokine receptor is integrated into the disrupted B2M locus, such as by HDR or other methods. In some embodiments, the cells selected to undergo differentiation are pluripotent stem cells (PSCs), e.g., iPSCs, that are further disrupted in a gene encoding FBP12 such as to reduce expression or knockout the gene encoding FBP12. In some embodiments, the cells selected to undergo differentiation are pluripotent stem cells (PSCs), e.g., iPSCs, are any of the engineered cells described in Section I.D.

In some embodiments, the provided methods include culturing the engineered PSCs (e.g. iPSCs) by incubation with a non-physiological ligand of the synthetic cytokine receptor (e.g. rapamycin or analog) under conditions to differentiate the stem cells to iCIL cells or to a progenitor thereof, such as HPs or CLPs. In some embodiments, the methods can include one more incubations in which different molecules are added to the culture media. In some embodiments, the methods can include replacement of media to supplement or add any one or more molecules to the culture media.

In some embodiments, the methods include a first incubation with a non-physiological ligand of the synthetic cytokine receptor (e.g. rapamycin or analog) under conditions to induce their differentiation into hematopoietic progenitor (HP) cells. In some embodiments, one or more certain additional molecules (e.g. small molecules) may be added to further promote or induce differentiation into hematopoietic progenitor (HP) cells.

In some embodiments, one or more of the above steps of producing HP cells can include addition of a non-physiological ligand of the synthetic cytokine receptor (e.g. rapamycin or analog) to the culture medium to induce differentiation. In some embodiments, the non-physiological ligand is rapamycin or a rapamycin analog. In some embodiments, the rapamycin analog is rapalog. In some embodiments, the non-physiological ligand (e.g., rapamycin or a rapamycin analog) is added to the media at a concentration of 2.5 nM and 200 nM, 2.5 nM and 150 nM, 2.5 nM and 100 nM, 2.5 nM and 50 nM, 2.5 nM and 20 nM, 2.5 nM and 10 nM, In some embodiments, the non-physiological ligand (e.g., rapamycin or a rapamycin analog) is added to the media at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM and 150 nM and 200 nM.

In some embodiments, the non-physiological ligand (e.g., rapamycin or a rapamycin analog) is added to the media at a concentration of at or about 10 nM. In some embodiments, the non-physiological ligand (e.g., rapamycin or a rapamycin analog) is added to the media at a concentration of at or about 100 nM. In some embodiments, rapamycin is added to the media at a concentration of at or about 100 nM. In some embodiments, rapalog is added to the media at a concentration of at or about 100 nM.

In some embodiments, it is surprisingly found that low concentrations of the non-physiological ligand (e.g., rapamycin or a rapamycin analog) is able to support differentiation and/or expansion. In some embodiments, the non-physiological ligand (e.g., rapamycin or a rapamycin analog) is added to the media at a concentration at or less than 10 nM. In some embodiments, the non-physiological ligand (e.g., rapamycin or a rapamycin analog) is added to the media at a concentration from 2.5 nM to 10 nM, such as 3 nM to 7 nM. In some embodiments, the non-physiological ligand (e.g., rapamycin or a rapamycin analog) is added to the media at a concentration of at or about 3 nM, at or about 4 nM, at or about 5 nM, at or about 6 nM, at or about 7 nM, at or about 8 nM, at or about 9 nM, or at or about 10 nM, or any value between any of the foregoing. In some embodiments, the non-physiological ligand (e.g., rapamycin or a rapamycin analog) is added to the media at a concentration of at or about 3.1 nM. In some embodiments, the non-physiological ligand (e.g., rapamycin or a rapamycin analog) is added to the media at a concentration of at or about 6.2 nM. In some embodiments, rapamycin is added to the media at a concentration of at or about 3.1 nM. In some embodiments, rapamycin is added to the media at a concentration of at or about 6.2 nM. In some embodiments, rapalog is added to the media at a concentration of at or about 3.1 nM. In some embodiments, rapalog is added to the media at a concentration of at or about 6.2 nM.

In some embodiments, the provided method includes culturing engineered stem cells, e.g. engineered with a synthetic cytokine receptor, with the non-physiological ligand for a first period of time sufficient to generate CLPs, and contacting the CLPs with a differentiation media for a second period of time sufficient to generate iCILs.

In some embodiments, conditions in addition to or other than activation with the synthetic cytokine receptor can be used in methods to differentiate the engineered stem cells to CILs.

In some embodiments, the provided stem cells, such as iPSCs, engineered with a synthetic cytokine receptor may instead or alternatively be differentiated via any other method known to differentiate CILs. In some embodiments, one or more growth factor or cytokine customarily used in connection with differentiation of CILs may be used in the provided methods in addition to the non-physiological ligand engagement of the synthetic cytokine receptor.

Various differentiation protocols for CIL cells are known in the art.

In some embodiments, the stem cells are adapted for feeder-free culture. As used herein, a “feeder-free” (FF) environment refers to an environment such as a culture condition, cell culture or culture media which is essentially free of feeder or stromal cells, and/or which has not been pre-conditioned by the cultivation of feeder cells. “Pre-conditioned” medium refers to a medium harvested after feeder cells have been cultivated within the medium for a period of time, such as for at least one day. Pre-conditioned medium contains many mediator substances, including growth factors and cytokines secreted by the feeder cells cultivated in the medium.

Broadly, techniques for differentiating a cell involve modulation of specific cellular pathways, either directly or indirectly, using polynucleotide-, polypeptide- and/or small molecule-based approaches. The developmental potency of a cell may be modulated, for example, by contacting a cell with one or more modulators. In some embodiments, cells are cultured in the presence of one or more agents to induce cell differentiation (such as, for example, small molecules, proteins, peptides, etc.). In some embodiments, the one or more differentiation agents are introduced to the cell during in vitro culture. The cell may be maintained in the culture medium comprising one or more agents for a period sufficient for the cell to achieve the differentiation phenotype that is desired.

In some embodiments, the culture platform comprises one or more of the following: nutrients, extracts, growth factors, hormones, cytokines and medium additives. Illustrative nutrients and extracts may include, for example, DMEM/F-12 (Dulbecco's Modified Eagle Medium/Nutrient Mixture F-12), which is a widely used basal medium for supporting the growth of many different mammalian cells; KOSR (knockout serum replacement); L-glut; NEAA (Non-Essential Amino Acids). Medium additives may include, but are not limited to, MTG, ITS, (ME, anti-oxidants (for example, ascorbic acid).

In some embodiments, the differentiation media contains supplements such as serums, extracts, growth factors, hormones, cytokines and the like.

In some embodiments, a culture medium of the present invention comprises one or more of the following cytokines or growth factors: epidermal growth factor (EGF), acidic fibroblast growth factor (aFGF), basic fibroblast growth factor (bFGF), leukemia inhibitory factor (LIF), hepatocyte growth factor (HGF), insulin-like growth factor 1 (IGF-1), insulin-like growth factor 2 (IGF-2), keratinocyte growth factor (KGF), nerve growth factor (NGF), platelet-derived growth factor (PDGF), transforming growth factor beta (TGF-β), bone morphogenetic protein (BMP4), vascular endothelial cell growth factor (VEGF) transferrin, various interleukins (such as IL-1 through IL-18), various colony-stimulating factors (such as granulocyte/macrophage colony-stimulating factor (GM-CSF)), various interferons (such as IFNγ) and other cytokines such as stem cell factor (SCF) and erythropoietin (EPO).

These cytokines may be obtained commercially and may be either natural or recombinant. In some other embodiments, the culture medium of the present disclosure comprises one or more of bone morphogenetic protein (BMP4), insulin-like growth factor-1 (IGF-1), basic fibroblast growth factor (bFGF), vascular endothelial growth factor (VEGF), hematopoictic growth factor (for example, SCF, GMCSF, GCSF, EPO, IL3, TPO, EPO), Fms-Related Tyrosine Kinase 3 Ligand (FLT3L); and one or more cytokines from Leukemia inhibitory factor (LIF), IL3, IL6, IL7, IL11, IL15. In some embodiments, the growth factors, mitogens, and cytokines are stage and/or cell type specific in concentrations that are determined empirically or as guided by the established cytokine art. Examples of exogenous cell culture media additives and supplements and cell selection kit components are provided in WO 2020/124256, the disclosure of which is incorporated by reference herein in its entirety.

In some other embodiments, the culture medium of the present disclosure comprises Roswell Park Memorial Institute (RPMI) media, cRPMI1640 media, fetal bovine serum (FBS), Glutamax, Penicillin, streptomycin, Rosuvastatin, BX795, protamine sulfate, brefeldin A, monensin, UM729, IL-2, IL-15, IL-21, IL-18, IL-7, or any combination thereof. In some other embodiments, the culture medium of the present disclosure comprises AIM V media, fetal bovine serum (FBS), Glutamax, Penicillin, streptomycin, Rosuvastatin, BX795, protamine sulfate, brefeldin A, monensin, UM729, IL-2, IL-15, IL-21, IL-18, IL-7, or any combination thereof. In some other embodiments, the culture medium of the present disclosure comprises AIM V media STEMdiff APEL 2 Medium (STEMCELL Technologies).

Examples of methods for differentiating stem cells (e.g., iPSCs) into multipotent hematopoietic progenitor cells are provided in U.S. Pat. No. 9,624,470, U.S. Patent Appl. No. 2020/0080059, as well as Mesquitta et al., Sci. Rep. 9:6622 (2019), the disclosures of which are incorporated by reference herein in their entireties.

In some embodiments, the method of producing CIL cells of the disclosure comprises: forming embryoid bodies (EBs) comprising aggregates of stem cells; differentiating the cells into hematopoietic stem cells in a first differentiation medium; differentiating the cells into lymphoid progenitor cells in a second differentiation medium; and/or differentiating the cells into differentiated CIL cells in a third differentiation medium. In some embodiments, the methods provided herein results in EBs that completely dissociate into pure hematopoietic progenitors or hematopoietic stem cells without the need for an additional purification step.

In some embodiments, provided is a method for generating cytotoxic innate lymphoid (iCIL) cells, comprising culturing a cell population comprising engineered iPSCs as described under conditions to differentiate the iPSCs to cytotoxic innate lymphoid (iCILs), wherein a non-physiological ligand of the synthetic cytokine receptor is added during at least a portion of the culturing.

In some embodiments, one or more of the above steps of producing CIL cells can include addition of a non-physiological ligand of the synthetic cytokine receptor (e.g. rapamycin or analog) to the culture medium to induce differentiation.

In some embodiments, the non-physiological ligand is rapamycin or a rapamycin analog. In some embodiments, the rapamycin analog is rapalog. In some embodiments, the non-physiological ligand (e.g., rapamycin or a rapamycin analog) is added to the media at a concentration of 2.5 nM and 200 nM, 2.5 nM and 150 nM, 2.5 nM and 100 nM, 2.5 nM and 50 nM, 2.5 nM and 20 nM, 2.5 nM and 10 nM, In some embodiments, the non-physiological ligand (e.g., rapamycin or a rapamycin analog) is added to the media at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM and 150 nM and 200 nM.

In some embodiments, the non-physiological ligand (e.g., rapamycin or a rapamycin analog) is added to the media at a concentration of at or about 10 nM. In some embodiments, the non-physiological ligand (e.g., rapamycin or a rapamycin analog) is added to the media at a concentration of at or about 100 nM. In some embodiments, rapamycin is added to the media at a concentration of at or about 100 nM. In some embodiments, rapalog is added to the media at a concentration of at or about 100 nM.

In some embodiments, it is surprisingly found that low concentrations of the non-physiological ligand (e.g., rapamycin or a rapamycin analog) is able to support differentiation and/or expansion. In some embodiments, the non-physiological ligand (e.g., rapamycin or a rapamycin analog) is added to the media at a concentration at or less than 10 nM. In some embodiments, the non-physiological ligand (e.g., rapamycin or a rapamycin analog) is added to the media at a concentration from 2.5 nM to 10 nM, such as 3 nM to 7 nM. In some embodiments, the non-physiological ligand (e.g., rapamycin or a rapamycin analog) is added to the media at a concentration of at or about 3 nM, at or about 4 nM, at or about 5 nM, at or about 6 nM, at or about 7 nM, at or about 8 nM, at or about 9 nM, or at or about 10 nM, or any value between any of the foregoing. In some embodiments, the non-physiological ligand (e.g., rapamycin or a rapamycin analog) is added to the media at a concentration of at or about 3.1 nM. In some embodiments, the non-physiological ligand (e.g., rapamycin or a rapamycin analog) is added to the media at a concentration of at or about 6.2 nM. In some embodiments, rapamycin is added to the media at a concentration of at or about 3.1 nM. In some embodiments, rapamycin is added to the media at a concentration of at or about 6.2 nM. In some embodiments, rapalog is added to the media at a concentration of at or about 3.1 nM. In some embodiments, rapalog is added to the media at a concentration of at or about 6.2 nM.

In some embodiments, the method for generating cytotoxic innate lymphoid (iCIL) cells comprises: a) culturing a cell population comprising engineered iPSCs as described under conditions to form an aggregate; b) culturing the cells produced in a) under conditions to induce mesoderm formation in a plurality of the cells, wherein the initiation of the culturing in b) is day 0; c) culturing the cells produced in b) under conditions to differentiate cells into a population of hematopoietic progenitors (HP); and d) culturing the cells produced in c) under conditions to generate iCIL cells, wherein at least a portion of one or more of steps a)-d) are carried out in the presence of a non-physiological ligand of the synthetic cytokine receptor.

In some embodiments, to promote aggregate formation engineered iPSCs are transferred to a suitable vessel. The vessel may be a 2D or a 3D vessel. Examples of suitable 2D vessels for culturing source cells include any petri dish or culture dish regularly used in the laboratory for culturing cells. The culturing vessel may be coated with a suitable culturing medium, for example an extracellular medium for the attachment and/or differentiation of cultured cells. In some embodiments, the vessel is treated to promote cell adhesion and growth. An example of a suitable medium for use in the inventive method is MATRIGEL™ membrane matrix (BD Biosciences, Franklin Lakes, N.J.).

In some embodiments, the vessel is suitable for 3-Dimensional (3D) culture. Without being bound to a particular theory or mechanism, it is believed that 3D culture may be more effective for providing a scaffold for cell differentiation than two dimensional (2D) culture. Suitable 3D culture systems may include, for example, a hanging drop 3D culture, e.g., hanging drop plates, a 3D microwell culture, e.g., ultra-low attachment multiwell plates, a 3D culture on a hydrophobic surface, a rotational culture, a static 3D suspension culture, or a bioreactor. Hanging drop plates are commercially available such as, for example, the PERFECTA3D hanging drop plate, available from Biospherix, Parish, N.Y. Ultra-low attachment multiwell plates (in some cases also referred to as non-adherent culture vessels) are also commercially available such as, for example, AGGREWELL™ ultra-low attachment, multi-well plate, available from Stemcell Technologies, Vancouver, Canada.

In some embodiments, the vessel is not treated to promote cell adhesion and growth. In some embodiments, the vessel is a standard tissue culture plate but is not treated to promote cell adhesion and growth. In some embodiments, the cells do not adhere or substantially adhere during the culturing. In some embodiments, the culturing is in suspension.

In some embodiments, the vessels are multi-well plates. The multi-well plates may be 96-well plates, 24-well plates or 6-well plates.

In some embodiments, the vessel is a bioreactor. In some embodiments, bioreactors are used for the process of iCIL generation and proliferation after the development of EBs. Bioreactors allow for the optimization of cell culture conditions to achieve optimal hydrogen production yields and process robustness. Environmental conditions that can be adjusted or monitored in a bioreactor include gas composition (e.g., air, oxygen, nitrogen, carbon dioxide), gas flow rates, temperature, pH, dissolved oxygen levels, and the agitation speed/circulation rate within the cell culture. Any type of bioreactor known in the art may be used for cell culture for the differentiation and expansion of cultured iCIL cells, including, but not limited to, a stirred-tank bioreactor, a pneumatic bioreactor (e.g., a bubble column or airlift bioreactor), a membrane bioreactor, a hollow-fiber bioreactor, a wave bioreactor, a vertical wheel bioreactor, a gas permeable rapid expansion (G-Rex) bioreactor, or a disposable bioreactor. In some embodiments, the bioreactor is a gas permeable rapid expansion (G-Rex) bioreactor. In some embodiments, the bioreactor is a vertical wheel bioreactor. In some embodiments, the bioreactor is a stirred-tank bioreactor. In some embodiments, the vertical wheel bioreactor is the PBS bioreactor. In some embodiments, the stirred-tank bioreactor is the Sartorius Ambr250 stirred tank bioreactor.

The expansion of the iCIL cells may be scaled to any desired volume to suit various purposes. For example, for high to medium throughput screening of various culture conditions, the process may be scaled to take place in a microbioreactor (e.g., about 15 mL to about 500 mL) or a benchtop scale bioreactor e.g., ranging from about 0.5 L to about 15 L). Alternatively, the process may be scaled up to a pilot scale bioreactor (e.g., ranging from about 15 L to about 15,000 L), or a manufacturing scale bioreactor (e.g., about 15,000 L to about 75,000 L or greater).

In some embodiments, pluripotent aggregates may be formed in a bioreactor by culturing the engineered iPSCs in suspension in the bioreactor. In some embodiments, the iPSC may spontaneously aggregate into spheroids directly in a bioreactor. In some embodiments, aggregates or spheroids formed directly in bioreactors may be approximately the same size as spheroids formed in other 3D culture systems, including e.g., ultra-low attachment microwell plates. In an embodiment, the method may comprise forming source cells into spheroids directly in a bioreactor without forming EBs by culturing the source cells in suspension in the bioreactor in xeno-free medium. The spheroids may comprise undifferentiated iPSC, such as engineered iPSC.

In some embodiments, the aggregate in a) is an embryoid body (EB).

In some embodiments, differentiation of the cells for generating CIL cells requires a change in a culturing system, such as changing the stimuli agents in the culture medium or the physical state of the cells. A conventional strategy utilizes the formation of embryoid bodies as a common and critical intermediate to initiate the lineage-specific differentiation. Embryoid bodies are aggregates of stem cells that are induced to differentiate by changes in environmental stimuli (e.g., exposure and/or removal of specific molecular/chemical factors; and/or exposure/interaction with three-dimensional structures). Formation of embryoid bodies induces the cells to differentiate cells to a mesoderm specification.

Hematopoietic cells may be generated from embryoid bodies derived from pluripotent cells. Pluripotent cells may be allowed to form embryoid bodies or aggregates as a part of the differentiation process. The formation of “embryoid bodies” (EBs), or clusters of growing cells, in order to induce differentiation generally involves in vitro aggregation of human pluripotent stem cells into EBs and allows for the spontaneous and random differentiation of human pluripotent stem cells into multiple tissue types that represent endoderm, ectoderm, and mesoderm origins. Without specific culture conditions, it may take about two weeks for EBs to differentiate toward any of the three germ layers, and the differentiation process is performed in a random pattern. In provided embodiments, certain growth factors or cytokines can be added to the culture conditions to guide or boost the differentiation of the EBs toward the hematopoietic lineage, which is formed through the mesoderm lineage. EB cells that have differentiated toward hematopoietic lineage can be identified by major hematopoietic lineage markers, such as, for example, any one or more of CD34, CD43, CD45, CD41, C235, and CD90.

In some embodiments, a single cell suspension of engineered iPSCs are cultured in an appropriate vessel to form EBs.

In some embodiments, pluripotent stem cell aggregates are transferred to differentiation medium that provides eliciting cues towards the lineage of choice (e.g., lymphoid). In some embodiments, once EBs have formed, they are dissociated and then cultured in media to induce mesoderm specificity of the cells.

In some embodiments, a suspension aggregate can be generated by culture in a non-treated vessel or under conditions for suspension culture. For instance, the vessel is a vessel that is not treated to promote cell adhesion and proliferation. In some embodiments, the step a) of culturing a cell population comprising engineered iPSCs under conditions to form an aggregate involves: (i) performing a first incubation comprising culturing the cell population of engineered stem cells under conditions to form a first aggregate; (ii) contacting the aggregate with a dissociating agent to form a population of dissociated cells; and (iii) performing a second incubation comprising culturing the population of dissociated cells under conditions to form the second aggregate. Methods of dissociating the cells are known to a skilled artisan. Any of a variety of methods can be used. In some embodiments, dissociation is with Gentle Cell Dissociation Reagent (GDCR; Stem Cell Technologies). In some embodiments, dissociation is with EDTA.

In some aspects of the invention, the embryoid body derivation and differentiation to hematopoietic progenitor cells platforms described above may be carried out under serum-free conditions. Examples of commercially available serum-free media suitable for cell attachment and/or induction include mTeSR™1, STEMdiff APEL 2 Medium, or TeSR™2 from Stem Cell Technologies (Vancouver, Canada), Primate ES/iPS cell medium from ReproCELL (Boston, Mass.), StemPro®-34 from Invitrogen (Carlsbad, Calif.), StemPro® hESC SFM from Invitrogen, and X-VIVO™ from Lonza (Basel, Switzerland).

In some embodiments, the media of the induction of mesoderm specificity step comprises one or more of Bone Morphogenic Protein 4 (BMP4), Vascular Endothelial Growth Factor (VEGF), Stem Cell Factor (SCF), Thrombopoietin (TPO), Fms-related Tyrosine Kinase 3 Ligand (FLT3L), basic Fibroblast Growth Factor (bFGF also known as FGF2). In some embodiments, the media of the induction of mesoderm specificity step comprises one or more of Bone Morphogenic Protein 4 (BMP4), Vascular Endothelial Growth Factor (VEGF), Stem Cell Factor (SCF), Thrombopoietin (TPO), Fms-related Tyrosine Kinase 3 Ligand (FLT3L), basic Fibroblast Growth Factor (bFGF also known as FGF2), and/or a ROCK inhibitor. In some embodiments, the media of the induction of mesoderm specificity step comprises one or more of Bone Morphogenic Protein 4 (BMP4), Vascular Endothelial Growth Factor (VEGF), Stem Cell Factor (SCF), basic Fibroblast Growth Factor (bFGF also known as FGF2), and/or a ROCK inhibitor. In some embodiments, the method comprises differentiating the mesoderm specified cells into hematopoietic stem cells in a first differentiation medium.

In some embodiments, the hematopoietic stem cells in a first differentiation media comprises one or more of Bone Morphogenic Protein 4 (BMP4), Fibroblast Growth Factor 2 (FGF2), Vascular Endothelial Growth Factor (VEGF), Stem Cell Factor (SCF), Fms-related Tyrosine Kinase 3 Ligand (FLT3L), Thrombopoietin (TPO), Interleukin-6 (IL-6), Interleukin-3 (IL-3), a TGF-B inhibitor, a PI3K inhibitor, or any combination thereof.

In some embodiments, the TGF-β inhibitor is GW788388. In some embodiments the PI3K inhibitor is LY294002. In some embodiments the ROCK inhibitor is Y27632.

In some embodiments, stem cells are differentiated into the mesoderm lineage in a differentiation medium comprising BMP4, FGF2, and VEGF. In some embodiments, stem cells are differentiated into the mesoderm lineage in a differentiation medium comprising BMP4, FGF2, VEGF, and a ROCK inhibitor. In some embodiments, stem cells are differentiated into the mesoderm lineage in a differentiation medium comprising BMP4, FGF2, VEGF, and Y27632.

In some embodiments, the culturing in b) is in a media comprising one or more of BMP4, FGF2, VEGF and a Rock Inhibitor. In some embodiments, the Rock Inhibitor is Y27632. In some embodiments, the culturing in b) is in a media comprising BMP4, FGF2, VEGF and Y27632. In some embodiments, the culturing in b) is in a media comprising BMP4, FGF2 and VEGF. In some embodiments, the culturing in b) is in a media comprising the non-physiological ligand. In some embodiments, the culturing in b) is in a media comprising the non-physiological ligand without any additional growth factors.

In some embodiments, the culturing in b) is for 2 to 4 days. In some embodiments, the cells are cultured in the differentiation media for 2 to 4 days. In some days, the culturing is for at or about 2 days, at or about 3 days or at or about 4 days. In some embodiments, the culturing is for at or about 3 days. In some embodiments, the concentration of the BMP4 in the media is from about 0.5 ng/mL-2.5 ng/mL, 0.5 ng/mL-5 ng/mL, 0.5 ng/mL-10 ng/mL, 0.5 ng/mL-15 ng/mL, 0.5 ng/mL-20 ng/mL, 0.5 ng/mL-30 ng/mL, 0.5 ng/mL-50 ng/mL, 2.5 ng/mL-5 ng/mL, 2.5 ng/mL-10 ng/mL, 2.5 ng/mL-15 ng/mL, 2.5 ng/mL-20 ng/mL, 2.5 ng/mL-30 ng/mL, 2.5 ng/mL-50 ng/mL, 5 ng/mL-10 ng/mL, 5 ng/mL-15 ng/mL, 5 ng/mL-20 ng/mL, 5 ng/mL-30 ng/mL, 5 ng/mL-50 ng/mL, 10 ng/mL-15 ng/mL, 10 ng/mL-20 ng/mL, 10 ng/mL-30 ng/mL, 10 ng/mL-50 ng/mL, each inclusive. In some embodiments, the concentration of BMP4 in the media is at least about 0.5 ng/mL, 2.5 ng/mL, 5 ng/mL, 10 ng/mL, 15 ng/mL, 20 ng/mL, 30 ng/mL, or 50 ng/mL, In some embodiments, the concentration of BMP4 in the media is about 10 ng/mL.

In some embodiments, the concentration of the FGF2 in the media is from about 0.5 ng/mL-2.5 ng/mL, 0.5 ng/mL-5 ng/mL, 0.5 ng/mL-10 ng/mL, 0.5 ng/mL-15 ng/mL, 0.5 ng/mL-20 ng/mL, 0.5 ng/mL-30 ng/mL, 0.5 ng/mL-50 ng/mL, 2.5 ng/mL-5 ng/mL, 2.5 ng/mL-10 ng/mL, 2.5 ng/mL-15 ng/mL, 2.5 ng/mL-20 ng/mL, 2.5 ng/mL-30 ng/mL, 2.5 ng/mL-50 ng/mL, 5 ng/mL-10 ng/mL, 5 ng/mL-15 ng/mL, 5 ng/mL-20 ng/mL, 5 ng/mL-30 ng/mL, 5 ng/mL-50 ng/mL, 10 ng/mL-15 ng/mL, 10 ng/mL-20 ng/mL, 10 ng/mL-30 ng/mL, 10 ng/mL-50 ng/mL, each inclusive. In some embodiments, the concentration of FGF2 in the media is at least about 0.5 ng/mL, 2.5 ng/mL, 5 ng/mL, 10 ng/mL, 15 ng/mL, 20 ng/mL, 30 ng/mL, or 50 ng/mL, In some embodiments, the concentration of FGF2 in the media is about 10 ng/mL.

In some embodiments, the concentration of the VEGF in the media is from about 0.5 ng/mL-2.5 ng/mL, 0.5 ng/mL-5 ng/mL, 0.5 ng/mL-10 ng/mL, 0.5 ng/mL-15 ng/mL. 0.5 ng/mL-20 ng/mL, 0.5 ng/mL-30 ng/mL, 0.5 ng/mL-50 ng/mL, 0.5 ng/mL-100 ng/mL, 2.5 ng/mL-5 ng/mL, 2.5 ng/mL-10 ng/mL, 2.5 ng/mL-15 ng/mL, 2.5 ng/mL-20 ng/mL, 2.5 ng/mL-30 ng/mL, 2.5 ng/mL-50 ng/mL, 2.5 ng/mL-100 ng/mL, 5 ng/ml-10 ng/mL, 5 ng/mL-15 ng/mL, 5 ng/mL-20 ng/mL, 5 ng/mL-30 ng/mL, 5 ng/mL-50 ng/mL, 5 ng/mL-100 ng/mL, 10 ng/mL-15 ng/mL, 10 ng/mL-20 ng/mL, 10 ng/mL-30 ng/mL, 10 ng/mL-50 ng/mL, 10 ng/mL-100 ng/mL, each inclusive. In some embodiments, the concentration of VEGF in the media is at least about 0.5 ng/mL, 2.5 ng/mL, 5 ng/mL, 10 ng/mL, 15 ng/mL, 20 ng/mL, 30 ng/mL, 50 ng/mL, or 100 ng/mL, In some embodiments, the concentration of VEGF in the media is about 50 ng/mL.

In some embodiments, the concentration of the Y27632 in the media is from about 0.5 μM-2.5 μM, 0.5 μM-5 μM, 0.5 μM-10 μM, 0.5 M-15 μM, 0.5 μM-20 μM, 0.5 μM-30 μM, 0.5 μM-50 μM, 2.5 μM-5 μM, 2.5 μM-10 μM, 2.5 μM-15 μM, 2.5 μM 20 μM, 2.5 μM-30 μM, 2.5 μM-50 μM, 5 M-10 μM, 5 μM-15 μM, 5 μM-20 μM, 5 μM-30 μM, 5 μM-50 μM, 10 μM-15 μM, 10 μM-20 μM, 10 μM-30 μM, 10 μM-50 μM, each inclusive. In some embodiments, the concentration of Y27632 in the media is at least about 0.5 μM, 2.5 μM, 5 μM, 10 μM, 15 μM, 20 μM, 30 μM, or 50 μM, In some embodiments, the concentration of Y27632 in the media is about 10 μM.

In some embodiments, mesoderm cells are differentiated into hematopoietic stem cells in a differentiation medium comprising BMP4, FGF2, VEGF, and SCF. In some embodiments, mesoderm cells are differentiated into hematopoietic stem cells in a differentiation medium comprising BMP4, FGF2, VEGF, SCF, TPO, and LDL. In some embodiments, mesoderm cells are differentiated into hematopoietic stem cells in a differentiation medium comprising BMP4, FGF2, and VEGF. In some embodiments, the differentiation media comprises a non-physiological ligand of the synthetic cytokine receptor (e.g. rapamycin or an analog).

In some embodiments, hematopoietic stem cells are differentiated into lymphoid progenitor cells in a differentiation medium comprising BMP4, FGF2, VEGF, and SCF. In some embodiments, hematopoietic stem cells are differentiated into lymphoid progenitor cells in a differentiation medium comprising BMP4, FGF2, and VEGF. In some embodiments, hematopoietic stem cells are differentiated into lymphoid progenitor cells in a differentiation medium comprising BMP4, FGF2, VEGF, SCF. TPO and LDL. In some embodiments, the differentiation media comprises a non-physiological ligand of the synthetic cytokine receptor (e.g. rapamycin or an analog).

In some embodiments, the culturing in c) is in a media comprising one or more of BMP4, FGF2, VEGF, TPO, SCF, and LDL. In some embodiments, the culturing in c) is in a media comprising one or more of BMP4, FGF2, VEGF and LDL. In some embodiments, the culturing in c) is in a media without SCF and TPO. In some of any embodiments, the culturing in c) is in a media comprising one or more of BMP4, FGF2, and a PI3K inhibitor. In some embodiments, the PI3K inhibitor is LY2940002. In some of any embodiments, the culturing in c) is in a media without LDL, VEGF, SCF and/or TPO. In some embodiments, the culturing in c) is in a media comprising the non-physiological ligand. In some embodiments, the culturing in c) is in a media comprising the non-physiological ligand without any additional growth factors, cytokines or both.

In some embodiments, the culturing in c) is on days 3 to 15 days. In some embodiments, during at least a portion of the culturing in c) the media comprises an aryl hydrocarbon receptor (AHR) antagonist (e.g. StemRegenin-1), a pyrimido-[4,5-b]-indole derivative (e.g. UM729) or both. In some embodiments, the portion of the culturing is on or about days 9-15. In some embodiments, the portion of the culturing is on or about days 6-15. In some embodiments, the culturing in c) is on day 6. In some embodiments, the culturing in c) is on day 9.

In some embodiments, the concentration of the BMP4 in the media is from about 0.5 ng/mL-2.5 ng/mL, 0.5 ng/mL-5 ng/mL, 0.5 ng/mL-10 ng/mL, 0.5 ng/mL-15 ng/mL, 0.5 ng/mL-20 ng/mL, 0.5 ng/mL-30 ng/mL, 0.5 ng/mL-50 ng/mL, 2.5 ng/mL-5 ng/mL. 2.5 ng/mL-10 ng/mL, 2.5 ng/mL-15 ng/mL, 2.5 ng/mL-20 ng/mL, 2.5 ng/mL-30 ng/mL, 2.5 ng/mL-50 ng/mL, 5 ng/mL-10 ng/mL, 5 ng/mL-15 ng/mL, 5 ng/mL-20 ng/mL, 5 ng/mL-30 ng/mL, 5 ng/mL-50 ng/mL, 10 ng/mL-15 ng/mL, 10 ng/mL-20 ng/mL, 10 ng/mL-30 ng/mL, 10 ng/mL-50 ng/mL, each inclusive. In some embodiments, the concentration of BMP4 in the media is at least about 0.5 ng/mL, 2.5 ng/mL, 5 ng/mL, 10 ng/mL, 15 ng/mL, 20 ng/mL, 30 ng/mL, or 50 ng/mL, In some embodiments, the concentration of BMP4 in the media is about 10 ng/mL.

In some embodiments, the concentration of the FGF2 in the media is from about 0.5 ng/mL-2.5 ng/mL, 0.5 ng/mL-5 ng/mL, 0.5 ng/mL-10 ng/mL, 0.5 ng/mL-15 ng/mL, 0.5 ng/mL-20 ng/mL, 0.5 ng/mL-30 ng/mL, 0.5 ng/mL-50 ng/mL, 2.5 ng/mL-5 ng/mL, 2.5 ng/mL-10 ng/mL, 2.5 ng/mL-15 ng/mL, 2.5 ng/mL-20 ng/mL, 2.5 ng/mL-30 ng/mL, 2.5 ng/mL-50 ng/mL, 5 ng/mL-10 ng/mL, 5 ng/mL-15 ng/mL, 5 ng/mL-20 ng/mL, 5 ng/mL-30 ng/mL, 5 ng/mL-50 ng/mL, 10 ng/mL-15 ng/mL, 10 ng/mL-20 ng/mL, 10 ng/mL-30 ng/mL, 10 ng/mL-50 ng/mL, each inclusive. In some embodiments, the concentration of FGF2 in the media is at least about 0.5 ng/mL, 2.5 ng/mL, 5 ng/mL, 10 ng/mL, 15 ng/mL, 20 ng/mL, 30 ng/mL, or 50 ng/mL, In some embodiments, the concentration of FGF2 in the media is about 10 ng/mL.

In some embodiments, the concentration of the SCF in the media is from about 0.5 ng/mL-2.5 ng/mL, 0.5 ng/mL-5 ng/mL, 0.5 ng/mL-10 ng/mL, 0.5 ng/mL-15 ng/mL, 0.5 ng/mL-20 ng/mL, 0.5 ng/mL-30 ng/mL, 0.5 ng/mL-50 ng/mL, 0.5 ng/mL-100 ng/mL, 2.5 ng/mL-5 ng/mL, 2.5 ng/mL-10 ng/mL, 2.5 ng/mL-15 ng/mL, 2.5 ng/mL-20 ng/mL, 2.5 ng/mL-30 ng/mL, 2.5 ng/mL-50 ng/mL, 2.5 ng/mL-100 ng/mL, 5 ng/mL-10 ng/mL, 5 ng/mL-15 ng/mL, 5 ng/mL-20 ng/mL, 5 ng/mL-30 ng/mL, 5 ng/mL-50 ng/mL, 5 ng/mL-100 ng/mL, 10 ng/mL-15 ng/mL, 10 ng/mL-20 ng/mL, 10 ng/mL-30 ng/mL, 10 ng/mL-50 ng/mL, 10 ng/mL-100 ng/mL, each inclusive. In some embodiments, the concentration of SCF in the media is at least about 0.5 ng/mL, 2.5 ng/mL, 5 ng/mL, 10 ng/mL, 15 ng/mL, 20 ng/mL, 30 ng/mL, 50 ng/mL, or 100 ng/mL, In some embodiments, the concentration of SCF in the media is about 50 ng/mL.

In some embodiments, the concentration of the UM729 in the media is from about 0.5 μM-1 μM, 0.5 M-5 μM, 0.5 μM-10 μM, 0.5 μM-15 μM, 0.5 μM-20 μM, 0.5 μM-30 μM, 0.5 μM-50 μM, 1 μM-5 μM, 1 μM-10 μM, 1 μM-15 μM, 1 μM-20 μM, 1 μM-30 μM, 1 μM-50 μM, 5 μM-10 μM, 5 μM-15 μM, 5 μM-20 μM, 5 μM-30 μM, 5 μM-50 μM, 10 μM-15 μM, 10 μM-20 μM, 10 μM-30 μM, 10 μM-50 μM, each inclusive. In some embodiments, the concentration of UM729 in the media is at least about 0.5 μM, 1 μM, 5 μM, 10 μM, 15 μM, 20 μM, 30 μM, or 50 μM, In some embodiments, the concentration of UM729 in the media is about 1 μM.

In some embodiments, the concentration of the SR1 in the media is about or less than about 0.5 μM-1 μM, 0.5 μM-5 μM, 0.5 μM-10 μM, 0.5 μM-15 μM, 0.5 μM-20 μM, 0.5 μM-30 μM, 0.5 MM-50 μM, 1 μM-5 μM, 1 μM-10 μM, 1 μM-15 μM, 1 μM-20 μM, 1 μM-30 μM, 1 μM-50 μM, 5 μM-10 μM, 5 μM-15 μM, 5 M-20 μM, 5 μM-30 μM, 5 μM-50 μM, 10 μM-15 μM, 10 μM-20 μM, 10 μM-30 μM, 10 μM-50 μM, each inclusive. In some embodiments, the concentration of SR1 in the media is at least about 0.5 μM, 1 μM, 5 μM, 10 μM, 15 μM, 20 μM, 30 μM, or 50 μM, In some embodiments, the concentration of SR1 in the media is about 1 μM

In some embodiments, lymphoid progenitor cells are differentiated into cytotoxic innate lymphoid cells in a differentiation medium comprising a non-physiological ligand, SCF, FLT3L, and UM729. In some embodiments, lymphoid progenitor cells are differentiated into cytotoxic innate lymphoid cells in a differentiation medium comprising a non-physiological ligand, SCF, and UM729 In some embodiments, the non-physiological ligand is a rapalog. In some embodiments, the non-physiological ligand is rapamycin.

In some embodiments, differentiating the mesoderm specified cells into hematopoietic stem cells in a first differentiation medium occurs for a period of time sufficient for mesoderm specified cells to become CD34+ hematopoietic stem cells.

In some embodiments, the method comprises differentiating the hematopoietic stem cells into lymphoid progenitor cells in a second differentiation medium.

In some embodiments, the lymphoid progenitor cell differentiation media comprises one or more of Bone Morphogenic Protein 4 (BMP4), Fibroblast Growth Factor 2 (FGF2), Vascular Endothelial Growth Factor (VEGF), Stem Cell Factor (SCF), Fms-related Tyrosine Kinase 3 Ligand (FLT3L), Thrombopoietin (TPO), Interleukin-6 (IL-6), Interleukin-3 (IL-3), a TGF-B inhibitor, a PI3K inhibitor, or any combination thereof.

In some embodiments, differentiating the hematopoietic stem cells into lymphoid progenitor cells in a second differentiation medium occurs for a period of time sufficient for hematopoietic stem cells to become Lin-CD34+ CD38−/lo CD45RA+ CD90-lymphoid progenitor cells.

In some embodiments, the first differentiation medium further comprises the non-physiological ligand of the disclosure and/or the second differentiation medium further comprises the non-physiological ligand of the disclosure.

In some embodiments, the first differentiation medium and/or the second differentiation medium is substantially free of at least one cytokine (e.g., IL-2, IL-15, and/or IL-7).

In some embodiments, the differentiation media comprises one or more of Stem Cell Factor (SCF), Interleukin-7 (IL-7), Interleukin-15 (IL-15), Fms-related Tyrosine Kinase 3 Ligand (FLT3L), a Pyrimido-Indole Derivative, or any combination thereof. The Pyrimido-Indole Derivative is UM 729 (pyrimido-[4,5-b]-indole derivative).

In some embodiments, the culturing in d) is in a media comprising one or more of FLT3L, IL-7, IL-12, IL-15, SR-1 and UM729. In some embodiments, the culturing in d) is in a media comprising one or more of IL-15, SCR, SR-1 and UM729. In some embodiments, the culturing in d) is in a media is without FLT3L, IL-7 and/or IL-12. In some embodiments, the culturing in d) is in a media comprising the non-physiological ligand. In some embodiments, the culturing in d) is in a media comprising the non-physiological ligand without any additional growth factors, cytokines or both. In some embodiments, the culturing in d) is for a time between days 15 and 40. In some embodiments, the culturing in d) is for days 15 and 35. In some embodiments, the culturing in d) is for days 15 and 30.

In some embodiments, differentiating the lymphoid progenitor cells into CIL cells in a differentiation medium occurs for a period of time sufficient for the lymphoid progenitor cells to become CD3− CD56+ CD45+ CD94+ CD122+/IL-2Rβ+ CD127/IL-7Rα− FcγRIII/CD16+ KIR+ NKG2A+ NKG2D+ NKp30+ NKp44+ NKP46+ NKp80+. In some embodiments, differentiating the lymphoid progenitor cells into CIL cells in a differentiation medium occurs for a period of time sufficient for the lymphoid progenitor cells to become CD45+ CD56+. In some embodiments, differentiating the lymphoid progenitor cells into CIL cells in a differentiation medium occurs for a period of time sufficient for the lymphoid progenitor cells to become CD45+ CD5− CD7+ CD56+.

In some embodiments, differentiating the lymphoid progenitor cells into CIL cells in a differentiation medium is performed for about 8 to about 18 days. In some embodiments, differentiating the lymphoid progenitor cells into CIL cells in a differentiation medium is performed for about 1, 2, 3, 4, 5, 6, 7, 8, 9, 10, 11, 12, 13, 14, 15, 16, 17, 18, 19, or 20 days.

In some embodiments, the CIL cell differentiation medium is substantially free of Interleukin-15 (IL-15). In some embodiments, the CIL cell differentiation medium is substantially free of Interleukin-7 (IL-7). In some embodiments, the CIL cell differentiation medium is substantially free of Interleukin-2 (IL-2). In some embodiments, the CIL cell differentiation medium is substantially free of IL-15, IL-7, and/or IL-2.

In some embodiments, following differentiation, the CIL cells are expanded in a medium comprising a non-physiological ligand and CD2/NKp46 stimulation. In some embodiments, the CIL cell expansion medium comprises activation beads comprising conjugated anti-CD2 and anti-NKp46 antibodies which stimulate and activate the CIL cells.

In some embodiments, following differentiation, the RACR-iCIL cells are expanded in a medium comprising a non-physiological ligand, membrane-bound IL-21 (mbIL21), and 41BBL K562 initiated feeder cells.

F. Induced Cytotoxic Innate Lymphoid Cells (iCIL) and Expansion of iCIL Cells

In some embodiments, CIL cells may be derived from iPSCs by sequentially differentiating the iPSCs into hematopoietic progenitor cells (HPCs); the HPCs into common lymphoid progenitor cells (CLPs); and then the CLPs into CIL cells—termed “iCIL” cells. In a variation, CIL cells may be derived from HPCs by sequentially differentiating the HPCs into CLPs; and then the CLPs into iCIL cells. In a further variation, CIL cells may be derived by differentiating CLPs into iCIL cells. Engineering of the cells to express the synthetic cytokine receptor may be performed at the iPSC, HPC, CLP, or iCIL cell step of the differentiation process.

In some embodiments, the iCIL cells are characterized as being CD3-CD56+ CD45+ CD94+ CD122+/IL-2Rβ+ CD127/IL-7Rα−FcγRIII/CD16+ KIR+ NKG2A+ NKG2D+ NKp30+ NKp44+ NKP46+ NKp80+. In some embodiments, the iCIL cells are characterized by being CD3-CD56+ CD45+ cells. In some embodiments, the iCIL cells are characterized by being CD3-CD56+ cells. In some embodiments, the iCIL cells comprise one or more cell markers selected from the group consisting of CD56+, CD45+, CD94+, CD122+/IL-2Rβ+, FcγRIII/CD16+, KIR+, NKG2A+, NKG2D+, NKp30+, NKp44+, NKP46+, NKp80+, or any combination thereof. The iCIL cells may be CD45+; CD45+ CD5−; or CD45+ CD5− CD56+.

In some embodiments, the iCIL cells are characterized by being CD45+ CD7+ CD56^{+/lo}.

In some embodiments, after iCIL cells have been transduced with a CAR, the cells are cultured under conditions that promote the activation of the cells.

Culture conditions may be such that the cells can be administered to a patient without concern for reactivity against components of the culture medium. For example, the culture conditions may omit bovine serum products, such as bovine serum albumin. In one illustrative aspect, the activation can be achieved by introducing known activators into the culture medium. In one aspect, the population of iCIL cells can be cultured under conditions promoting activation for about 1 to about 4 days. In one embodiment, the appropriate level of activation can be determined by cell size, proliferation rate, or activation markers determined by flow cytometry. In some embodiments, any of the culturing methods disclosed herein may be used to promote activation of the iCIL cells.

In some embodiments, provided herein is a method of making and/or expanding a population of engineered cells comprising a synthetic cytokine receptor for a non-physiological ligand. In some embodiments, the method comprises:
providing engineered cells comprising a synthetic cytokine receptor, optionally by introducing into source cells a polynucleotide encoding a synthetic cytokine receptor, and
incubating the engineered cells in media comprising a non-physiological ligand.

In this embodiment, the cytokine receptor comprises a synthetic gamma chain polypeptide comprising a first dimerization domain, a first transmembrane domain, and an interleukin-2 receptor subunit gamma (IL-2RG) intracellular domain, and a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain; wherein the non-physiological ligand activates the synthetic cytokine receptor in the engineered cells to induce expansion and/or activation of the engineered cells.

In some embodiments, engineered CIL cells are expanded without IL-2 in the expansion medium.

In some embodiments, CIL cells are engineered to express a synthetic cytokine receptor, such as RACR, and expanded with a rapalog in the expansion medium and without recombinant cytokines in the expansion medium.

In some embodiments, the CIL cell expanding step is performed in cell culture media that is substantially free of recombinant cytokines.

In some embodiments, the expanding step is performed in a feeder-free cell culture.

In some embodiments, the CIL cell expanding step is performed in culture vessels not coated with recombinant ligands for CIL cell expansion.

In some embodiments, provided is a method of administering an effective amount of non-physiological ligand to the subject, wherein the non-physiological ligand causes the iPSC cells to differentiate to iCIL cells according to any of the foregoing embodiments.

In some embodiments, provided is a method of administering an effective amount of non-physiological ligand to the subject, wherein the non-physiological ligand causes the common lymphoid progenitor cells to differentiate to CIL cells according to any of the foregoing embodiments.

In some embodiments, provided are methods of differentiating and expanding iCIL cells by culturing in a suitable vessel. The vessel may be a 2D or a 3D vessel. Examples of suitable 2D vessels for culturing source cells include any petri dish or culture dish regularly used in the laboratory for culturing cells. The culturing vessel may be coated with a suitable culturing medium, for example an extracellular medium for the attachment and/or differentiation of cultured cells. In some embodiments, the vessel is treated to promote cell adhesion and growth. An example of a suitable medium for use in the inventive method is MATRIGEL™ membrane matrix (BD Biosciences, Franklin Lakes, N.J.).

In some embodiments, the vessel is suitable for 3-Dimensional (3D) culture. Without being bound to a particular theory or mechanism, it is believed that 3D culture may be more effective for providing a scaffold for cell differentiation than two dimensional (2D) culture. Suitable 3D culture systems may include, for example, a hanging drop 3D culture, e.g., hanging drop plates, a 3D microwell culture, e.g., ultra-low attachment multiwell plates, a 3D culture on a hydrophobic surface, a rotational culture, a static 3D suspension culture, or a bioreactor. Hanging drop plates are commercially available such as, for example, the PERFECTA3D hanging drop plate, available from Biospherix, Parish, N.Y. Ultra-low attachment multiwell plates (in some cases also referred to as non-adherent culture vessels) are also commercially available such as, for example, AGGREWELL™ ultra-low attachment, multi-well plate, available from Stemcell Technologies, Vancouver, Canada.

In some embodiments, the vessel is not treated to promote cell adhesion and growth. In some embodiments, the vessel is a standard tissue culture plate but is not treated to promote cell adhesion and growth. In some embodiments, the cells do not adhere or substantially adhere during the culturing. In some embodiments, the culturing is in suspension.

In some embodiments, the vessels are multi-well plates. The multi-well plates may be 96-well plates, 24-well plates or 6-well plates.

In some embodiments, the vessel is a bioreactor. In some embodiments, bioreactors are used for the process of iCIL generation and proliferation after the development of EBs. Bioreactors allow for the optimization of cell culture conditions to achieve optimal hydrogen production yields and process robustness. Environmental conditions that can be adjusted or monitored in a bioreactor include gas composition (e.g., air, oxygen, nitrogen, carbon dioxide), gas flow rates, temperature, pH, dissolved oxygen levels, and the agitation speed/circulation rate within the cell culture. Any type of bioreactor known in the art may be used for cell culture for the differentiation and expansion of cultured iCIL cells, including, but not limited to, a stirred-tank bioreactor, a pneumatic bioreactor (e.g., a bubble column or airlift bioreactor), a membrane bioreactor, a hollow-fiber bioreactor, a wave bioreactor, a vertical wheel bioreactor, a gas permeable rapid expansion (G-Rex) bioreactor, or a disposable bioreactor. In some embodiments, the bioreactor is a gas permeable rapid expansion (G-Rex) bioreactor. In some embodiments, the bioreactor is a vertical wheel bioreactor. In some embodiments, the bioreactor is a stirred-tank bioreactor. In some embodiments, the vertical wheel bioreactor is the PBS bioreactor. In some embodiments, the stirred-tank bioreactor is the Sartorius Ambr250 stirred tank bioreactor.

The expansion of the iCIL cells may be scaled to any desired volume to suit various purposes. For example, for high to medium throughput screening of various culture conditions, the process may be scaled to take place in a microbioreactor (e.g., about 15 mL to about 500 mL) or a benchtop scale bioreactor e.g., ranging from about 0.5 L to about 15 L). Alternatively, the process may be scaled up to a pilot scale bioreactor (e.g., ranging from about 15 L to about 15,000 L), or a manufacturing scale bioreactor (e.g., about 15,000 L to about 75,000 L or greater).

In some embodiments, one or more vessels may be used for iCIL expansion. In some embodiments, the cells may be cultured in the vessel from days 0-3, days 0-10, days 0-15, days 0-20, days 0-25, days 0-30, days 0-35, days 0-40, days 0-50, days 0-60, days 0-100, days 3-10, days 3-15, days 3-20, days 3-25, days 3-30, days 3-35, days 3-40, days 3-50, days 3-60, days 3-100, days 10-20, days 10-25, days 10-30, days 10-35, days 10-40, days 10-50, days 10-60, days 10-100, days 15-20, days 15-25, days 15-30, days 15-35, days 15-40, days 15-50, days 15-60, days 15-100, days 20-25, days 20-30, days 20-35, days 20-40, days 20-50, days 20-60, days 20-100, days 25-30, days 25-35, days 25-40, days 25-50, days 25-60, days 25-100, days 30-35, days 30-40, days 30-50, days 30-60, days 30-100, days 35-40, days 35-50, days 35-60, days 35-100, days 40-50, days 40-60, days 40-100, each inclusive. In some embodiments, the cells may be cultured in the vessel from days 0-35. In some embodiments, the cells may be cultured in the vessel from days 3-35. In some embodiments, the vessel is a bioreactor. In some embodiments, one or more bioreactors may be used for iCIL expansion. In some embodiments, the one or more bioreactors are different types of bioreactors. In some embodiments, the one or more bioreactors are different sizes of bioreactors.

G. Characteristics of Engineered Stem Cells and iCIL Cells

In some embodiments, the CIL cells are CD3-CD5−, CD16+, CD56+, CD57+, NKp30+, NKp46+, NKG2A+, and/or NKG2D+.

In some embodiments, the population of engineered cells is 40% to 60% CD16+, 50% to 70% CD16+, 60% to 80% CD16+, 70% to 90% CD16+, 80% to 100% CD16+, or any percentage within a range defined by any two aforementioned values.

In some embodiments, the population of engineered cells is 60% to 80% CD56+, 65% to 85% CD56+, 70% to 90% CD56+, 75% to 95% CD56+, 80% to 99% CD56+, or any percentage within a range defined by any two aforementioned values.

In some embodiments, the population of engineered CIL cells is CD56lo. In some embodiments, the population of engineered CIL cells is CD56high.

In some embodiments, the population of engineered CIL cells is 60% to 80% CD16+ CD56+, 65% to 85% CD16+ CD56+, 70% to 90% CD16+ CD56+, 75% to 95% CD16+ CD56+, 80% to 99% CD16+ CD56+, or any percentage within a range defined by any two aforementioned values.

In some embodiments, the population of engineered CIL cells is at least 40% CD16+, at least 50% CD16+, at least 60% CD16+, at least 70% CD16+, at least 80% CD16+, at least 90% CD16+, or 100% CD16+.

In some embodiments, the population of engineered CIL cells is at least 80% CD56+, at least 85% CD56+, at least 90% CD56+, at least 95% CD56+, or 100% CD56+.

In some embodiments, the population of engineered CIL cells is at least 40% CD16+ CD56+, at least 50% CD16+ CD56+, at least 60% CD16+ CD56+, at least 70% CD16+ CD56+, at least 80% CD16+ CD56+, at least 90% CD16+ CD56+, or 100% CD16+CD56+.

In some embodiments, CIL cells are characterized by being CD45+ CD56+.

In some embodiments, the population of engineered CIL cells is 40% to 60% CD45+, 50% to 70% CD45+, 60% to 80% CD45+. 70% to 90% CD45+, 80% to 100% CD45+, or any percentage within a range defined by any two aforementioned values.

In some embodiments, the population of engineered CIL cells is 60% to 80% CD45+ CD56+, 65% to 85% CD45+ CD56+, 70% to 90% CD45+ CD56+, 75% to 95% CD45+ CD56+, 80% to 99% CD45+ CD56+, or any percentage within a range defined by any two aforementioned values.

In some embodiments, the population of engineered CIL cells is at least 40% CD45+, at least 50% CD45+, at least 60% CD45+, at least 70% CD45+, at least 80% CD45+, at least 90% CD45+, or 100% CD45+.

In some embodiments, the population of engineered CIL cells is at least 40% CD45+ CD56+, at least 50% CD45+ CD56+, at least 60% CD45+ CD56+, at least 70% CD45+ CD56+, at least 80% CD45+ CD56+, at least 90% CD45+ CD56+, or 100% CD45+CD56+.

K562 cells are a highly sensitive target for an in vitro cytotoxic innate lymphoid cell cytotoxic activity assay. In the assay, CIL cells are co-incubated at different ratios with K562 target tumor cells known to be sensitive to CIL cell-mediated cytotoxicity. The target cells (K562) are pre-labeled with a fluorescent dye to allow their discrimination from the effector cells (CIL cells). After the incubation period, killed target cells are identified by a nucleic acid stain, which specifically permeates dead cells. % Dead is calculated by comparing the total number of viable cells in each experimental assay well to a non-effector control well. As used herein, the term “activity” refers to a measurement of the CIL cells' cytotoxic capacity against target cells.

In some embodiments, the engineered CIL cells secrete CD107a in response to an antigen recognized by the engineered CIL cells.

Innate CIL cell receptor stimulation causes CIL cell secretion of CD107a. CD107a expression correlates with both cytokine secretion and CIL cell-mediated lysis of target cells and thus, as used herein, CD107a secretion is a marker of CIL cell functional activity.

In some embodiments, the engineered CIL cells secrete interferon gamma (IFNγ) and/or tumor necrosis factor alpha (TNF-α) in response to an antigen recognized by the engineered CIL cells.

CIL cell activation leads to the secretion of IFNγ and TNF-α which synergistically enhance CIL cell cytotoxicity. In engineered CIL cells, expression of IFNγ and/or TNF-α are markers of CIL cell activity.

In some embodiments, the engineered CIL cells may be fresh or frozen. In some embodiments, the engineered CIL cells are fresh. In some embodiments, the engineered CIL cells are frozen. In some embodiments, when the frozen CIL cells are thawed, they retain viability and cytotoxic function compared to fresh CIL cells. In some embodiments, frozen/thawed CIL cells perform as well as controlling tumors as fresh CIL cells.

In some embodiments, the cells can be frozen by methods of cryopreservation. In some embodiments, iCILs are subjected to cryopreservation after their differentiation in accord with provided methods. In some embodiments, iCILs are subjected to cryopreservation after their engineering. In some embodiments, engineered iCILs produced according to provided methods are subjected to cryopreservation. In some embodiments, the method includes cryopreserving the cells in the presence of a cryoprotectant, thereby producing a cryopreserved composition. Any of a variety of known freezing solutions and parameters in some aspects may be used. In some embodiments, the cryoprotectant is DMSO. In some embodiments, the cells are frozen, e.g., cryopreserved, in a solution with a final concentration of between 1% and 15%, between 6% and 12%, between 5% and 10%, or between 6% and 8% DMSO. In some embodiments, the cryopreservation medium is between at or about 5% and at or about 10% DMSO (v/v). In some embodiments, the cryopreservation media contains one or more additional excipients, such as plasmalyte A or human serum albumin (HSA). In some embodiments, the solution for cryopreservation may also include human serum albumin (HSA). In particular embodiments, the cells are frozen, e.g., cryopreserved, in a solution with a final concentration of between 0.1% and 5%, between 0.25% and 4%, between 0.5% and 2%, or between 1% and 2% HSA. In some embodiments, the cryopreservation medium contains a commercially available cryopreservation solution (CryoStor™ CS10 or CS5). CryoStor™ CS10 is a cryopreservation medium containing 10% dimethyl sulfoxide (DMSO). CryoStor™ CS5 is a cryopreservation medium containing 5% dimethyl sulfoxide (DMSO). In some embodiments, the cells are generally then frozen to or to about −80° C. at a rate of or of about 1° per minute and stored in the vapor phase of a liquid nitrogen storage tank. In some aspects, the engineered iCILs are thawed prior to their use, such as in connection with methods of treatment described herein. In some embodiments, after thawing the cells the method includes washing the cryopreserved composition under conditions to reduce or remove the cyroprotectant.

### III. GENE EDITING AND ENGINEERING

In some embodiments the pluripotent stems cells (e.g. iPSCs) or iCILs may be modified by gene editing. In some embodiments the pluripotent stems cells (e.g. iPSCs) or iCILs may be modified by genetic engineering, such as by introducing an exogenous nucleic acid encoding a transgene, such as a chimeric antigen receptor (CAR). In some embodiments, the gene edited iPSCs as described may be used as source cells for differentiation into iCILs.

Genome editing generally refers to the process of editing or changing the nucleotide sequence of a genome, preferably in a precise, desirable and/or pre-determined manner. Examples of compositions, systems, and methods of genome editing described herein use site-directed nucleases to cut or cleave DNA at precise target locations in the genome, thereby creating a double-strand break (DSB) in the DNA. Such breaks can be repaired by endogenous DNA repair pathways, such as homology directed repair (HDR) and/or non-homologous end-joining (NHEJ) repair (see e.g., Cox et al., (2015) Nature Medicine 21 (2):121-31).

In some embodiments, the cells described herein (e.g., stem cells, CILs) are genetically modified. In some embodiments, the modification involves knocking out one or more endogenous genes using a DNA-targeted protein and a nuclease or an RNA-guided nuclease and/or knocking in one or more exogenous genes of interest. In some embodiments, a gene of interest is knocked into a particular locus of interest. In some embodiments, the gene of interest is a synthetic cytokine receptor complex. In some embodiments, the synthetic cytokine receptor complex is activated by rapamycin. In some embodiments, the synthetic cytokine receptor complex is a rapamycin activated cytokine receptor (RACR). In some embodiments, a RACR is knocked into a locus of interest. In some embodiments, the gene of interest is a chimeric antigen receptor.

In some embodiments, the modification comprises contacting a cell with a DNA-targeted protein and a nuclease or an RNA-guided nuclease. In some embodiments, a DNA-targeted protein and a nuclease or an RNA-guided nuclease includes zinc finger protein (ZFP), a clustered regularly interspaced short palindromic nucleic acid (CRISPR), or a TAL-effector nuclease (TALEN). In some embodiments, CRISPR-Cas9 is used. In some embodiments, CRISPR-Mad7 is used.

Rejection of cellular therapeutics (e.g., CAR T cells) is due at least to mismatches of human leukocyte antigen (HLA) between donor and recipient. One solution recently identified is disrupting expression of genes involved in this rejection, such as T cell receptor alpha constant (TRAC), beta-2-microglobulin (B2M), and signal regulatory protein alpha (SIRPA). Accordingly, in some embodiments, the cells described herein (e.g., iPSCs, CILs), are genetically engineered to knockout a B2M locus, a TRAC locus, and/or a SIRPA locus. In some embodiments, the cells described herein are genetically engineered to knockout a B2M locus. In some embodiments, the cells described herein are genetically engineered to knockout a TRAC locus. In some embodiments, the cells described herein are genetically engineered to knockout a SIRPA locus.

In some embodiments, the cells described herein are genetically engineered to be rapamycin resistant. Rapamycin is small molecule drug that inhibits the mTOR pathway, which is a pathway that is essential for cell growth and expansion. Thus, contacting a cell with rapamycin could, in some cases, inhibit or reduce cell growth and expansion. In some embodiments, to eliminate or reduce rapamycin-mediated growth inhibition of a source cell or CIL using the provided methods, the provided cells are disrupted in an endogenous gene involved in rapamycin function, thereby rendering such cells “rapamycin resistant.” It is understood that reference to a “rapamycin resistant” cell refers to the ability of a cell's endogenous mTOR pathway not to be affected or impacted by the presence of rapamycin or a rapamycin analog. However, it is further understood that a “rapamycin resistant” cell may nevertheless be responsive to rapamycin via a pathway that does not involve mTOR, such as due to activation of a synthetic RACR as described herein.

In some embodiments, the cells are genetically engineered to disrupt a gene associated with rapamycin recognition. In some embodiments, the cells are genetically engineered to disrupt the mTOR gene. In some embodiments, the mTOR gene is FKBP-12 (also known as FKBP-1A, FKBP1, FKBP12, PKC12, PKCI2, PPIASE). FKBP12 is an essential binder of rapamycin and required for its function. In some embodiments, the cells are genetically engineered to disrupt the FKBP12 gene. In some embodiments, the cells are genetically engineered to knockout the FKB12 gene to induce rapamycin resistance. In some embodiments, the disruption of the endogenous FKBP12 gene of the source stem cell (e.g. iPSC) is through genetic knock out with CRISPR-Cas system. In a normal cell without a genetic disruption of FKBP12, FKBP12 is the primary binder of rapamycin, and the FKBP12-rapamycin complex then binds to the FRB subunit of mTOR and blocks mTOR signaling. By disrupting expression of the FKBP12 gene such as by FKBP12 knockout, results herein demonstrate successful rapamycin suppression activity because rapamycin has no function without first complexing with FKBP1A. Thus, genetic disruption of FKBP12, such as by gene knock out, renders the stem cells (e.g. iPSCs) highly resistant to rapamycin-mediated mTOR inhibition, enabling robust growth of the stem cells (e.g. iPSC) even in the presence of high doses of rapamycin. In some embodiments, the ability to render cells resistant to rapamycin growth suppression permits engagement of the RACR by rapamycin during cell differentiation without deleterious effects. Further, knock out of FKBP12 avoids competition of FKBP12 with the RACR for binding to rapamycin. Thus, in some embodiments, the ability to render cells resistant to rapamycin growth by FKBP12 knock out also permits activation of RACR-containing cells in vivo and suppresses potential allogeneic anti-graft responses through mTOR suppression of the host immune system.

In some embodiments, the cells described herein are genetically engineered to comprise a nucleotide sequence encoding a synthetic cytokine receptor in an endogenous gene. In some embodiments, the synthetic cytokine receptor is engineered into a gene such that expression of the endogenous gene is not disrupted. In some embodiments, the cells described herein are genetically engineered to comprise a nucleotide sequence encoding a synthetic cytokine receptor complex in a disrupted gene, such as a gene that has been inactivated or knocked-out in the cell.

In some embodiments, the cells described herein are genetically engineered to comprise a nucleotide sequence encoding a synthetic cytokine receptor in a target endogenous gene. In some embodiments, the synthetic cytokine receptor is engineered into a safe-harbor locus. In some embodiments, the target endogenous gene is a housekeeping gene. In some embodiments, the housekeeping gene is eukaryotic translation elongation factor 1 alpha (EEF1A), glylceraldehyde-3-phosphate dehydrogenase (GAPDH), ubiquitin C (UBC), or actin beta (ACTB). In some embodiments, the target endogenous gene is a B2M, TRAC or SIRPalpha.

In some embodiments, the gene of interest inserted into an endogenous locus is a synthetic cytokine receptor complex. In some embodiments, the endogenous promoter of the particular locus is used.

In some embodiments, an exogenous promoter is operably connected to the gene encoding the synthetic cytokine receptor complex to drive expression. In some embodiments, the promoter is an EF1A promoter (also known as EEF1A promoter). In some embodiments, the promoter is an MND promoter. In some embodiments, additional promoter(s) may be included such that two or more promoters drive expression of the exogenous gene of interest. In some embodiments, the two or more promoters may be the same or different. In some embodiments, the promoter is a dual promoter in which the synthetic cytokine receptor is under the operable control of two promoters. In some embodiments, the dual promoter is a dual EF1α promoter.

For example, in some embodiments the cells comprise a disrupted B2M gene and a nucleotide sequence encoding the synthetic cytokine receptor in the disrupted B2M gene.

In some embodiments, the cells described herein (e.g., iPSCs, CILs) comprise (i) a disrupted B2M locus, and (ii) a nucleotide sequence encoding a synthetic cytokine receptor complex (e.g., a RACR) under control of the endogenous B2M promoter and an EEF1A promoter.

In some embodiments, the cells described herein (e.g., iPSCs, CILs) comprise (i) a disrupted B2M locus, and (ii) a nucleotide sequence encoding a synthetic cytokine receptor complex (e.g., a RACR) inserted into the endogenous B2M gene and under control of the endogenous B2M promoter and an EEF1A promoter.

In some embodiments, cells comprising (i) a disrupted B2M locus and (ii) a nucleotide sequence encoding a synthetic cytokine receptor complex (e.g., a RACR) are produced by any of the methods described below.

### A. Systems for Genome Editing

In some embodiments, a system for editing a cell described herein comprises a site-directed nuclease, such as a CRISPR/Cas system and optionally a gRNA. In some embodiments, the system comprises an engineered nuclease. In some embodiments, the system comprises a site-directed nuclease. In some embodiments, the site-directed nuclease comprises a CRISPR/Cas nuclease system. In some embodiments, the Cas nuclease is Cas9. In some embodiments, the nuclease is Mad7. In some embodiments, the guide RNA comprising the CRISPR/Cas system is a single guide RNA (sgRNA).

Sequences herein sets forth exemplary gRNA targeting sequences. In some embodiments, the gRNA targeting sequence may contain one or more thymines in the complementary portion sequence substituted with uracil. It will be understood by one of ordinary skill in the art that uracil and thymine can both be represented by ‘t’, instead of ‘u’ for uracil and ‘t’ for thymine; in the context of a ribonucleic acid, it will be understood that ‘t’ is used to represent uracil unless otherwise indicated.

### 1. Nuclease

a. CRISPR/Cas Nuclease Systems

Naturally-occurring CRISPR/Cas systems are genetic defense systems that provides a form of acquired immunity in prokaryotes. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see e.g., Koonin et al., (2017) Curr Opin Microbiol 37:67-78).

Engineered versions of CRISPR/Cas systems has been developed in numerous formats to mutate or edit genomic DNA of cells from other species. The general approach of using the CRISPR/Cas system involves the heterologous expression or introduction of a site-directed nuclease (e.g., Cas nuclease) in combination with a guide RNA (gRNA) into a cell, resulting in a DNA cleavage event (e.g., the formation a single-strand or double-strand break (SSB or DSB)) in the backbone of the cell's genomic DNA at a precise, targetable location. The manner in which the DNA cleavage event is repaired by the cell provides the opportunity to edit the genome by the addition, removal, or modification (substitution) of DNA nucleotide(s) or sequences (e.g., genes).

In some embodiments, a system for editing a cell described herein comprises a nuclease capable of inducing a DNA break within an endogenous target gene in the cell. In some embodiments, the DNA break comprises a double stranded break (DSB), which is induced by a nuclease capable of inducing a DSB by cleaving both strands of double stranded DNA at a cleavage site. In some embodiments, the DNA break comprises a single strand break (SSB) at a cleavage site in the sense strand or the antisense strand of the endogenous target gene. In some embodiments, the DNA break comprises a SSB at a cleavage site in the sense strand, and a SSB at a cleavage site in the antisense strand, thereby resulting in a DSB. In some embodiments, the DSB is induced by a pair of recombinant nucleases, e.g., nickases, that are each capable of inducing a single strand break (SSB) in opposite DNA strands at different cleavage sites, e.g., at a cleavage site upstream of the gene variant in one strand and at a cleavage site downstream of the gene variant in the other strand of the target gene. In some embodiments, a first of the pair of nickases forms a complex with a first guide RNA, e.g., a first sgRNA, for targeting cleavage to one strand, e.g., the sense strand, and the second of the pair of nickases forms a complex with a second guide RNA, e.g., a second sgRNA, for targeting cleavage to the other strand, e.g., the antisense strand. In some embodiments, a DSB is induced through a SSB on each of the opposite strands, i.e., the sense strand and the antisense strand, of an endogenous target gene in the cell.

In general, genes are located in double stranded DNA that includes a sense strand and an antisense strand, which are complementary to one another. The sense strand is also referred to as the coding strand because its sequence is the DNA version of the RNA sequence that is transcribed. The antisense strand is also referred to as the template strand because its sequence is complementary to the RNA sequence that is transcribed.

i. Guide RNAs (gRNAs)

Engineered CRISPR/Cas systems comprise at least two components: 1) a guide RNA (gRNA) molecule and 2) a Cas nuclease, which interact to form a gRNA/Cas nuclease complex. A gRNA comprises at least a user-defined targeting domain termed a “spacer” comprising a nucleotide sequence and a CRISPR repeat sequence. In engineered CRISPR/Cas systems, a gRNA/Cas nuclease complex is targeted to a specific target sequence of interest within a target nucleic acid (e.g., a genomic DNA molecule) by generating a gRNA comprising a spacer with a nucleotide sequence that is able to bind to the specific target sequence in a complementary fashion (See Jinek et al., Science, 337, 816-821 (2012) and Deltcheva et al., Nature, 471, 602-607 (2011)). Thus, the spacer provides the targeting function of the gRNA/Cas nuclease complex.

In naturally-occurring type II-CRISPR/Cas systems, the “gRNA” is comprised of two RNA strands: 1) a CRISPR RNA (crRNA) comprising the spacer and CRISPR repeat sequence, and 2) a trans-activating CRISPR RNA (tracrRNA). In Type II-CRISPR/Cas systems, the portion of the crRNA comprising the CRISPR repeat sequence and a portion of the tracrRNA hybridize to form a crRNA: tracrRNA duplex, which interacts with a Cas nuclease (e.g., Cas9). As used herein, the terms “split gRNA” or “modular gRNA” refer to a gRNA molecule comprising two RNA strands, wherein the first RNA strand incorporates the crRNA function(s) and/or structure and the second RNA strand incorporates the tracrRNA function(s) and/or structure, and wherein the first and second RNA strands partially hybridize.

Accordingly, in some embodiments, a gRNA comprises two RNA molecules. In some embodiments, the gRNA comprises a CRISPR RNA (crRNA) and a trans-activating CRISPR RNA (tracrRNA). In some embodiments, the gRNA is a split gRNA. In some embodiments, the gRNA is a modular gRNA. In some embodiments, the split gRNA comprises a first strand comprising, from 5′ to 3′, a spacer, and a first region of complementarity; and a second strand comprising, from 5′ to 3′, a second region of complementarity; and optionally a tail domain.

In some embodiments, the crRNA comprises a spacer comprising a nucleotide sequence that is complementary to and hybridizes with a sequence that is complementary to the target sequence on a target nucleic acid (e.g., a genomic DNA molecule). In some embodiments, the crRNA comprises a region that is complementary to and hybridizes with a portion of the tracrRNA.

In some embodiments, the target nucleic acid (e.g., endogenous gene) is B2M. In some embodiments, the crRNA comprises the nucleotide sequence set forth in SEQ ID NO: 18, or a nucleotide sequence having at least 80%, 85%, 90%, or 95% sequence identity to the nucleotide sequence set forth in SEQ ID NO: 18. In some embodiments, the crRNA comprises the nucleotide sequence set forth in SEQ ID NO:18.

In some embodiments, the target nucleic acid (e.g., endogenous gene) is FKBP12. In some embodiments, the crRNA comprises the nucleotide sequence set forth in any one of SEQ ID NOS: 19, 20, and 21, or a nucleotide sequence having at least 80%, 85%, 90%, or 95% sequence identity to a nucleotide sequence set forth in any one of SEQ ID NOS: 19, 20, and 21. In some embodiments, the crRNA comprises the nucleotide sequence set forth in any one of SEQ ID NOS: 19, 20, and 21. In some embodiments, the crRNA comprises the nucleotide sequence set forth in SEQ ID NO: 19, or a nucleotide sequence having at least 80%, 85%, 90%, or 95% sequence identity to the nucleotide sequence set forth in SEQ ID NO: 19. In some embodiments, the crRNA comprises the nucleotide sequence set forth in SEQ ID NO:19 In some embodiments, the crRNA comprises the nucleotide sequence set forth in SEQ ID NO:20, or a nucleotide sequence having at least 80%, 85%, 90%, or 95% sequence identity to the nucleotide sequence set forth in SEQ ID NO:20. In some embodiments, the crRNA comprises the nucleotide sequence set forth in SEQ ID NO:20. In some embodiments, the crRNA comprises the nucleotide sequence set forth in SEQ ID NO: 21, or a nucleotide sequence having at least 80%, 85%, 90%, or 95% sequence identity to the nucleotide sequence set forth in SEQ ID NO:21. In some embodiments, the crRNA comprises the nucleotide sequence set forth in SEQ ID NO:21.

In some embodiments, the tracrRNA may comprise all or a portion of a wild-type tracrRNA sequence from a naturally-occurring CRISPR/Cas system. In some embodiments, the tracrRNA may comprise a truncated or modified variant of the wild-type tracr RNA. The length of the tracr RNA may depend on the CRISPR/Cas system used. In some embodiments, the tracrRNA may comprise 5, 6, 7, 8, 9, 10, 11, 12, 13, 14, 15, 16, 17, 18, 19, 20, 25, 30, 40, 50, 60, 70, 80, 90, 100, or more than 100 nucleotides in length. In certain embodiments, the tracrRNA is at least 26 nucleotides in length. In additional embodiments, the tracrRNA is at least 40 nucleotides in length. In some embodiments, the tracrRNA may comprise certain secondary structures, such as, e.g., one or more hairpins or stem-loop structures, or one or more bulge structures.

Single Guide RNA (sgRNA)

Engineered CRISPR/Cas nuclease systems often combine a crRNA and a tracrRNA into a single RNA molecule, referred to herein as a “single guide RNA” (sgRNA), by adding a linker between these components. Without being bound by theory, similar to a duplexed crRNA and tracrRNA, an sgRNA will form a complex with a Cas nuclease (e.g., Cas9), guide the Cas nuclease to a target sequence and activate the Cas nuclease for cleavage the target nucleic acid (e.g., genomic DNA). Accordingly, in some embodiments, the gRNA may comprise a crRNA and a tracrRNA that are operably linked. In some embodiments, the sgRNA may comprise a crRNA covalently linked to a tracrRNA. In some embodiments, the crRNA and the tracrRNA is covalently linked via a linker. In some embodiments, the sgRNA may comprise a stem-loop structure via base pairing between the crRNA and the tracrRNA. In some embodiments, a sgRNA comprises, from 5′ to 3′, a spacer, a first region of complementarity, a linking domain, a second region of complementarity, and, optionally, a tail domain.

The sgRNA can be unmodified or modified. For example, modified sgRNAs can comprise one or more 2′-O-methyl phosphorothioate nucleotides.

By way of illustration, guide RNAs used in the CRISPR/Cas system, or other smaller RNAs can be readily synthesized by chemical means, as illustrated herein and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

### Spacers

In some embodiments, the gRNAs comprise a spacer sequence. A spacer sequence is a sequence that defines the target site of a target nucleic acid (e.g. DNA). The target nucleic acid is a double-stranded molecule: one strand comprises the target sequence adjacent to a PAM sequence and is referred to as the “PAM strand,” and the second strand is referred to as the “non-PAM strand” and is complementary to the PAM strand and target sequence. Both gRNA spacer and the target sequence are complementary to the non-PAM strand of the target nucleic acid. In some embodiments, a spacer sequence corresponding to a target sequence adjacent to a PAM sequence is complementary to the non-PAM strand of the target nucleic acid. Thus, in some embodiments, a spacer sequence which corresponds to a target sequence adjacent to a PAM sequence is identical to the PAM strand. The gRNA spacer sequence hybridizes to the complementary strand (e.g.: the non-PAM strand of the target nucleic acid/target site). In some embodiments, the spacer is sufficiently complementary to the complementary strand of the target sequence (e.g.: non-PAM strand), as to target a Cas nuclease to the target nucleic acid. In some embodiments, the spacer is at least 80%, 85%, 90% or 95% complementary to the non-PAM strand of the target nucleic acid. In some embodiments, the spacer is 100% complementary to the non-PAM strand of the target nucleic acid. In some embodiments, the spacer comprises 1, 2, 3, 4, 5, 6 or more nucleotides that are not complementary with the non-PAM strand of the target nucleic acid. In some embodiments, the spacer comprises 1 nucleotide that is not complementary with the non-PAM strand of the target nucleic acid. In some embodiments, the spacer comprises 2 nucleotides that are not complementary with the non-PAM strand of the target nucleic acid.

In some embodiments, the 5′ most nucleotide of gRNA comprises the 5′ most nucleotide of the spacer. In some embodiments, the spacer is located at the 5′ end of the crRNA. In some embodiments, the spacer is located at the 5′ end of the sgRNA. In some embodiments, the spacer is about 15-50, about 20-45, about 25-40 or about 30-35 nucleotides in length. In some embodiments, the spacer is about 19-22 nucleotides in length. In some embodiments the spacer is about 15, 16, 17, 18, 19, 20, 21, 22, 23, 24, 25, 26, 27, 28, 29 or 30 nucleotides in length. In some embodiments the spacer is 19 nucleotides in length. In some embodiments, the spacer is 20 nucleotides in length, in some embodiments, the spacer is 21 nucleotides in length.

In some embodiments, the nucleotide sequence of the spacer is designed or chosen using a computer program. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, and/or presence of SNPs.

In some embodiments, the spacer comprise at least one or more modified nucleotide(s) such as those described herein. The disclosure provides gRNA molecules comprising a spacer which may comprise the nucleobase uracil (U), while any DNA encoding a gRNA comprising a spacer comprising the nucleobase uracil (U) will comprise the nucleobase thymine (T) in the corresponding position(s).

ii. Methods of Making gRNAs

Methods for making gRNAs are known to those of skill in the art and include but not limited to in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in International Application PCT/US2013/30062. Accordingly, the present disclosure also includes polynucleotides, e.g., DNA, constructs and vectors are used to in vitro transcribe a gRNA described herein.

In some embodiments, non-natural modified nucleobases are introduced into polynucleotides, e.g., gRNA, during synthesis or post-synthesis. In certain embodiments, modifications are on internucleoside linkages, purine or pyrimidine bases, or sugar. In some embodiments, the modification is introduced at the terminal of a polynucleotide; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

In some embodiments, enzymatic or chemical ligation methods are used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

In some embodiments, the disclosure provides nucleic acids, e.g., vectors, encoding gRNAs described herein. In some embodiments, the nucleic acid is a DNA molecule. In other embodiments, the nucleic acid is an RNA molecule. In some embodiments, the nucleic acid comprises a nucleotide sequence encoding a crRNA. In some embodiments, the nucleotide sequence encoding the crRNA comprises a spacer flanked by all or a portion of a repeat sequence from a naturally-occurring CRISPR/Cas system. In some embodiments, the nucleic acid comprises a nucleotide sequence encoding a tracrRNA. In some embodiments, the crRNA and the tracrRNA is encoded by two separate nucleic acids. In other embodiments, the crRNA and the tracrRNA is encoded by a single nucleic acid. In some embodiments, the crRNA and the tracrRNA is encoded by opposite strands of a single nucleic acid. In other embodiments, the crRNA and the tracrRNA is encoded by the same strand of a single nucleic acid.

In some embodiments, the gRNAs provided by the disclosure are chemically synthesized by any means described in the art (see e.g., WO/2005/01248). While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together.

In some embodiments, more than one guide RNA can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

The guide RNA may target any sequence of interest via the targeting sequence (e.g.: spacer sequence) of the crRNA. In some embodiments, the degree of complementarity between the targeting sequence of the guide RNA and the target sequence on the target nucleic acid molecule is about 60%, 65%, 70%, 75%, 80%, 85%, 90%, 95%, 97%, 98%, 99%, or 100%. In some embodiments, the targeting sequence of the guide RNA and the target sequence on the target nucleic acid molecule is 100% complementary. In other embodiments, the targeting sequence of the guide RNA and the target sequence on the target nucleic acid molecule may contain at least one mismatch. For example, the targeting sequence of the guide RNA and the target sequence on the target nucleic acid molecule may contain 1, 2, 3, 4, 5, 6, 7, 8, 9, or 10 mismatches. In some embodiments, the targeting sequence of the guide RNA and the target sequence on the target nucleic acid molecule may contain 1-6 mismatches. In some embodiments, the targeting sequence of the guide RNA and the target sequence on the target nucleic acid molecule may contain 5 or 6 mismatches.

The length of the targeting sequence may depend on the CRISPR-Cas system and components used. For example, different Cas9 proteins from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence may comprise 18-24 nucleotides in length. In some embodiments, the targeting sequence may comprise 19-21 nucleotides in length. In some embodiments, the targeting sequence may comprise 20 nucleotides in length.

In some embodiments of the present disclosure, a CRISPR/Cas nuclease system includes at least one guide RNA. In some embodiments, the guide RNA and the Cas protein may form a ribonucleoprotein (RNP), e.g., a CRISPR/Cas complex. The guide RNA may guide the Cas protein to a target sequence on a target nucleic acid molecule (e.g., a genomic DNA molecule), where the Cas protein cleaves the target nucleic acid. In some embodiments, the CRISPR/Cas complex is a Cpf1/guide RNA complex. In some embodiments, the CRISPR complex is a Type-II CRISPR/Cas9 complex. In some embodiments, the Cas protein is a Cas9 protein. In some embodiments, the CRISPR/Cas9 complex is a Cas9/guide RNA complex. In some embodiments, the CRISPR/Cas complex is an engineered Class 2 Type V CRISPR system. In some embodiments, the endonuclease is Mad7.

iii. Cas Nuclease

In some embodiments, the disclosure provides compositions and systems (e.g., an engineered CRISPR/Cas system) comprising a site-directed nuclease, wherein the site-directed nuclease is a Cas nuclease. The Cas nuclease may comprise at least one domain that interacts with a guide RNA (gRNA). Additionally, the Cas nuclease are directed to a target sequence by a guide RNA. The guide RNA interacts with the Cas nuclease as well as the target sequence such that, once directed to the target sequence, the Cas nuclease is capable of cleaving the target sequence. In some embodiments, the guide RNA provides the specificity for the cleavage of the target sequence, and the Cas nuclease are universal and paired with different guide RNAs to cleave different target sequences.

In some embodiments, the CRISPR/Cas system comprise components derived from a Type-I. Type-II, or Type-III system. Updated classification schemes for CRISPR/Cas loci define Class 1 and Class 2 CRISPR/Cas systems, having Types I to V or VI (Makarova et al., (2015) Nat Rev Microbiol, 13 (11): 722-36; Shmakov et al., (2015) Mol Cell, 60:385-397). Class 2 CRISPR/Cas systems have single protein effectors. Cas proteins of Types II, V. and VI are single-protein, RNA-guided endonucleases, herein called “Class 2 Cas nucleases.” Class 2 Cas nucleases include, for example, Cas9, Cpf1, C2c1, C2c2, and C2c3 proteins. The Cpf1 nuclease (Zetsche et al., (2015) Cell 163:1-13) is homologous to Cas9, and contains a RuvC-like nuclease domain.

In some embodiments, the Cas nuclease are from a Type-II CRISPR/Cas system (e.g., a Cas9 protein from a CRISPR/Cas9 system). In some embodiments, the Cas nuclease are from a Class 2 CRISPR/Cas system (a single-protein Cas nuclease such as a Cas9 protein or a Cpf1 protein). The Cas9 and Cpf1 family of proteins are enzymes with DNA endonuclease activity, and they can be directed to cleave a desired nucleic acid target by designing an appropriate guide RNA, as described further herein.

A Type-II CRISPR/Cas system component are from a Type-IIA, Type-IIB, or Type-IIC system. Cas9 and its orthologs are encompassed. Non-limiting exemplary species that the Cas9 nuclease or other components are from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, or Acaryochloris marina. In some embodiments, the Cas9 protein are from Streptococcus pyogenes (SpCas9). In some embodiments, the Cas9 protein are from Streptococcus thermophilus (StCas9). In some embodiments, the Cas9 protein are from Neisseria meningitides (NmCas9). In some embodiments, the Cas9 protein are from Staphylococcus aureus (SaCas9). In some embodiments, the Cas9 protein are from Campylobacter jejuni (CjCas9).

In some embodiments, a Cas nuclease may comprise more than one nuclease domain. For example, a Cas9 nuclease may comprise at least one RuvC-like nuclease domain (e.g., Cpf1) and at least one HNH-like nuclease domain (e.g., Cas9). In some embodiments, the Cas9 nuclease introduces a DSB in the target sequence. In some embodiments, the Cas9 nuclease is modified to contain only one functional nuclease domain. For example, the Cas9 nuclease is modified such that one of the nuclease domains is mutated or fully or partially deleted to reduce its nucleic acid cleavage activity. In some embodiments, the Cas9 nuclease is modified to contain no functional RuvC-like nuclease domain. In other embodiments, the Cas9 nuclease is modified to contain no functional HNH-like nuclease domain. In some embodiments in which only one of the nuclease domains is functional, the Cas9 nuclease is a nickase that is capable of introducing a single-stranded break (a “nick”) into the target sequence. In some embodiments, a conserved amino acid within a Cas9 nuclease domain is substituted to reduce or alter a nuclease activity. In some embodiments, the Cas nuclease nickase comprises an amino acid substitution in the RuvC-like nuclease domain. Exemplary amino acid substitutions in the RuvC-like nuclease domain include D10A (based on the S. pyogenes Cas9 nuclease). In some embodiments, the nickase comprises an amino acid substitution in the HNH-like nuclease domain. Exemplary amino acid substitutions in the HNH-like nuclease domain include E762A, H840A, N863A, H983A, and D986A (based on the S. pyogenes Cas9 nuclease). In some embodiments, the nuclease system described herein comprises a nickase and a pair of guide RNAs that are complementary to the sense and antisense strands of the target sequence, respectively. The guide RNAs directs the nickase to target and introduce a DSB by generating a nick on opposite strands of the target sequence (i.e., double nicking). Chimeric Cas9 nucleases are used, where one domain or region of the protein is replaced by a portion of a different protein. For example, a Cas9 nuclease domain is replaced with a domain from a different nuclease such as Fok1. A Cas9 nuclease is a modified nuclease.

In some embodiments, the Cas nuclease is from a Type-I CRISPR/Cas system. In some embodiments, the Cas nuclease is a component of the Cascade complex of a Type-I CRISPR/Cas system. For example, the Cas nuclease is a Cas3 nuclease. In some embodiments, the Cas nuclease is derived from a Type-III CRISPR/Cas system. In some embodiments, the Cas nuclease is derived from Type-IV CRISPR/Cas system. In some embodiments, the Cas nuclease is derived from a Type-V CRISPR/Cas system. In some embodiments, the Cas nuclease is derived from a Type-VI CRISPR/Cas system.

In some embodiments, the Cas nuclease is a Mad endonuclease. CRISPR/Mad systems are closely related to the Type V (Cpf1-like) of Class-2 family of Cas enzymes. In some embodiments, the CRISPR-Mad system employs an Eubacterium rectale Mad7 endonuclease or variant thereof. The Mad7-crRNA complex cleaves target DNA by identification of a PAM 5′-YTTN.

b. Engineered Nucleases

In some embodiments, the cells described herein are genetically engineered with a site-directed nuclease, wherein the site-directed nuclease is an engineered nuclease. Exemplary engineered nucleases are meganuclease (e.g., homing endonucleases), ZFN, TALEN, and megaTAL.

Naturally-occurring meganucleases may recognize and cleave double-stranded DNA sequences of about 12 to 40 base pairs and are commonly grouped into five families. In some embodiments, the meganuclease are chosen from the LAGLIDADG family, the GIY-YIG family, the HNH family, the His-Cys box family, and the PD-(D/E) XK family. In some embodiments, the DNA binding domain of the meganuclease are engineered to recognize and bind to a sequence other than its cognate target sequence. In some embodiments, the DNA binding domain of the meganuclease are fused to a heterologous nuclease domain. In some embodiments, the meganuclease, such as a homing endonuclease, are fused to TAL modules to create a hybrid protein, such as a “megaTAL” protein. The megaTAL protein have improved DNA targeting specificity by recognizing the target sequences of both the DNA binding domain of the meganuclease and the TAL modules.

ZFNs are fusion proteins comprising a zinc-finger DNA binding domain (“zinc fingers” or “ZFs”) and a nuclease domain. Each naturally-occurring ZF may bind to three consecutive base pairs (a DNA triplet), and ZF repeats are combined to recognize a DNA target sequence and provide sufficient affinity. Thus, engineered ZF repeats are combined to recognize longer DNA sequences, such as, e.g., 9-, 12-, 15-, or 18-bp, etc. In some embodiments, the ZFN comprise ZFs fused to a nuclease domain from a restriction endonuclease. For example, the restriction endonuclease is FokI. In some embodiments, the nuclease domain comprises a dimerization domain, such as when the nuclease dimerizes to be active, and a pair of ZFNs comprising the ZF repeats and the nuclease domain is designed for targeting a target sequence, which comprises two half target sequences recognized by each ZF repeats on opposite strands of the DNA molecule, with an interconnecting sequence in between (which is sometimes called a spacer in the literature). For example, the interconnecting sequence is 5 to 7 bp in length. When both ZFNs of the pair bind, the nuclease domain may dimerize and introduce a DSB within the interconnecting sequence. In some embodiments, the dimerization domain of the nuclease domain comprises a knob-into-hole motif to promote dimerization. For example, the ZFN comprises a knob-into-hole motif in the dimerization domain of FokI.

The DNA binding domain of TALENs usually comprises a variable number of 34 or 35 amino acid repeats (“modules” or “TAL modules”), with each module binding to a single DNA base pair. A. T. G, or C. Adjacent residues at positions 12 and 13 (the “repeat-variable di-residue” or RVD) of each module specify the single DNA base pair that the module binds to. Though modules used to recognize G may also have affinity for A. TALENs benefit from a simple code of recognition—one module for each of the 4 bases—which greatly simplifies the customization of a DNA-binding domain recognizing a specific target sequence. In some embodiments, the TALEN may comprise a nuclease domain from a restriction endonuclease. For example, the restriction endonuclease is FokI. In some embodiments, the nuclease domain may dimerize to be active, and a pair of TALENS is designed for targeting a target sequence, which comprises two half target sequences recognized by each DNA binding domain on opposite strands of the DNA molecule, with an interconnecting sequence in between. For example, each half target sequence is in the range of 10 to 20 bp, and the interconnecting sequence is 12 to 19 bp in length. When both TALENs of the pair bind, the nuclease domain may dimerize and introduce a DSB within the interconnecting sequence. In some embodiments, the dimerization domain of the nuclease domain may comprise a knob-into-hole motif to promote dimerization. For example, the TALEN may comprise a knob-into-hole motif in the dimerization domain of FokI.

c. Target Sites

In some embodiments, the site-directed nucleases described herein are directed to and cleave (e.g., introduce a DSB) a target nucleic acid molecule (e.g. endogenous gene). In some embodiments, the target nucleic acid molecule is a housekeeping gene. In some embodiments, the housekeeping gene is eukaryotic translation elongation factor 1 alpha (EEF1A), glylceraldehyde-3-phosphate dehydrogenase (GAPDH), ubiquitin C (UBC), or actin beta (ACTB). In some embodiments, the target nucleic acid molecule is a blood-lineage gene. In some embodiments, the blood-lineage gene is protein tyrosine phosphatase receptor type C (PTPRC), IL2RG, or IL2RB. In some embodiments, the target nucleic acid is a gene associated with rapamycin response. In some embodiments, the target nucleic acid is FKBP12. In some embodiments, the target nucleic acid is B2M, TRAC or SIRPA.

The target nucleic acid molecule is any DNA molecule that is endogenous or exogenous to a cell. As used herein, the term “endogenous sequence” refers to a sequence that is native to the cell. In some embodiments, the target nucleic acid molecule is a genomic DNA (gDNA) molecule or a chromosome from a cell or in the cell. In some embodiments, the target sequence of the target nucleic acid molecule is a genomic sequence from a cell or in the cell. In some embodiments, the target sequence may be located in a coding sequence of a gene, an intron sequence of a gene, a transcriptional control sequence of a gene, a translational control sequence of a gene, or a non-coding sequence between genes. In some embodiments, the gene may be a protein coding gene. In other embodiments, the gene may be a non-coding RNA gene. In some embodiments, the target sequence may comprise all or a portion of a disease-associated gene.

In some embodiments, the target sequence may be located in a non-genic functional site in the genome that controls aspects of chromatin organization, such as a scaffold site or locus control region. In some embodiments, the target sequence may be a genetic safe harbor site, i.e., a locus that facilitates safe genetic modification.

In some embodiments, the target sequence may be adjacent to a protospacer adjacent motif (PAM), a short sequence recognized by a CRISPR/Cas complex. In some embodiments, the PAM may be adjacent to or within 1, 2, 3, or 4, nucleotides of the 3′ end of the target sequence. In some embodiments, the target sequence may include the PAM. The length and the sequence of the PAM may depend on the Cas protein used. For example, the PAM may be selected from a consensus or a particular PAM sequence for a specific Cas nuclease or Cas ortholog, including those disclosed in FIG. 1 of Ran et al., (2015) Nature, 520:186-191 (2015), which is incorporated herein by reference. In some embodiments, the PAM may comprise 2, 3, 4, 5, 6, 7, 8, 9, or 10 nucleotides in length. Non-limiting exemplary PAM sequences include NGG (SpCas9 WT, SpCas9 nickase, dimeric dCas9-Fok1, SpCas9-HF1, SpCas9 K855A, eSpCas9 (1.0), eSpCas9 (1.1)), NGAN or NGNG (SpCas9 VQR variant), NGAG (SpCas9 EQR variant), NGCG (SpCas9 VRER variant), NAAG (SpCas9 QQR1 variant), NNGRRT or NNGRRN (SaCas9), NNNRRT (KKH SaCas9), NNNNRYAC (CjCas9), NNAGAAW (St1Cas9), NAAAAC (TdCas9), NGGNG (St3Cas9), NG (FnCas9), NAAAAN (TdCas9), NNAAAAW (StCas9), NNNNACA (CjCas9), GNNNCNNA (PmCas9), and NNNNGATT (NmCas9) (scc e.g., Cong et al., (2013) Science 339:819-823; Kleinstiver et al., (2015) Nat Biotechnol 33:1293-1298; Kleinstiver et al., (2015) Nature 523:481-485; Kleinstiver et al., (2016) Nature 529:490-495; Tsai et al., (2014) Nat Biotechnol 32:569-576; Slaymaker et al., (2016) Science 351:84-88; Anders et al., (2016) Mol Cell 61:895-902; Kim et al., (2017) Nat Comm 8:14500; Fonfara et al., (2013) Nucleic Acids Res 42:2577-2590; Garneau et al., (2010) Nature 468:67-71; Magadan et al., (2012) PLOS ONE 7:040913; Esvelt et al., (2013) Nat Methods 10 (11): 1116-1121 (wherein N is defined as any nucleotide, W is defined as either A or T. R is defined as a purine (A) or (G), and Y is defined as a pyrimidine (C) or (T)). In some embodiments, the PAM sequence is NGG. In some embodiments, the PAM sequence is NGAN. In some embodiments, the PAM sequence is NGNG. In some embodiments, the PAM is NNGRRT. In some embodiments, the PAM sequence is NGGNG. In some embodiments, the PAM sequence may be NNAAAAW.

In some embodiments, the PAM sequence that is recognized by a nuclease, e.g., Cas9, differs depending on the particular nuclease and the bacterial species it is from. In some embodiments, the PAM sequence recognized by SpCas9 is the nucleotide sequence 5′-NGG-3′, where “N” is any nucleotide. In some embodiments, a PAM sequence recognized by SaCas9 is the nucleotide sequence 5′-NGRRT-3′ or the nucleotide sequence 5′-NGRRN-3′, where “N” is any nucleotide and “R” is a purine (e.g., guanine or adenine). In some embodiments, a PAM sequence recognized by NmeCas9 is the nucleotide sequence 5′-NNNNGATT-3′, where “N” is any nucleotide. In some embodiments, a PAM sequence recognized by CjCas9 is the nucleotide sequence 5′-NNNNRYAC-3′, where “N” is any nucleotide, “R” is a purine (e.g., guanine or adenine), and “Y” is a pyrimidine (e.g., cytosine or thymine). In some embodiments, a PAM sequence recognized by StCas9 is the nucleotide sequence 5′-NNAGAAW-3′, where “N” is any nucleotide and “W” is adenine or thymine.

In some embodiments, the recombinant nuclease is Cas9 and the PAM sequence is the nucleotide sequence: (a) 5′-NGG-3′; (b) 5′-NGRRT-3′ or 5′-NGRRN-3′; (c) 5′-NNNNGATT-3′; (d) 5′-NNNNRYAC-3′; or (c) 5′-NNAGAAW-3′; where “N” is any nucleotide, “R” is a purine (e.g., guanine or adenine), “Y” is a pyrimidine (e.g., cytosine or thymine), and “W” is adenine or thymine. In some embodiments, the recombinant nuclease is Cas9, e.g., SpCas9, and the PAM sequence is 5′-NGG-3′, where “N” is any nucleotide. In some embodiments, the recombinant nuclease is Cas9, e.g., SaCas9, and the PAM sequence is 5′-NGRRT-3′ or 5′-NGRRN-3′, where “N” is any nucleotide and “R” is a purine, such as guanine or adenine. In some embodiments, the recombinant nuclease is Cas9, e.g., NmeCas9, and the PAM sequence is 5′-NNNNGATT-3′, where “N” is any nucleotide. In some embodiments, the recombinant nuclease is Cas9, e.g., CjCas9, and the PAM sequence is 5′-NNNNRYAC-3′, where “N” is any nucleotide, “R” is a purine, such as guanine or adenine, and “Y” is a pyrimidine, such as cytosine or thymine. In some embodiments, the recombinant nuclease is Cas9, e.g., StCas9, and the PAM sequence is 5′-NNAGAAW-3′, where “N” is any nucleotide and “W” is adenine or thymine.

d. Ribonucleoproteins

In some embodiments, the site-directed polypeptide (e.g., Cas nuclease) and genome-targeting nucleic acid (e.g., gRNA or sgRNA) may each be administered separately to a cell or a subject. In some embodiments, the site-directed polypeptide may be pre-complexed with one or more guide RNAs, or one or more sgRNAs. Such pre-complexed material is known as a ribonucleoprotein particle (RNP). In some embodiments, the nuclease system comprises a ribonucleoprotein (RNP). In some embodiments, the nuclease system comprises a Cas9 RNP comprising a purified Cas9 protein in complex with a gRNA. In some embodiments, the nuclease system comprises a Mad7 RNP comprising a purified Mad7 protein in complex with a gRNA. Cas9 and Mad7 protein can be expressed and purified by any means known in the art. Ribonucleoproteins are assembled in vitro and can be delivered directly to cells using standard electroporation or transfection techniques known in the art.

### B. Targeted Gene Insertions

In some embodiments, the synthetic cytokine receptor (e.g. RACR) is integrated into a target nucleic acid molecule (e.g. an endogenous gene). In some embodiments, the integration into a target endogenous gene can disrupt expression of the target endogenous gene in the cells. In some embodiments, the target nucleic acid molecule is a housekeeping gene. In some embodiments, the housekeeping gene is eukaryotic translation elongation factor 1 alpha (EEF1A), glyceraldehyde-3-phosphate dehydrogenase (GAPDH), ubiquitin C (UBC), or actin beta (ACTB). In some embodiments, the target nucleic acid is B2M, TRAC or SIRPA. In some embodiments, the target nucleic acid is B2M. In some embodiment, a nucleic acid encoding the engineered cytokine receptor is integrated into a disrupted B2M locus, such as by HDR or other methods. In some embodiments, HDR can be used to integrate a donor template comprising a nucleic acid encoding a synthetic cytokine receptor (e.g., a RACR) into a target nucleic acid molecule (e.g. an endogenous gene). For instance, by HDR methods a construct encoding the synthetic cytokine receptor further comprises a first homology arm and a second homology arm homologous to a target gene locus for CRISPR-based homology directed repair.

In some embodiments, one or more additional genes can be knocked-in or inserted into the genome of a cell. In some embodiments, a gene encoding a chimeric antigen receptor (CAR), such as described in Section IV, is inserted into the genome of a cell. In some embodiments, a gene encoding FRB, such as described in Section C, is inserted into the genome of a cell. In some embodiments, each of the one or more additional gene may be individual integrated into an endogenous gene. In some embodiments, the integration into a target endogenous gene can disrupt expression of the target endogenous gene in the cells. In some embodiments, the target nucleic acid molecule is a housekeeping gene. In some embodiments, the housekeeping gene is eukaryotic translation elongation factor 1 alpha (EEF1A), glylceraldehyde-3-phosphate dehydrogenase (GAPDH), ubiquitin C (UBC), or actin beta (ACTB). In some embodiments, the target nucleic acid is B2M, TRAC or SIRPA. In some embodiments, HDR can be used to integrate a donor template comprising a nucleic acid encoding an additional gene (e.g. CAR or FRB) into an endogenous gene. For instance, by HDR methods a construct encoding the additional gene (e.g. nucleic acid encoding CAR or FRB) further comprises a first homology arm and a second homology arm homologous to a target gene locus for CRISPR-based homology directed repair. In some embodiments, a nucleic acid construct encoding the synthetic cytokine receptor is integrated into the B2M locus and a nucleic acid encoding FRB or a CAR is integrated into the ACTB or EF1A locus. In some embodiments, a nucleic acid construct encoding the synthetic cytokine receptor is integrated into the B2M locus, a nucleic acid encoding FRB is integrated into one of the ACTB or EF1A locus, and a nucleic acid encoding a CAR is integrated into the other of the ACTB and EF1A locus.

In some embodiments, transient BCL-XL overexpression is carried out in a cell that is disrupted for certain endogenous genes that are essential genes (Li et al. (2018) Nucleic Acids Research, 46:10195-10215). For instance, in some cases, editing essential genes requires anti-apoptotic support to enable clone selection and this can be achieved by providing transient overexpression of BCL-2 during editing. In some embodiments, transient BCL-XL overexpression can be achieved by introduction of a BCL-XL mRNA in the cell.

In some of embodiments, a stem cell, such as an iPSC, is engineered with the targeted gene insertion or insertions. In some embodiments, a progenitor cells, such as a CLP or HP, is engineered with the targeted gene insertion or insertions. In some embodiments, an iCIL is engineered with the targeted gene insertion or insertions.

Methods of introducing an exogenous gene, such as the synthetic cytokine receptor, into a target nucleic acid molecule (e.g. endogenous gene) are well known in the art (see for example Menke D. Genesis (2013) 51:618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties) and include targeted homologous recombination, site specific recombinases, PB transposases and genome editing by engineered nucleases. Agents for introducing the synthetic cytokine receptor into a target nucleic acid molecule can be designed via publicly available sources or obtained commercially from Transposagen, Addgene and Sangamo Biosciences.

In some embodiments the gene editing technologies can be used for knock-out or knock-down of genes. In some embodiments, the gene-editing technologies can be used for knock-in or integration of DNA into a region of the genome. In some embodiments, the gene editing technology mediates double-strand breaks (DSB), including in connection with non-homologous end-joining (NHEJ) or homology-directed repair (HDR). In some embodiments, the a DNA base editing or prime-editing gene editing technology can be used. In some embodiments, a Programmable Addition via Site-specific Targeting Elements (PASTE) gene editing technology can be used.

Exemplary methods used to introduce the synthetic cytokine receptor into a target nucleic acid molecule include genome editing using endonucleases, meganucleases, zinc-finger nucleases and transcriptional activator-like effector nucleases (TALENs).

In some embodiments, methods to introduce an exogenous gene, such as a gene encoding the synthetic cytokine receptor. into a target nucleic acid molecule involves genome editing using engineered endonucleases. In some embodiments, this approach involves a reverse genetics method using artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a donor template for regenerating the missing DNA sequence at the break point. In order to introduce specific nucleotide modifications (e.g., mutations, such as amino acid substitutions) to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and RNA-guided nucleases (RGNs) such as Type II and Type V RGNs.

It will be apparent to one skilled in the art upon reading the present disclosure that various editing mechanisms can be used to create the cells, systems and methods of manufacture disclosed. Multiple different nuclease-based systems exist for providing edits into an organism's genome, and each can be used in either single editing systems, sequential editing systems (e.g., using different nuclease-directed systems sequentially to provide two or more genome edits in a cell) and/or recursive editing systems, (e.g., utilizing a single nuclease-directed system to introduce two or more genome edits in a cell). Thus, a person of skill in the art would recognize upon reading the present disclosure that various enzyme-directed editing systems are useful for the disclosed embodiments.

In some embodiments, the targeted insertion may be by target-primed reverse transcription (TPRT) or “prime editing”. In some embodiments, prime editing mediates targeted insertions in human cells without requiring DSBs or donor DNA templates. Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit

In some embodiments, targeted insertion is by Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks, but allowed for integration of sequences as large as ˜36 kb. In some embodiments, the serine integrase can be any known in the art. In some embodiments, the serine integrase has sufficient orthogonality such that PASTE can be used for multiplexed gene integration, simultaneously integrating at least two different genes at at least two genomic loci. In some embodiments, PASTE has editing efficiencies comparable to or better than those of homology directed repair or non-homologous end joining based integration, with activity in nondividing cells and fewer detectable off-target events.

### 1. Homology-Directed Repair (HDR)

In some aspects, the provided embodiments involve targeted integration of a nucleic acid sequence, such as a donor template, at a target nucleic acid sequence, e.g. an endogenous gene. In some embodiments, the target nucleic acid molecule is a housekeeping gene. In some embodiments, the housekeeping gene is eukaryotic translation elongation factor 1 alpha (EEF1A), glylceraldehyde-3-phosphate dehydrogenase (GAPDH), ubiquitin C (UBC), or actin beta (ACTB). In some embodiments, the target nucleic acid is B2M, TRAC or SIRPA. In some embodiments, the target nucleic acid is B2M.

In some embodiments, DNA repair mechanisms can be induced by a nuclease after (i) two SSBs, where there is a SSB on each strand, thereby inducing single strand overhangs; or (ii) a DSB occurring at the same cleavage site on both strands, thereby inducing a blunt end break.

In some embodiments, HDR is utilized for targeted integration or insertion of a nucleic acid sequence(s), e.g., a donor template, in one or more target nucleic acid molecules (e.g., endogenous gene(s)). In some embodiments, HDR can be used to integrate a donor template comprising a synthetic cytokine receptor (e.g., a RACR) into a target nucleic acid molecule (e.g. an endogenous gene). For example, HDR can be used to integrate a donor template encoding a RACR into the B2M gene locus.

Agents capable of inducing a DSB, such as Cas nucleases (e.g. Cas9), TALENs, and ZFNs, promote genomic editing by inducing a DSB at a cleavage site within a target nucleic acid molecule such as an endogenous gene, e.g., B2M, as discussed in preceding sections.

Agents capable of inducing a SSB, also sometimes referred to as a nick, include recombinant nucleases, e.g., Cas9, having nickase activity, such as, e.g., those described in preceding sections. Examples of agents having nickase activity includes, e.g., a Cas9 from Streptococcus pyogenes that comprises a mutation selected from the group consisting of D10A, H840A, H854A, and H863A.

Upon cleavage by one of these agents, the target endogenous gene, e.g., B2M, with the SSBs or the DSB undergoes one of two major pathways for DNA damage repair: (1) the error-prone non-homologous end joining (NHEJ), or (2) the high-fidelity homology-directed repair (HDR) pathway.

In some embodiments, cells in which SSBs or a DSB was previously induced by one or more agent(s) comprising a nuclease, are obtained, and a donor template, e.g., ssODN, is introduced to result in HDR and integration of the donor template into the target endogenous gene, e.g., B2M.

In general, in the absence of a repair template, e.g., a donor template, such as a ssODN., the NHEJ process re-ligates the ends of the cleaved DNA strands, which frequently results in nucleotide deletions and insertions at the cleavage site.

Alteration of nucleic acid sequences at target endogenous gene locus, such as the B2M gene locus, can occur by HDR by integrating an exogenously provided donor template that encodes for a synthetic cytokine receptor (e.g., a RACR). The HDR pathway can occur by way of the canonical HDR pathway or the alternative HDR pathway. Unless otherwise indicated, the term “HDR” or “homology-directed repair” as used herein encompasses both canonical HDR and alternative HDR.

Canonical HDR or “canonical homology-directed repair” or cHDR,” are used interchangeably, and refers to the process of repairing DNA damage using a homologous nucleic acid (e.g., an endogenous homologous sequence, such as a sister chromatid; or an exogenous nucleic acid, such as a donor template). Canonical HDR typically acts when there has been a significant resection at the DSB, forming at least one single-stranded portion of DNA. In a normal cell, canonical HDR typically involves a series of steps such as recognition of the break, stabilization of the break, resection, stabilization of single-stranded DNA, formation of a DNA crossover intermediate, resolution of the crossover intermediate, and ligation. The canonical HDR process requires RAD51 and BRCA2, and the homologous nucleic acid, e.g., donor template, is typically double-stranded. In canonical HDR, a double-stranded polynucleotide, e.g., a double stranded donor template, is introduced, which comprises a sequence that is homologous to the targeting sequence within the target endogenous gene locus, and which will either be directly integrated into the targeting sequence or will be used as a template to insert the sequence, or a portion the sequence, of the donor template into the target endogenous gene, e.g., B2M After resection at the break, repair can progress by different pathways, e.g., by the double Holliday junction model (also referred to as the double strand break repair, or DSBR, pathway), or by the synthesis-dependent strand annealing (SDSA) pathway.

In the double Holliday junction model, strand invasion occurs by the two single stranded overhangs of the targeting sequence to the homologous sequences in the double-stranded polynucleotide, e.g., double stranded donor template, which results in the formation of an intermediate with two Holliday junctions. The junctions migrate as new DNA is synthesized from the ends of the invading strand to fill the gap resulting from the resection. The end of the newly synthesized DNA is ligated to the resected end, and the junctions are resolved, resulting in the insertion at the targeting sequence, or a portion of the targeting sequence that includes the gene variant. Crossover with the polynucleotide, e.g., donor template, may occur upon resolution of the junctions.

In the SDSA pathway, only one single stranded overhang invades the polynucleotide, e.g., donor template, and new DNA is synthesized from the end of the invading strand to fill the gap resulting from resection. The newly synthesized DNA then anneals to the remaining single stranded overhang, new DNA is synthesized to fill in the gap, and the strands are ligated to produce the modified DNA duplex.

Alternative HDR, or “alternative homology-directed repair,” or “alternative HDR,” are used interchangeably, and refers, in some embodiments, to the process of repairing DNA damage using a homologous nucleic acid (e.g., an endogenous homologous sequence, such as a sister chromatid; or an exogenous nucleic acid, such as a donor template). Alternative HDR is distinct from canonical HDR in that the process utilizes different pathways from canonical HDR, and can be inhibited by the canonical HDR mediators, RAD51 and BRCA2. Moreover, alternative HDR is also distinguished by the involvement of a single-stranded or nicked homologous nucleic acid template, e.g., donor template, whereas canonical HDR generally involves a double-stranded homologous template. In the alternative HDR pathway, a single strand template polynucleotide, e.g., donor template, is introduced. A nick, single strand break, or DSB at the cleavage site, for altering a desired target site, e.g., a target endogenous gene, e.g., B2M, is mediated by a nuclease molecule, e.g., any of the nucleases as described herein, and resection at the break occurs to reveal single stranded overhangs. Incorporation of the sequence of the template polynucleotide, e.g., donor template, to alter the target site of the DNA typically occurs by the SDSA pathway, as described herein.

In some embodiments, HDR is carried out by introducing, into a cell, one or more agent(s) capable of inducing a DSB, such as any of those as described herein, and a donor template, e.g., ssODN, such as any of those described herein. The introducing can be carried out by any suitable delivery means, such as any of those as described herein. The conditions under which HDR is allowed to occur can be any conditions suitable for carrying out HDR in a cell.

In some embodiments, HDR is carried out by introducing, into a cell, one or more agent(s) capable of inducing a SSB in each stand, such as any of those as described herein, and a donor template, e.g., ssODN, such as any of those described herein. The introducing can be carried out by any suitable delivery means, such as any of those as described herein. The conditions under which HDR is allowed to occur can be any conditions suitable for carrying out HDR in a cell.

a. Donor Templates

In some embodiments, the provided methods include the use of a donor template, e.g., a donor template encoding a synthetic cytokine receptor, e.g., a RACR, that is homologous to a portion(s) of the targeting sequence in the target gene, e.g., B2M. In some embodiments, the targeting sequence is comprised within the sense strand. In some embodiments, the targeting sequence is comprised within the antisense strand. Also provided, in some embodiments, are donor templates for use in the methods provided herein, e.g., as templates for HDR-mediated integration of a nucleic acid sequence encoding a RACR.

In some embodiments, the donor template is used in conjunction with the one or more agent(s) capable of inducing a DNA break, e.g., a SSB or a DSB. In some embodiments, the donor template is used in conjunction with the one or more agent(s) capable of inducing a DSB and a guide RNA, e.g., sgRNA, to knock in a nucleic acid sequence encoding a synthetic cytokine receptor (e.g., a RACR) at a target endogenous gene locus (e.g., B2M). In some embodiments, the donor template is used in conjunction with the one or more agent(s) capable of inducing a SSB; the first guide RNA, e.g., the first sgRNA; and the second guide RNA, e.g., the second sgRNA, to knock in a nucleic acid sequence encoding a synthetic cytokine receptor (e.g., a RACR) at a target endogenous gene locus (e.g., B2M).

In some embodiments, the donor template comprises a nucleic acid sequence that is homologous to the cleavage site in the target gene, e.g., B2M. In some embodiments, the donor template comprises a nucleic acid sequence that is homologous to the cleavage site in the sense strand of the target gene, e.g., B2M. In some embodiments, the donor template comprises a nucleic acid sequence that is homologous to the cleavage site in the antisense strand of the target gene, e.g., B2M. In some embodiments, the target gene, e.g., B2M, includes a sense strand and an antisense strand, and the sense strand comprises the targeting sequence. In some embodiments, the target gene, e.g., B2M, includes a sense strand and an antisense strand, and the antisense strand comprises the targeting sequence.

In some embodiments, the donor template, e.g., ssODN, comprises a nucleic acid sequence comprising a PAM sequence that is homologous to the PAM sequence in the targeting sequence.

In some embodiments, the donor template is single-stranded. In some embodiments, the donor template is a single-stranded DNA oligonucleotide (ssODN). In some embodiments, the donor template is double-stranded.

In some embodiments, the ssODN comprises a 5′ ssODN arm and a 3′ sSODN arm. In some embodiments, the 5′ ssODN arm is directly linked to the 3′ ssODN arm. In some embodiments, the 5′ ssODN arm is homologous to the sequence of the target gene, e.g., B2M, that is immediately upstream of the cleavage site, and the 3′ ssODN arm is homologous to the sequence of the target gene that is immediately downstream of the cleavage site.

In some embodiments, the 5′ ssODN arm and/or the 3′ ssODN arm has a length that is between 250 and 750 nucleotides in length. In some embodiments, the 5′ ssODN arm has a length that is between 250 and 750 nucleotides in length. In some embodiments, the 3′ ssODN arm has a length that is between 250 and 750 nucleotides in length. In some embodiments, each of the 5′ ssODN arm and the 3′ ssODN arm has a length that is between 250 and 750 nucleotides in length. In some embodiments, the 5′ ssODN arm and/or the 3′ ssODN arm has a length that is about 500 nucleotides in length. In some embodiments, the 5′ ssODN arm has a length that is about 500 nucleotides in length. In some embodiments, the 3′ ssODN arm has a length that is about 500 nucleotides in length. In some embodiments, each of the 5′ ssODN arm and the 3′ ssODN arm has a length that is about 500 nucleotides in length.

In some embodiments, the target gene is B2M and the donor template comprises a nucleic acid sequence that is homologous to the cleavage site in the B2M gene. In some embodiments, the donor template comprises a nucleic acid sequence that is homologous to the cleavage site in the sense strand of the B2M target gene. In some embodiments, the donor template comprises a nucleic acid sequence that is homologous to the cleavage site in the antisense strand of the B2M target gene. In some embodiments, the donor template is a ssODN and the 5′ ssODN arm is homologous to the sequence of the B2M target gene that is immediately upstream of the cleavage site, and the 3′ ssODN arm is homologous to the sequence of the B2M target gene that is immediately downstream of the cleavage site.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO:22. In some embodiments, the 5′ ssODN arm comprises the nucleic acid sequence set forth in SEQ ID NO: 22.

In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 23 In some embodiments, the 3′ ssODN arm comprises the nucleic acid sequence set forth in SEQ ID NO: 23.

In some embodiments, the 5′ ssODN arm comprises the nucleic acid sequence as set forth in SEQ ID NO: 22, and the 3′ ssODN arm comprises the nucleic acid sequence as set forth in SEQ ID NO: 23.

Also provided herein is an isolated nucleic acid, e.g., an isolated nucleic acid for use in a method of knocking in a synthetic cytokine receptor (e.g., a RACR) into a target gene (e.g., B2M), comprising the nucleic acid sequence of any of the donor templates, e.g., ssODNs, or portions thereof, e.g., or 5′ ssODN arms, or 3′ ssODN arms, described herein. In some embodiments, the 5′ ssODN comprises the nucleic acid sequence as set forth in SEQ ID NO: 22. In some embodiments, the 3′ ssODN arm comprises the nucleic acid sequence as set forth in SEQ ID NO: 23.

In some embodiments, the crRNA comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 18; the 5′ ssODN arm comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 22; and the 3′ ssODN arm comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 23. In some embodiments, the crRNA comprises the nucleic acid sequence set forth in any one of SEQ ID NOS: 18; the 5′ ssODN comprises the nucleic acid sequence set forth in SEQ ID NO: 22; and the 3′ ssODN comprises the nucleic acid sequence set forth in SEQ ID NO: 23.

In some embodiments, the donor template, e.g., ssODN, comprises a nucleic acid sequence encoding a transgene sequence encoding the synthetic cytokine receptor. In some embodiments, the synthetic cytokine receptor is a rapamycin-activated cytokine receptor (RACR) that is responsive to rapamycin or an analog (e.g. rapalog). In some embodiments, the transgene sequence is a tandem cassette that encodes both polypeptides of the synthetic cytokine receptor.

In some embodiments, the transgene encoding the synthetic cytokine receptor (e.g. RACR) can be inserted so that its expression is driven by the endogenous promoter at the integration site, for example the promoter that drives expression of the endogenous B2M gene. In some embodiments in which the polypeptide encoding sequences are promoterless, expression of the integrated transgene is then ensured by transcription driven by an endogenous promoter or other control element in the region of interest. For example, the transgene encoding a portion of the synthetic cytokine receptor (e.g. RACR) can be inserted without a promoter, but in-frame with the coding sequence of the endogenous locus (e.g. B2M locus) such that expression of the integrated transgene is controlled by the transcription of the endogenous promoter and/or other regulatory elements at the integration site. In some embodiments, a multi-cistronic element such as a ribosome skipping element/self-cleavage element (e.g., a 2A element or an internal ribosome entry site (IRES)), is placed upstream of the transgene, such that the multi-cistronic element is placed in-frame with one or more exons of the endogenous open reading frame at the endogenous locus (e.g. B2M locus), such that the expression of the transgene is operably linked to the endogenous promoter.

In some embodiments, each nucleic acid encoding a polypeptide of the synthetic cytokine receptor in the “tandem” cassettes is independently controlled by a regulatory element or all controlled as a multi-cistronic (e.g. bicistronic) expression system. In other embodiments, each nucleic acid encoding a polypeptide of the synthetic cytokine receptor in the “tandem” cassettes can be operatively linked to a promoter, which can be the same or different. In some embodiments, the nucleic acid molecule can contain a promoter that drives the expression of two or more different polypeptide chains. In some embodiments, such nucleic acid molecules can be multi-cistronic (bicistronic or tricistronic, see e.g., U.S. Pat. No. 6,060,273). In some embodiments, transcription units can be engineered as a bicistronic unit containing an IRES (internal ribosome entry site), which allows coexpression of gene products by a message from a single promoter. Alternatively, in some cases, a single promoter may direct expression of an RNA that contains, in a single open reading frame (ORF), two polypeptides separated from one another by sequences encoding a cleavable linker as described herein. The ORF thus encodes a single polypeptide, which, cither during or after translation, is processed into the individual polypeptide chains. In some embodiments, the promoter is selected from among human elongation factor 1 alpha (EF1α) promoter (such as set forth in SEQ ID NO:24, 25 or 26). In some embodiments, the promoter is an MND promoter (such as set forth in SEQ ID NO:27).

In some embodiments, the donor template, e.g., ssODN, comprises a nucleic acid sequence encoding a synthetic cytokine receptor (e.g. RACR). In some embodiments, the nucleic acid sequence encoding the synthetic cytokine receptor (e.g. RACR) is located between the 5′ ssODN arm and the 3′ ssODN arm. In some embodiments, the nucleic acid sequence encoding the synthetic cytokine receptor (e.g. RACR) comprises an EF1-alpha promoter (e.g., SEQ ID NO:24, 25 or 26). In some embodiments, the nucleic acid sequence encoding the synthetic cytokine receptor (e.g. RACR) comprises a MND promoter (e.g., SEQ ID NO: 27). In some embodiments, the synthetic cytokine receptor is a rapamycin-activated cytokine receptor (RACR). The RACR can be any as described, such as in Section II.B. In some embodiments, the nucleic acid molecule is a tandem cassette encoding the first polypeptide sequence of RACR and the second polypeptide sequence of RACR.

In some embodiments, the first nucleic acid sequence encoding the RACR comprises a nucleic acid sequence encoding a RACR-gamma chain (e.g., SEQ ID NO:28), and a nucleic acid sequence encoding a RACR-beta chain (e.g., SEQ ID NO:33). In some embodiments, the first nucleic acid sequence encodes a RACR-gamma chain that has at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 28. In some embodiments, the first nucleic acid sequence encodes the RACR-gamma chain sequence set forth in SEQ ID NO:28. In some embodiments, the nucleic acid sequence encoding the RACR-gamma chain further encodes a signal peptide at the N-terminus of the nascent protein to prompt transport of the protein when expressed. In some embodiments, the signal peptide has the sequence set forth in SEQ ID NO: 29. In some embodiments, the second nucleic acid sequence encodes a RACR-beta chain that has at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 33. In some embodiments, the second nucleic acid sequence encodes the RACR-beta chain set forth in SEQ ID NO:33. In some embodiments, the nucleic acid sequence encoding the RACR-beta chain further encodes a signal peptide at the N-terminus of the nascent protein to prompt transport of the protein when expressed. In some embodiments, the signal peptide has the sequence set forth in SEQ ID NO: 34.

In some embodiments, the first nucleic acid sequence encoding the RACR-gamma chain has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 37. In some embodiments, the first nucleic acid sequence encoding the RACR-gamma chain has the sequence set forth in SEQ ID NO:37. In some embodiments, the second nucleic acid sequence encoding the RACR-beta chain has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 38. In some embodiments, the second nucleic acid sequence encoding the RACR-beta chain is set forth in SEQ ID NO:38.

In some embodiments, the nucleic acid sequence encoding the RACR-gamma chain and the nucleic acid sequence encoding the RACR-beta chain are separated by a nucleic acid sequence encoding a cleavable linker. In some embodiments, a further nucleic acid sequence encoding a cleavable linker is located downstream of the nucleic acid sequence encoding the RACR-beta chain

In some embodiments, the linker is a protein quantitation reporter linker (PQR; e.g., SEQ ID NO:42), including any as described in Canadian Patent Application No. CA2970093, incorporated by reference in its entirety herein. In some embodiments, the PQR linker has the sequence set forth in SEQ ID NO:42. In some embodiments, the PQR linker is encoded by a sequence of nucleotides set forth in SEQ ID NO:41.

In some embodiments, the cleavable linker is a self-cleaving peptide, such as a 2A ribosomal skip element. In some cases, the cleavable linker, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and nucleic acids disclosed herein, without limitation, 2A sequences from the foot-and-mouth disease virus (F2A, e.g., SEQ ID NO: 43), equine rhinitis A virus (E2A, e.g., SEQ ID NO: 44), Thosea asigna virus (T2A, e.g., SEQ ID NO: 45 or 46), and porcine teschovirus-1 (P2A, e.g., SEQ ID NO: 47 or 48) as described in U.S. Patent Publication No. 20070116690.

In some embodiments, by virtue of the cleavable element located between the first nucleic acid sequence and the second nucleic acid sequence, expression of a nucleic acid sequence encoding a RACR yields a first peptide (i.e., the RACR-gamma chain) and a separate, second peptide (i.e., the RACR-beta chain).

In some embodiments, the transgene sequences may also include sequences required for transcription termination and/or polyadenylation signal. In some aspects, exemplary polyadenylation signal is selected from SV40, hGH, BGH, and rbGlob transcription termination sequence and/or polyadenylation signal. In some embodiments, the transgene includes an SV40 polyadenylation signal. In some embodiments, if present within the transgene, the transcription termination sequence and/or polyadenylation signal is typically the most 3′ sequence within the transgene, and is linked to one of the homology arm. In some embodiments, transgene sequence includes the polyadenylation sequence set forth in SEQ ID NO:39.

In some embodiments, the ssODN comprises, in order: a 5′ ssODN arm, a EF1-alpha promoter, a nucleic acid sequence encoding the RACR-gamma chain, a nucleic acid sequence encoding a cleavable linker (e.g., a PQR linker), a nucleic acid sequence encoding the RACR-beta chain, a poly A sequence, and the 3′ ssODN arm.

In some embodiments, the ssODN comprises the sequence set forth in SEQ ID NO: 40 or a sequence that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 40. In some embodiments, the ssODN is set forth in SEQ ID NO:40.

In some embodiments, after the integration of the ssODN into the target gene, the target gene is knocked out. In some embodiments, the target gene is human B2M, and, after the integration of the ssODN into B2M, B2M is knocked out. In some embodiments, a nucleic acid sequence encoding the synthetic cytokine receptor is integrated into the B2M locus. In some embodiments, the engineered iPSC and iCIL has a modified B2M locus in which the endogenous B2M gene is genetically disrupted by knockout of the B2M gene and knock in by targeted integration of a nucleic acid encoding the synthetic cytokine receptor. In some embodiments, the synthetic cytokine receptor is a RACR encoded by a nucleic acid sequence that contains in order: a EF1-alpha promoter, a nucleic acid sequence encoding the RACR-gamma chain, a nucleic acid sequence encoding a cleavable linker (e.g., a PQR linker), a nucleic acid sequence encoding the RACR-beta chain, and a polyA sequence. In some embodiments, the nucleic acid sequence encoding RACR that is integrated into the B2M locus has the sequence set forth in SEQ ID NO:32 or a sequence that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 32. In some embodiments, the nucleic acid encoding RACR that is integrated into the B2M locus is set forth in SEQ ID NO:32.

### IV. CHIMERIC ANTIGEN RECEPTOR

In some cases, the stem cells or CIL cells of the present disclosure comprise a polynucleotide encoding a chimeric antigen receptor (CAR), thereby generating stem cells or CIL cells expressing the CAR.

In some embodiments, the disclosure contemplates a chimeric antigen receptor (CAR) system for use in the treatment of subjects with cancer. In some embodiments, the CIL cells of the disclosure comprise a CAR sequence (CAR-CIL cells or CAR-iCIL cells).

In some embodiments, stem cells or CIL cells are engineered to express CAR constructs by transfecting a population of cells with an expression vector encoding the CAR construct. Illustrative examples of populations of cells that may be transfected include HSCs, blood progenitor cells, common lymphoid progenitor cells, or CIL cells. Appropriate means for preparing a transduced population of CIL cells expressing a selected CAR construct will be well known to the skilled artisan, and includes retrovirus, lentivirus (viral mediated CAR gene delivery system), sleeping beauty, and piggyback (transposon/transposase systems that include a non-viral mediated CAR gene delivery system), to name a few examples. In some embodiments, any of the transduction methods contemplated in the disclosure may be used to generate CAR-expressing stem cells or CIL cells.

In some embodiments, stem cells or CIL cells are engineered to express CAR constructs by genetically engineering (e.g., via CRISPR) a population of cells to express the CAR construct. In some embodiments, a nucleic acid molecule encoding a CAR, such as by introduction of a vector construct encoding the CAR, is introduced into the cell. In some embodiments, the construct is designed for insertion of the nucleic acid encoding the CAR into an endogenous locus in the cell. Methods of gene insertion or knock-in are known, including any of the methods described in Section III. In some embodiments, insertion of a CAR-encoding construct is by homology directed repair, such as by using a CRISPR-Cas system.

In some embodiments, the CAR construct contains an extracellular binding portion, a transmembrane domain and an intracellular signaling domain. In some embodiments, the intracellular signaling domain contains a costimulatory signaling domain and/or an activation signaling domain. In some embodiments, the CAR construct contains an extracellular binding portion, a transmembrane domain and an intracellular signaling domain comprising a costimulatory signaling domain. In some embodiments, the CAR construct contains an extracellular binding portion, a transmembrane domain and an intracellular signaling domain comprising an activation signaling domain. In some embodiments, the CAR construct contains an extracellular binding portion, a transmembrane domain and an intracellular signaling domain comprising a costimulatory signaling domain and an activation signaling domain.

In some embodiments, the CARs may include additional elements, such a signal peptide to ensure proper export of the fusion protein to the cells surface, a transmembrane domain to ensure the fusion protein is maintained as an integral membrane protein, and a hinge domain that imparts flexibility to the recognition region and allows strong binding to the targeted moiety.

### A. Extracellular Binding Portion

Conventionally, CARs are generated by fusing a polynucleotide encoding a VL, VH, or scFv to the 5′ end of a polynucleotide encoding transmembrane and intracellular domains, and transducing cells with that polynucleotide as well as with the corresponding VH or VL, if needed. Numerous variations on CARs well known in the art and the disclosure contemplates using any of the known variations. Additionally, VL/VH pairs and scFv's for innumerable haptens are known in the art or can be generated by conventional methods routinely. Accordingly, the present disclosure contemplates using any known hapten-binding domain.

In any embodiments described herein, the binding portion of the CAR can be, for example, a single chain fragment variable region (scFv) of an antibody, a Fab, Fv, Fc, or (Fab′) 2 fragment, and the like. The use of unaltered (i.e., full size) antibodies, such as IgG, IgM, IgA, IgD or IgE, in the CAR or as the CAR is excluded from the scope of the invention.

In some embodiments, the binding portion of the CAR can be directed to any antigen that is desired to be targeted, such as due to its overexpression on cells or association with a disease or conditions like cancer.

In some embodiments, the binding portion of the CAR is specific to a tumor antigen. The selection of the antigen binding domain will depend on the particular type of cancer to be treated. Tumor antigens are well known in the art and include, for example, a glioma-associated antigen, carcinoembryonic antigen (CEA), EGFRvIII, IL-11Ra, IL-13Ra, EGFR, FAP, B7H3, Kit, CA LX, CS-1, MUC1, BCMA, bcr-abl, HER2, B-human chorionic gonadotropin, alphafetoprotein (AFP), ALK, CD19, CD123, cyclin B1, lectin-reactive AFP, Fos-related antigen 1, ADRB3, thyroglobulin, EphA2, RAGE-1, RU1, RU2, SSX2, AKAP-4, LCK, OY-TES1, PAXS, SART3, CLL-1, fucosyl GM1, GloboH, MN-CA IX, EPCAM, EVT6-AML, TGS5, human telomerase reverse transcriptase, plysialic acid, PLAC1, RU1, RU2 (AS), intestinal carboxyl esterase, lewis Y, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-1a, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP. WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, cphnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRCSD, GPR20, CXORF61, folate receptor (FRa), folate receptor beta, ROR1, Flt3, TAG72, TN Ag. Tic 2, TEM1, TEM7R, CLDN6, TSHR, UPK2, and mesothelin. Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, IL13Ra2, CA 19-9, CA 72-4, CAM 17.1. NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag. MOV18, NB/70K, NY-CO-1, RCAS1, SDCCAG1 6, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

In some embodiments, a CAR comprises an extracellular domain comprising a FMC63 scFv binding domain for CD19 binding. In some embodiments, the CAR is a second-generation CAR comprised of the FMC63 mouse anti-human CD19 scFv linked to the 4-1BB costimulatory domain and the CD3zeta intracellular signaling domain. In some embodiments, a CAR comprises a binding domain for CD19, a CD8a hinge, a CD8a transmembrane domain, a 4-1BB costimulatory domain, and a CD3zeta signaling domain. In some embodiments, a CAR comprises a binding domain for CD19, an IgG4 hinge, a CD28 transmembrane domain, a 4-1BB costimulatory domain, and a CD3zeta signaling domain. In some embodiments, a CAR comprises a binding domain for CD19, a CD28 hinge, a CD28 transmembrane domain, a CD28 costimulatory domain, and CD3zeta signaling domain. In some embodiments, a CAR comprises an extracellular domain comprising a FMC63 scFv binding domain for CD19 binding, a CD8a hinge, a CD8a transmembrane domain, a 4-1BB costimulatory domain, and a CD3zeta signaling domain. In some embodiments, a CAR comprises an extracellular domain comprising a FMC63 scFv binding domain for CD19 binding, an IgG4 hinge, a CD28 transmembrane domain, a 4-1BB costimulatory domain, and a CD3zeta signaling domain. In some embodiments, a CAR comprises an extracellular domain comprising a FMC63 scFv binding domain for CD19 binding, a CD28 hinge, a CD28 transmembrane domain, a CD28 costimulatory domain, and CD3zeta signaling domain.

In some embodiments, the CAR is a second-generation CAR comprised of the FMC63 mouse anti-human CD19 scFv linked to the CD28 costimulatory domain and the CD3zeta intracellular signaling domain. In some embodiments, the CAR is a second-generation CAR comprised of the FMC63 mouse anti-human CD19 scFv linked to a CD8 transmembrane domain, 4-1BB costimulatory domain, and the CD3zeta intracellular signaling domain.

In some embodiments, the antigen is BCMA. CAR T therapies targeting BCMA have been approved by the FDA and include Abecma and Carvykti. CARs targeting BCMA are described, for example, in US Publication No. 2020/0246381; U.S. Pat. No. 10,918,665; US Publication No. 2019/0161553, each of which is herein incorporated by reference. In some embodiments, a CAR comprises a binding domain for BCMA, a CD8a hinge, a CD8a transmembrane domain, a 4-1BB costimulatory domain, and a CD3zeta signaling domain. In some embodiments, a CAR comprises a binding domain for BCMA, an IgG4 hinge, a CD28 transmembrane domain, a 4-1BB costimulatory domain, and a CD3zeta signaling domain. In some embodiments, a CAR comprises a binding domain for BCMA, a CD28 hinge, a CD28 transmembrane domain, a CD28 costimulatory domain, and CD3zeta signaling domain.

In some embodiments, the antigen is G protein-coupled receptor class C group 5 member D (GPRC5D). CARs targeting GRC5D are described, for example, in US Publication Nos. 2018/0118803 and 2021/10393689, each of which is herein incorporated by reference. In some embodiments, a CAR comprises a binding domain for GRC5D, a CD8a hinge, a CD8a transmembrane domain, a 4-1BB costimulatory domain, and a CD3zeta signaling domain. In some embodiments, a CAR comprises a binding domain for GRC5D, an IgG4 hinge, a CD28 transmembrane domain, a 4-1BB costimulatory domain, and a CD3zeta signaling domain. In some embodiments, a CAR comprises a binding domain for GRC5D, a CD28 hinge, a CD28 transmembrane domain, a CD28 costimulatory domain, and CD3zeta signaling domain.

In some embodiments, the antigen is Fc Receptor-like 5 (FcRL5). CARs targeting FcRL5 are described, for example, in US Publication No. US 2017/0275362, which is herein incorporated by reference. In some embodiments, a CAR comprises a binding domain for FcRL5, a CD8a hinge, a CD8a transmembrane domain, a 4-1BB costimulatory domain, and a CD3zeta signaling domain. In some embodiments, a CAR comprises a binding domain for FcRL5, an IgG4 hinge, a CD28 transmembrane domain, a 4-1BB costimulatory domain, and a CD3zeta signaling domain. In some embodiments, a CAR comprises a binding domain for FcRL5, a CD28 hinge, a CD28 transmembrane domain, a CD28 costimulatory domain, and CD3zeta signaling domain.

In some embodiments, the antigen is receptor tyrosine kinase-like orphan receptor 1 (ROR1). CARs targeting ROR1 are described, for example, in US Publication No. 2022/0096651, which is herein incorporated by reference. In some embodiments, a CAR comprises a binding domain for ROR1, a CD8a hinge, a CD8a transmembrane domain, a 4-1BB costimulatory domain, and a CD3zeta signaling domain. In some embodiments, a CAR comprises a binding domain for ROR1, an IgG4 hinge, a CD28 transmembrane domain, a 4-1BB costimulatory domain, and a CD3zeta signaling domain. In some embodiments, a CAR comprises a binding domain for ROR1, a CD28 hinge, a CD28 transmembrane domain, a CD28 costimulatory domain, and CD3zeta signaling domain.

In some embodiments, the CAR is a second-generation CAR comprised an anti-BCMA scFv linked to the 4-1BB costimulatory domain and the CD3zeta intracellular signaling domain. In some embodiments, the CAR is a second-generation CAR comprised an anti-GPRC5D scFv linked to the 4-1BB costimulatory domain and the CD3zeta intracellular signaling domain. In some embodiments, the CAR is a second-generation CAR comprised an anti-ROR1 scFv linked to the 4-1BB costimulatory domain and the CD3zeta intracellular signaling domain.

A skilled artisan is readily familiar with CARs against diverse tumor antigens. Any one of such CARs can be employed as the CAR. Numerous CARs have been approved by the FDA and include, but are not limited to, anti-CD19 and anti-BCMA CAR T cells such as tisagenlecleucel (Kymriah), axicabtagene ciloleucel (Yescarta), brexucabtagene autoleucel (Tecartus), lisocabtagene maraleucel (Breyanzi), or idecabtagene vicleucel (Abecma). It is within the level of a skilled artisan to generate similar constructs for specific targeting of a desired tumor antigen.

In some embodiments, the binding portion of the CAR can be directed to a universal antigen to target a wide variety of tumors without the need to prepare separate CAR constructs. The targeted moiety recognized by the CAR may also remain constant. In some embodiments, a ligand may be administered to the subject to allow interaction with target cells and interaction with the binding portion of the CAR. It is only the ligand portion of the small conjugate molecule that needs to be altered to allow the system to target cancer cells of different identity. Exemplary universal CAR systems are described in the section above.

In some embodiments, the CAR is an anti-hapten CAR, such as any described in Section IV.A. above. In some embodiments, the anti-hapten CAR can be selectively targeted to a target cell labeled by a small molecule conjugate composed of a hapten and a cell-targeting moiety, such as any described above. In some embodiments, the CAR is an anti-fluorescein/FITC chimeric antigen receptor that can be selectively targeted to a target cell labeled by a small molecule conjugate composed of fluorescein or fluorescein isothiocyanate (FITC) and a cell-targeting moiety. In variations, other haptens recognized by CARs may be used in place of fluorescein/FITC. The CAR may be generated using various scFv sequences known in the art, or scFv sequences generated by conventional and routine methods. Further illustrative scFv sequences for fluorescein/FITC and for other haptens are provided in, for example, WO 2021/076788, the disclosure of which is incorporated by reference herein.

### Targeting Agents for Universal CARs

In some embodiments, the CAR system of the disclosure makes use of CARs that target a moiety that is not produced or expressed by cells of the subject being treated. This CAR system thus allows for focused targeting of the CIL cells to target cells, such as cancer cells. By administration of a small conjugate molecule along with the CAR-expressing CIL cells, the CIL cell response can be targeted to only those cells expressing the tumor receptor, thereby reducing off-target toxicity, and the activation of CIL cells can be more easily controlled due to the rapid clearance of the small conjugate molecule. As an added advantage, the CAR-expressing CIL cells can be used as a “universal” cytotoxic cell to target a wide variety of tumors without the need to prepare separate CAR constructs. The targeted moiety recognized by the CAR may also remain constant. It is only the ligand portion of the small conjugate molecule that needs to be altered to allow the system to target cancer cells of different identity.

Various methods to target CARs and CAR-expressing cells have been described in the art, including, for example in US 2020/0123224, the disclosure of which is incorporated by reference herein. For example, a fluorescein or fluorescein isothiocyanate (FITC) moiety may be conjugated to an agent that binds to a desired target cell (such as a cancer cell), and thereby a CAR-CIL cell expressing an anti-fluorescein/FITC chimeric antigen receptor may be selectively targeted to the target cell labeled by the conjugate. In variations, other haptens recognized by CARs may be used in place of fluorescein/FITC. The CAR may be generated using various scFv sequences known in the art, or scFv sequences generated by conventional and routine methods. Further illustrative scFv sequences for fluorescein/FITC and for other haptens are provided in, for example, WO 2021/076788, the disclosure of which is incorporated by reference herein.

In one embodiment, the disclosure provides an illustration of this conjugate molecule/CAR system.

In some embodiments, the CAR system of the disclosure utilizes conjugate molecules as the bridge between CAR-expressing cells and targeted cancer cells. The conjugate molecules are conjugates comprising a hapten and a cell-targeting moiety, such as any suitable tumor cell-specific ligand. Illustrative haptens that can be recognized and bound by CARs, include small molecular weight organic molecules such as DNP (2,4-dinitrophenol), TNP (2,4,6-trinitrophenol), biotin, and digoxigenin, along with fluorescein and derivatives thereof, including FITC (fluorescein isothiocyanate), NHS-fluorescein, and pentafluorophenyl ester (PFP) and tetrafluorophenyl ester (TFP) derivatives, a knottin, a centyrin, and a DARPin. Suitable cell-targeting moiety that may themselves act as a hapten for a CAR include knottins (see Kolmar H. et al., The FEBS Journal. 2008. 275(11):26684-90), centyrins, and DARPins (see Reichert, J. M. MAbs 2009. 1(3):190-209).

In some embodiments, the cell-targeting moiety is DUPA (DUPA-(99m) Tc), a ligand bound by PSMA-positive human prostate cancer cells with nanomolar affinity (K_{D}=14 nM; see Kularatne, S. A. et al., Mol Pharm. 2009. 6 (3): 780-9). In one embodiment, a DUPA derivative can be the ligand of the small molecule ligand linked to a targeting moiety, and DUPA derivatives are described in WO 2015/057852, incorporated herein by reference.

In some embodiments, the cell-targeting moiety is CCK2R ligand, a ligand bound by CCK2R-positive cancer cells (e.g., cancers of the thyroid, lung, pancreas, ovary, brain, stomach, gastrointestinal stroma, and colon; see Wayua. C. et al., Molecular Pharmaceutics. 2013. ePublication).

In some embodiments, the cell-targeting moiety is folate, folic acid, or an analogue thereof, a ligand bound by the folate receptor on cells of cancers that include cancers of the ovary, cervix, endometrium, lung, kidney, brain, breast, colon, and head and neck cancers; see Sega, E. I. et al., Cancer Metastasis Rev. 2008. 27 (4): 655-64).

In some embodiments, the cell-targeting moiety is an NK-1R ligand. Receptors for NK-1R the ligand are found, for example, on cancers of the colon and pancreas. In some embodiments, the NK-1R ligand may be synthesized according the method disclosed in Int'l Patent Appl. No. PCT/US2015/044229, incorporated herein by reference.

In some embodiments, the cell-targeting moiety may be a peptide ligand, for example, the ligand may be a peptide ligand that is the endogenous ligand for the NK1 receptor. In some embodiments, the small conjugate molecule ligand may be a regulatory peptide that belongs to the family of tachykinins which target tachykinin receptors. Such regulatory peptides include Substance P (SP), neurokinin A (substance K), and neurokinin B (neuromedin K), (see Hennig et al., International Journal of Cancer: 61, 786-792).

In some embodiments, the cell-targeting moiety is a CAIX ligand. Receptors for the CAIX ligand found, for example, on renal, ovarian, vulvar, and breast cancers. The CAIX ligand may also be referred to herein as CA9.

In some embodiments, the cell-targeting moiety is a ligand of gamma glutamyl transpeptidase. The transpeptidase is overexpressed, for example, in ovarian cancer, colon cancer, liver cancer, astrocytic gliomas, melanomas, and leukemias.

In some embodiments, the cell-targeting moiety is a CCK2R ligand. Receptors for the CCK2R ligand found on cancers of the thyroid, lung, pancreas, ovary, brain, stomach, gastrointestinal stroma, and colon, among others.

In one embodiment, the cell-targeting moiety may have a mass of less than about 10,000 Daltons, less than about 9000 Daltons, less than about 8,000 Daltons, less than about 7000 Daltons, less than about 6000 Daltons, less than about 5000 Daltons, less than about 4500 Daltons, less than about 4000 Daltons, less than about 3500 Daltons, less than about 3000 Daltons, less than about 2500 Daltons, less than about 2000 Daltons, less than about 1500 Daltons, less than about 1000 Daltons, or less than about 500 Daltons. In another embodiment, the small molecule ligand may have a mass of about 1 to about 10,000 Daltons, about 1 to about 9000 Daltons, about 1 to about 8,000 Daltons, about 1 to about 7000 Daltons, about 1 to about 6000 Daltons, about 1 to about 5000 Daltons, about 1 to about 4500 Daltons, about 1 to about 4000 Daltons, about 1 to about 3500 Daltons, about 1 to about 3000 Daltons, about 1 to about 2500 Daltons, about 1 to about 2000 Daltons, about 1 to about 1500 Daltons, about 1 to about 1000 Daltons, or about 1 to about 500 Daltons.

In one illustrative embodiment, the linkage in a conjugate described herein can be a direct linkage (e.g., a reaction between the isothiocyanate group of FITC and a free amine group of a small molecule ligand) or the linkage can be through an intermediary linker. In one embodiment, if present, an intermediary linker can be any biocompatible linker known in the art, such as a divalent linker. In one illustrative embodiment, the divalent linker can comprise about 1 to about 30 carbon atoms. In another illustrative embodiment, the divalent linker can comprise about 2 to about 20 carbon atoms. In other embodiments, lower molecular weight divalent linkers (i.e., those having an approximate molecular weight of about 30 to about 300 Da) are employed. In another embodiment, linkers lengths that are suitable include, but are not limited to, linkers having 2, 3, 4, 5, 6, 7, 8, 9. 10, 11, 12, 13, 14, 15, 16, 17, 18, 19, 20, 21, 22, 23, 24, 25, 26, 27, 28, 29, 30, 31, 32, 33, 34, 35, 36, 37. 38, 39 or 40, or more atoms.

In some embodiments, the hapten and the cell-targeting moiety can be directly conjugated through such means as reaction between the isothiocyanate group of FITC and free amine group of small ligands (e.g., folate, DUPA, and CCK2R ligand). However, the use of a linking domain to connect the two molecules may be helpful as it can provide flexibility and stability. Examples of suitable linking domains include: 1) polyethylene glycol (PEG); 2) polyproline; 3) hydrophilic amino acids; 4) sugars; 5) unnatural peptideoglycans; 6) polyvinylpyrrolidone; 7) pluronic F-127. Linker lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

In some embodiments, the linker may be a divalent linker that may include one or more spacers.

An illustrative conjugate of the disclosure is FITC-Folate

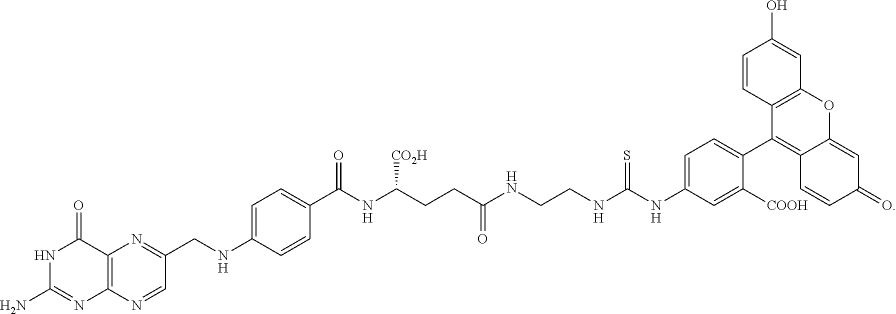 [SMILES: Nc1nc2ncc(CNc3ccc(C(=O)N[C@@H](CCC(=O)NCCNC(=S)Nc4ccc(-c5c6ccc(=O)cc-6oc6cc(O)ccc56)c(C(=O)O)c4)C(=O)O)cc3)nc2c(=O)[nH]1]

An illustrative conjugate of the disclosure is FITC-CA9

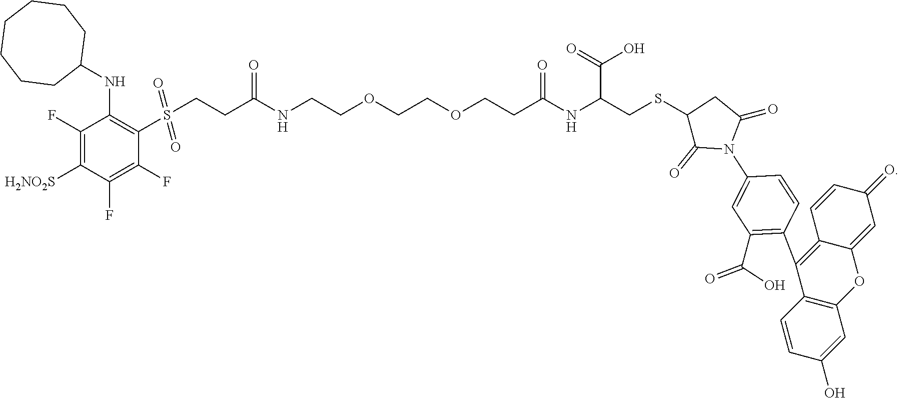 [SMILES: NS(=O)(=O)c1c(F)c(F)c(S(=O)(=O)CCC(=O)NCCOCCOCCC(=O)NC(CSC2CC(=O)N(c3ccc(-c4c5ccc(=O)cc-5oc5cc(O)ccc45)c(C(=O)O)c3)C2=O)C(=O)O)c(NC2CCCCCCC2)c1F]

Illustrative conjugates of the disclosure include the following molecules: FITC-(PEG)₁₂-Folate, FITC-(PEG)₂₀-Folate, FITC-(PEG)₁₀₈-Folate, FITC-DUPA, FITC-(PEG)₁₂-DUPA, FITC-CCK2R ligand, FITC-(PEG)₁₂-CCK2R ligand, FITC-(PEG)₁₁-NK1R ligand and FITC-(PEG)₂-CA9.

While the affinity at which the ligands and cancer cell receptors bind can vary, and in some cases low affinity binding may be preferable (such as about 1 μM), the binding affinity of the ligands and cancer cell receptors will generally be at least about 100 μM, 1 nM, 10 nM, or 100 nM, preferably at least about 1 pM or 10 pM, even more preferably at least about 100 pM.

Examples of conjugates and methods of making them are provided in U.S. patent applications US 2017/0290900, US 2019/0091308, and US 2020/0023009, all of which are incorporated herein by reference.

### B. Co-Stimulatory Domain

In some embodiments, a co-stimulation domain serves to enhance the proliferation and survival of the lymphocytes upon binding of the CAR to a targeted moiety. The identity of the co-stimulation domain is limited only in that it has the ability to enhance cellular proliferation and survival activation upon binding of the targeted moiety by the CAR. Suitable co-stimulation domains include, but are not limited to: CD28 (see, e.g., Alvarez-Vallina, L. et al., Eur J Immunol. 1996. 26 (10): 2304-9); CD137 (4-1BB), a member of the tumor necrosis factor (TNF) receptor family (scc, e.g., Imai, C. et al., Leukemia. 2004. 18:676-84); and CD134 (OX40), a member of the TNFR-superfamily of receptors (see, e.g., Latza, U. et al., Eur. J. Immunol. 1994. 24:677). A skilled artisan will understand that sequence variants of these co-stimulation domains can be used, where the variants have the same or similar activity as the domain on which they are modeled. In various embodiments, such variants have at least about 80%, at least about 90%, at least about 95%, at least about 97%, at least about 98%, at least about 99%, or at least about 99.5% sequence identity to the amino acid sequence of the domain from which they are derived.

In some embodiments of the invention, the CAR constructs comprise two co-stimulation domains. While the particular combinations include all possible variations of the four noted domains, specific examples include: 1) CD28+ CD137 (4-1BB) and 2) CD28+ CD134 (OX40).

### C. Activation Signaling Domain

In some embodiments, the activation signaling domain serves to activate cells upon binding of the CAR to a targeted moiety. The identity of the activation signaling domain is limited only in that it has the ability to induce activation of the selected cell upon binding of the targeted moiety by the CAR. Suitable activation signaling domains include the CD35 chain and Fc receptor γ. In some embodiments, the signaling domain is a signaling domain of NKG2C or NKp44. The skilled artisan will understand that sequence variants of these noted activation signaling domains can be used without adversely impacting the invention, where the variants have the same or similar activity as the domain on which they are modeled. Such variants may have at least about 80%, at least about 90%, at least about 95%, at least about 97%, at least about 98%, at least about 99%, or at least about 99.5% sequence identity to the amino acid sequence of the domain from which they are derived.

### D. Exemplary CARs

Illustrative CAR constructs suitable for CAR-CIL cells are provided below:
(1) scFv-CD8_{TM}-4-1BB_{Ic}-CD3ζs (see, e.g., Liu E, Tong Y, Dotti G, et al., Leukemia. 2018; 32: 520-531);
(2) scFv-CD28_{TM+IC}-CD3ζs (see, e.g., Han J, Chu J, Keung C W et al., Sci Rep. 2015; 5: 11483; Kruschinski A, Moosmann A, Poschke I et al., Proc Natl Acad Sci USA. 2008; 105: 17481-17486; and Chu J, Deng Y, Benson D M et al., Leukemia. 2014; 28:917-927);
(3) scFv-DAP12_{TM+IC} (see, e.g., Muller N, Michen S, Tietze S et al., J Immunother. 2015; 38: 197-210);
(4) scFv-CD8_{TM}-2B4_{IC}-CD3ζs (see, e.g., Xu Y, Liu Q, Zhong M et al., J Hematol Oncol. 2019; 12:49);
(5) scFv-2B4_{TM+IC}-CD3ζs (see, e.g., Altvater B, Landmeier S, Pscherer S et al., Clin Cancer Res. 2009; 15: 4857-4866);
(6) scFv-CD28_{TM+IC}-4-1BB_{IC}-CD3ζs (see, e.g., Kloss S, Oberschmidt O, Morgan M et al., Hum Gene Ther. 2017; 28: 897-913);
(7) scFv-CD16_{TM}-2B4_{IC}-CD3ζs (see, e.g., Li Y, Hermanson D L, Moriarity B S Kaufman D S, Cell Stem Cell. 2018; 23: 181-192);
(8) scFv-NKp44_{TM}-DAP10_{IC}-CD3ζs (see, e.g., Li Y, Hermanson D L, Moriarity B S Kaufman D S, Cell Stem Cell. 2018; 23: 181-192);
(9) scFv-NKp46_{TM}-2B4_{IC}-CD3ζs (see, e.g., Li Y, Hermanson D L, Moriarity B S Kaufman D S, Cell Stem Cell. 2018; 23: 181-192);
(10) scFv-NKG2D_{TM}-2B4_{IC}-CD3ζs (see, e.g., Li Y, Hermanson D L, Moriarity B S Kaufman D S, Cell Stem Cell. 2018; 23: 181-192);
(11) scFv-NKG2D_{TM}-4-1BB_{IC}-CD3ζs (see, e.g., Li Y, Hermanson D L, Moriarity B S Kaufman D S, Cell Stem Cell. 2018; 23: 181-192);
(12) scFv-NKG2D_{TM}-2B4_{IC}-DAP12_{IC}-CD3ζs (see, e.g., Li Y, Hermanson D L, Moriarity B S Kaufman D S, Cell Stem Cell. 2018; 23: 181-192);
(13) scFv-NKG2DTM-2B4IC-DAP10Ic-CD3ζs (see, e.g., Li Y, Hermanson D L, Moriarity B S Kaufman D S, Cell Stem Cell. 2018; 23: 181-192);
(14) scFv-NKG2D_{TM}-4-1BB_{IC}-2B4_{IC}-CD3ζS (see, e.g., Li Y, Hermanson D L, Moriarity B S Kaufman D S, Cell Stem Cell. 2018; 23: 181-192); and
(15) scFv-NKG2D_{TM}-CD3ζS (see, e.g., Li Y, Hermanson D L, Moriarity B S Kaufman D S, Cell Stem Cell. 2018; 23:181-192).

In some embodiments, the CAR is a anti-FITC CAR and the ligand is composed of a fluorescein or fluorescein isothiocyanate (FITC) moiety conjugated to an agent that binds to a desired target cell (such as a cancer cell). Exemplary ligands are described in the section above. In some embodiments, the ligand is FITC-folate.

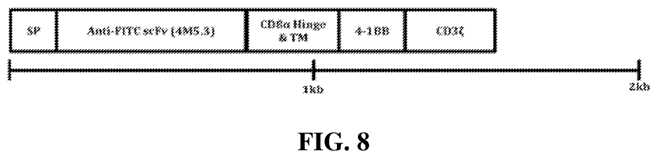

An illustrative CAR of the disclosure is shown in FIG. 8 where the fusion protein is encoded by a lentivirus expression vector and where “SP” is a signal peptide, the CAR is an anti-FITC CAR, a CD8a hinge is present, a transmembrane domain is present (“TM”), the co-stimulation domain is 4-1BB, and the activation signaling domain is CD35.

An illustrative nucleotide sequence encoding a CAR may comprise SEQ ID NO: 13:

(SEQ ID NO: 13)
ATGGCCTTACCAGTGACCGCCTTGCTCCTGCCGCTGGCCTTGCTGCTCC
ACGCCGCCAGGCCGGATGTCGTGATGACCCAGACCCCCCTCAGCCTCCC
AGTGTCCCTCGGTGACCAGGCTTCTATTAGTTGCAGATCCAGCCAGTCC
CTCGTGCACTCTAACGGTAATACCTACCTGAGATGGTATCTCCAGAAGC
CCGGACAGAGCCCTAAGGTGCTGATCTACAAAGTCTCCAACCGGGTGTC
TGGAGTCCCTGACCGCTTCTCAGGGAGCGGTTCCGGCACCGACTTCACC
CTGAAGATCAACCGGGTGGAGGCCGAAGACCTCGGCGTCTATTTCTGCT
CTCAGAGTACACATGTGCCCTGGACCTTCGGCGGAGGGACCAAGCTGGA
GATCAAAAGCTCCGCAGACGATGCCAAGAAAGATGCCGCTAAGAAAGAC
GATGCTAAGAAAGACGATGCAAAGAAAGACGGTGGCGTGAAGCTGGATG
AAACCGGAGGAGGTCTCGTCCAGCCAGGAGGAGCCATGAAGCTGAGTTG
CGTGACCAGCGGATTCACCTTTGGGCACTACTGGATGAACTGGGTGCGA
CAGTCCCCAGAGAAGGGGCTCGAATGGGTCGCTCAGTTCAGGAACAAAC
CCTACAATTATGAGACATACTATTCAGACAGCGTGAAGGGCAGGTTTAC
TATCAGTAGAGACGATTCCAAATCTAGCGTGTACCTGCAGATGAACAAT
CTCAGGGTCGAAGATACAGGCATCTACTATTGCACAGGGGCATCCTATG
GTATGGAGTATCTCGGTCAGGGGACAAGCGTCACAGTCAGTTTCGTGCC
GGTCTTCCTGCCAGCGAAGCCCACCACGACGCCAGCGCCGCGACCACCA
ACACCGGCGCCCACCATCGCGTCGCAGCCCCTGTCCCTGCGCCCAGAGG
CGTGCCGGCCAGCGGCGGGGGGCGCAGTGCACACGAGGGGGCTGGACTT
CGCCTGTGATATCTACATCTGGGCGCCCTTGGCCGGGACTTGTGGGGTC
CTTCTCCTGTCACTGGTTATCACCCTTTACTGCAACCACAGGAACCGTT
TCTCTGTTGTTAAACGGGGCAGAAAGAAACTCCTGTATATATTCAAACA
ACCATTTATGAGACCAGTACAAACTACTCAAGAGGAAGATGGCTGTAGC
TGCCGATTTCCAGAAGAAGAAGAAGGAGGATGTGAACTGAGAGTGAAGT
TCAGCAGGAGCGCAGACGCCCCCGCGTACCAGCAGGGCCAGAACCAGCT
CTATAACGAGCTCAATCTAGGACGAAGAGAGGAGTACGATGTTTTGGAC
AAGAGACGTGGCCGGGACCCTGAGATGGGGGGAAAGCCGAGAAGGAAGA
ACCCTCAGGAAGGCCTGTACAATGAACTGCAGAAAGATAAGATGGCGGA
GGCCTACAGTGAGATTGGGATGAAAGGCGAGCGCCGGAGGGGCAAGGGG
CACGATGGCCTTTACCAGGGTCTCAGTACAGCCACCAAGGACACCTACG
ACGCCCTTCACATGCAGGCCCTGCCCCCTCGCTAA

An illustrative CAR amino acid sequence may comprise SEQ ID NO: 14:

(SEQ ID NO: 14)
MALPVTALLLPLALLLHAARPDVVMTQTPLSLPVSLGDQASISCRSSQS
LVHSNGNTYLRWYLQKPGQSPKVLIYKVSNRVSGVPDRFSGSGSGTDFT
LKINRVEAEDLGVYFCSQSTHVPWTFGGGTKLEIKSSADDAKKDAAKKD
DAKKDDAKKDGGVKLDETGGGLVQPGGAMKLSCVTSGFTFGHYWMNWVR
QSPEKGLEWVAQFRNKPYNYETYYSDSVKGRFTISRDDSKSSVYLQMNN
LRVEDTGIYYCTGASYGMEYLGQGTSVTVSFVPVFLPAKPTTTPAPRPP
TPAPTIASQPLSLRPEACRPAAGGAVHTRGLDFACDIYIWAPLAGTCGV
LLLSLVITLYCNHRNRFSVVKRGRKKLLYIFKQPFMRPVQTTQEEDGCS
CRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKG
HDGLYQGLSTATKDTYDALHMQALPPR

An illustrative nucleotide insert may comprise SEQ ID NO: 15:

(SEQ ID NO: 15)
GCCACCATGGCCTTACCAGTGACCGCCTTGCTCCTGCCGCTGGCCTTGC
TGCTCCACGCCGCCAGGCCGGATGTCGTGATGACCCAGACCCCCCTCAG
CCTCCCAGTGTCCCTCGGTGACCAGGCTTCTATTAGTTGCAGATCCAGC
CAGTCCCTCGTGCACTCTAACGGTAATACCTACCTGAGATGGTATCTCC
AGAAGCCCGGACAGAGCCCTAAGGTGCTGATCTACAAAGTCTCCAACCG
GGTGTCTGGAGTCCCTGACCGCTTCTCAGGGAGCGGTTCCGGCACCGAC
TTCACCCTGAAGATCAACCGGGTGGAGGCCGAAGACCTCGGCGTCTATT
TCTGCTCTCAGAGTACACATGTGCCCTGGACCTTCGGCGGAGGGACCAA
GCTGGAGATCAAAAGCTCCGCAGACGATGCCAAGAAAGATGCCGCTAAG
AAAGACGATGCTAAGAAAGACGATGCAAAGAAAGACGGTGGCGTGAAGC
TGGATGAAACCGGAGGAGGTCTCGTCCAGCCAGGAGGAGCCATGAAGCT
GAGTTGCGTGACCAGCGGATTCACCTTTGGGCACTACTGGATGAACTGG
GTGCGACAGTCCCCAGAGAAGGGGCTCGAATGGGTCGCTCAGTTCAGGA
ACAAACCCTACAATTATGAGACATACTATTCAGACAGCGTGAAGGGCAG
GTTTACTATCAGTAGAGACGATTCCAAATCTAGCGTGTACCTGCAGATG
AACAATCTCAGGGTCGAAGATACAGGCATCTACTATTGCACAGGGGCAT
CCTATGGTATGGAGTATCTCGGTCAGGGGACAAGCGTCACAGTCAGTTT
CGTGCCGGTCTTCCTGCCAGCGAAGCCCACCACGACGCCAGCGCCGCGA
CCACCAACACCGGCGCCCACCATCGCGTCGCAGCCCCTGTCCCTGCGCC
CAGAGGCGTGCCGGCCAGCGGCGGGGGGCGCAGTGCACACGAGGGGGCT
GGACTTCGCCTGTGATATCTACATCTGGGCGCCCTTGGCCGGGACTTGT
GGGGTCCTTCTCCTGTCACTGGTTATCACCCTTTACTGCAACCACAGGA
ACCGTTTCTCTGTTGTTAAACGGGGCAGAAAGAAACTCCTGTATATATT
CAAACAACCATTTATGAGACCAGTACAAACTACTCAAGAGGAAGATGGC
TGTAGCTGCCGATTTCCAGAAGAAGAAGAAGGAGGATGTGAACTGAGAG
TGAAGTTCAGCAGGAGCGCAGACGCCCCCGCGTACCAGCAGGGCCAGAA
CCAGCTCTATAACGAGCTCAATCTAGGACGAAGAGAGGAGTACGATGTT
TTGGACAAGAGACGTGGCCGGGACCCTGAGATGGGGGGAAAGCCGAGAA
GGAAGAACCCTCAGGAAGGCCTGTACAATGAACTGCAGAAAGATAAGAT
GGCGGAGGCCTACAGTGAGATTGGGATGAAAGGCGAGCGCCGGAGGGGC
AAGGGGCACGATGGCCTTTACCAGGGTCTCAGTACAGCCACCAAGGACA
CCTACGACGCCCTTCACATGCAGGCCCTGCCCCCTCGCTAA

In some embodiments, the CAR may be encoded by a nucleic acid sequence that encodes a signal peptide to signal transport of the CAR in the cell. It is understood that typically the signal peptide is removed from the protein.

An illustrative CAR amino acid sequence without a signal peptide may comprise SEQ ID NO: 16:

(SEQ ID NO: 16)
DVVMTQTPLSLPVSLGDQASISCRSSQSLVHSNGNTYLRWYLQKPGQSP
KVLIYKVSNRVSGVPDRFSGSGSGTDFTLKINRVEAEDLGVYFCSQSTH
VPWTFGGGTKLEIKSSADDAKKDAAKKDDAKKDDAKKDGGVKLDETGGG
LVQPGGAMKLSCVTSGFTFGHYWMNWVRQSPEKGLEWVAQFRNKPYNYE
TYYSDSVKGRFTISRDDSKSSVYLQMNNLRVEDTGIYYCTGASYGMEYL
GQGTSVTVSFVPVFLPAKPTTTPAPRPPTPAPTIASQPLSLRPEACRPA
AGGAVHTRGLDFACDIYIWAPLAGTCGVLLLSLVITLYCNHRNRFSVVK
RGRKKLLYIFKQPFMRPVQTTQEEDGCSCRFPEEEEGGCELRVKFSRSA
DAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHM
QALPPR

An illustrative CAR amino acid sequence signal peptide may comprise SEQ ID NO: 17:

(SEQ ID NO: 17)
MALPVTALLLPLALLLHAARP

In various embodiments, CAR-expressing cells comprising the nucleic acid of SEQ ID NO: 13 or 15 are provided. In some embodiments, a chimeric antigen receptor polypeptide comprising SEQ ID NO: 14 is contemplated. In some embodiments, a chimeric antigen receptor polypeptide comprising SEQ ID NO: 16 is contemplated. In some embodiments, a vector is contemplated comprising SEQ ID NO: 13 or 15. In some embodiments, a lentiviral vector is contemplated comprising SEQ ID NO: 13 or 15. In some embodiments, SEQ ID NO: 14 can comprise or consist of human or humanized amino acid sequences. In some embodiments, SEQ ID NO: 16 can comprise or consist of human or humanized amino acid sequences.

In some embodiments, variant nucleic acid sequences or amino acid sequences having at least about 80%, at least about 90%, at least about 95%, at least about 97%, at least about 98%, at least about 99%, or at least about 99.5% sequence identity to SEQ ID NO: 13, SEQ ID NO: 14, SEQ ID NO: 15 or SEQ ID NO: 16 are contemplated.

While the affinity at which the CARs, expressed by the lymphocytes, bind to the targeted moiety can vary, and in some cases low affinity binding may be preferable (such as about 50 nM), the binding affinity of the CARs to the targeted ligand will generally be at least about 100 nM, 1 μM, or 10 μM, preferably at least about 100 μM, 1 fM or 10 fM, even more preferably at least about 100 fM.

### V. NUCLEIC ACID VECTORS

As used herein, the term “nucleic acid vector” is intended to mean any nucleic acid that functions to carry, harbor or express a nucleic acid of interest. Nucleic acid vectors can have specialized functions such as expression, packaging, pseudotyping, transduction or sequencing, for example. Nucleic acid vectors also can have, for example, manipulatory functions such as a cloning or shuttle vector. The structure of the vector can include any desired form that is feasible to make and desirable for a particular use. Such forms include, for example, circular forms such as plasmids and phagemids, as well as linear or branched forms. A nucleic acid vector can be composed of, for example, DNA or RNA, as well as contain partially or fully, nucleotide derivatives, analogs and mimetics. Such nucleic acid vectors can be obtained from natural sources, produced recombinantly or chemically synthesized.

Non-limiting examples of vector systems of the present disclosure include a retrovirus, a lentivius, a foamy virus, and a Sleeping Beauty transposon.

### Retroviral Particles

Retroviruses include lentiviruses, gamma-retroviruses, and alpha-retroviruses, each of which may be used to deliver polynucleotides to cells using methods known in the art. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV-1 and HIV-2) and the Simian Immunodeficiency Virus (SIV). Retroviral vectors have been generated by multiply attenuating the HIV virulence genes, for example, the genes env, vif, vpr, vpu and nef are deleted, making the vector biologically safe.

Illustrative lentiviral vectors include those described in Naldini et al. (1996) Science 272:263-7; Zufferey et al. (1998) J. Virol. 72:9873-9880; Dull et al. (1998) J. Virol. 72:8463-8471; U.S. Pat. Nos. 6,013,516; and 5,994,136, which are each incorporated herein by reference in their entireties. In general, these vectors are configured to carry the essential sequences for selection of cells containing the vector, for incorporating foreign nucleic acid into a lentiviral particle, and for transfer of the nucleic acid into a target cell.

A commonly used lentiviral vector system is the so-called third-generation system. Third-generation lentiviral vector systems include four plasmids. The “transfer plasmid” encodes the polynucleotide sequence that is delivered by the lentiviral vector system to the target cell. The transfer plasmid generally has one or more transgene sequences of interest flanked by long terminal repeat (LTR) sequences, which facilitate integration of the transfer plasmid sequences into the host genome. For safety reasons, transfer plasmids are generally designed to make the resulting vector replication incompetent. For example, the transfer plasmid lacks gene elements necessary for generation of infective particles in the host cell. In addition, the transfer plasmid may be designed with a deletion of the 3′ LTR, rendering the virus “self-inactivating” (SIN). See Dull et al. (1998) J. Virol. 72:8463-71; Miyoshi et al. (1998) J. Virol. 72:8150-57. The viral particle may also comprise a 3′ untranslated region (UTR) and a 5′ UTR. The UTRs comprise retroviral regulatory elements that support packaging, reverse transcription and integration of a proviral genome into a cell following contact of the cell by the retroviral particle.

Third-generation systems also generally include two “packaging plasmids” and an “envelope plasmid.” The “envelope plasmid” generally encodes an Env gene operatively linked to a promoter. In an illustrative third-generation system, the Env gene is VSV-G and the promoter is the CMV promoter. The third-generation system uses two packaging plasmids, one encoding gag and pol and the other encoding rev as a further safety feature; an improvement over the single packaging plasmid of so-called second-generation systems. Although safer, the third-generation system can be more cumbersome to use and result in lower viral titers due to the addition of an additional plasmid. Illustrative packing plasmids include, without limitation, pMD2.G, pRSV-rev, pMDLG-pRRE, and pRRL-GOI.

Many retroviral vector systems rely on the use of a “packaging cell line.” In general, the packaging cell line is a cell line whose cells are capable of producing infectious retroviral particles when the transfer plasmid, packaging plasmid(s), and envelope plasmid are introduced into the cells. Various methods of introducing the plasmids into the cells may be used, including transfection or electroporation. In some cases, a packaging cell line is adapted for high-efficiency packaging of a retroviral vector system into retroviral particles.

As used herein, the terms “retroviral vector” or “lentiviral vector” is intended to mean a nucleic acid that encodes a retroviral or lentiviral cis nucleic acid sequence required for genome packaging and one or more polynucleotide sequence to be delivered into the target cell. Retroviral particles and lentiviral particles generally include an RNA genome (derived from the transfer plasmid), a lipid-bilayer envelope in which the Env protein is embedded, and other accessory proteins including integrase, protease, and matrix protein. As used herein, the terms “retroviral particle” and “lentiviral particle” refers a viral particle that includes an envelope, has one or more characteristics of a lentivirus, and is capable of invading a target host cell. Such characteristics include, for example, infecting non-dividing host cells, transducing non-dividing host cells, infecting or transducing host immune cells, containing a retroviral or lentiviral virion including one or more of the gag structural polypeptides, containing a retroviral or lentiviral envelope including one or more of the env encoded glycoproteins, containing a genome including one or more retrovirus or lentivirus cis-acting sequences functioning in replication, proviral integration or transcription, containing a genome encoding a retroviral or lentiviral protease, reverse transcriptase or integrase, or containing a genome encoding regulatory activities such as Tat or Rev. The transfer plasmids may comprise a cPPT sequence, as described in U.S. Pat. No. 8,093,042.

The efficiency of the system is an important concern in vector engineering. The efficiency of a retroviral or lentiviral vector system may be assessed in various ways known in the art, including measurement of vector copy number (VCN) or vector genomes (vg) such as by quantitative polymerase chain reaction (qPCR), or titer of the virus in infectious units per milliliter (IU/mL). For example, the titer may be assessed using a functional assay performed on the cultured tumor cell line HT1080 as described in Humbert et al. Development of third-generation Cocal Envelope Producer Cell Lines for Robust Retroviral Gene Transfer into Hematopoietic Stem Cells and T-cells. Molecular Therapy 24:1237-1246 (2016). When titer is assessed on a cultured cell line that is continually dividing, no stimulation is required and hence the measured titer is not influenced by surface engineering of the retroviral particle. Other methods for assessing the efficiency of retroviral vector systems are provided in Gaererts et al. Comparison of retroviral vector titration methods. BMC Biotechnol. 6:34 (2006).

In some embodiments, the retroviral particles and/or lentiviral particles of the disclosure comprise a polynucleotide comprising a sequence encoding a receptor that specifically binds to the gating adaptor. In some embodiments, a sequence encoding a receptor that specifically binds to the gating adaptor is operatively linked to a promoter. Illustrative promoters include, without limitation, a cytomegalovirus (CMV) promoter, a CAG promoter, an SV40 promoter, an SV40/CD43 promoter, and a MND promoter.

In some embodiments, the retroviral particles comprise transduction enhancers. In some embodiments, the retroviral particles comprise tagging proteins.

In some embodiments, each of the retroviral particles comprises a polynucleotide comprising, in 5′ to 3′ order: (i) a 5′ long terminal repeat (LTR) or untranslated region (UTR), (ii) a promoter, (iii) a sequence encoding a receptor that specifically binds to a ligand, and (iv) a 3′ LTR or UTR.

In some embodiments, the retroviral particles comprise a cell surface receptor that binds to a surface marker on a target host cell, allowing host cell transduction. The viral vector may comprise a heterologous viral envelope glycoprotein giving a pseudotyped viral vector. For example, the viral envelope glycoprotein may be derived from RD114 or one of its variants, VSV-G, Gibbon-ape leukaemia virus (GALV), or is the Amphotropic envelope, Measles envelope or baboon retroviral envelope glycoprotein. In some embodiments, the cell-surface receptor is a VSV G protein from the Cocal strain or a functional variant thereof.

Various fusion glycoproteins can be used to pseudotype lentiviral vectors. While the most commonly used example is the envelope glycoprotein from vesicular stomatitis virus (VSVG), many other viral proteins have also been used for pseudotyping of lentiviral vectors. See Joglekar et al. Human Gene Therapy Methods 28:291-301 (2017). The present disclosure contemplates substitution of various fusion glycoproteins. Notably, some fusion glycoproteins result in higher vector efficiency.

In some embodiments, pseudotyping a fusion glycoprotein or functional variant thereof facilitates targeted transduction of specific cell types, including, but not limited to, innate lymphoid cells, cytotoxic innate lymphoid cells, or NK cells. In some embodiments, the fusion glycoprotein or functional variant thereof is/are full-length polypeptide(s), functional fragment(s), homolog(s), or functional variant(s) of Human immunodeficiency virus (HIV) gp160, Murine leukemia virus (MLV) gp70, Gibbon ape leukemia virus (GALV) gp70, Feline leukemia virus (RD114) gp70, Amphotropic retrovirus (Ampho) gp70, 10A1 MLV (10A1) gp70, Ecotropic retrovirus (Eco) gp70, Baboon ape leukemia virus (BaEV) gp70, Measles virus (MV) H and F. Nipah virus (NiV) H and F, Rabies virus (RabV) G, Mokola virus (MOKV) G, Ebola Zaire virus (EboZ) G, Lymphocytic choriomeningitis virus (LCMV) GP1 and GP2, Baculovirus GP64, Chikungunya virus (CHIKV) E1 and E2, Ross River virus (RRV) E1 and E2, Semliki Forest virus (SFV) E1 and E2, Sindbis virus (SV) E1 and E2, Venezualan equine encephalitis virus (VEEV) E1 and E2, Western equine encephalitis virus (WEEV) E1 and E2, Influenza A, B, C, or D HA, Fowl Plague Virus (FPV) HA, Vesicular stomatitis virus VSV-G, or Chandipura virus and Piry virus CNV-G and PRV-G.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

The disclosure further provides various retroviral vectors, including but not limited to gamma-retroviral vectors, alpha-retroviral vectors, and lentiviral vectors. In some embodiments, the vector may be a viral vector, a retroviral vector, a lentiviral vector, a gamma-retroviral vector. In some embodiments, the viral vector comprises a VSV G-protein or functional variant thereof. In some embodiments, the viral vector comprises a Cocal G-protein or functional variant thereof.

### VI. METHODS OF TREATMENT

The present disclosure provides methods of treating a subject in need thereof with the compositions, therapeutic compositions, cells, vectors, and polynucleotides disclosed herein. In some embodiments, the disclosure provides a method of treating cancer and/or killing cancer cells in a subject, comprising administering a therapeutically effective amount of the disclosed cells to the subject. Also provided is a method of treating a tumor and/or killing tumor cells in a subject, comprising administering an effective amount of non-physiological ligand to the subject, wherein the non-physiological ligand causes the cells to proliferate according to any of the foregoing embodiments.

Also provided are uses and compositions for use in in any of the provided methods. The methods and uses may include use of any composition as described herein, including those produced by methods herein or a pharmaceutical composition provided herein, such as described below. In some embodiments, provided herein is a method of treating a condition in an individual, comprising administering any of the provided iCILs to an individual in need thereof. Uses include uses of the cells or pharmaceutical compositions thereof in such methods and treatments, and in the preparation of a medicament in order to carry out such therapeutic methods.

Such methods and uses include therapeutic methods and uses, for example, involving administration of the therapeutic cells, or compositions containing the same, to a subject having a disease, condition, or disorder. In some cases, the disease or disorder is a tumor or cancer. In some embodiments, the disease or disorder is a virus infection. In some embodiments, the cells or pharmaceutical composition thereof is administered in an effective amount to effect treatment of the disease or disorder. In some embodiments, the methods thereby treat the disease or condition or disorder in the subject.

Among cells in the compositions or for use in the methods or uses herein are engineered iCIL cells that comprise a heterologous nucleic acid encoding an antigen receptor (e.g. CAR). In some embodiments, the CAR is able to target an antigen expressed by a cell associated with a disease or condition, such as a tumor cell. Targeting to the antigen directs the iCIL to the cell to trigger target cell death by cytotoxic killing, thereby treating the disease or condition. In some embodiments, the provided methods can be used to treat any disease or disorder in which targeted cell killing mediates a treatment of the disease or condition. For instance, in the case of a CAR, the disease or condition to be treated is any disease or condition that is associated with expression of an antigen that is recognized or targeted by the CAR. In some embodiments as described, the CAR may be a “universal” CAR that can be targeted to a target cell by labeling with a separately administered conjugate molecule that acts as a bridge between the CAR-expressing cells and targeted cancer cells. In some embodiments, the conjugate molecules are conjugates comprising a hapten and a cell-targeting moiety, such as any suitable tumor cell-specific ligand.

In some embodiments, provided is a method of administering an effective amount of non-physiological ligand (e.g. rapamycin or rapalog) to the subject, wherein the non-physiological ligand causes the cells to proliferate according to any of the foregoing embodiments. In some embodiments, the non-physiological ligand is administered in combination with the CIL cells. The non-physiological ligand can be administered concurrently or sequentially with administration of CILs to the subject. In some embodiments, the non-physiological ligand is first administered before administration of the CILs or concurrently with administration of the CILs. In some embodiments, the non-physiological ligand is administration after administration of the CILs. In some embodiments, the non-physiological ligand may be administered intermittently or at various intervals, such as for a period of time after administration of CIL cells, such as CAR-CILs, to a subject.

In some embodiments, the non-physiological ligand is present or provided at an amount effective to cause expansion of the CIL cells ex vivo. In some embodiments, the non-physiological ligand is present or provided at an amount effective to cause expansion of the CIL cells in vivo. In some embodiments, the non-physiological ligand is present or provided at an amount effective to cause CIL cell cytokine secretion ex vivo. In some embodiments, the non-physiological ligand is present or provided at an amount effective to cause CIL cell cytokine secretion in vivo. In some embodiments, the non-physiological ligand is present or provided at an amount effective to cause CIL cell secretion of CD107a, interferon gamma (IFNγ) and/or tumor necrosis factor alpha (TNF-α) ex vivo. In some embodiments, the non-physiological ligand is present or provided at an amount effective to cause CIL cell secretion of CD107a, interferon gamma (IFNγ) and/or tumor necrosis factor alpha (TNF-α) in vivo. In some embodiments, the non-physiological ligand is present or provided at an amount effective to cause tumor cell killing.

In some embodiments, the non-physiological ligand is present or provided at a therapeutically effective amount.

In some embodiments, the non-physiological ligand is rapamycin or a rapamycin analog. In some embodiments, the rapamycin analog is rapalog.

In some embodiments, the non-physiological ligand (e.g. rapamycin or an analog) is administered at a dose of 1 mg to 100 mg, optionally between 10-100 mg, optionally at or about 10 mg, 20 mg, 25 mg, 30 mg, 40 mg, 50 mg or any value between any of the foregoing.

In some embodiments, the malignancy is a solid tumor, sarcoma, carcinoma, lymphoma, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), chronic or acute leukemia, acute myeloid leukemia, chronic myeloid leukemia, acute lymphoblastic leukemia (ALL) (including non T-cell ALL), chronic lymphocytic leukemia (CLL), T-cell lymphoma, one or more of B-cell acute lymphoid leukemia (“BALL”), T-cell acute lymphoid leukemia (“TALL”), acute lymphoid leukemia (ALL), chronic myelogenous leukemia (CML), B cell prolymphocytic leukemia, blastic plasmacytoid dendritic cell neoplasm, Burkitf's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, myelodysplasia and myelodysplastic syndrome, plasmablastic lymphoma, plasmacytoid dendritic cell neoplasm, Waldenstrom macroglobulinemia, a plasma cell proliferative disorder (e.g., asymptomatic myeloma (smoldering multiple myeloma or indolent myeloma)), monoclonal gammapathy of undetermined significance (MGUS), plasmacytomas (e.g., plasma cell dyscrasia, solitary myeloma, solitary plas acyto a, extramedullary plasmacytoma, and multiple plasmacytoma), systemic amyloid light chain amyloidosis, POEMS syndrome (also known as Crow-Fukase syndrome, Takatsuki disease, and PEP syndrome), or a combination thereof.

In some embodiments, a method disclosed herein may be used to treat cancer and/or kill cancer cells in a subject by administering a therapeutically effective amount of the cells according to any of the foregoing embodiments.

The present disclosure also provides a method of treating cancer and/or killing cancer cells in a subject, comprising administering the system of any of the foregoing embodiments to the subject.

In some embodiments, the present disclosure provides a method of treating cancer with any of the compositions provided herein. “Cancer” has its plain and ordinary meaning when read in light of the specification, and may include but is not limited to, for example, a group of diseases involving abnormal cell growth with the potential to invade or spread to other parts of the body. Subjects that can be addressed using the methods described herein include subjects identified or selected as having cancer, including but not limited to colon, lung, liver, breast, renal, prostate, ovarian, skin (including melanoma), bone, and brain cancer, etc. Such identification and/or selection can be made by clinical or diagnostic evaluation. In some embodiments, the tumor associated antigens or molecules are known, such as melanoma, breast cancer, brain cancer, squamous cell carcinoma, colon cancer, leukemia, myeloma, and/or prostate cancer. Examples include but are not limited to B cell lymphoma, breast cancer, brain cancer, prostate cancer, and/or leukemia. In some embodiments, one or more oncogenic polypeptides are associated with kidney, uterine, colon, lung, liver, breast, renal, prostate, ovarian, skin (including melanoma), bone, brain cancer, adenocarcinoma, pancreatic cancer, chronic myelogenous leukemia or leukemia. In some embodiments, a method of treating, ameliorating, or inhibiting a cancer in a subject is provided. In some embodiments, the cancer is breast, ovarian, lung, pancreatic, prostate, melanoma, renal, pancreatic, glioblastoma, neuroblastoma, medulloblastoma, sarcoma, liver, colon, skin (including melanoma), bone or brain cancer.

In some embodiments, the target cell is a tumor cell. In some embodiments, the target cell exists in a tumor microenvironment.

In some embodiments, the present disclosure provides a method of treating cancer with any of the compositions provided herein without prior conditioning of the subject. In some embodiments, a subject of the present disclosure does not need to receive a lymphodepleting therapy. A person skilled in the art will understand the common lymphodepleting therapies available, such as chemotherapy. In some embodiments, a subject of the present disclosure has not received a lymphodepleting therapy. In some embodiments, a differentiated cell is provided to a subject that has not received a lymphodepleting therapy. In some embodiments, the subject has not received a lymphodepleting therapy for 1, 2, 3, 4, 5, 6, 7, 8, 9 or 10 days prior to administration of the differentiated cell.

In some embodiments, a differentiated cell is provided to the subject 1, 2, 3, 4, 5, 6, 7, 8, 9, 10, 11, 12, 15, 20, 24, 36, 48, 60 or 72 hours after administration of a ligand composition, or any time within a range defined by any two aforementioned values. In some embodiments, the cell is provided to the subject 1, 2, 3, 4, 5, 6, 7, 8, 9, 10, 11, 12, 15, 20, 24, 36 or 48 hours before administration of the ligand composition, or any time within a range defined by any two aforementioned values. In some embodiments, the cell is provided to the subject within seconds or minutes, such as less than an hour, of providing the composition to the subject. In some embodiments, a boost of the cell and/or the composition is provided to the subject.

In some aspects, the present disclosure provides a method of treating a cancer as described in WO 2019/144095, which is incorporated herein by reference in its entirety.

In some embodiments, the present disclosure provides a method of treating cancer comprising administering a chimeric antigen receptor cell (e.g., an NK cell), wherein the CAR comprises an E2 anti-fluorescein antibody fragment. In some embodiments, the method of treating cancer further comprises administering a small molecule linked to a targeting moiety by a linker.

In some embodiments, the targeting moiety is determined by the type of cancer being treated in the subject. As an example, folate receptor is highly expressed on the surface of a wide variety of solid tumor cells including breast (e.g., triple negative breast cancer), ovarian, endometrial, kidney, lung, brain, pancreatic, gastric, prostate, acute myelocytic leukemia, and non small cell lung cancers. Thus, in some embodiments, the targeting moiety comprises a folate, which would bind folate receptor expressed on a cancer or tumor cell. In other embodiments, the folate can be folic acid, a folic acid analog or any folate-receptor binding molecule. In some embodiments, the small molecule comprises fluorescein, fluorescein isothiocyanate (FITC), NHS-fluorescein, or any other fluorophore. In some embodiments, the small molecule linked to a targeting moiety is FITC-folate.

In some embodiments, the disclosure contemplates engraftment of engineered stem cells for use in the treatment of subjects with cancer. In some embodiments, the iPSC-derived engineered cytotoxic innate lymphoid cells stably engraft in the subject. In some embodiments, the iPSC-derived engineered cytotoxic innate lymphoid cells display long-term engraftment in the subject. In some embodiments, administration of the iPSC-derived engineered cytotoxic innate lymphoid cells allowed for engraftment and further differentiation within the subject. In some embodiments, the iPSC-derived engineered natural killer cells display long-term persistence in the subject.

### Combination Therapy

In some embodiments, the provide methods can be used in a combination with an additional cancer therapy.

In some embodiments, the additional cancer therapy is a small molecule, e.g., a chemical compound, an antibody therapy, e.g., a humanized monoclonal antibody with or without conjugation to a radionuclide, toxin, or drug, surgery, and/or radiation.

In some embodiments, the subject is selected to receive an additional cancer therapy, which can include a cancer therapeutic, radiation, chemotherapy, or a drug for the treatment of cancer. In some embodiments, the drugs comprise Abiraterone, Alemtuzumab, Anastrozole, Aprepitant, Arsenic trioxide, Atezolizumab, Azacitidine, Bevacizumab, Bleomycin, Bortezomib, Cabazitaxel, Capecitabine, Carboplatin, Cetuximab, Chemotherapy drug combinations, Cisplatin, Crizotinib, Cyclophosphamide, Cytarabine, Denosumab, Docetaxel, Doxorubicin, Eribulin, Erlotinib, Etoposide, Everolimus, Exemestane, Filgrastim, Fluorouracil, Fulvestrant, Gemcitabine, Imatinib, Imiquimod, Ipilimumab, Ixabepilone, Lapatinib, Lenalidomide, Letrozole, Leuprolide, Mesna, Methotrexate, Nivolumab, Oxaliplatin, Paclitaxel, Palonosetron, Pembrolizumab, Pemetrexed, Prednisone, Radium-223, Rituximab, Sipuleucel-T, Sorafenib, Sunitinib, Talc Intrapleural, Tamoxifen, Temozolomide, Temsirolimus, Thalidomide, Trastuzumab, Vinorelbine or Zoledronic acid.

In some embodiments, the subject is selected to receive an antibody therapy. In some embodiments, the antibody therapy comprises an anti-CD38 therapy. In some embodiments, the anti-CD38 antibody is daratumumab (Darzalex), isatuximab (Sarclisa) or MOR202. In some embodiments, the anti-CD38 antibody is daratumumab (Darzalex). In some embodiments, the anti-CD38 antibody is isatuximab (Sarclisa). In some embodiments, the anti-CD38 antibody is MOR202. In some embodiments, the subject is selected to receive a composition comprising any of the iCIL cells provided herein and daratumumab. In some embodiments, the subject is selected to receive a composition comprising any of the iCIL cells provided herein and isatuximab. In some embodiments, the subject is selected to receive a composition comprising any of the iCIL cells provided herein and MOR202.

### Modes of Administration and Dosing

In some embodiments, transduced CIL cells may be grown in conditions that are suitable for a population of cells that will be introduced into a subject such as a human. Specific considerations include the use of culture media that lacks any animal products, such as bovine serum. Other considerations include sterilized-condition to avoid contamination of bacteria, fungi and mycoplasma. In some embodiments, any of the cell culturing methods contemplated in the disclosure may be used to grow CAR-expressing CIL cells.

In some embodiments, after transfection, the cells can be immediately administered to the patient or the cells can be cultured for at least about 1, 2, 3, 4, 5, 6, 7, 8, 9, 10, 11, 12, 13, 14, 15, 16, 17, 18 or more days, or between about 5 and about 12 days, between about 6 and about 13 days, between about 7 and about 14 days, or between about 8 and about 15 days, for example, to allow time for the cells to recover from the transfection. Suitable culture conditions can be similar to the conditions under which the cells were cultured for activation either with or without the agent that was used to promote activation. In some embodiments, any of the methods of administering CIL cells contemplated in the disclosure may be used to administer CAR-expressing CIL cells to the patient.

The disclosed cells may be administered in a number of ways depending upon whether local or systemic treatment is desired.

In the case of adoptive cell therapy, methods for administration of cells for adoptive cell therapy are known and may be used in connection with the provided methods and compositions.

In general, administration may be topical, parenteral, or enteral. The compositions of the disclosure are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrastemal, intravenous, intraarterial, intrathecal, intraventricular, intraurethral, intracranial, intratumoral, intrasynovial injection or infusions; and kidney dialytic infusion techniques. In an embodiment, parenteral administration of the compositions of the present disclosure comprises intravenous administration.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Illustrative parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

The present compositions of viral particles, adaptor molecules, and/or immune cells may be administered in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of infusing differentiated cells or transgenic differentiated cells according to the disclosure, a subject is administered the range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges, and/or such a number of cells per kilogram of body weight of the subject. For example, in some embodiments the administration of the cells or population of cells can comprise administration of about 103 to about 109 cells per kg body weight including all integer values of cell numbers within those ranges. In some embodiments, the dose is administered one time. In some embodiments, administration of the dose is repeated a plurality of times in a multi-dose administration regimen.

In some embodiments, greater than at or about 5×10⁹ iCILs (e.g. iCIL-CAR) is administered per dose. In some embodiments, from about 5×10⁹ iCILs to about 100×10⁹ iCILs is administered per dose, from about 5×10⁹ iCILs to about 50×10⁹ iCILs is administered per dose, from about 5×10⁹ iCILs to about 25×10⁹ iCILs is administered per dose, from about 5×10⁹ iCILs to 10×10⁹ iCILs is administered per dose, from about 10×10⁹ iCILs to about 100×10⁹ iCILs is administered per dose, from about 10×10⁹ iCILs to about 50×10⁹ iCILs is administered per dose, from about 10×10⁹ iCILs to about 25×10⁹ iCILs is administered per dose, from about 25×10⁹ iCILs to about 100×10⁹ iCILs is administered per dose, from about 25×10⁹ iCILs to about 50×10⁹ iCILs is administered per dose, or from about 50×10⁹ iCILs to about 100×10⁹ iCILs is administered per dose. In some embodiments, at or about 5×10⁹ iCILs, at or about 10×10⁹ iCILs, at or about 20×10⁹ iCILs, at or about 30×10⁹ iCILs, at or about 40×10⁹ iCILs, at or about 50×10⁹ iCILs, at or about 60×10⁹ iCILs, at or about 70×10⁹ iCILs, at or about 80×10⁹ iCILs, at or about 90×10⁹ iCILs, at or about 100×10⁹ iCILs, or any value between any of the foregoing is administered per dose. In some embodiments, the dose is administered one time. In some embodiments, administration of the dose is repeated a plurality of times in a multi-dose administration regimen.

In some embodiments, the therapeutic use of the iCIL cells is a single-dose treatment.

In some embodiments, the therapeutic use of the iCIL cells is a multi-dose treatment. In some embodiments, the multi-dose treatment is one dose every day, every 3 days, every 5 days, every 7 days, every 10 days, every 15 days, every 20 days, every 25 days, every 30 days, every 35 days, every 40 days, every 45 days, or every 50 days, or any number of days in-between. In some embodiments, the composition containing iCILs cells may be administered once weekly. The frequency and duration of the multi-dose therapy can be empirically determined by a skilled physician or clinician, such as based on factors that include signs or symptoms of disease or symptoms in the subject or the pharmacokinetics or expansion of iCILs in the subject.

In some embodiments, the composition containing iCILs cells may be administered as a multi-dose treatment for a predetermined number of doses. In some embodiments, the composition containing iCILs may be administered as two doses, three doses, four doses, five doses, six doses, seven doses, eight doses, nine doses, ten doses, eleven doses or twelve doses. In some embodiments, the doses are administered for 4 weeks, 6 weeks, 8 weeks, 10 weeks, 12 weeks, 16 weeks, 20 weeks, 24 weeks, 28 weeks, 32 weeks, 36 weeks or more.

In some embodiments, the provided compositions containing iCILs can be administered to a subject by any convenient route including parenteral routes such as subcutaneous, intramuscular, intravenous, and/or epidural routes of administration. In particular embodiments, the provided compositions are administered by intravenous infusion.

In some embodiments, the compositions containing iCILs is administered in combination with a non-physiological ligand of the synthetic cytokine receptor. In some embodiments, the non-physiological ligand is rapamycin or a rapamycin analog. In some embodiments, the rapamycin analog is rapalog. In some embodiments, the non-physiological ligand (e.g. rapamycin or an analog) is administered at a dose of 1 mg to 100 mg, optionally between 10-100 mg, optionally at or about 10 mg, 20 mg, 25 mg, 30 mg, 40 mg, 50 mg or any value between any of the foregoing.

In some embodiments, the dose of the non-physiological ligand is administered as a single dose or as multiple doses.

In some embodiments, the non-physiological ligand is administered in multiple doses. In some embodiments, each dose of the multiple doses are for administration every day, every 3 days, every 5 days, every 7 days, every 10 days, every 15 days, every 20 days, every 25 days, every 30 days, every 35 days, every 40 days, every 45 days, or every 50 days, or any number of days in-between. In some embodiments, the non-physiological ligand may be administered once weekly. The frequency and duration of administration of the non-physiological ligand can be empirically determined by a skilled physician or clinician, such as based on the pharmacokinetics or expansion of iCILs in the subject.

In some embodiments, the non-physiological ligand may be administered as a multi-dose treatment for a predetermined number of doses. In some embodiments, the non-physiological ligand may be administered as two doses, three doses, four doses, five doses, six doses, seven doses, eight doses, nine doses, ten doses, eleven doses or twelve doses. In some embodiments, the doses are administered for 4 weeks, 6 weeks, 8 weeks, 10 weeks, 12 weeks, 16 weeks, 20 weeks, 24 weeks, 28 weeks, 32 weeks, 36 weeks or more.

In some embodiments, the pharmacokinetics of the CILs following administration to a subject can be monitored. In come embodiments, the concentration of CILs in the plasma following administration can be measured using any method known in the art suitable for assessing concentrations of cells or of transgenes (e.g. CAR or synthetic cytokine receptor) expressed by such cells in samples of blood, or any methods described herein. For example, nucleic acid-based methods, such as quantitative PCR (qPCR) or flow cytometry-based methods, or other assays, such as an immunoassay, ELISA, or chromatography/mass spectrometry-based assays can be used. In some embodiments, qPCR can be used to detect copy number of nucleic acid encoding the transgene (e.g. CAR or synthetic cytokine receptor) compared to total amount of nucleic acid or DNA in the particular sample, e.g., blood, serum, plasma or tissue, such as a tumor sample. In some embodiments, flow cytometric assays can be used for detecting cells expressing an engineered surface protein, such as a CAR or the synthetic cytokine receptor, generally using antibodies specific for the protein. Cell-based assays may also be used to detect the number or percentage or concentration of functional cells, such as cells capable of binding to and/or neutralizing and/or inducing responses, e.g., cytotoxic responses, against target cells of the disease or condition or expressing the antigen targeted by the CAR.

### VII. EXEMPLARY EMBODIMENTS

Among the provided embodiments are:
1. An engineered stem cell comprising a synthetic cytokine receptor for a non-physiological ligand,
wherein the cytokine receptor comprises:
a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.
2. The engineered stem cell of embodiment 1, wherein the first dimerization domain and the second dimerization domain are extracellular domains.
3. The engineered stem cell of embodiment 1 or embodiment 2, wherein the synthetic gamma chain polypeptide comprises, in N- to C-terminal order, the first dimerization domain, the first transmembrane domain, and the interleukin-2 receptor subunit gamma (IL-2RG) intracellular domain, and
the synthetic beta chain polypeptide comprises, in N- to C-terminal order, the second dimerization domain, the second transmembrane domain, and the intracellular domain.
4. The engineered stem cell of any of embodiments 1-3, wherein the IL-2RG intracellular domain comprises a polypeptide sequence at least 95% identical to SEQ ID NO: 1, or a polypeptide sequence as set forth in SEQ ID NO: 1.
5. The engineered stem cell of any of embodiments 1-4, wherein the first transmembrane domain comprises the IL-2RG transmembrane domain.
6. The engineered stem cell of embodiment 5, wherein the IL-2RG transmembrane domain comprises a polypeptide sequence at least 95% identical to SEQ ID NO: 8 or 31, or a polypeptide sequence as set forth in SEQ ID NO: 8 or 31.
7. The engineered stem cell of any of embodiments 1-6, wherein the beta chain intracellular domain comprises the IL-2RB intracellular domain.
8. The engineered stem cell of embodiment 7, wherein the IL-2RB intracellular domain comprises a polypeptide sequence at least 95% identical to SEQ ID NO: 2, or a polypeptide sequence as set forth in SEQ ID NO: 2.
9. The engineered stem cell of any of embodiments 1-6, wherein the beta chain intracellular domain comprises the IL-7RB intracellular domain.
10. The engineered stem cell of embodiment 9, wherein the IL-7RB intracellular domain comprises a polypeptide sequence at least 95% identical to SEQ ID NO: 3, or a polypeptide sequence as set forth in SEQ ID NO: 3.
11. The engineered stem cell of any of embodiments 1-6, wherein the beta chain intracellular domain comprises the IL-21RB intracellular domain.
12. The engineered stem cell of embodiment 11, wherein the IL-21RB intracellular domain comprises a polypeptide sequence at least 95% identical to SEQ ID NO: 4, or a polypeptide sequence as set forth in SEQ ID NO: 4.
13. The engineered stem cell of any of embodiments 1-12, wherein the second transmembrane domain comprises a transmembrane domain from the same beta chain intracellular domain.
14. The engineered stem cell of embodiment 1-8 and 13, wherein the second transmembrane domain is a transmembrane domain of IL-2RB comprising a polypeptide sequence at least 95% identical to SEQ ID NO: 35 or 36, or a polypeptide sequence as set forth in SEQ ID NO: 35 or 36.
15. The engineered stem cell of any of embodiments 1-8, 13 and 14, wherein:
the synthetic gamma chain polypeptide contains an IL-2RG TM domain comprising the sequence set forth in SEQ ID NO: 8 or 31 and a IL-2RG intracellular domain comprising the sequence set forth in SEQ ID NO:1; and
the synthetic beta chain polypeptide contains an IL-2RB TM domain comprising the sequence set forth in SEQ ID NO: 35 or 36 and a IL-2RB intracellular domain comprising the sequence set forth in SEQ ID NO:2.
16. The engineered stem cell of any one of embodiments 1 to 15, wherein the first dimerization domain and the second dimerization domain are heterodimerization domains selected from selected from FK506-Binding Protein of size 12 kD (FKBP) and a FKBP12-rapamycin binding (FRB) domain; and/or
wherein the non-physiological ligand is rapamycin or a rapalog.
17. The engineered stem cell of embodiment 16, wherein the FRB domain comprises a polypeptide sequence at least 95% identical to SEQ ID NO: 6 or SEQ ID NO: 7.
18. The engineered stem cell of embodiment 16, wherein the FRB domain comprises the polypeptide sequence set forth in SEQ ID NO: 6 or SEQ ID NO: 7.
19. The cell population of any one of embodiments 1 to 15, wherein the first dimerization domain and the second dimerization domain are heterodimerization domains selected from selected from FK506-Binding Protein of size 12 kD (FKBP) and a calcineurin domain; and/or
wherein the non-physiological ligand is FK506 or an analogue thereof.
20. The engineered stem cell of any one of embodiments 1 to 16, wherein the FKBP domain comprises a polypeptide sequence at least 95% identical to SEQ ID NO: 5 or SEQ ID NO:30.
21. The engineered stem cell of any one of embodiments 1 to 16, wherein the FKBP domain comprises the polypeptide sequence set forth in SEQ ID NO: 5 or SEQ ID NO: 30.
22. The engineered stem cell of any of embodiments 1-8 and 13-21, wherein the synthetic gamma chain polypeptide has the sequence of amino acids set forth in SEQ ID NO: 28 or a sequence of amino acids that is at least 90%, at least 95%, at least 96%, at least 97%, at least 98%, at least 99%, or 100% identical to SEQ ID NO: 28, and the synthetic beta chain polypeptide has the sequence of amino acids set forth in SEQ ID NO:33 or a sequence of amino acids that is at least 90%, at least 95%, at least 96%, at least 97%, at least 98%, at least 99%, or 100% identical to SEQ ID NO: 33.
23. The engineered stem cell of any of embodiments 1-8 and 13-22, wherein the synthetic gamma chain polypeptide has the sequence of amino acids set forth in SEQ ID O: 28 and the synthetic beta chain polypeptide has the sequence of amino acids set forth in SEQ ID NO: 33.
24. The engineered stem cell of any one of embodiments 1 to 15, wherein the first dimerization domain and the second dimerization domain are homodimerization domains selected from:
   i) FK506-Binding Protein of size 12 kD (FKBP);
   ii) cyclophiliA (CypA); or
   iii) gyrase B (CyrB);
and the non-physiological ligand is, respectively:
   i) FK1012, AP1510, AP1903, or AP20187 or an analog thereof;
   ii) cyclosporin-A (CsA) or an analog thereof; or
   iii) coumermycin or an analog thereof.
25. The engineered stem cell of any of embodiments 1-24, wherein the stem cell is a pluripotent stem cell.
26. The engineered stem cell of any one of embodiments 1 to 25, wherein the stem cells are induced pluripotent stem cells (iPSCs).
27. The engineered stem cell of any of embodiments 1-26, wherein the stem cell is resistant to rapamycin-mediated mTOR inhibition.
28. The engineered stem cell of any one of embodiments 1 to 27, wherein the stem cells express a cytosolic polypeptide that binds to the non-physiological ligand.
29. The engineered stem cell of any one of embodiments 1 to 28, wherein the non-physiological ligand is rapamycin or a rapalog, and the stem cells express a cytosolic FRB domain or variant thereof.
30. The engineered stem cell of embodiment 29, wherein the cytosolic FRB domain comprises a polypeptide sequence at least 95% identical to SEQ ID NO: 6 or SEQ ID NO: 7.
31. The engineered stem cell of embodiment 29, wherein the cytosolic FRB domain comprises a polypeptide sequence at least 98% identical to SEQ ID NO: 6 or SEQ ID NO: 7.
32. The engineered stem cell of any of embodiments 1-31, wherein the stem cell comprises a disrupted FKBP12 gene that reduces expression of FKBP12.
33. The engineered stem cell of any of embodiments 1-32, wherein the stem cell comprises knock out of the FKBP12 gene.
34. The engineered stem cell of any one of embodiments 1 to 33, wherein the stem cells comprise a nucleotide sequence encoding the synthetic cytokine receptor inserted into the genome of the stem cell.
35. The engineered stem cell of embodiment 34, wherein the nucleotide sequence encoding the synthetic cytokine receptor is inserted into a non-target locus in the genome of the stem cell.
36. The stem cell of embodiment 34, wherein the nucleotide sequence encoding the synthetic cytokine receptor is inserted into an endogenous gene of the stem cell.
37. The stem cell of embodiment 36, wherein the insertion reduces expression of the endogenous gene in the locus.
38. The stem cell of embodiment 36 or embodiment 37, wherein the insertion knocks out the endogenous gene in the locus.
39. The engineered stem cell of any of embodiments 36-38 wherein the insertion is by homology-directed repair.
40. The engineered stem cell of any of embodiments 36-39, wherein the endogenous gene is a housekeeping gene, a blood-lineage specific loci or an immune-related gene.
41. The engineered stem cell of embodiment 40, wherein endogenous gene is a housekeeping gene and the housekeeping gene is selected from eukaryotic translation elongation factor 1 alpha (EEF1A), glylceraldehyde-3-phosphate dehydrogenase (GAPDH), ubiquitin C (UBC), and actin beta (ACTB).
42. The engineered stem cell of embodiment 40, wherein the endogenous gene is a blood-lineage specific loci and the blood-lineage specific loci is selected from protein tyrosine phosphatase receptor type C (PTPRC), IL2RG, and IL2RB.
43. The engineered stem cell of embodiment 40, wherein the immune-related gene is selected from a beta-2-microglobulin (B2M) gene, a T cell receptor alpha constant (TRAC) gene, and a signal regulatory protein alpha (SIRPA) gene.
44. The engineered stem cell of any one of embodiments 1-43, wherein the stem cells comprise a B2M knockout.
45. The engineered stem cell of any of embodiments 1-44, wherein the stem cells comprise a B2M knockout and a FKBP12 knockout.
46. The engineered stem cell of any one of embodiments 1-45, comprising a chimeric antigen receptor (CAR).
47. The engineered stem cell of embodiment 46, wherein the CAR is an anti-FITC CAR.
48. The engineered stem cell of any of embodiments 1-47, wherein binding of the non-physiological ligand to the synthetic cytokine receptor activates the synthetic cytokine receptor in the stem cells to induce differentiation of the engineered stem cells in the cell population.
49. A cell population comprising engineered stem cells of any of embodiments 1-48.
50. A method of genetically engineering stem cells to express a synthetic cytokine receptor, comprising:
contacting a population of stem cells with (i) a guide RNA (gRNA) targeting a target site in an endogenous gene, (ii) an RNA-guided endonuclease, and (iii) a recombinant vector comprising a nucleotide sequence encoding a synthetic cytokine receptor for a non-physiological ligand,
thereby inserting the nucleotide sequence into the endogenous gene;
wherein the cytokine receptor comprises:
a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.
51. The method of embodiment 50, wherein the nucleotide sequence is inserted via homology directed repair (HDR).
52. The method of embodiment 51, wherein the vector comprises a nucleic acid comprising from 5′ to 3′ (a) a nucleotide sequence homologous with a region located upstream of the target site. (b) the nucleotide sequence encoding a synthetic cytokine receptor for a non-physiological ligand, and (c) a nucleotide sequence homologous with a region located downstream.
53. The method of embodiment 50, wherein the nucleotide sequence is inserted via non-homologous end joining (NHEJ).
54. The method of any one of embodiments 50-53, wherein the RNA-guided endonuclease is selected from a Cas endonuclease, a Mad endonuclease, and a Cpf1 endonuclease.
55. The method of any one of embodiments 50-54, wherein the RNA-guided endonuclease is Cas9 or Mad7.
56. The method of any of embodiments 50-55, wherein the endogenous gene is selected from B2M, TRAC and SIRPA.
57. The method of any of embodiments 50-56, wherein the endogenous gene is B2M.
58. The method of any of embodiments 50-57, wherein the gRNA comprises the sequence set forth in SEQ ID NO: 18.
59. The method of any of embodiments 52-58, wherein the nucleotide sequence homologous with a region located upstream of the target site comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 22; and the nucleotide sequence homologous with a region located downstream comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 23.
60. The method of any of embodiments 50-59, wherein the nucleotide sequence encoding the synthetic cytokine receptor comprises a first nucleic acid sequence encoding a gamma chain that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 37, and a second nucleic acid sequence encoding a beta chain that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 38.
61. The method of embodiment 60, wherein the first nucleic acid sequence and second nucleic acid sequence are separated by a cleavable linker or an IRES.
62. The method of embodiment 61, wherein the cleavable linker is a protein quantitation reporter linker (PQR), optionally set forth in SEQ ID NO:42.
63. The method of any of embodiments 50-62, wherein the nucleotide sequence encoding a synthetic cytokine receptor for a non-physiological ligand is under the operable control of a heterologous promoter.
64. The method of embodiment 63, wherein the heterologous promoter is the EF1α promoter or the MND promoter.
65. The method of any of embodiments 50-64, wherein the nucleotide sequence encoding the synthetic cytokine receptor comprises a polyadenylation sequence.
66. The method of any of embodiments 50-65, wherein the recombinant vector comprises the sequence set forth in SEQ ID NO:40 or a sequence that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 40.
67. The method of any one of embodiments 50-66, comprising engineering the population of stem cells to be resistant to rapamycin-mediated mTOR inhibition.
68. The method of embodiment 67, wherein engineering the population of stem cells to be resistant to rapamycin comprises knocking out a FKBP12 gene.
69. The method of embodiment 68, wherein the method further comprises contacting the population of stem cells with a guide RNA (gRNA) targeting a target site in the FKBP12 gene.
70. The method of embodiment 69, wherein the gRNA comprises one or more gRNA selected from a gRNA comprising the sequence set forth in SEQ ID NO: 19, SEQ ID NO: 20 or SEQ ID NO:21.
71. The method of embodiment 70, wherein the one or more gRNA is a pool of gRNA comprising 2 or 3 gRNA.
72. The method of any of embodiments 50-71, further comprising introducing into the population of stem cells a chimeric antigen receptor (CAR).
73. The method of embodiment 72, wherein the CAR is an anti-FITC CAR.
74. The method of any of embodiments 50-73, wherein the stem cells are pluripotent stem cells.
75. The method of any one of embodiments 50-74, wherein the stem cells are iPSCs.
76. A cell population produced by the method of any one of embodiments 50-75.
77. A pharmaceutical composition comprising the cell population of embodiment 49 or embodiment 76.
78. A method for generating hematopoietic progenitor (HP) cells, the method comprising:
a) culturing a cell population comprising engineered iPSCs of any of embodiments 1-48 under conditions to form an aggregate;
b) culturing the cells produced in a) under conditions to induce mesoderm formation in a plurality of the cells, wherein the initiation of the culturing in b) is day 0; and
c) culturing the cells produced in b) under conditions to differentiate cells into a population of hematopoietic progenitors (HP),
wherein at least a portion of one or more of steps a)-c) are carried out in the presence of a non-physiological ligand of the synthetic cytokine receptor.
79. A method for generating cytotoxic innate lymphoid (iCIL) cells, the method comprising culturing a cell population comprising engineered iPSCs of any of embodiments 1-48 under conditions to differentiate the iPSCs to cytotoxic innate lymphoid (iCILs), wherein a non-physiological ligand of the synthetic cytokine receptor is added during at least a portion of the culturing.
80. The method of embodiment 79, wherein the culturing comprises:
a) culturing the cell population comprising engineered iPSCs under conditions to form an aggregate;
b) culturing the cells produced in a) under conditions to induce mesoderm formation in a plurality of the cells, wherein the initiation of the culturing in b) is on day 0;
c) culturing the cells produced in b) under conditions to differentiate cells into a population of hematopoietic progenitors (HP); and
d) culturing the cells produced in c) under conditions to generate iCIL cells,
wherein at least a portion of one or more of steps a)-d) are carried out in the presence of the non-physiological ligand of the synthetic cytokine receptor.
81. A method for generating cytotoxic innate lymphoid (iCIL) cells, the method comprising:
a) culturing a cell population comprising engineered iPSCs of any of embodiments 1-48 under conditions to form an aggregate;
b) culturing the cells produced in a) under conditions to induce mesoderm formation in a plurality of the cells, wherein the initiation of the culturing in b) is day 0;
c) culturing the cells produced in b) under conditions to differentiate cells into a population of hematopoietic progenitors (HP); and
d) culturing the cells produced in c) under conditions to generate iCIL cells,
wherein at least a portion of one or more of steps a)-d) are carried out in the presence of a non-physiological ligand of the synthetic cytokine receptor.
82. The method of any of embodiments 78-81, wherein the culturing is carried out in a vessel treated to promote cell adhesion and growth.
83. The method of embodiment 82, wherein the vessel is a Matrigel.
84. The method of any of embodiments 78-81, wherein the culturing is carried out in a non-adherent culture vessel.
85. The method of embodiment 84, wherein the non-adherent culture vessel is Aggrewell™ plate.
86. The method of any of embodiments 78 and 80-85, wherein the aggregate in a) is an Embryoid body (EB).
87. The method of any of embodiments 78-81, wherein the culturing is carried out in suspension.
88. The method of any of embodiments 78-81 and 86, wherein the culturing is carried out in culture vessel that is not treated to promote cell adhesion and proliferation.
89. The method of embodiment 86 or embodiment 87, wherein step a) comprises:
(i) performing a first incubation comprising culturing the cell population of engineered stem cells under conditions to form a first aggregate;
(ii) contacting the aggregate with a dissociating agent to form a population of dissociated cells; and
(iii) performing a second incubation comprising culturing the population of dissociated cells under conditions to form the second aggregate.
90. The method of any of embodiments 78-89, wherein the culturing in b) is in a media comprising one or more of BMP4, FGF2, VEGF and a Rock Inhibitor, optionally wherein the Rock Inhibitor is Y27632.
91. The method of any of embodiments 78-90, wherein the culturing in b) is in a media comprising BMP4, FGF2, VEGF and Y27632.
92. The method of any of embodiments 78-90, wherein the culturing in b) is in a media comprising BMP4, FGF2 and VEGF.
93. The method of any of embodiments 78-92, wherein the culturing in b) is in a media comprising the non-physiological ligand.
94. The method of any of embodiments 78-89 and 93, wherein the culturing in b) is in a media comprising the non-physiological ligand without any additional growth factors.
95. The method of any of embodiments 78-94, wherein the culturing in b) is for 2 to 4 days, optionally for at or about 3 days
96. The method of any of embodiments 78-95, wherein the culturing in c) is in a media comprising one or more of BMP4, FGF2, VEGF. TPO, SCF, and LDL.
97. The method of any of embodiments 78-95, wherein the culturing in c) is in a media comprising one or more of BMP4, FGF2, VEGF and LDL.
98. The method of any of embodiments 78-97, wherein the culturing in c) is in a media without SCF and TPO.
99. The method of any of embodiments 78-98, wherein the culturing in c) is in a media comprising the non-physiological ligand.
100. The method of any of embodiments 78-94 and 97, wherein the culturing in c) is in a media comprising the non-physiological ligand without any additional growth factors, cytokines or both.
101. The method of any of embodiments 78-98, wherein the culturing in c) is on days 3 to 15 days.
102. The method of any of embodiments 78-101, wherein during at least a portion of the culturing in c) the media comprises an aryl hydrocarbon receptor (AHR) antagonist (e.g. StemRegenin-1), a pyrimido-[4,5-b]-indole derivative (e.g. UM729) or both.
103. The method of embodiment 102, wherein the portion of the culturing is on or about days 9-15.
104. The method of any of embodiments 80-103, wherein the culturing in d) is in a media comprising one or more of FLT3L, IL-7, IL-12, IL-15, SR-1 and UM729.
105. The method of any of embodiments 80-104, wherein the culturing in d) is in a media comprising the non-physiological ligand.
106. The method of any of embodiments 80-103 and 105, wherein the culturing in d) is in a media comprising the non-physiological ligand without any additional growth factors, cytokines or both.
107. The method of any of embodiments 80-106, wherein the culturing in d) is for a time between days 15 and 40.
108. The method of any of embodiments 80-106, wherein the culturing in d) is for days 15 and 30.
109. A method for generating cytotoxic innate lymphoid (iCIL) cells, comprising contacting a cell population comprising an engineered stem cell of any one of embodiments 1-48 with the non-physiological ligand for a first period of time sufficient to generate CLPs, and contacting the CLPs with a differentiation media for a second period of time sufficient to generate iCILs.
110. The method of embodiment 109, wherein the differentiation media comprises stem cell factor (SCF), FLT3L, IL-7, IL-12, IL-15, SR-1 and UM729.
111. The method of embodiment 110, wherein the differentiation media comprises the non-physiological ligand.
112. The method of any one of embodiments 109-111, wherein the first period of time is 1-15 days, and wherein the second period of time is 1-15 days.
113. The method of any one of embodiments 109-112, comprising contacting the iCILs with a pre-activation media comprising IL-7, IL-12, IL-15, IL-18 and IL-21 for a third period of time sufficient to generate mature iCILs.
114. The method of embodiment 113, wherein the pre-activation media comprises the non-physiological ligand.
115. The method of embodiment 113 or 114, wherein the third period of time is 1-10 days.
116. The method of embodiment 113 or embodiment 114, wherein mature iCILs express NKp46, NKG2D, LFA1, DNAM1, CD16 and CD56.
117. The method of any of embodiments 78-116, wherein the non-physiological ligand is rapamycin or a rapamycin analog.
118. The method of embodiment 117, wherein the rapamycin analog is rapalog.
119. The method of any of embodiments 78-118, wherein the non-physiological ligand is added to the media at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM. 10 nM and 50 nM, 10 nM and 20 nM, 20 nM and 200 nM. 20 nM and 150 nM 20 nM and 100 nM, 20 nM and 50 nM, 50 nM and 200 nM, 50 nM and 150 nM, 50 nM and 100 nM. 100 nM and 200 nM, 100 nM and 150 nM and 150 nM and 200 nM.
120. The method of any of embodiments 78-119, wherein the non-physiological ligand is added to the media at a concentration of at or about 10 nM.
121. The method of any of embodiments 78-119, wherein the non-physiological ligand is added to the media at a concentration of at or about 100 nM.
122. A hematopoietic progenitor (HP) cell produced by the method of any of embodiments 78 and 82-103.
123. The HP cell of embodiment 122, wherein the HP cells comprise lower expression of HLF, HOXA9, and/or CD133 compared to a CD34+ cord blood cell.
124. The HP cell of embodiment 123, wherein the expression of HLF, HOXA9, and/or CD133 in HP cells is 8-fold, 7-fold, 6-fold, 5-fold, 4-fold, 3-fold, 2-fold, or 1-fold lower compared to a CD34+ cord blood cell.
125. The HP cell of embodiment 123 or embodiment 124, wherein the CD34+ cord blood cell comprises a hematopoietic stem cell (HSC).
126. A cytotoxic innate lymphoid (iCIL) cell produced by the method of any of embodiments 79-116.
127. A hematopoietic progenitor (HP) cell that has been differentiated from a pluripotent stem cell of any of embodiments 1-48, wherein the HP comprises a synthetic cytokine receptor.
128. A cytotoxic innate lymphoid (iCIL) that has been differentiated from a pluripotent stem cell of any of embodiments 1-48, wherein the iCIL comprises a synthetic cytokine receptor.
129. A population of hematopoietic progenitor (HP) cells produced by the method of any of embodiments 78 and 82-103.
130. The population of embodiment 129, wherein the population of HP cells comprise lower expression of HLF, HOXA9, and/or CD133 compared to a population of CD34+ cord blood cells.
131. The population of embodiment 130, wherein the expression of HLF, HOXA9, and/or CD133 in HP cells is 8-fold, 7-fold, 6-fold, 5-fold, 4-fold, 3-fold, 2-fold, or 1-fold lower compared to a population of CD34+ cord blood cells.
132. The population of embodiment 130 or embodiment 131, wherein the population of CD34+ cord blood cell comprises a hematopoietic stem cell (HSC).
133. A population of cytotoxic innate lymphoid (iCIL) cells produced by the method of any of embodiments 79-121.
134. A population of cells comprising the iCILs of embodiment 126 or embodiment 128.
135. The iCIL of embodiment 126 or embodiment 128 or the population of iCIL of embodiment 123 or embodiment 120, wherein the iCIL comprise a B2M knockout.
136. The iCIL of embodiment 126 or embodiment 128 or the population of iCIL of embodiment 133 or embodiment 134, wherein the iCIL comprise a B2M knockout and a FKBP12 knockout.
137. A pharmaceutical composition comprising the iCIL or population of iCILs of any of embodiments 126-136.
138. A method of expanding a cytotoxic innate lymphoid cell (iCIL), the method comprising contacting an iCIL or population of iCILs of any of embodiments 126-136 or the pharmaceutical composition of embodiment 137 with the non-physiological ligand of the synthetic cytokine receptor.
139. A method of killing or inhibiting the proliferation of cancer cells, comprising contacting cancer cells with the iCIL or population of iCILs of any of embodiments 126-136, or the pharmaceutical composition of embodiment 137 with the non-physiological ligand of the synthetic cytokine receptor.
140. The method of embodiment 138 or embodiment 139 that is performed in vitro or ex vivo.
141. The method of any of embodiments 138-140, wherein the non-physiological ligand is rapamycin or a rapamycin analog.
142. The method of embodiment 141, wherein the rapamycin analog is rapalog.
143. The method of any of embodiments 138-142, wherein the non-physiological ligand is contacted at a concentration of between 5 nM and 200 nM, 5 nM and 150 nM. 5 nM and 100 nM. 5 nM and 50 nM. 5 nM and 20 nM, 5 nM and 10 nM, 10 nM and 200 nM, 10 nM and 150 nM. 10 nM and 100 nM. 10 nM and 50 nM, 10 nM and 20 nM, 20 nM and 200 nM, 20 nM and 150 nM 20 nM and 100 nM. 20 nM and 50 nM, 50 nM and 200 nM, 50 nM and 150 nM, 50 nM and 100 nM, 100 nM and 200 nM, 100 nM and 150 nM and 150 nM and 200 nM.
144. The method of any of embodiments 138-143, wherein the non-physiological ligand is contacted at a concentration of at or about 10 nM.
145. The method of any of embodiments 138-143, wherein the non-physiological ligand is contacted at a concentration of at or about 100 nM.
146. The method of any of embodiments 138, 139 and 141-145, wherein the method is performed in vivo in a subject and the non-physiological ligand is administered to the subject.
147. A method of treating a cancer in a subject, comprising administering to the subject an effective amount of the cell population of any one of embodiments 1-48 and 76, or the pharmaceutical composition of embodiment 77.
148. A method of treating a cancer in a subject, comprising administering to the subject an effective amount of the iCIL or population of iCILs of any of embodiments 126-136, or the pharmaceutical composition of embodiment 137.
149. The method of any of embodiments 146-148, wherein the subject has not been administered a lymphodepleting therapy prior to the administering the iCIL, population of iCILs or the pharmaceutical composition.
150. The method of any of embodiments 138-149, wherein the iCIL express a CAR targeting cancer cells in the subject.
151. The method of embodiment 150, wherein the CAR is an anti-FITC CAR and the subject has been administered a FITC-ligand to tag a cancer cell in the subject, wherein the ligand specifically binds a molecule expressed on a tumor.
152. The method of embodiment 151, wherein the FITC-ligand is FITC-folate.
153. The method of any of embodiments 146-152, comprising administering to the subject the non-physiological ligand of the synthetic cytokine receptor.
154. The method of any of embodiments 146-153, wherein the non-physiological ligand is rapamycin or a rapamycin analog.
155. The method of embodiment 154, wherein the rapamycin analog is rapalog.
156. The method of any of embodiments 146-155, wherein the non-physiological ligand is administered at a dose of 1 mg to 100 mg, optionally between 10-100 mg, optionally at or about 10 mg, 20 mg, 25 mg, 30 mg, 40 mg, 50 mg or any value between any of the foregoing.
157. The method of any of embodiments 146-156, wherein multiple doses of the non-physiological ligand are administered to the subject.
158. The method of embodiment 157, wherein the multiple doses are administered intermittently or at regular intervals after administration of the iCIL population or composition thereof to the subject, optionally for a predetermined period of time.
159. The method of embodiment 146-158, wherein 2 to 8 doses of the non-physiological ligand are administered to the subject.
160. The method of any of embodiments 146-156, wherein a single dose of the non-physiological ligand is administered to the subject.
161. The method of any of embodiments 147-160, wherein the iCIL population or composition thereof is administered at a dose that is from at or about 1×108 iCIL cells to at or about 100×109 iCIL cells.
162. The method of any of embodiments 147-161, wherein the iCIL population or composition thereof is administered at a dose that is greater than at or about 5×109 iCIL cells, optionally wherein the dose is from at or about 5×109 iCIL cells to at or about 100×109 iCIL cells.
163. The method of any of embodiments 146-162, wherein multiple doses of the iCIL cells are administered to the subject.
164. The method of embodiment 163, wherein the multiple doses of iCIL cells are administered intermittently or at regular intervals, optionally for a predetermined period of time.
165. The method of embodiment 146-164, wherein 2 to 8 doses of the iCIL cells are administered to the subject.
166. The method of any of embodiments 146-162, wherein a single dose of the iCIL cells is administered to the subject.
167. A kit comprising the engineered stem cells of any one of embodiments 1-48 and 76 and instructions for differentiating the cell population to cytotoxic innate lymphoid cells.
168. A kit comprising the iCIL or the population of iCIL of 126-136 or the pharmaceutical composition of embodiment 137 and instructions for administering to a subject in need thereof.
169. The kit of embodiment 167 or embodiment 168, further comprising a container comprising the non-physiological ligand and instructions for administering the non-physiological ligand to the subject after administration of the cell population.
170. The kit of any of embodiments 167-169, wherein the subject has a cancer.

### VII. EXAMPLES

The following examples are put forth so as to provide those of ordinary skill in the art with a description of how the compositions and methods described herein may be used, made, and evaluated, and are intended to be purely illustrative of the invention and are not intended to limit the scope of what is regarding as the invention.

### Example 1: RACR-Expansion of Highly Functional CD19CAR Blood-Derived NK Cells

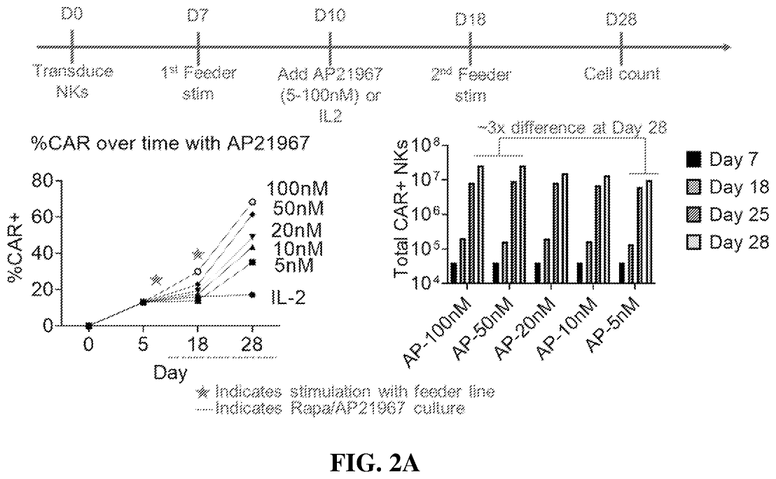

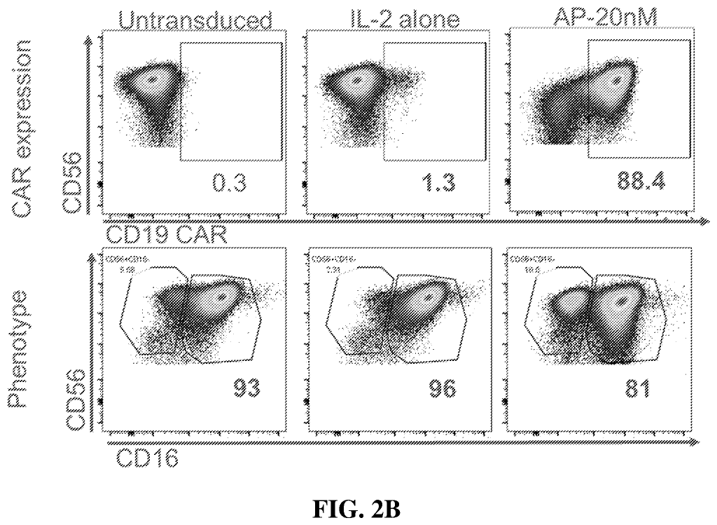

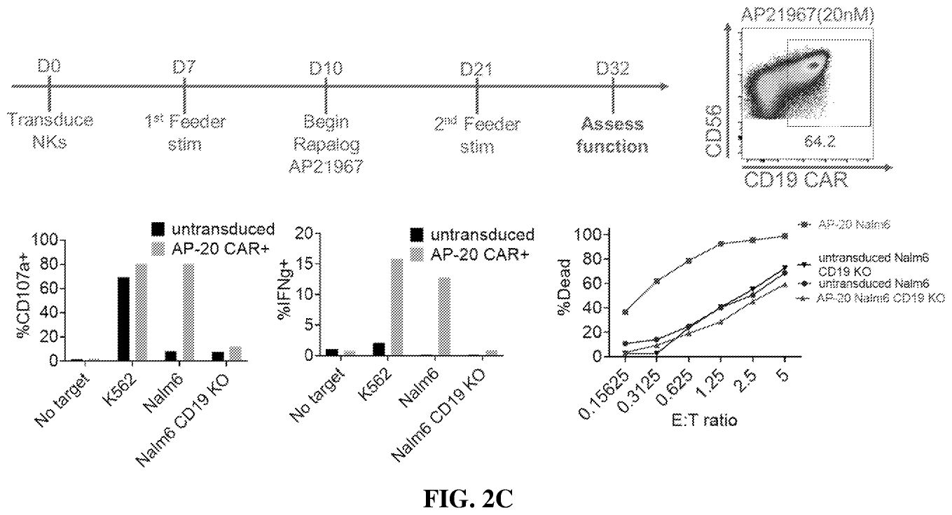

This example demonstrates RACR function in fully differentiated blood-derived NK cells (bdNK). This example also demonstrates that the RACR signal can drive NK cell expansion. A timeline of the experiment is shown in FIG. 2A. On Day −2, NK cells were isolated from peripheral blood mononuclear cells (PBMCs) using the Biolegend® human NK isolation kit and incubated in RPMI media containing 10% FBS, Glutamax, Pen-strep, IL-2 (1000 U/ml), IL-21 (20 ng/ml), and Rosuvastatin® (5 M) for two days. On Day 0, the cells were washed in RPMI media and transduced with CD19-RACR/VSVG lentiviral vector at an MOI of 30 in the presence of IL-2 (1000 U/ml), IL-21 (20 ng/ml), BX795 (6 μM), and protamine sulfate (8 μg/ml). Three days later, the media was replaced with fresh RPMI media containing IL-2 (1000 U/ml) and IL-21 (20 ng/ml). On Day 5, cells were assessed for CD19 CAR expression by flow cytometry. On Day 7, cells were counted using the TC-20 cell counter (Biorad®) and stimulated with irradiated (10,000 rads) K562 cells expressing membrane-bound IL-21 and 41BBL at a ratio of ˜10 feeder K562 cells:1 NK cell. On Day 10, the cells were washed and resuspended in fresh RPMI media containing either IL-2 (1000 U/ml) or varying concentrations of AP21967 (5-100 nM) (A/C Heterodimerizer-Takara®). On Day 18, cells were counted and CAR expression was assessed by flow cytometry and the cells were stimulated again with K562 feeder cells at a ratio of 10 feeder cells:1 NK cell in RPMI media containing either IL-2 or AP21967 using the same concentrations as on Day 10. The cells were cultured for an additional 10 days, counted, and CAR expression was analyzed using flow cytometry. As seen in FIG. 2A, percent CAR expression over time for each group is shown in the left panel and total CAR+ NK cells over time are shown in the right panel. RACR-NK cells showed dose-responsive AP21967-driven feeder-based expansion resulting in high purity and high yield of RACR-NK cells.

As seen in FIG. 2B, immunophenotyping of IL-2 vs. RACR-expanded bdNK cells was performed to determine the activity of NK cells after RACR-expansion. bdNK cells were isolated from PBMCs, transduced and expanded in the presence of K562 feeder cells and either IL-2 or AP21967 using a similar protocol as described above. 39 days after transduction, CAR expression was analyzed by flow cytometry. CAR expression is shown on the top panels for NK cells expanded in the presence of either IL-2 (1000 U/ml) or AP21967 (20 nM). CD56 and CD16 expression on the CAR+ cells is shown on the bottom panels. RACR-Expanded NK cells showed a CD56 high phenotype similar to IL-2 expanded cells, with a slight decrease in the CD16 high population (81% vs. 96%) (FIG. 2B).

Comparing the functionality of IL-2 vs. RACR-expanded bdNK cells was determined by their ability to recognize and target tumor cells (FIG. 2C). NK cells naturally target tumors via innate stress ligands that are overexpressed on transformed cancer cells. Thus, innate- as well as CAR-mediated killing of tumor cells by CD19-CAR-RACR-expanded bdNK cells (AP-20) was measured. NK cells were isolated from PBMCs, transduced and expanded in the presence of K562 feeder cells and AP21967 (20 nM) using a similar protocol as described above with slight alterations in timing of AP21967 and K562 feeder cell addition (FIG. 2C—top panel experimental timeline). CAR expression is shown on the upper right panel at Day 31 after transduction. On Day 32, CAR+ NK cell function was assessed using intracellular cytokine production after tumor cell stimulation. 1×10⁵ NK cells were incubated with 1×10⁵ K562 cells, Nalm6 cells, or Nalm6 CD19 knock out cells in the presence of brefeldin A, monensin, and anti-CD107 for 5 hours in 100 μl RPMI media in a 96 well plate. CAR+ NK cells were gated on viable, CD3−, CD56+, and 2A+ cells. Shown are levels of CD107a (FIG. 2C bottom left panel) and IFNγ (FIG. 2C bottom middle panel) expression on AP21967-expanded CAR+ cells (AP-20) or untransduced controls. AP-20 cells released CD107a in response to innate receptor stimulation from highly reactive K562 tumor line cells similar to IL-2 expanded NK cell controls. Nalm6 cells induced lower CD107a secretion compared to K562 cells due to the lower level of innate stimulating ligands present. Nalm6 cells were then used to test the function of the CD19CAR targeting. Nalm6 cells expressing CD19 induced high levels of CD107a secretion from CD19-CAR-RACR-expanded bdNK cells (AP-20) compared to Nalm6 CD19 KO controls indicating robust CD19-CAR function in AP-20 NK cells. IFNγ release was also increased in response to CD19-Nalm6 cells compared to CD19 KO controls, further demonstrating CAR activity of AP-20 expanded NK cells.

Direct clearance of tumor cells via both innate receptor and CAR receptor targeting was measured. AP-20 CAR+ NK cell function was assessed using a flow cytometry-based killing assay. Target Nalm6 or Nalm6-CD19 knock out cells were labeled with Cell Trace Violet for 20 min at 37° C. at 0.5 mM in PBS and then washed. 5×10⁴ target cells were added to a 96 well plate and NK effector cells were added at varying Effector cell: Target cell (E:T) ratios in a total volume of 100 μl. After 5 hours 5×10⁴ counting beads were added to each well as a normalization factor. The cell and bead mixture was washed and viable, CD56−, Cell Trace Violet+ cells were analyzed. % Dead was calculated by comparing the total number of Viable, Cell trace violet+ cells in each well to a non-effector control well (FIG. 2C bottom right panel). AP-20 bdNK cells were able to recognize and kill Nalm6 cells via innate receptor targeting similar to IL-2 expanded untransduced bdNK cells showing no loss in cytotoxicity or function. Increased killing of CD19-expressing Nalm6 cells was observed with AP-20 cells compared to untransduced controls or CD19 knock out cells indicating synergistic increases in targeting of tumors via both innate and CAR-mediated killing.

### Example 2: Generation of iPSC-Derived CIL Cells

iPSC-derived CIL cells (iCIL cells) were generated using a synthetic cytokine receptor, an IL-2-type rapamycin-activated cell cytokine receptor (RACR2), to drive expansion of these cells with an exogenous cytokine. A diagram of the process can be found in FIG. 1A and FIG. 1B.

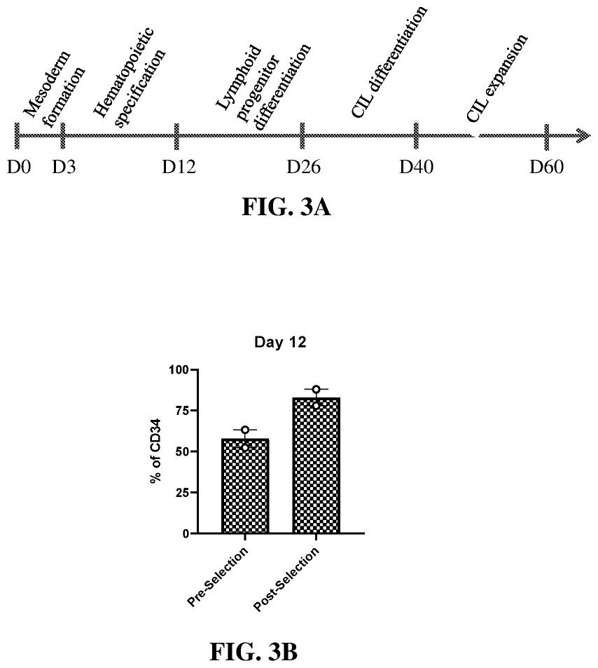

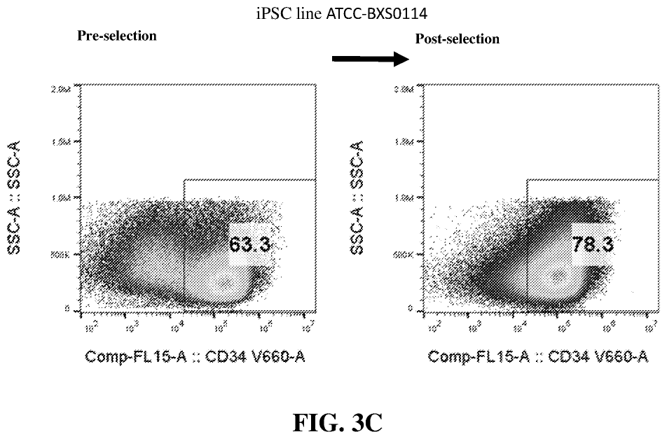

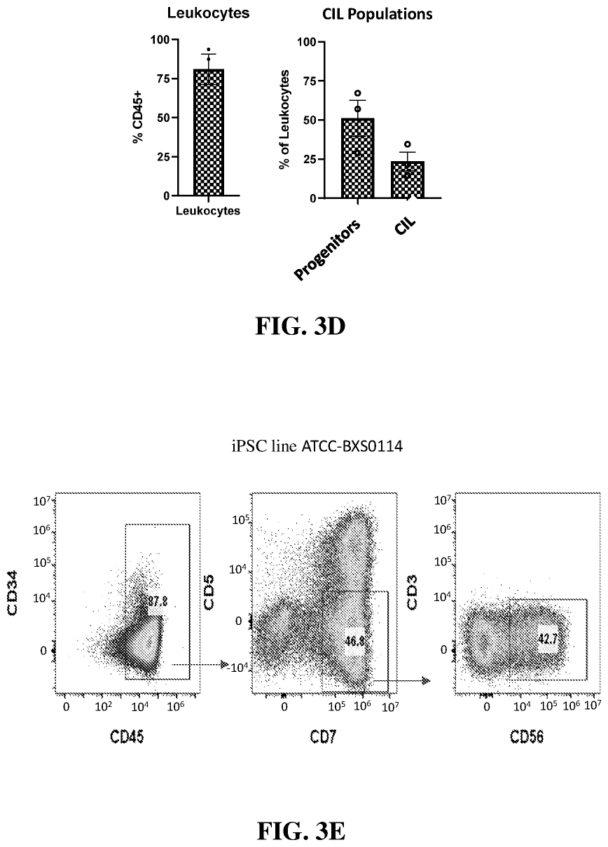

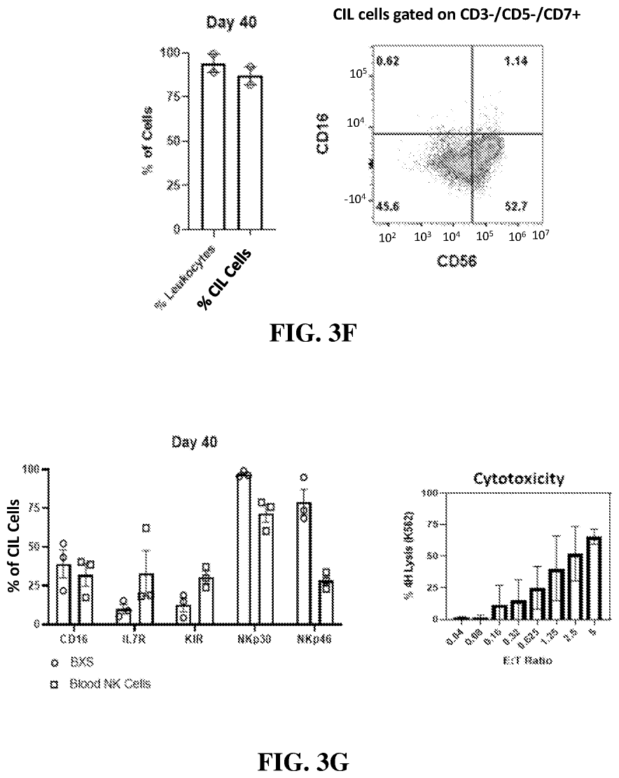

Undifferentiated human iPSCs were cultured in mTeSR Plus media (STEMCELL® Technologies) on hESC-qualified Matrigel (Corning®) and passaged using EDTA (Thermo Fisher Scientific®). iPSCs were differentiated into CIL cells using the STEMdiff NK Cell Kit (STEMCELL® Technologies) following the manufacturer's instructions. Briefly, at Day 0 a single-cell suspension of undifferentiated iPSCs was seeded into microwells (500 cells/microwell) to generate embryoid bodies (EBs) in STEMdiff Hematopoietic-EB Basal Medium supplemented with EB Supplement A (Days 0-3) and then EB Supplement B (Days 3-12). At Day 5, EBs were harvested from microwells and re-seeded onto non-tissue culture-treated plates. At Day 12, EBs were harvested and dissociated into a single-cell suspension, CD34+ cells selected using an EasySep Human CD34 Positive Selection Kit II (STEMCELL® Technologies) and analyzed using flow cytometry. An example selection (FIG. 3B) and flow cytometry analysis (FIG. 3C) are shown using a representative iPSC line, ATCC-BXS0114 (ACS-1028) (ATCC). The CD34+ selection process significantly enriched the cultures for CD34+ cells. Selected cells were re-seeded onto non-tissue culture-treated plates coated with StemSpan® Lymphoid Progenitor Differentiation Coating and cultured with StemSpan® SFEM II media supplemented with StemSpan® Lymphoid Progenitor Expansion Supplement (Days 12-26). Between Days 15-19, cells were harvested and re-seeded onto a newly coated plate. At Day 26, lymphoid progenitor cells were harvested and analyzed using flow cytometry (FIG. 3D and FIG. 3E). Cells at this timepoint were found to primarily be leukocytes (CD45+), and this population contained smaller populations of both progenitors (CD45+/CD5−/CD7+) as well as CIL cells (CD45+/CD5−/CD7+/CD56+). Cells were re-seeded onto tissue culture-treated, non-coated plates and cultured with StemSpan® SFEM II media supplemented with StemSpan® Cell Differentiation Supplement and UM729 (1 μM) (Days 26-40). At Day 40, CIL cells were harvested for further studies.

As seen in FIGS. 3F and 3G, at Day 40, iCIL cells were harvested and immunophenotyped by flow cytometry using the following antibodies: CD16 BUV496 (368), CD3 BUV661 (UCHT1), CD56 BV421 (HCD56), CD45 BV510 (2D1), CD5 BV605 (L17F12), NKG2D-BV785 (1D11), NKp30-BB700 (p30-15), CD7-PE (CD7-6B7), NKp46-PECy7 (9E2), CD127-AF700 (A019D5), KIR-APCFire750 (HP-MA4). iCIL cells were collected, pelleted, and resuspended in ZombieUV, diluted in PBS, and stained for 10 min. Cells were then resuspended in BD FACs buffer containing antibodies and stained for 20 min at 4° C. Cells were then processed on a Beckman Coulter CytoFLEX LX flow cytometer. Resulting FCS files were analyzed using BD FlowJo V10.8. The percentage of CD45+ cells are plotted as % Leukocytes and CD56+ cells are plotted as % CIL cells (FIG. 3F).

The resulting CIL cells displayed a CIL purity of >90% (FIG. 3G left panel, “BXS”). The CIL cells were largely CD56bright with low CD16 expression (FIG. 3F right panel). Across many activation markers, it was found that the iCIL cells had a similar phenotype to blood derived NK cells (FIG. 3G left panel, “BXS”). The percentage of cells expressing CD16 was comparable between the iCIL and blood-derived NK populations (FIG. 3G left panel, “BXS”). However, blood-derived NK cells express higher amounts of IL-7RB, denoting a more immature and inhibitory phenotype, while Day 40 iCIL cells express higher amounts of activating receptors NKp30 and NKp46. These markers also suggest increased cytotoxic potential for the iCIL cells (FIG. 3G left panel, “BXS”).

The functionality of the iCILs was assessed through their ability to recognize and target tumor cells via recognition of innate stress ligands that are increased in transformed cells. iCIL cells were co-cultured with K562-mCherry target cells at different effector target ratios (E:T ratio) in cRPMI+IL-2 (RPMI+10% FBS+1× Glutamax+1×P/S+100 IU human IL-2) (FIG. 3G right panel). Cells were co-cultured for 4 hours at 37° C. followed by antibody-staining for CD56-BV421. % Lysis was calculated using Precision Counting Beads: #CD56− mCherry+ Target cells in CIL co-culture/#CD56-mCherry+ Target cells alone×100. iCIL cells were able to target and clear K562 tumor cells with high reproducibility across iCIL cell preparations showing robust functionality (FIG. 3G right panel).

### Example 3: Differentiation of RACR-iCIL Cells from Lymphoid Progenitor and Precursor Cells

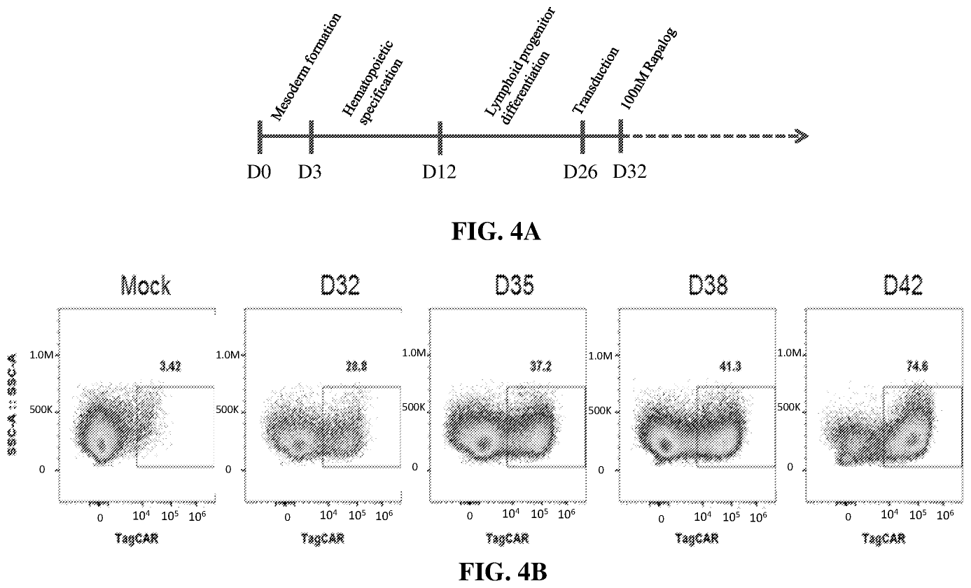

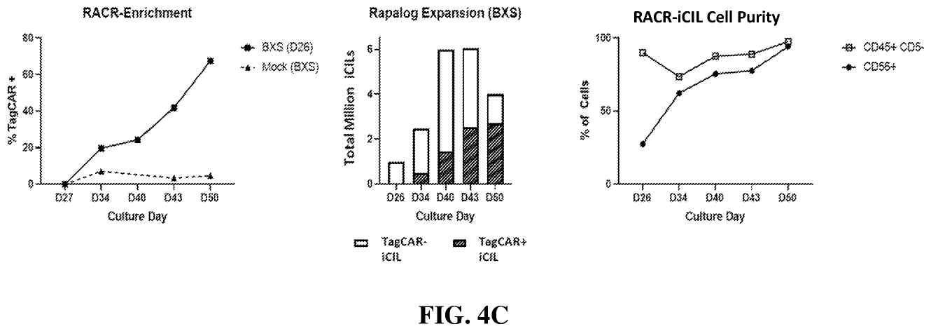

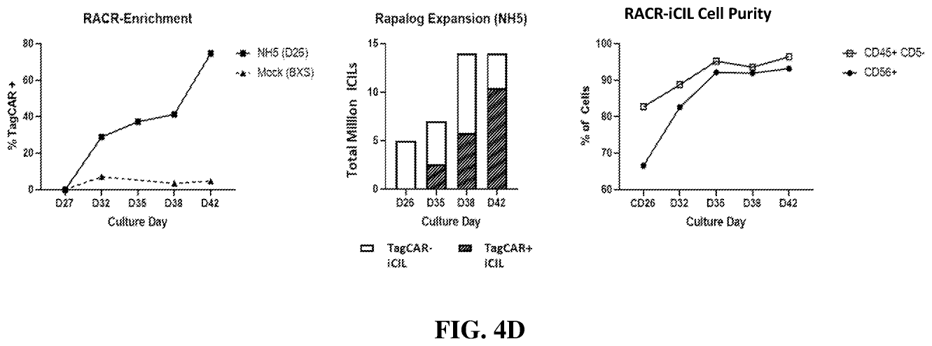

The RACR-system was utilized for differentiation to iPSC-derived CIL cells (iCIL cells). A timeline of the experiment is shown in FIG. 4A. The iPSC cell line ATCC-BXS0117 (ATCC #ACS-1031) was differentiated into lymphoid progenitors through the STEMdiff NK cell kit (STEMCELL® Technologies, #100-0170) as described in Example 2 above, up until the Day 26 timepoint. The percentage of cells containing common lymphoid progenitors (CD45+/CD5+/CD7+), CIL precursors (CD45+/CD5−/CD7+), and early CIL cells (CD45+/CD5−/CD7+/CD56+) is seen in FIG. 3D with ˜50% of the cells presenting as CIL precursors and ˜25% of total cells presenting as early CIL cells. In this study, Day 26 cells were pelleted and placed in a CIL expansion media at 2×10⁶ cells/mL containing (IL-2, IL-15, IL-21, IL-18, IL-7 in complete RPMI media: RPMI1640, 10% FBS, 1× Pen-Strep, and 1× Glutamax). Activation beads (#130-094-483) were added to cells at a 2:1 ratio and cells were placed in 37° C. incubator overnight to increase transduction of cells.

At Day 27, the cells were transduced with either VSV-G lentivirus with the payload of Tag (anti-FITC) CAR-P2A-FRB-P2A-RACR at an MOI 10 with 10 g/mL of protamine sulfate or treated with 10 μg/mL or protamine sulfate alone (Mock). The next day a 50% media change was performed, and cells were given cRPMI1640 media supplemented with IL-2, IL-7, and IL-21. On Day34, media was removed, and Mock cells were placed in cRPMI1640 media supplemented with CIL expansion media and RACR-transduced cells were then placed in cRPMI1640 media supplemented with AP21967 (A/C Heterodimerizer-Takara) at 100 nM to engage RACR-signaling. Subsequently, 50% media changes were performed weekly with 2x media. An aliquot of cells was collected weekly, counted on the Vicell Blu automated cell counter, and stained for CD16 clone 3G8, CD56 clone HCD56, CD45 clone 2D1, CD5 clone L17F12, CD7 clone CD7-6B7 and anti-FITC CAR (TagCAR) detection through FITC-AF647. In response to rapalog treatment the RACR-iCIL cells demonstrated increased purity of TagCAR-RACR iCIL cells as indicated by increased % FITC-AF647 positive, expansion of TagCAR-RACR iCIL cells as indicated by the total number of TagCAR positive cells (FIG. 4B), as well as robust iCIL cell differentiation as indicated by increased percentage of CD56 positive cells over time as shown in FIG. 4C. This data demonstrates that rapalog AP21967 and the RACR system are sufficient to drive iCIL expansion in the absence of any other cytokine signaling. A similar process was followed for an additional iPSC line (NH5 NIH iPSC cell line) with very similar results as seen in FIG. 4D.

### Example 4: RACR-iCIL Cells Generated on Day 26 Display a Similar Phenotype and Cytotoxicity to Cytokine-Generated iCIL Cells

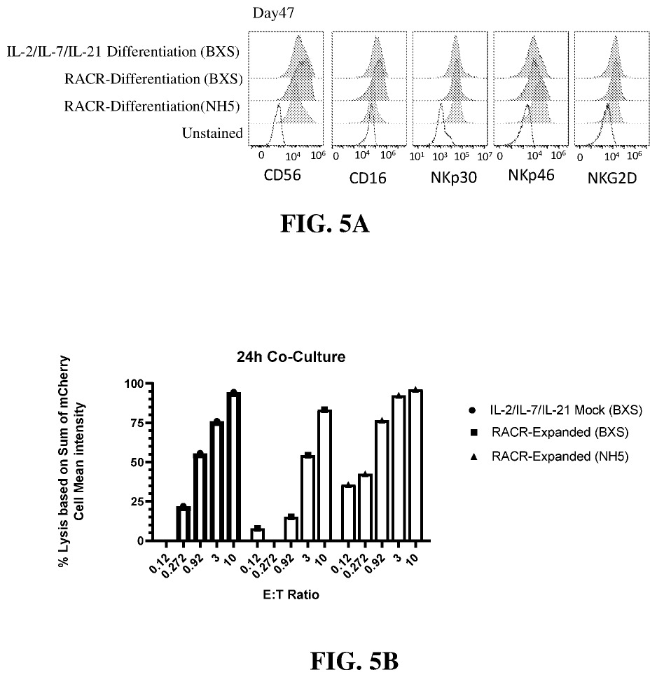

Functional assays were performed on RACR-expanded cells and stained cells for phenotypic markers CD7, CD5, CD45, CD56, NKG2D, NKp30, NKp46, and CD16 as shown in FIG. 5A. On Day 47, IL-2/IL-7/IL-21 Mock iCIL cells as well as RACR-expanded iCIL cells were collected and pelleted and resuspended in ZombieUV diluted in PBS and stained for 10 min. Cells were then resuspended in BD FACs buffer containing antibodies and stained for 20 min at 4° C. Cells were then washed in BD FACs buffer 2× and analyzed on the Cytoflex flow cytometer. RACR-expanded iCIL cells showed similar phenotype to cytokine expanded Mock cells with high expression of CD56, NKp30, NKp46, and NKG2D (FIG. 5A). iPSC line differences were observed with the NH5 cell line showing lower expression of CD16 than the BXS cells. This data demonstrates that RACR-iCIL cells have a highly activated phenotype with no decrease in activation receptors due to RACR-expansion as compared to cytokine expanded cells. The cytotoxicity of RACR-expanded cells was tested against the MDA breast tumor cell line. Mock expanded or RACR-expanded iCIL cells were mixed with MDA-mCherry cells for 24H and then the plate was washed prior to imaging on an Opera Phenix® microscope (FIG. 5B). The total mCherry signal per well was calculated using the Opera Phenix® analysis tools. The graph is normalized to no-iCIL cell wells to generate a % of cells killed. RACR-expanded cells showed comparable toxicity to Mock cytokine expanded cells further demonstrating their highly activated phenotype and function (FIG. 5B).

### Example 5: Expansion of RACR-iCIL Cells

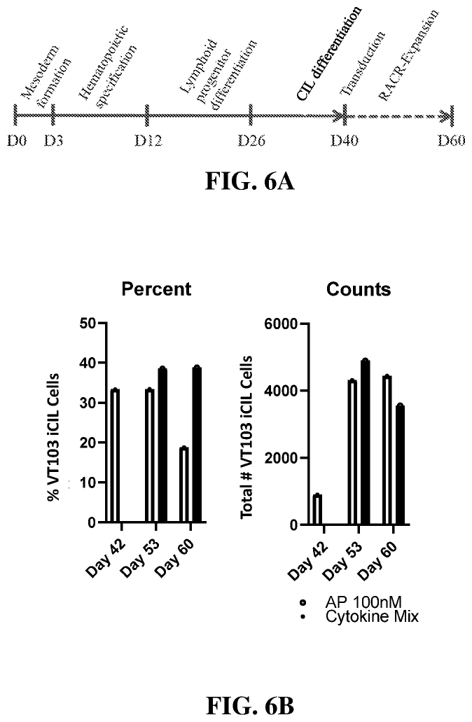

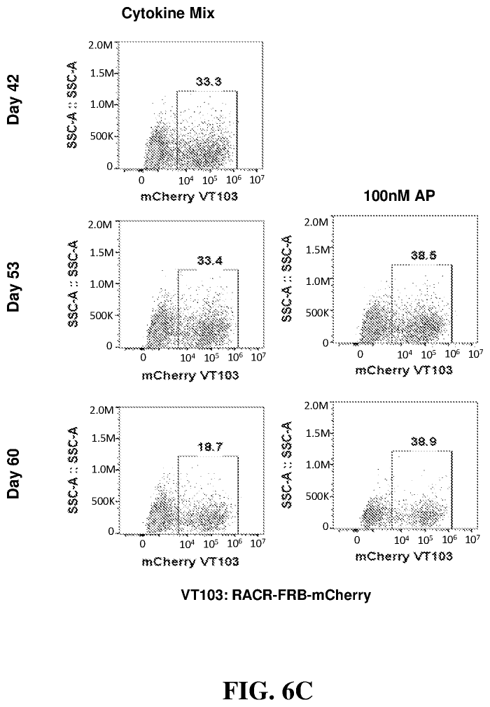

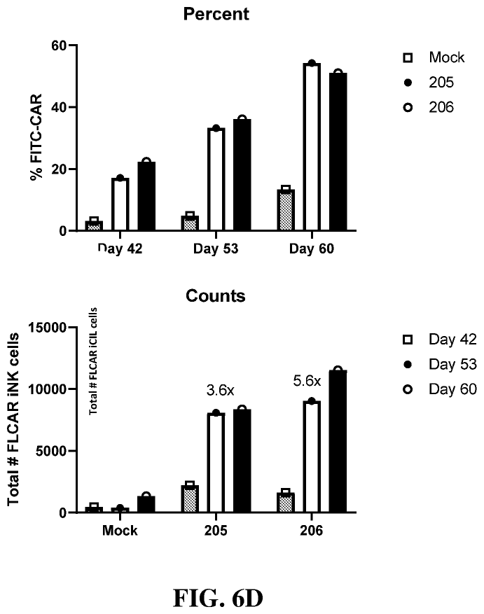

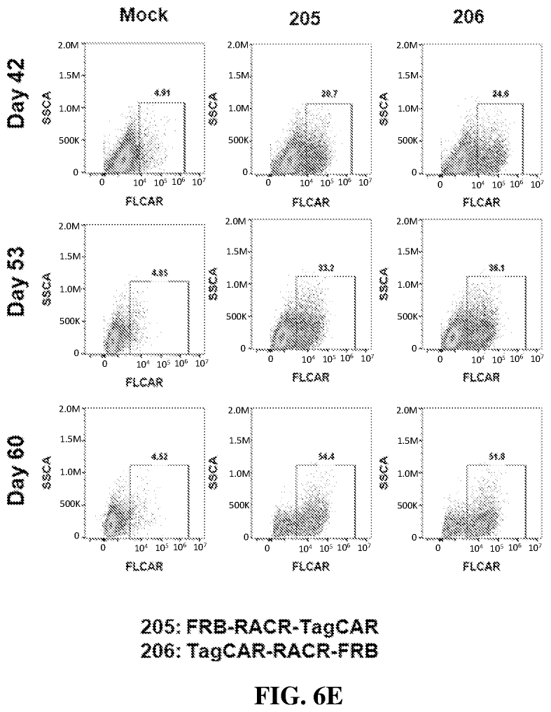

To investigate the ability of RACR to drive the expansion of iCIL cells, transduction and expansion of iCIL cells post differentiation at Day 40 was examined. iCIL cells were generated as described in Example 2. A timeline of the experiment is shown in FIG. 6A. Activation beads (#130-094-483) were added to CIL cells at 2:1 ratio and cells were placed in 37° C. incubator overnight to increase transduction of cells. At Day 42, the cells were transduced with either VSV-G lentivirus with the payload of RACR-FRB-mCherry at an MOI 10 with 10 μg/mL of protamine sulfate or treated with 10 μg/mL of protamine sulfate alone (Mock) (FIG. 6B). The next day a 50% media change was performed, and cells were given CIL expansion media containing IL-2, IL-15, IL-21, IL-18, IL-7 in complete RPMI media: RPMI1640, 10% FBS, 1x Pen-Strep, and 1× Glutamax. On Day 45, media was removed, and Mock cells were placed in cRPMI1640 media supplemented with CIL expansion media and RACR-transduced cells were then placed in cRPMI1640 media supplemented with AP21967 (A/C Heterodimerizer-Takara®) at 100 nM to engage RACR-signaling. 50% media changes were performed weekly with 2x media, respectively. An aliquot of cells was collected weekly, counted on the Vicell Blu automated cell counter, and stained for CD56 clone HCD56 and RACR-FRB positive cells were detected through mCherry (FIG. 6C). RACR-iCIL cells increased both their purity and yield through AP21967 expansion (FIG. 6B and FIG. 6C). The experiment was repeated with cells examined similarly as described above, however, different viral constructs containing TagCAR-RACR-FRB (206) and FRB-RACR-TagCAR (205) were observed (FIG. 6D and FIG. 6E).

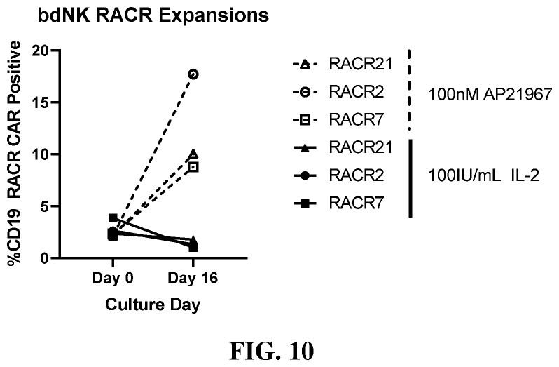

In another experiment, previously isolated blood-derived NK cells were thawed and placed in RPMI media containing 10% FBS, Glutamax, Pen-strep, IL-2 (100 U/ml) on Day-1. On Day 0, the cells were washed in RPMI media and transduced with a CD19-RACR/VSVG lentiviral vector at an MOI of 20 in the presence of IL-2 (100 U/ml), and protamine sulfate (8 ug/ml). Three RACR constructs were transduced: RACR21 (IL-2RG/IL-21RB), RACR2 (IL-2RG/IL-2RB), and RACR7 (IL-2RG/IL-7RB). Three days later, the media was replaced with fresh RPMI media containing IL-2 (100 U/ml). On Day 5, blood-derived NK cells were assessed for CD19 CAR expression by flow cytometry. On Day 7, cells were counted using the Vi-Cell® cell counter (Beckman) and divided into two equal groups. These two groups represented different expansion conditions for each RACR transduction construct. The two groups were 100 U/mL IL2 and Rapamycin. Next, after expansion additives were added to the cultures, the blood-derived NK cells were stimulated with irradiated (10,000 rads) K562 feeder cells expressing membrane-bound IL-21 and 41BBL at a ratio of ˜2 feeder cells:1 NK cell. On Day 10, the transduced cells received a half volume media change and were resuspended in fresh RPMI media containing either 2×IL-2 (100 U/ml) or 2× Rapamycin. Beginning on Day 10, cells were stimulated with irradiated feeder cells weekly a half volume media change was performed as needed. Cell counting and RACR-CAR expression analysis was performed a weekly basis. Cells were stained with a mix including: CD19 FMC63 (FITC), IL7Ra (APC700), CD56 (BV421), CD25 (V660), CD16 (NUV525), and Zombie UV (NUV450). As seen in FIG. 10, RACR2 treated with Rapamycin supported the highest RACR-expansion and both RACR7 and RACR21 treated with Rapamycin supported RACR expansion to a lesser extent. However, all three cell cultures transduced with RACR constructs and treated with Rapamycin showed significantly higher expansion in comparison to the three cell cultures transduced with RACR constructs and treated with IL-2 (FIG. 10).

### Example 6: Function of RACR-iCIL Cells

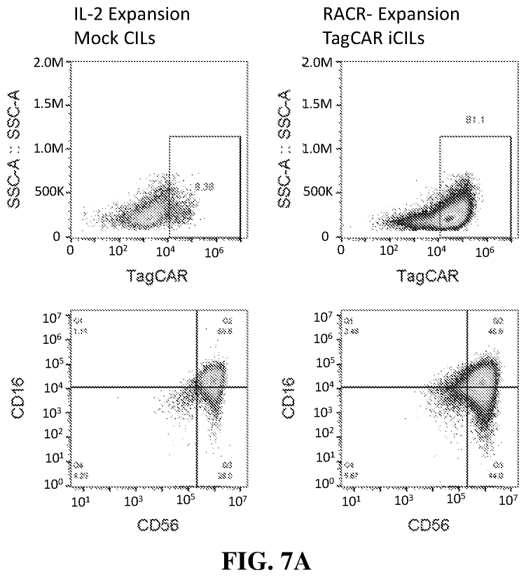

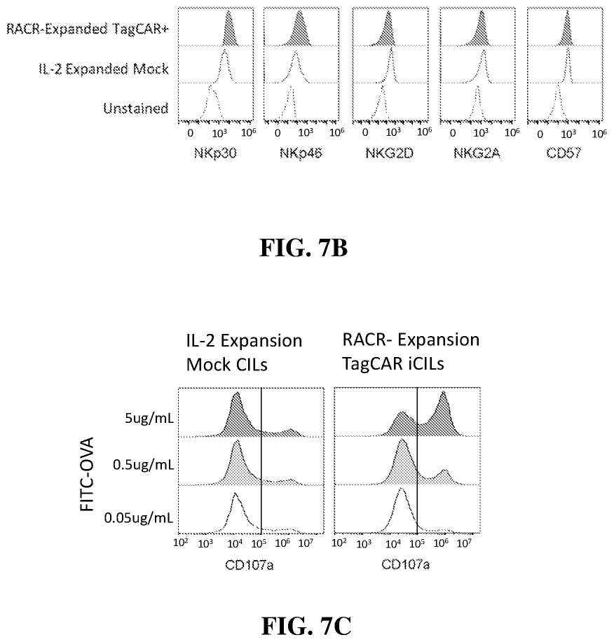

To investigate the functionality of Day 40 expanded RACR-iCIL cells in comparison to iCIL cells expanded in IL-2 immunophenotyping was performed as well as a post-expansion CD107a activity assay. Day 40 iCIL cells were generated with the same process as shown in Example 5 and these cells were transduced with TagCAR-RACR-FRB (206). RACR-iCIL cells were expanded in 100 nM AP21967 and Mock iCIL cells were expanded in 100 IU/mL huIL-2 until Day 60 and then harvested for functional assays. Cells were stained for CD16, CD56, NKp46, NKp30, NKG2A, NKG2D, CD3, CD5 and CD57 and flow cytometry staining was performed as described above. Day 60 RACR-expanded iCIL cells expressed high levels of TagCAR and these cells displayed similar phenotype to IL-2 expanded Mock cells with high expression of CD56 and CD16 (FIG. 7A). RACR-expanded TagCAR iCIL cells expressed higher levels of NKp46 and NKp30 than IL-2 expanded Mock iCIL cells, suggesting a more cytotoxic phenotype (FIG. 7B). Expression of NKG2A, NKG2D, and CD57 were unchanged between RACR-iCIL cells and IL-2 expanded control cells. The ability of RACR-TagCAR expressing cells to secrete CD107a in response to antigen exposure was assessed. TagCAR antigen was coated on 96 plates in PBS at 5. 0.5, and 0.05 μg/mL for 1H at 37° C. Plates were washed and RACR-iCIL cells and IL-2 Mock iCIL cells were then added to the well in the presence of anti-CD107a PE in cRPMI media. Cells were incubated for 4 hours with 1× monesin followed by flow cytometry staining for CD56. Cells were pelleted, washed in BD FACs wash and then collected on Cytoflex flow cytometer. RACR-expanded TagCAR iCIL cells showed robust CD107a secretion in response to antigen indicating high cytotoxicity of RACR-expanded cells (FIG. 7C).

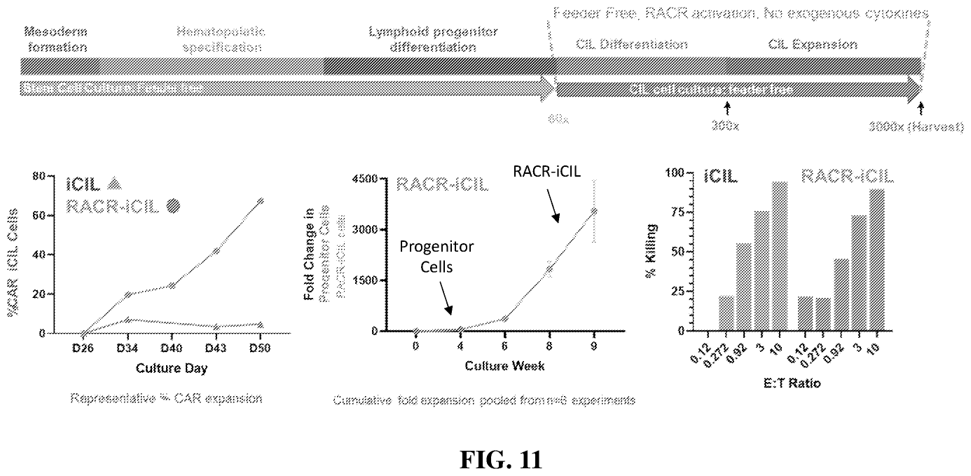

These data show that over the course of iCIL differentiation and feeder-free expansion, approximately 3000-fold increase of iCILs was observed (FIG. 11). Because the synthetic receptor induces a signal akin to IL-2 or IL-15, the iCILs possess a selective growth advantage, resulting in a highly pure iCIL population (FIGS. 3, 4, and 7). iCILs exhibit multifunctional properties, including cytotoxic activity against tumor target cells (FIGS. 3 and 11) and the secretion of IFNγ and TNF-α, mediated by the synergistic activity of innate activating receptors and chimeric antigen receptor targeting (FIG. 7).

### Example 7: RACR-Signaling to Replace Common Gamma Chain Cytokines

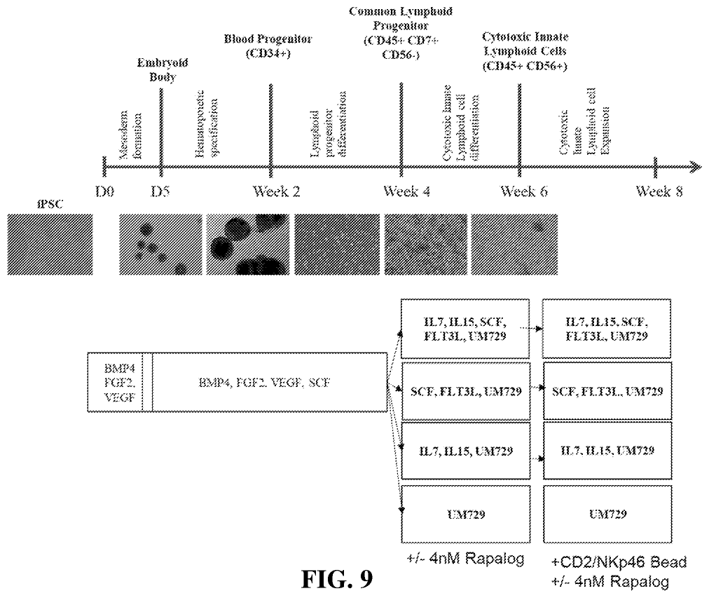

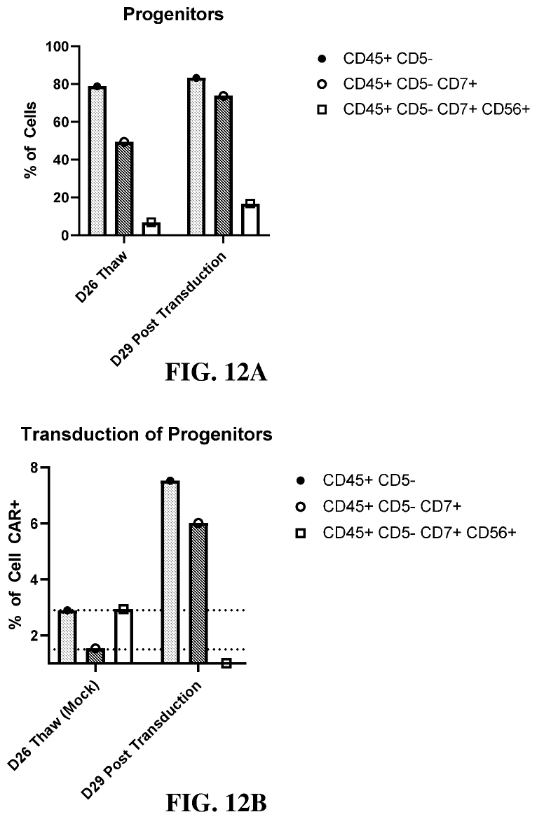

To investigate the ability of RACR-signaling to replace common gamma chain cytokines during differentiation from CLP into CIL cells, CLP cells were transduced at Day 26, prior to exposure to differentiation media. The transition from CLP to CIL generally requires the common gamma chain cytokines IL-7 and IL-15 with differentiation factors SCF. FLT3L, and UM729. A timeline of the experiment is shown in FIG. 9. The iPSC line WTC11 was differentiated into lymphoid progenitors through the STEMdiff® cell kit (STEMCELL Technologies, #100-0170) as described above up until the Day 26 timepoint. The percent containing common lymphoid progenitors (CD45+/CD7+) was 49.4% in this preparation, with very few cells (approximately 10%) expressing CD56 (FIG. 12A). A 50% media change was performed using StemSpan® SFEM II media supplemented with StemSpan® Lymphoid Progenitor Expansion Supplement. The Day 26 cells were then transduced with VSV-G lentivirus with the payload of TagCAR-P2A-FRB-P2A-RACR at an MOI 10 and treated with 10 μg/mL of protamine sulfate or treated with 10 μg/mL of protamine sulfate alone without transduction (mock).

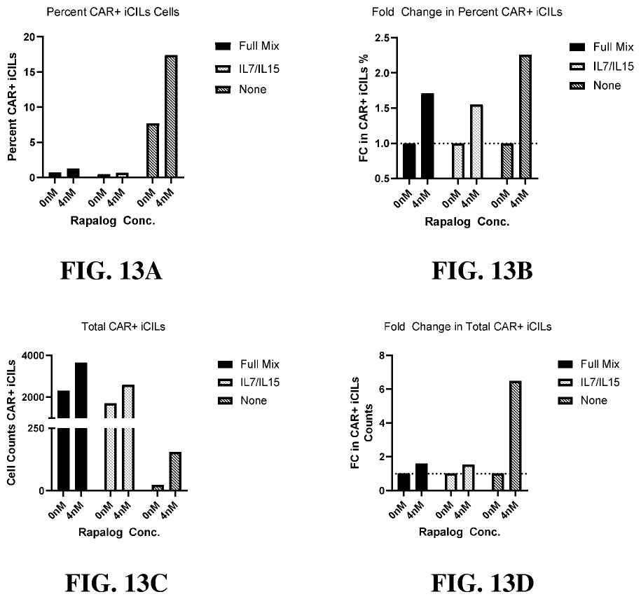

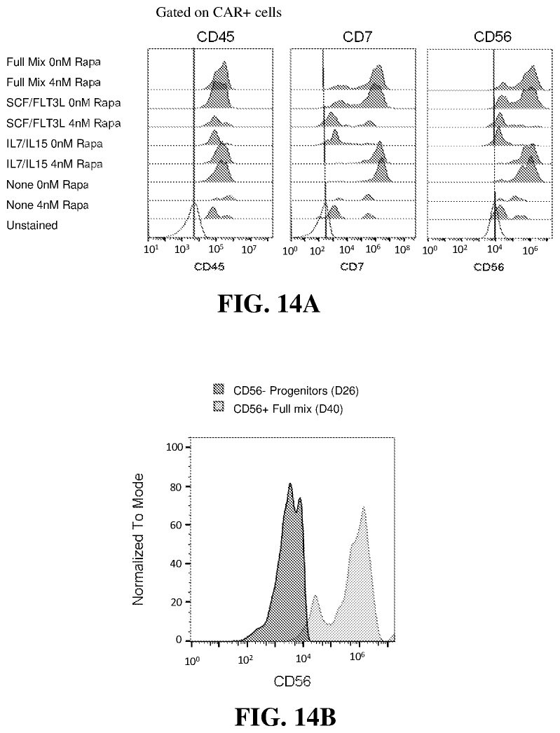

On Day 29, transduction of progenitors was measured and found that approximately 8% of CD45+ cells contained the construct (FIG. 12B). No transduction was detected in the CD56+ cell population (FIG. 12B). CD45+ cells were collected and reseeded into four conditions to induce differentiation from the transduced CLP to a CIL cell. Four culture conditions were examined: 1) StemSpan® SFEM II media supplemented SCF, FLT3L, IL7, IL15, and UM729 (Full Mix); 2) StemSpan® SFEM II media supplemented SCF, FLT3L, and UM729 (SCF/FLT3L); 3) StemSpan® SFEM II media supplemented IL7, IL15, and UM729 (IL7/IL15); and 4) StemSpan® SFEM II media with UM729 alone (None). Cells were then further split into two treatment populations: with 4 nM AP21967 (rapalog) or without (0 nM rapalog). The largest fold change in RACR-CAR+ cells was observed in the condition with AP21697 alone (None), demonstrating the cell's dependence on the RACR-signal for cell growth (FIG. 13D). On Day 33, 50% well-volume of the same media was added and then a 50% media change was performed on Day 34. On Day 40, an aliquot of cells was collected and stained with CD16 clone 3G8, CD56 clone HCD56, CD45 clone 2D1, CD5 clone L17F12, CD7 clone CD7-6B7 and TagCAR detection through FITC-AF647 to analyze CIL cell differentiation and TagCAR expression (FIG. 14A). An increase in the percent of RACR-CAR+CD56+ cells in response to AP21967 (rapalog) was observed in all conditions with the highest percent increase observed in conditions lacking IL7/IL15, because in those conditions the RACR provided the common gamma chain signal and RACR-CAR+ cell expansion (FIG. 14A). All RACR-CAR+ cells were observed to be CD45+. Lower levels of CD7 and CD56 were observed in cells grown in media not containing cytokines as compared to cytokine exposed cells (FIG. 14A). Progenitor cells analyzed on Day 26 displayed lower levels of CD56 as compared to cells exposed to the Full Mix analyzed on Day 40 (FIG. 14B). This data demonstrates that rapalog AP21967 and the RACR system may replace common gamma chain cytokines IL-7 and IL-15 during iCIL differentiation from the common lymphoid progenitors.

The synthetic cytokine receptor system may be employed ex vivo to generate immune effector cells in a manufacturing setting and has the potential to enable selective expansion and survival of the engineered cells in vivo. These data demonstrate the capacity of the disclosed methods and associated compositions to produce innate immune effector cells with significant potential as cancer therapies. This approach has the potential to improve manufacturing process control and product consistency, as well as decrease complexity of iPSC-based allogeneic cell therapy manufacturing. By rationally designing and deploying different types of synthetic receptors, the disclosed methods and associated compositions will be amenable to the production of immune effector cells for therapeutic application in hematological and solid tumor settings.

### Example 8: In Vivo Anti-Tumor Activity

The engineered CIL cells of the disclosure will be investigated in a NSG MHC knockout mice to evaluate the iCIL cell in vivo anti-tumor activity. This humanized mouse model is an immunodeficient NOD scid gamma mouse, which carries the strain NOD.Cg-Prkdc^{scid} Il2rg^{tmIWjI}/SzJ. To establish a xenograft tumor model, tumors will be injected intraperitoneally or subcutaneously into the mice. RACR-iCILs will subsequently be injected either intraperitoneally or intravenously. To analyze the effect of RACR-iCILs on the xenograft tumors, tumor size will be measured by bioluminescence imaging (BLI) over time.

### Example 9: Gene Editing of iPSCS

To increase the proliferation of cell populations of the disclosure, iPSCs were genetically engineered using methods well known in the art, such as in Ran et al. Nature Protocols, Vol. 8, pgs. 2281-2308 (2013), Liu et al. The CRISPR Journal, Vol. 3, Issue 3 (2020), and General CRISPR RNP Transfection Guidelines by Thermo Fisher Scientific, which are incorporated by reference in their entirety. In this example, electroporation was used. Generally, iPSCs were engineered to modulate expression and knockout various endogenous loci as described using CRISPR-Cas9-mediated gene editing. The cells were electroporated with ribonucleoprotein (RNP) complexes comprising a Cas9 and a guide RNA that targeted the specific locus indicated.

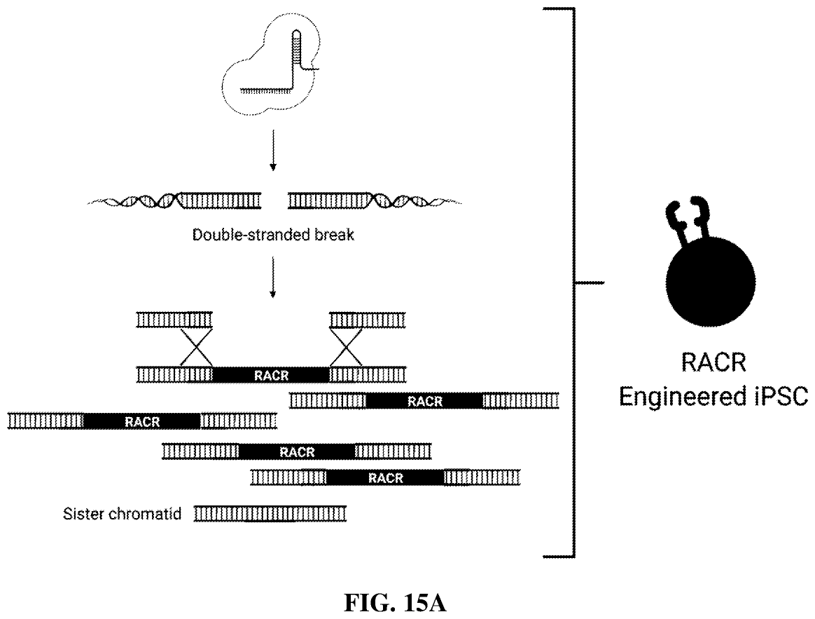

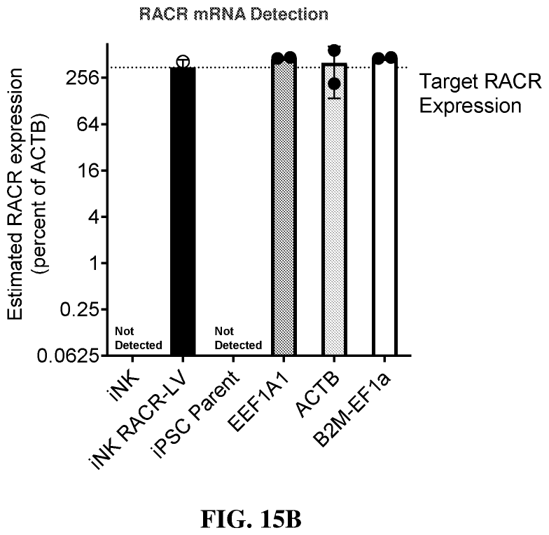

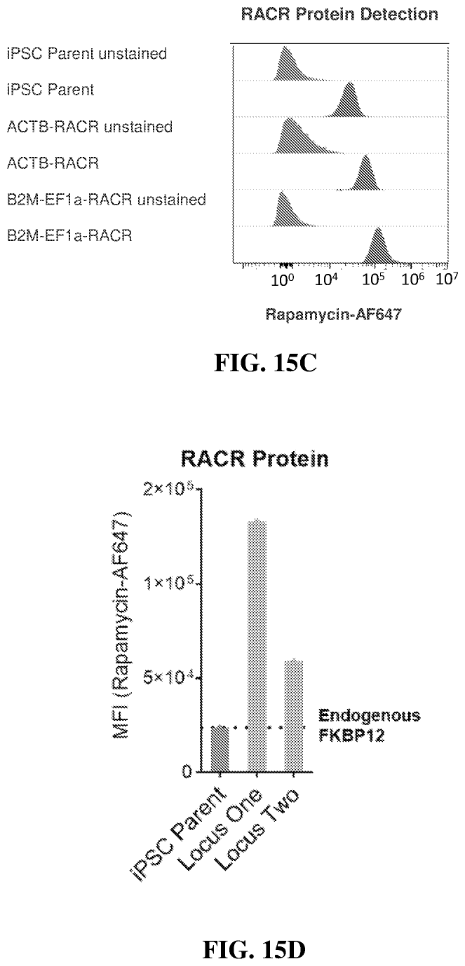

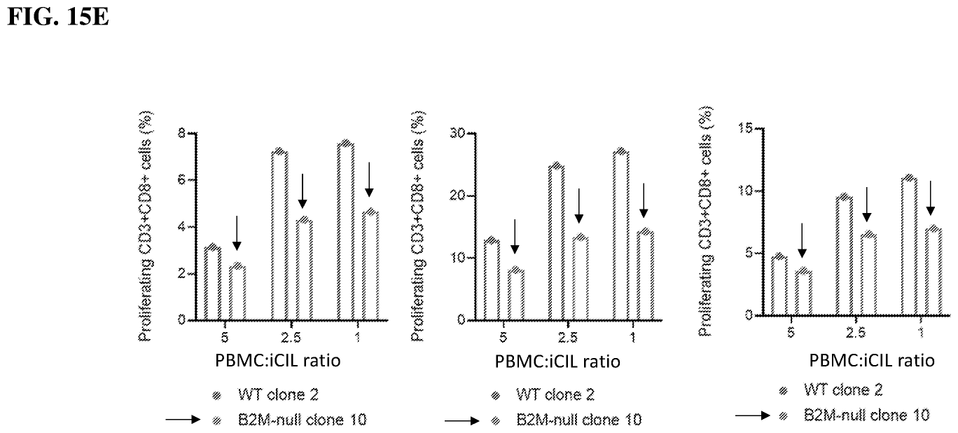

CRISPR-Cas9 was used to generate site-specific knock-ins of constructs encoding RACR via homology-directed repair (HDR) at various loci and is shown schematically in FIG. 15A. Specifically, RACR was knocked-in at the EEF Al locus, ACTB locus, or B2M locus with the addition of an EEF IA promoter. The following table (Table E1) shows exemplary genes and regions for knocking-in a gene.

TABLE E1

| | | Target | Example | Example |
|---|---|---|---|---|
| | Chromosome | Region | Transcript | Target Exon |
| Examples of | | | | |
| housekeeping loci | | | | |
| ACTB | Chr7 | 7p22.1 | NM_001101.5 | Exon 2 or 6 |
| UBC | Chr12 | 12q24.31 | NM_021009.7 | Exon 1 or 2 |
| GAPDH | Chr12 | 12p13.31 | NM_002046.7 | Exon 2 or 9 |
| EEF1A1 | Chr6 | 6q13 | NM_001402.6 | Exon 2 or 8 |
| Examples of blood | | | | |
| lineage-specific loci | | | | |
| PTPRC | Chr1 | 1q31.3-q32.1 | NM_002838.5 | Exon 2 or 33 |
| IL2RG | ChrX | Xq13.1 | NM_000206.3 | Exon 1 or 8 |
| IL2RB | Chr22 | 22q12.3 | NM_000878.5 | Exon 2 or 10 |
| Examples of simultaneous | | | | |
| knockout and knockin | | | | |
| B2M (for any cell type) | Chr15 | 15q21.1 | NM_004048.4 | Exon 1 or 2 |
| TRAC (for T cells) | Chr14 | 14q11.2 | NA (Gene ID: 28755) | Exon 1, 2 or 3 |
| SIRPA (for macrophages) | Chr20 | 20p13 | NM_001040023.2 | Exon 1 or 2 |

After expanding the knock-in cell lines, mRNA levels of RACR were detected through quantitative PCR of cDNAs (FIG. 15B) and protein levels of RACR were detected through flow cytometry after staining with rapamycin-AF647 (FIG. 15D). The expressed levels of RACR were normalized to actin B (ACTB) mRNA expression for either lentiviral transduced iPSC-derived NK cells or gene-edited iPSCs. Lentiviral levels (INK RACR-LV) were used as a benchmark for the desired level of RACR expression. RACR was first knocked into the B2M locus with simultaneous B2M knock out and expressed using a using a dual promoter system containing both the endogenous B2M promoter as well as an exogenously provided EF1a promoter. The endogenous B2M promoter alone was insufficient to produce high levels of RACR. This dual promoter system resulted in RACR levels that were equivalent or higher than what can be reached through lentiviral transduction of RACR with a strong exogenous MND promoter. Removing B2M through gene knock out also reduces allogeneic anti-graft responses of cell therapies by reducing CD8 T cell mis-match responses (FIG. 15E).

Next, protein levels of RACR were detected through flow cytometry analysis of iPSC cells stained with rapamycin conjugated to Alexa Fluor 647 (Rapa-AF647). Unstained cells are shown as a control. The stained signal in the iPSC parent is likely due to Rapa-AF647 binding to endogenous FKBP12, with further increases in signal observed in cells expressing RACR from either ACTB or B2M-EF1a modified loci (FIG. 15C). Additionally, iPSCs constitutively expressing RACR display normal karyotype, as assessed up to 30 passages (data not shown).

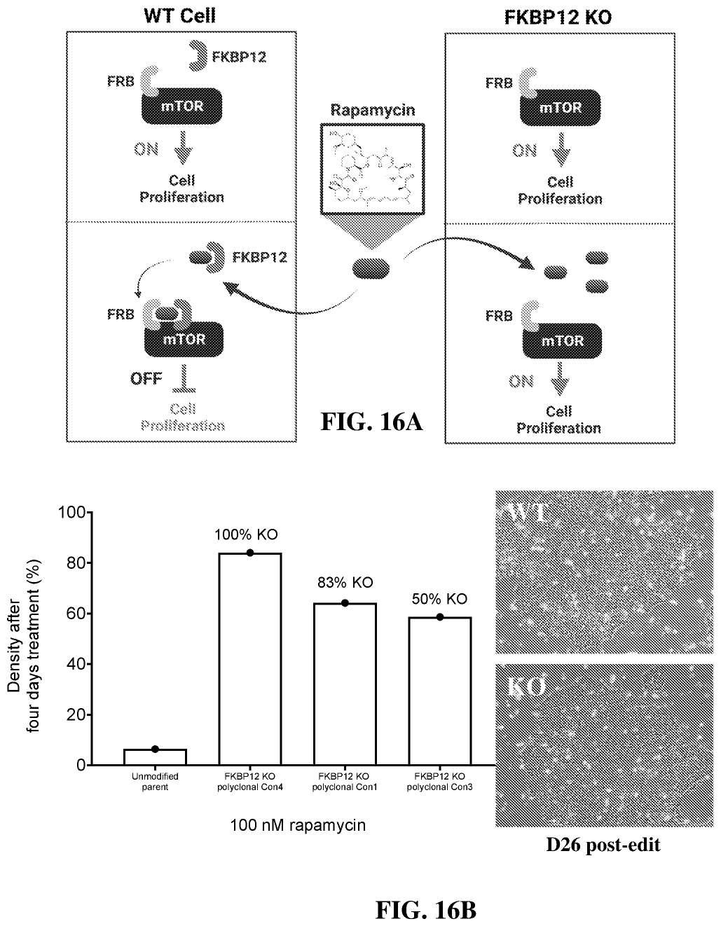

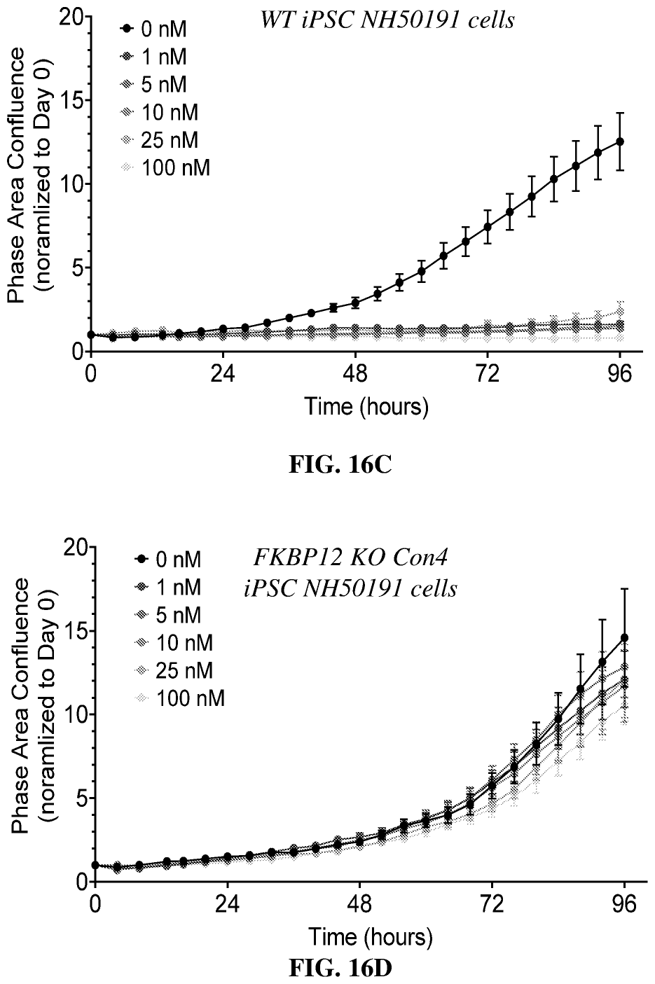

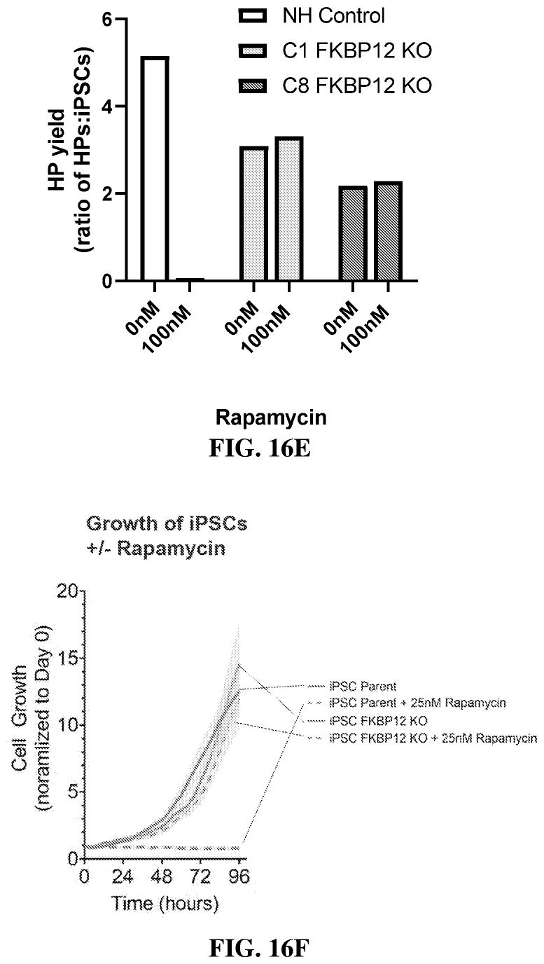

RACR requires rapamycin for activation; however, rapamycin inhibits cell growth. To generate rapamycin resistant cells, the FKBP12 gene was knocked-out. A schematic depicting the role of FKBP12 in the inhibition of proliferation by rapamycin via mTOR is shown in FIG. 16A. In wildtype cells, FKBP12 binds to rapamycin creating a novel binding surface that enables FKBP12-rapamycin to dimerize with the FRB domain of mTOR and subsequently inhibit mTOR. In the absence of FKBP12, rapamycin cannot interact with the mTOR complex and thus loses its inhibitory capacity. Rapamycin-mediated inhibition of iPSC proliferation in polyclonal FKBP12 knock-out (KO) lines is shown in FIG. 16B, left panel. The efficiency of the knockout was determined by Sanger sequencing. Con1, Con2, or Con3 indicate different electroporation conditions for CRISPR-Cas9 based editing resulting in differing levels of knockout. Phase-contrast images reveal normal iPSC morphology in both wild type and FKBP12 KO cells, FIG. 16B, right panel. The confluency of wildtype iPSCs after four days of treatment with varying doses of rapamycin with inhibition of growth at 1 nM rapamycin or higher is shown in FIG. 16C. The growth of FKBP12 KO iPSCs during four days of treatment with varying doses of rapamycin demonstrated robust growth even at doses up to 100 nM rapamycin, confirming rapamycin resistance of FKBP12 KO lines is shown in FIG. 16D and FIG. 16F. The ability of clonal FKBP12 KO iPSCs to differentiate to hematopoietic progenitors (HPs) in the presence of rapamycin is shown in FIG. 16E. In contrast, rapamycin fully inhibits HP differentiation of the parental wildtype iPSCs.

This experiment demonstrates that FKBP12 KO cell lines have enhanced HP differentiation compared to parental WT iPSCs.

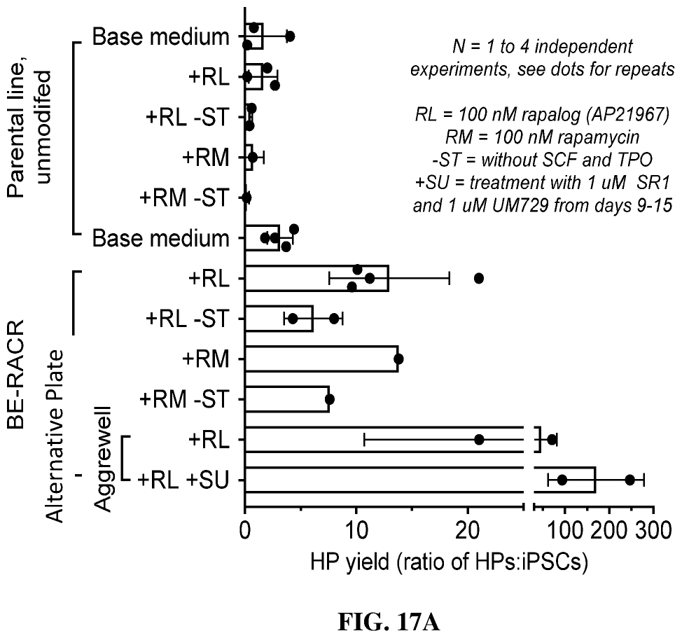

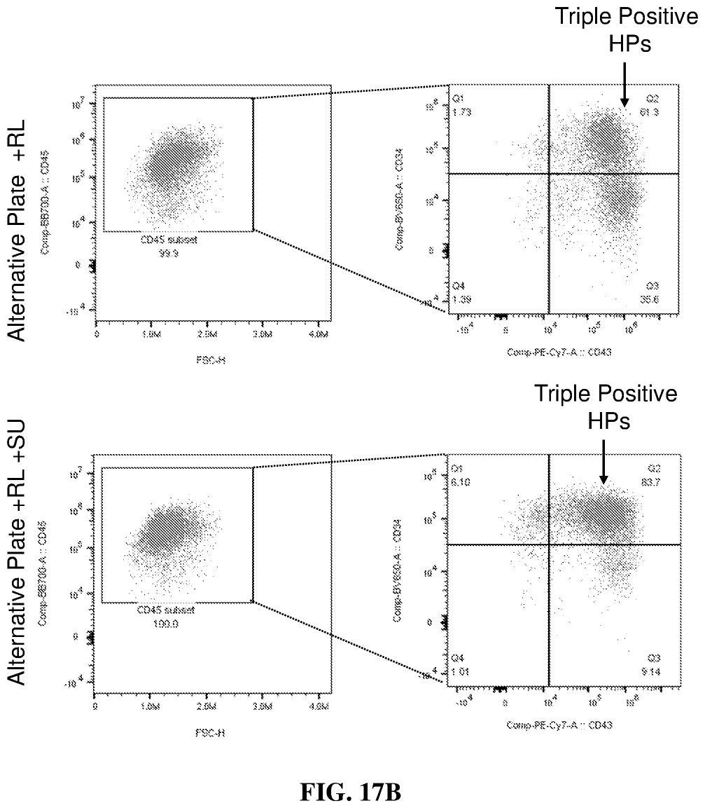

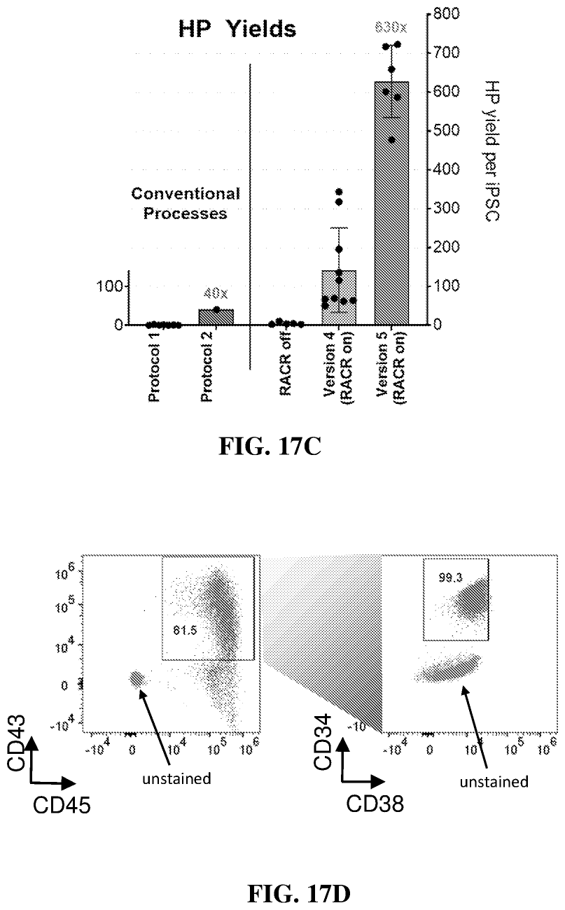

Next, the development and expansion of hematopoictic progenitors (HPs) was examined. Undifferentiated human iPSCs (parental NH50191 iPSC line and edited B2M-EF1a-RACR [BE-RACR] clones) were cultured in mTeSR Plus (STEMCELL Technologies) or iPS-Brew (Miltenyi Biotec) on hESC-qualified Matrigel (Coming). At day 0, embryoid bodies (EBs) were generated from a single cell suspension of iPSCs in STEMdiff APEL 2 Medium (STEMCELL Technologies) supplemented with BMP4, FGF2, VEGF, Y27632, and +/− rapalog (RL; AP21967) or +/− rapamycin (RM). Every 3 days from days 3-12, half of the media was replaced with HP Medium (StemSPAN SFEM II Medium (STEMCELL Technologies) supplemented with BMP4, FGF2, VEGF, TPO, SCF, and LDL) and +/− rapalog or +/− rapamycin. HPs were defined as CD34+/CD43+/CD45+ cells as determined by flow cytometry. EBs were formed using ultra-low attachment 96-well plates (Coming) at 4000 iPSCs per EB. RACR activation by the addition of RL or RM in BE-RACR clones significantly improves HP yield. The removal of the growth factors SCF and TPO (denoted “-ST”) did not strongly impair HP yield, suggesting RACR may be functioning via similar pathways as SCF and TPO to enable the emergence of HPs. EBs were alternatively formed using plates comprising aggregates of microwells (e.g. Aggrewell™ 800 plates) at 500 iPSC/EB or 1000 iPSC/EB. In this protocol, the differentiating EBs were moved to non-tissue culture treated 6-well plates (Corning) at day 6 and fresh HP Medium was added. From days 9-15, StemRegenin-1 and UM729 (denoted “+SU”) were added to half of the groups, yielding four groups total (500 or 1000 iPSC/well, +/− SU from days 9-15). This alternative method of HP differentiation results in markedly higher HP yields after 12-15 days of differentiation. The higher yields observed in the alternative format compared to the 96-well plate format may be due in part to the smaller EB size (500 or 1000 cells per well compared to 4000 cells per well) allowing for better diffusion of rapalog. SU treatment starting at day 9 improved HP yield, as shown in FIG. 17A, and purity, as shown in FIG. 17B.

In further experiments, the protocol for HP generation was further optimized by changing cell culture media components and cell seeding densities. Version 4 is identical to “BE-RACR Alternative Plate+RL+SU” in FIG. 17A with the exception that SR-1 and UM729 (SU) were added on Day 6 rather than on Day 9, 100 nM rapamycin was used instead of 100 nM rapalog. Version 5 is identical to Version 4, with the exception that the initial cell seeding density was 200-250 iPSCs/EB and VEGF, TPO, and SCF were removed after Day 3 of differentiation.

Under these optimized conditions, HP yield increased more than 150 times on average (as shown in Version 4 protocol of FIG. 17C) compared to RACR engineered iPSCs not treated with rapalog or rapamycin. Further, HP yield increased more than 630 times as shown in Version 5 protocol of FIG. 17C compared to RACR engineered iPSCs not treated with rapalog or rapamycin.

The optimized Version 4 protocol resulted in HP cells that were more than 81% CD43+/CD45+ and more than 99% CD34+/CD38− (FIG. 17D).

These experiments demonstrate that RACR activation drives the development and expansion of hematopoictic progenitors (HPs).

### Example 10: Synthetic Receptor Enabled Differentiation from RACR Engineered iPSC

Directed differentiation of iPSC to lymphoid and myeloid progenitor cell types, including hematopoictic progenitors (HP), common lymphoid progenitors (CLP), common myeloid progenitors (CMP) can be achieved by engaging the precision engineered RACR system with rapamycin or rapamycin analogs (rapalog). Engagement of the RACR system with rapamycin or rapalog drives synthetic receptor enabled differentiation through inducing STATS signaling, eliminating requirements for other growth factors that may provide a similar STATS signal, and is sufficient to drive iPSC to the aforementioned progenitor cell types, this is a surprising observation as more conventional approaches incorporate complex milieu of media additives to achieve differentiation to HP. Furthermore, differentiation to CLP/CMP and cytotoxic innate lymphoid (iCIL) cells is enabled by continued engagement of RACR system. In addition, HP and CLP represent progenitor populations for other lymphoid cell types, including alpha beta T cells, gamma delta T cells, NKT cells, NK cells. HP and CMP represent progenitor populations for other cell types including NK cells, monocytes, macrophages, dendritic cells, granulocytes.

Engineered RACR iPSCs were engineered as described in Example 9, including by knock-in of RACR into the B2M locus.

RACR-iPSCs were treated with 100 nM Rapalog starting at DO to generate HPs as described in FIG. 17A and FIG. 17B described in Example 9 above.

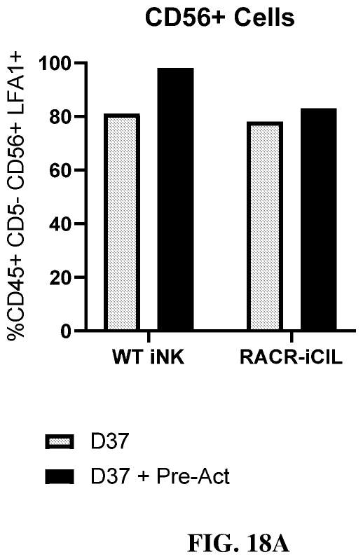

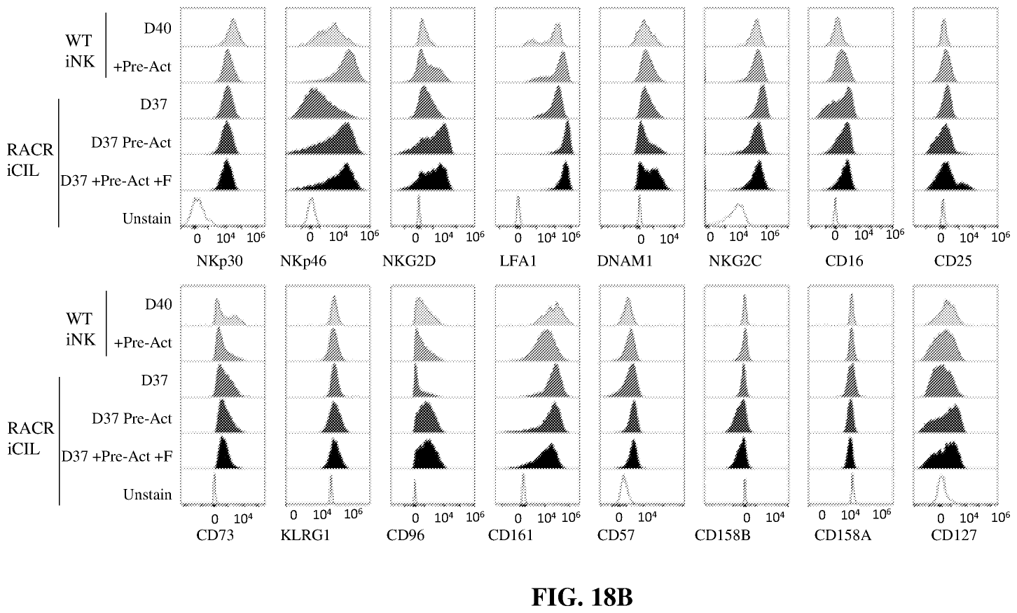

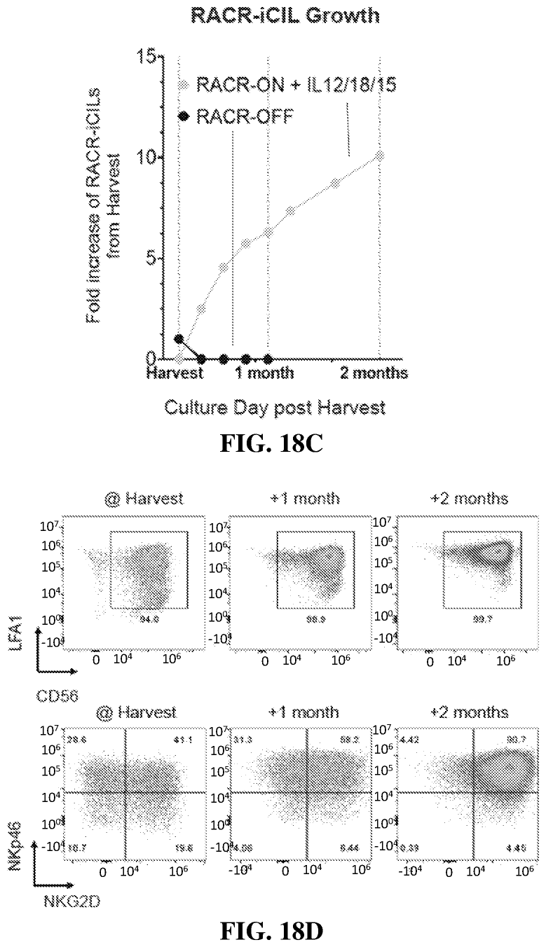

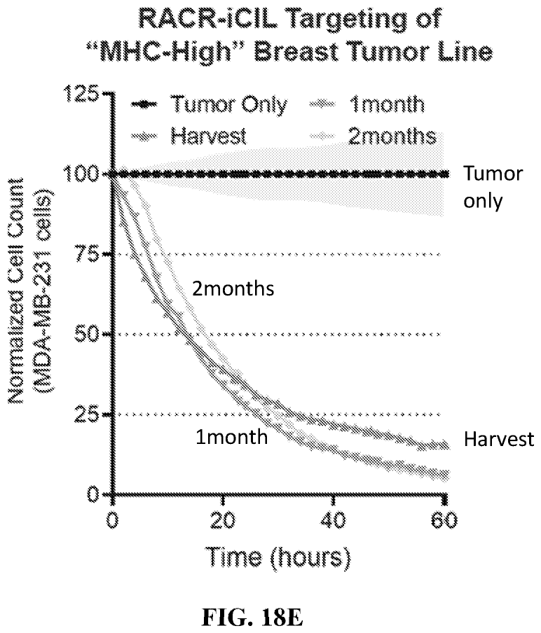

To generate RACR-iCILs, HPs differentiated from Rapalog-treated RACR-iPSCs were then treated with 100 nM Rapalog in NK medium (StemSPAN SFEM II medium supplemented with SCF, FLT3L, IL-7, IL-12, IL-15, SR-I and UM729) from D15 to D30. At D30 cells were either continued in NK medium or placed in a Pre-Activation medium to mature the cells. Pre-Activation medium (Pre-Act) was comprised of BO Medium supplemented with rapalog, IL-7, IL-12, IL-15, IL-18 and IL-21 with or without irradiated K562 feeders (+F). Immunophenotyping comparing iPSC-derived NK cells to RACR-iCILs. Pre-activation increased CD56% purity of both iPSC-NK and RACR-iCILs (FIG. 18A). Pre-activation also increased activation markers NKp46, NKG2D, LFAI, DNAMI, CD16 and decreased expression of inhibitory markers CD161 and CD73, as shown in FIG. 18B.

RACR-iCILs stimulated with cytokines continued to expand one and two months after harvest (FIG. 18C), without feeder cells or rapamycin+cytokine activation. Additionally, phenotypic analysis of iCIL markers CD56, LFA1, NKG2D, and NKp46 show that iCIL purity is maintained one to two months post harvest (FIG. 18D). Further, these RACR-iCILs were capable of targeting MDA-MB-231 cells (FIG. 18E). These data indicate that RACR-iCILs are capable of expanding more than 100 days in feeder-free culture and maintain functionality.

This experiment demonstrates that RACR-iCILs generated using the method described above are phenotypically like NK cells derived from iPSCs with conventional cytokines and growth factors.

Next, the effect of RACR-iCILs cytotoxicity against a solid tumor cell line was examined.

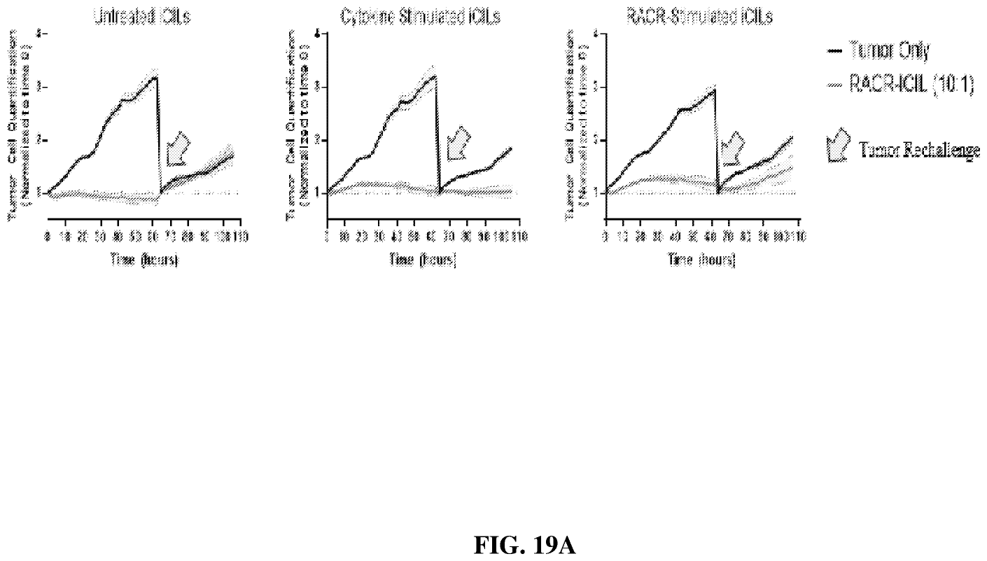

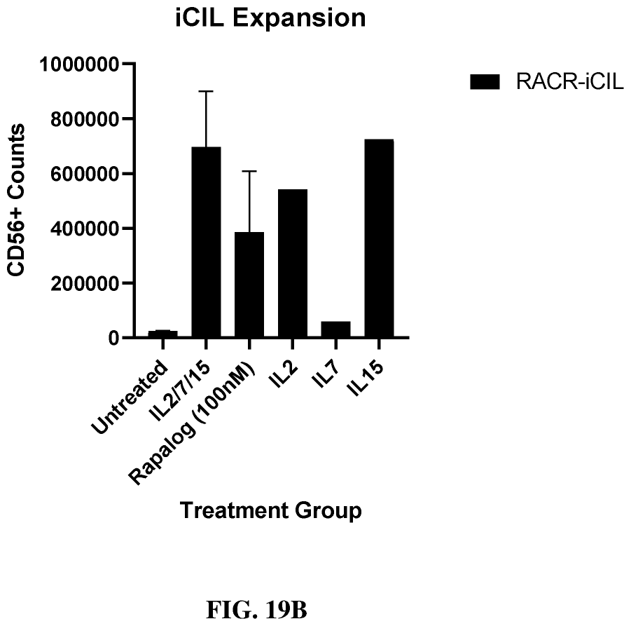

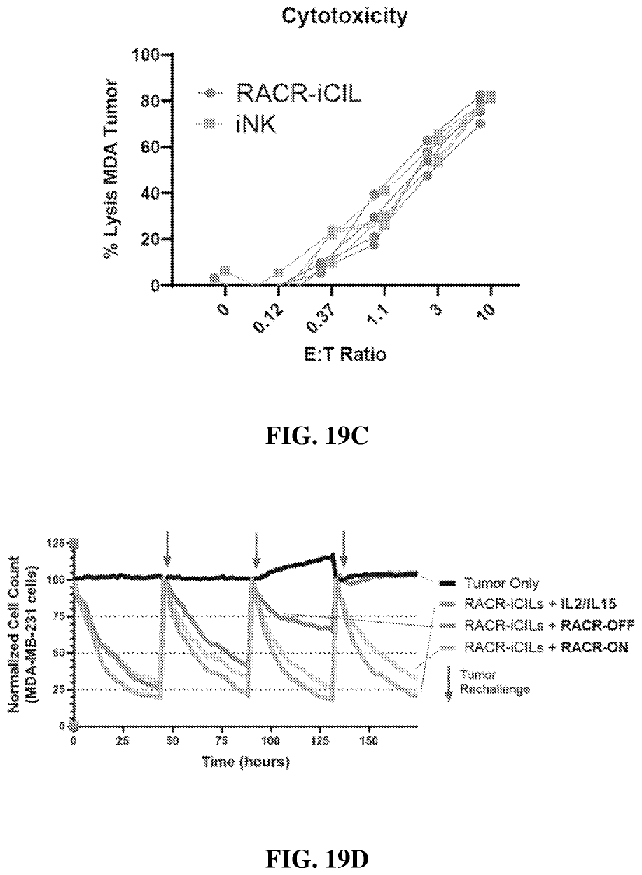

RACR-iCILs were generated similar to as described in FIG. 18A and FIG. 18B except without SCF and TPO included in the HP medium as described in Example 9 and FIG. 17A. Data shown are from two BE-RACR iPSC clones. At D40 RACR-iCILs were incubated for one week in a maturation medium containing IL-2, IL-7, IL-12, IL-15, IL-18 and activation beads (anti-CD2/NKp46). RACR-iCILs were then incubated with breast adenocarcinoma MDA-MB23 1 cells expressing a nuclear fluorescent protein at a 10:1 T:E ratio in the presence of medium alone, cytokines (IL-2/IL-7/IL-15), or rapalog (100 nM). Cell mixtures were placed into an IncuCyte fluorescent microscope and imaged every 2 hours. The number of MDA-MB23 1 cells was quantified over time via a fluorescent marker and graphed as the ratio of MDA-MB23 1 cells compared to time 0 (time 0 is equal to one). RACR-iCILs reduced MDA-MB23 1 growth in all conditions at first tumor exposure. RACR-iCILs were then collected, placed in fresh media, and then re-exposed to MDA-MB23 1 cells for a rechallenge (FIG. 19A). Proliferation of D47 RACR-iCILs was also evaluated in different cytokine treatments or rapalog (100 nM). Rapalog supported RACR-iCIL growth as shown by total RACR-iCILs present one week after stimulation (FIG. 19B).

This experiment demonstrates that engineered RACR-iCILs are cytotoxic against a solid tumor cell line, and continued stimulation by RACR can support their capacity to serial kill tumor cells.

In a similar experiment, RACR-CILs or iPSC-derived NK cells were incubated with breast adenocarcinoma MDA-MB-231 cells expressing a nuclear fluorescent protein at serially increasing T:E ratios (0.12 to 10). FIG. 19C shows that the cytotoxicity of RACR-iCILs was equivalent to that of NK cells generated from iPSCs (iNKs) with conventional differentiation protocols, indicating that resultant RACR-iCILs were highly functional and cytotoxic.

In a serial killing assay, RACR-iCILs were co-cultured with MDA-MB-231 cells, which is a “MHC-high” breast tumor cell line (FIG. 19D). At the start of each new round, RACR-iCILs cells were harvested and re-plated at the initial seeding density with fresh media and target cells, including where applicable cytokines (IL2/IL15) or rapalog (“RACR-ON”). RACR activation (“RACR-ON”) increased cell killing similar to RACR-iCILs stimulated with cytokines. These results demonstrate that RACR-activation increased RACR-iCIL serial killing in this model.

Without wishing to be bound by theory, these results-including high function and cytotoxicity of RACR-iCILs even after re-challenge-support that RACR may permit in vivo persistence even in the absence of lymphodepleting therapy. The results support that the RACR-iCILs could be used to avoid lymphodepletion in patients receiving cell therapy and allow for in vivo cytokine support of adoptively transferred iCILs.

### Example 11: Generation of RACR and CAR-Engineered CILs and Assessment of Cytotoxic Killing Activity

An NK cell line (NK92) was transduced with lentiviral vectors containing free-FKBP12-rapamycin binding (FRB) as well as RACR (SEQ ID NO:32) and anti-FITC CAR (TagCAR). The nucleotide sequence encoding the TagCAR is set forth in SEQ ID NO: 13 and encodes the amino acid sequence set forth in SEQ ID NO: 14; amino acids when the CD8a signal peptide MALPVTALLLPLALLLHAARP is removed (see also e.g. published PCT Appl. No. WO2019144095).

Cells were expanded in 100 nM rapamycin or 100 IU/mL of IL-2 for months to enrich for RACR+ cells. RACR-NK cells were then incubated with different solid human tumor cells expressing a nuclear fluorescent protein at a 10:1 T:E ratio in the presence of media only (unstimulated NK), media supplemented with IL-2 and IL-15 (cytokine-stimulated NK), or supplemented with 100 nM Rapamycin (RACR-stimulated NK).

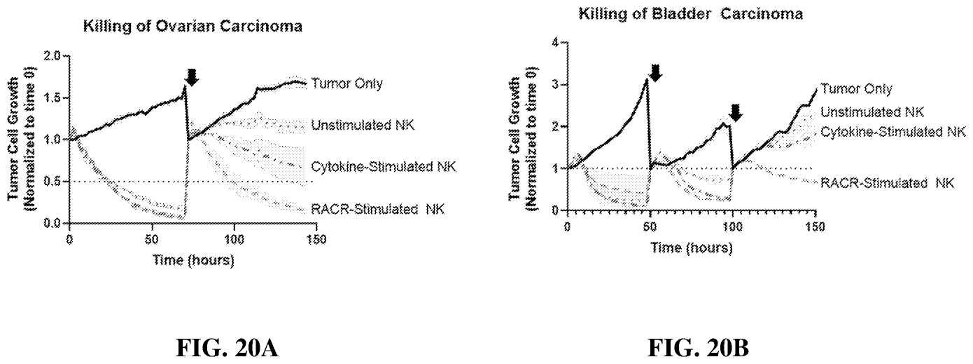

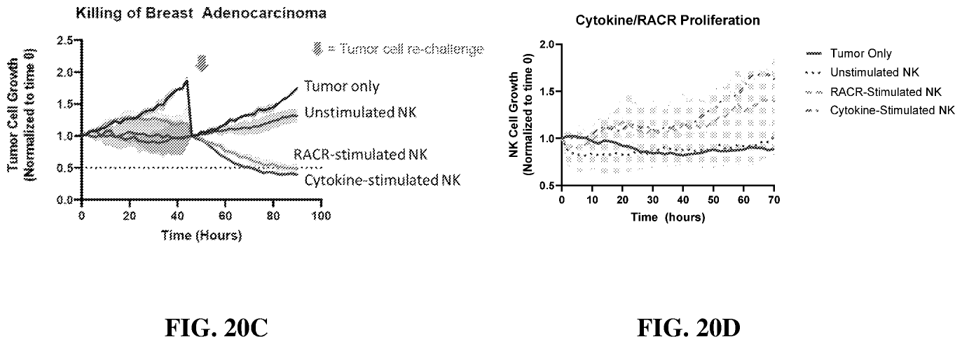

RACR-NK cells and tumor cells were placed into an IncuCyte fluorescent microscope and imaged every 2 hours during co-culture for up to 150 hours. The number of tumor cells was quantified over time via a fluorescent marker and graphed as the ratio of tumor cells compared to time 0 (time 0 is equal to one). At about 40 hours, 50 hours or 100 hours of co-culture, the RACR-NK cells were collected, placed in fresh media and then re-exposed to the tumor cells for a rechallenge. The RACR-NK cells exhibited functional killing of different solid tumor lines including bladder, Heme, colon, breast, ovarian an uterine solid tumor lines. Exemplary results for cytotoxic killing are shown for an ovarian carcinoma (FIG. 20A), a bladder carcinoma cell line (FIG. 20B) and a breast adenocarcinoma cell line (FIG. 20C). The extent of tumor-cell killing, particularly after rechallenge, was substantially greater for the RACR-stimulated NK cells than the cytokine-stimulated NK cells or unstimulated NK cells. Specifically, at first exposure to the tumor all RACR-NK cells cleared regardless of stimulation; however, upon repeated tumor challenge RACR-stimulation via rapamycin supported much higher levels of killing.

NK cell proliferation was also monitored over time during the co-culture. Tracking the expansion of NK cells during tumor-killing of the bladder carcinoma cell line showed that the RACR-stimulated NK cells exhibited the greatest proliferation (FIG. 20D), which may contribute to the increase in the ability of the RACR-stimulated NK cells to serially kill the tumor cells.

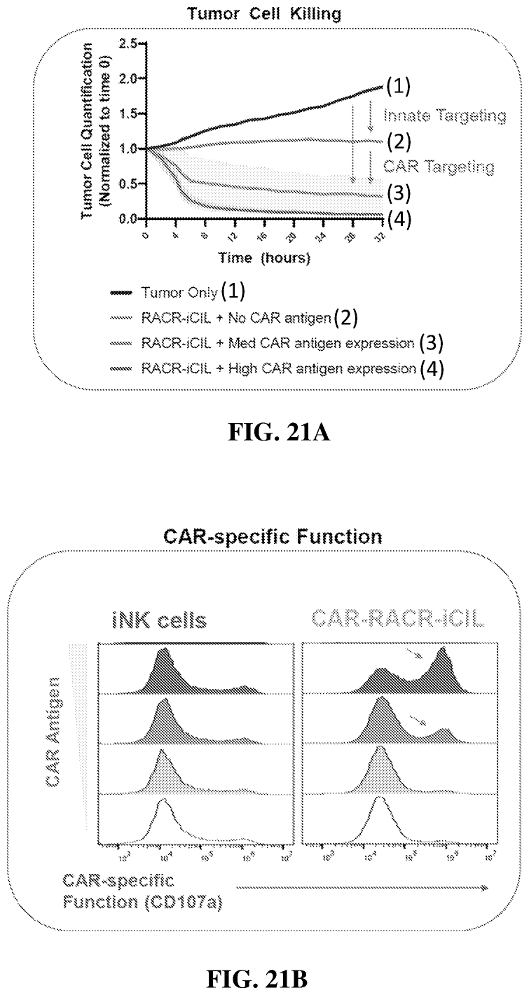

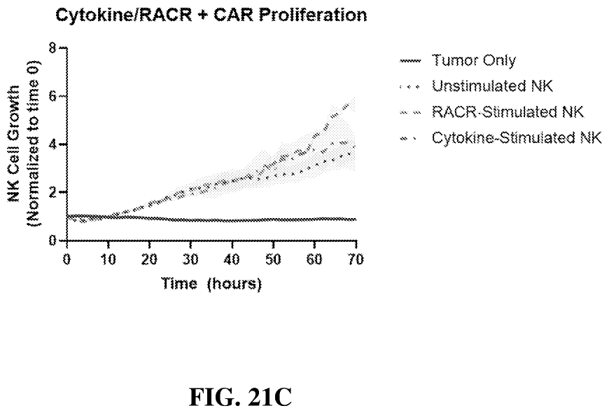

To assess the activity of the CAR expressed by the RACR-NK cells, target cells, a tumor killing assay was performed. As shown in FIG. 21A, RACR-NK cells stimulated with rapamycin exhibited tumor cell killing even in the absence of CAR antigen (“No CAR antigen”). Killing activity was substantially increased in the presence of the Folate-Fluorescein bound to folate receptor expressed on the tumor cells, and the level of activity correlated with antigen expression (medium, “Med CAR antigen expression”; or high, “High CAR antigen expression.) (FIG. 21A).

As an alternative to cell killing, functional activity of NK cells also can be monitored by the marker CD107a, which is a degranulation marker of NK cell functional activity. The CAR antigen-expressing target cells were then cultured with unstimulated NK cells or CAR-RACR-NK cells. As shown in FIG. 21B, the extent of CD107a directly correlated with the level of CAR antigen, indicating a CAR-specific functional activity of the CAR-RACR-NK cells.

NK cell proliferation was also monitored over time during the co-culture. Tracking the expansion of NK cells during tumor-killing of the target cells showed that CAR targeting further increases RACR-NK proliferation, suggesting that RACR and CAR activation can work synergistically to increase RACR-NK proliferation and CAR-mediated targeting (FIG. 21C).

### Example 12: CAR-RACR-iCILS Display Potent and Specific Car-Driven and Innate Tumor Killing

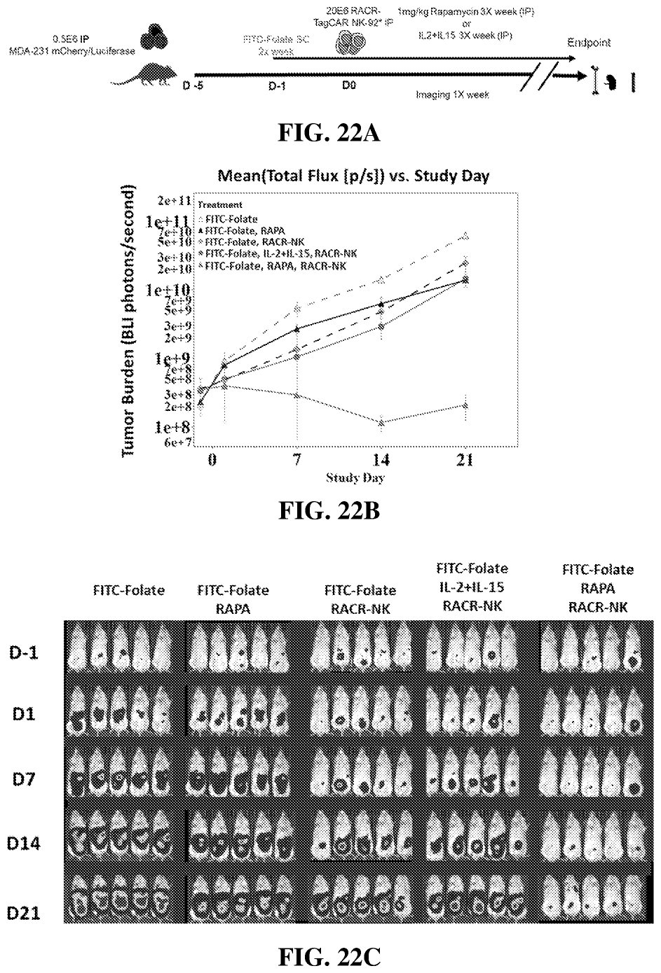

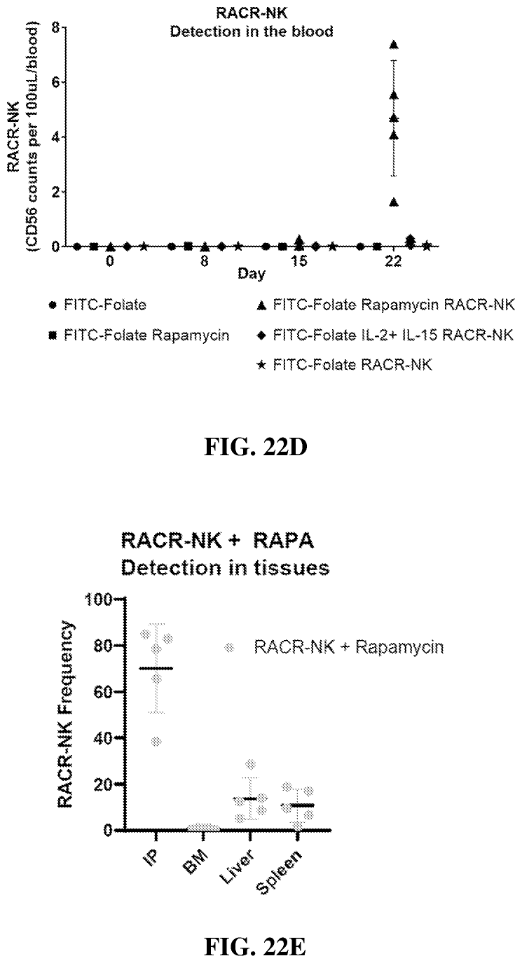

RACR-TagCAR-NK cells, generated as described in Example 11, were tested for their ability to respond to RACR stimulation and control tumors in vivo in a mouse model (FIG. 22A). On Day −5, 5×10⁶ MDA-MB231 mCherry-FFLUC breast cancer cells were injected into the intraperitoneal region of NSG-mice. On Day-1, mice were injected with 100 nM Folate-Fluorescein (Folate-FITC; injected SC two times per week) to tag the tumor cells via the folate receptor. Mice were randomized for tumor size and on Day 0, 20×10⁶ RACR-TagCAR-NK cells were injected into the intraperitoneal region and mice were either given no stimulation, huIL-2+huIL-15 (injected IP three times per week), or rapamycin (injected IP three times per week). The tumor was imaged via bioluminescence imaging (BLI) weekly to monitor tumor progression.

All control mice, other than those treated with RACR-TagCAR-NK cells, had rapid tumor growth as shown by BLI imaging regardless of rapamycin dosing (quantification of luminescence shown in FIG. 22B, and raw images shown in FIG. 22C). There was a reduction in tumor growth in groups dosed with RACR-TagCAR-NK cells. Results also demonstrated an increase in RACR-NK cells detected in the blood (FIG. 22D) and peritoneum of mice (FIG. 22E), which shows the ability of NK cells to target the tumor. However, by day 7 post-RACR-TagCAR-NK injection the rapamycin-treated group began to show improved tumor control compared to the unstimulated or cytokine-stimulated groups. This data demonstrates the superior ability of RACR-stimulation to support tumor killing in vivo, compared even to the “best in class” of cytokine stimulation.

### Example 13: Differentiation of Human Pluripotent Stem Cells into Cytotoxic Innate Lymphocyte Cells in Suspension

RACR iPSCs (edited B2M-EF1a-RACR and knocked out for FKBP12), generated as described in Example 9, were seeded between 8×10⁴ cells/well or 1×10⁶ cells/well in a non-tissue culture treated 6 well plate in 2 mL and were adapted to 3D culture by 1 passage to form aggregates. Non-tissue culture treated plates do not promote cell attachment. Thus, the cells grow in the 3D space of the culture medium. The cells were incubated with Gentle Cell Dissociation Reagent (GCDR; STEMCell Technologies) or EDTA in mTeSR™ 3D medium (in serum-free media; STEMCELL Technologies) or brew supplements (Myltenyi Biotec; Cat #130-127-865) with a support reagent (Support Myltenyi Biotec; Cat #130-127-287) to dislodge the cells and disaggregate them into small clumps to form suspended pluripotent aggregates for suspension culture in a 3D culture vessel. The suspended pluripotent aggregates were then passed to a second non-tissue culture treated 6 well plate on Day 4 for 3D culture in medium to induce mesoderm formation by culture in STEMdiff APEL 2 Medium (STEMCELL Technologies; media is fully defined, serum- and animal-component-free) supplemented with BMP4, FGF2, and VEGF+/− rapamycin (RM). The cells were cultured for 3 days. Then, the media was replaced with HP Medium (StemSPAN SFEM II Medium (STEMCELL Technologies) supplemented with BMP4, FGF2, VEGF, TPO, SCF, and LDL) and +/− rapamycin for induction of HP and cultured from days 3-15.

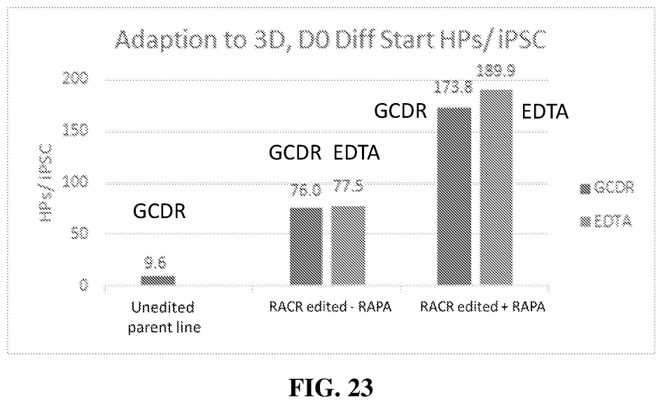

At day 14 or 15 of culture, the differentiated cells were monitored for HP generation. Table E2 and FIG. 23 sets forth the results of the analysis. As shown in FIG. 23, differentiation of the RACR iPSC edited cells produce more HPs than non-edited parental iPSC. This method results in a high yield of HPs based on CD34+/CD43+/CD45+ marker expression; >75% HPs and >9 fold expansion of HP/iPSC. Furthermore, HPs generated in dynamic suspension demonstrated improved yield and purity.

The culture is continued for iCIL cell generation for up to 40 days in culture containing FLT3L, IL-7, IL-12, IL-15, SR-1 and UM729.

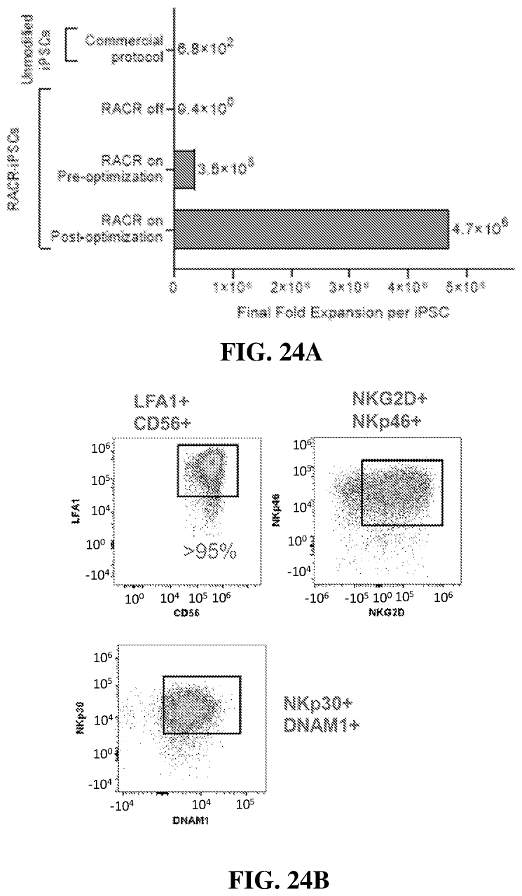

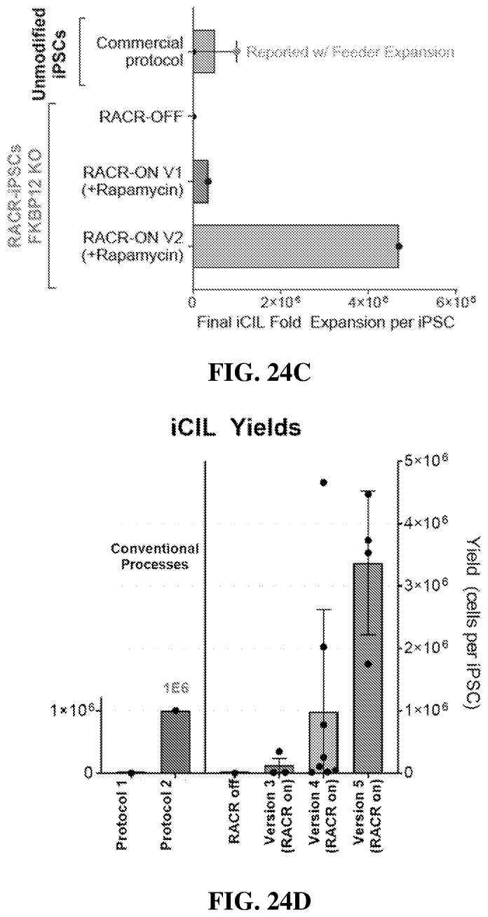

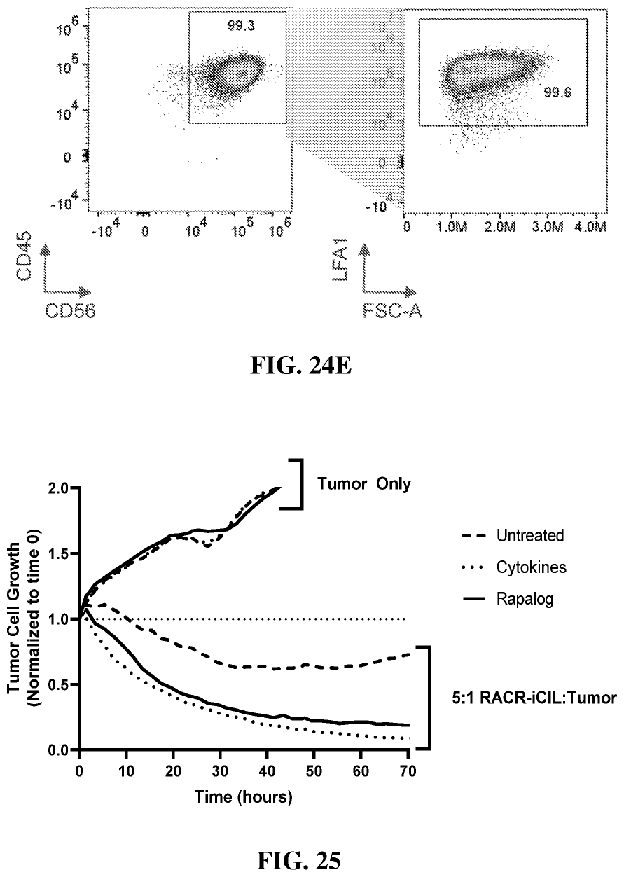

Following FLT3L, IL-7, IL-12, IL15, SR-1 and UM729 cytokine stimulation, RACR-iPSCs were activated with rapamycin, which resulted in a greater than 4-fold production of RACR-iCILs compared to unactivated RACR-iPSCs (FIG. 24A). When RACR-iPSCs were stimulated with rapamycin, there was a greater than 4 million fold expansion of RACR-iCILs, from one initial RACR-iPSC (FIG. 24C). Furthermore, the RACR-iCILs displayed a highly pure and mature phenotype as indicated by the LFA1+/CD56+, NKp46+/NKG2D+ and NKp30+/DNAM1+ staining in FIG. 24B.

TABLE E2

| | | | Viable | | % | | | | |
|---|---|---|---|---|---|---|---|---|---|
| | # Cells | | Cells/ml | Total | CD43+ | % | | Absolute | |
| Sample | Seeded | Viability | (×10\hat{ }6) | Cells | CD45+ | CD34+ | % HP | HP | HP/iPSC |
| Unedited | 8.00E+04 | 83.68 | 1.94 | 2.13E+06 | 98.00% | 36.80% | 36.06% | 7.70E+05 | 9.62 |
| parental | | | | | | | | | |
| iPSC | | | | | | | | | |
| RACR+/ | 8.00E+04 | 75.15 | 3.01 | 6.62E+06 | 94.80% | 96.90% | 91.86% | 6.08E+06 | 76.04 |
| B2M | | | | | | | | | |
| KO/FKB | | | | | | | | | |
| P12KO − | | | | | | | | | |
| Rapa | | | | | | | | | |
| RACR+/ | 8.00E+04 | 63.44 | 6.62 | 1.46E+07 | 96.80% | 98.60% | 95.44% | 1.39E+07 | 173.76 |
| B2M | | | | | | | | | |
| KO/FKB | | | | | | | | | |
| P12KO + | | | | | | | | | |
| Rapa | | | | | | | | | |
| RACR+/ | 8.00E+04 | 67.93 | 3.58 | 7.52E+06 | 91.50% | 90.10% | 82.44% | 6.20E+06 | 77.47 |
| B2M | | | | | | | | | |
| KO/FKB | | | | | | | | | |
| P12KO | | | | | | | | | |
| EDTA − | | | | | | | | | |
| Rapa | | | | | | | | | |
| RACR+/ | 8.00E+04 | 56.03 | 6.6 | 1.58E+07 | 96.60% | 99.30% | 95.92% | 1.52E+07 | 189.93 |
| B2M | | | | | | | | | |
| KO/FKB | | | | | | | | | |
| P12KO | | | | | | | | | |
| EDTA + | | | | | | | | | |
| Rapa | | | | | | | | | |

In a separate experiment, iCILs were generated as described above and final iCIL expansion is shown in FIG. 24D. In FIG. 24D, Versions 3, 4 and 5 represent three iterations of the iCIL differentiation protocol that underwent successive optimization to increase iCIL yields, purity and cytotoxicity. The main differences between Version 3 and Version 4 were the day of seeding GRex bioreactors and the GRex seeding density. In Version 3, the GRex biroeactor was seeded on Day 24, whereas in Version 4, the GRex bioreactor was seeded on Day 41. The main difference between Version 4 and Version 5 was that the seeding of GRex bioreactors was moved up to Day 21. Aim V was the base medium and cytokines IL7 and FLT3L were removed.

By Day 40 of differentiation, there was more than a 3 million fold-expansion of iCIL cells generated by the RACR differentiation process compared to the conventional process (FIG. 24D). Further, more than 99% of the iCILs were CD45+/CD56+ or LFA1+/FSC-+ (FIG. 24E).

### Example 14: RACR-iCILS Demonstrate Early and Potent Tumor Killing

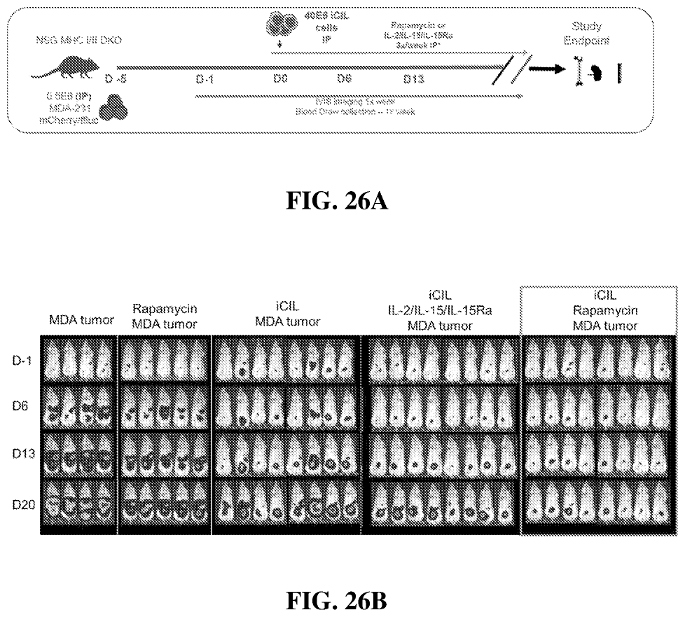

RACR-iCIL tumor killing ability was determined in vitro (FIG. 25) and in vivo (FIGS. 26A and 26B) using the RACR-iCILs generated in Example 10. In vitro tumor killing was determined by co-culturing RACR-iCILs with the breast adenocarcinoma cell line MDA-MB-231. RACR-iCILs were co-cultured with MDA-MB-231 cells at a ratio of 5:1 and either untreated, treated with cytokines, or treated with rapalog. Rapalog and cytokine activation of RACR-iCILs enhanced tumor killing compared to untreated RACR-iCILs (FIG. 25).

In vivo tumor killing was determined in a mouse model. A timeline of the experiment is shown in (FIG. 26A). On Day −5, 5×10⁶ MDA-MB-231 mCherry-FFLUC breast cancer cells were injected into the intraperitoneal region of NSG MHC I/II DKO mice. Beginning on Day-1, mice were imaged once per week and their blood was collected once per week until the end of the experiment. Mice were randomized for tumor size and on Day 0, 40×10⁶ RACR-iCIL cells were injected into the intraperitoneal region and mice were either given no stimulation, IL-2/IL-15/IL-15Ra (injected IP three times per week), or rapamycin (injected IP three times per week). Control mice received either MDA-MB-231 cells alone, MDA-MB-231 cells and rapamycin, or MDA-MB-231 and iCIL. The tumor was imaged via bioluminescence imaging (BLI) weekly to monitor tumor progression.

All control mice, other than those treated with RACR-iCIL cells, had rapid tumor growth by Day 6 as shown by BLI imaging (first and second panel from the left in FIG. 26B). There was a reduction in tumor growth in mice administered RACR-iCILs only (third panel in FIG. 26B), which was further reduced at Day 6 with administration of IL-2/IL-15/IL-15Ra or rapamycin (fourth and fifth panels in FIG. 26B). However, over 20 days, the greatest reduction in tumor growth was achieved with administration of rapamycin. This data demonstrates the superior ability of RACR-iCIL stimulation to support early tumor killing in vivo. This data also demonstrates the superior ability of RACR-iCIL stimulation to support tumor killing in vivo compared even to the “best in class” of cytokine stimulation.

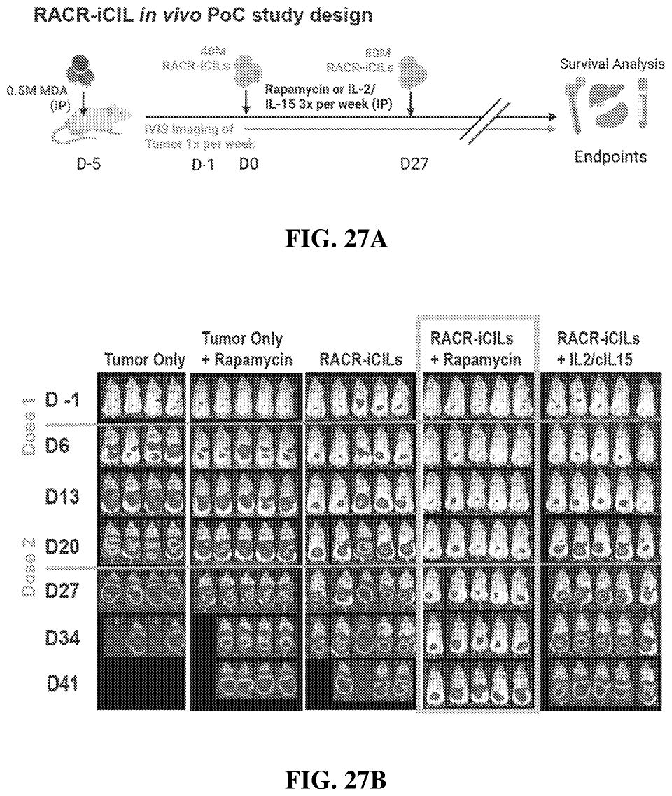

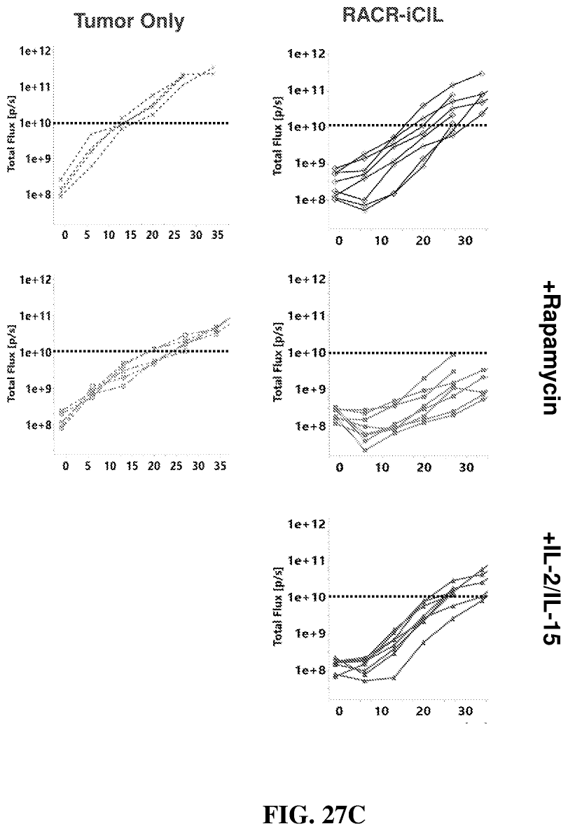

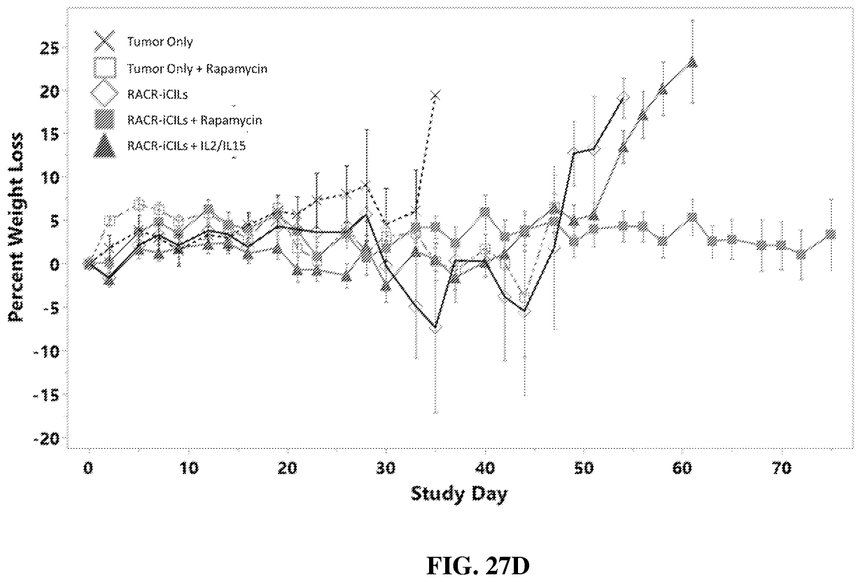

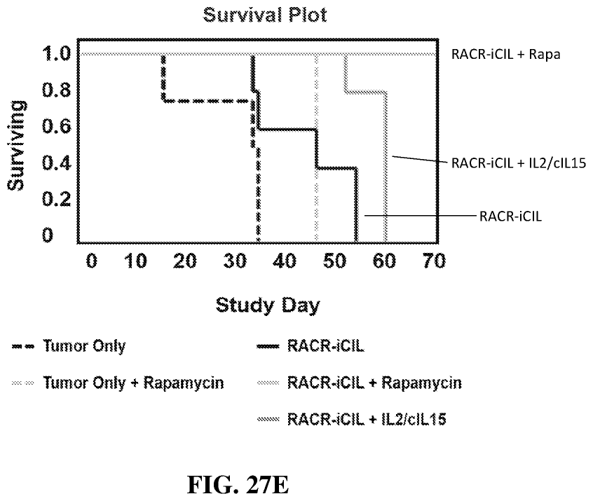

In a separate experiment, in vivo tumor killing was determined in a mouse model where mice received two separate doses of RACR-iCILs on the first day of the experiment and day 27 (FIG. 27A). All control mice, other than those treated with RACR-iCIL cells, had rapid tumor growth by Day 6 as shown by BLI imaging (first and second panel from the left in FIG. 27B and FIG. 27C). There was a reduction in tumor growth in mice administered RACR-iCILs only on Day 6 (third panel in FIG. 27B and FIG. 27C), which was further reduced at Day 6 with administration of IL-2/IL-15/IL-15Ra or rapamycin (fourth and fifth panels in FIG. 27B and FIG. 27C). However, over 41 days, the greatest reduction in tumor growth was achieved with administration of rapamycin. Furthermore, mice receiving RACR-iCIL and rapamycin showed no signs of toxicity, remained alert and had no eight loss for >75 days (FIG. 27D). Additionally, mice receiving RACR-iCIL and rapamycin had 100% survival on Day 70 (FIG. 27E). This data demonstrates the superior ability of RACR-iCIL stimulation to support early tumor killing in vivo. This data also demonstrates the superior ability of RACR-iCIL stimulation to support tumor killing in vivo compared even to the “best in class” of cytokine stimulation.

### Example 15: Marker Expression in Hematopoietic Cells Derived from RACR-Engineered Hematopoietic Stem Cells

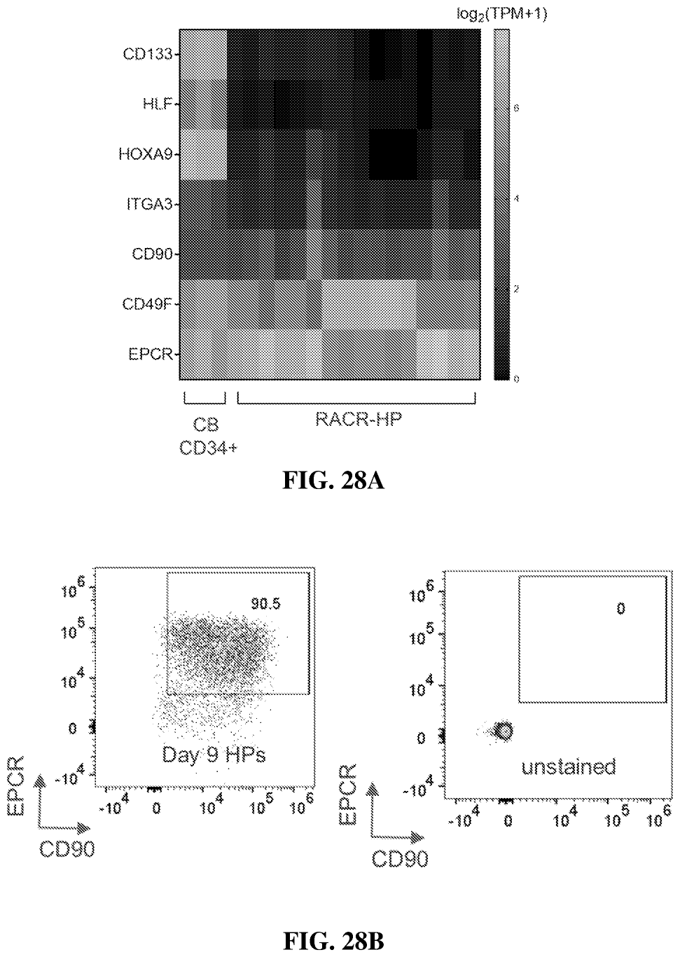

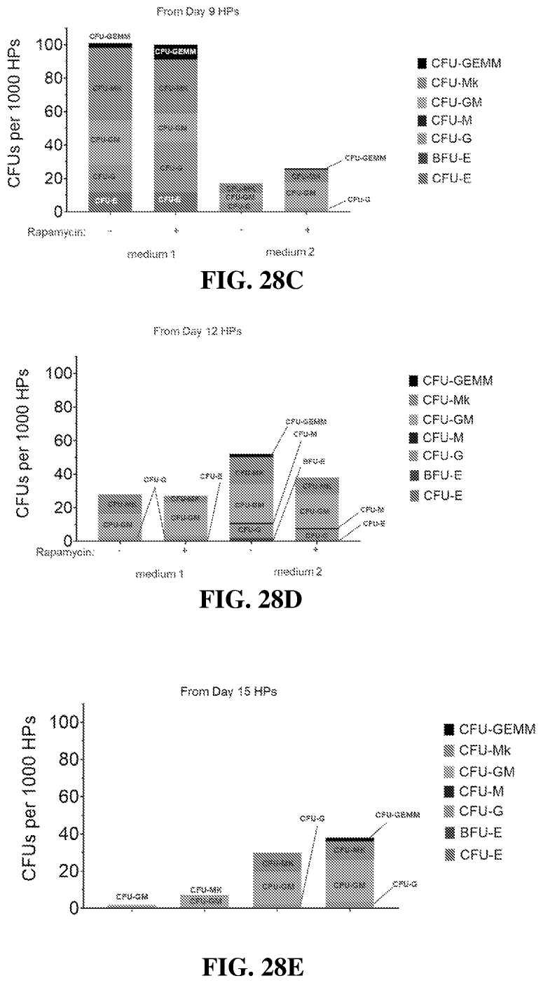

The expression of six classic hematopoietic stem cell (HSC) markers (CD133, HLF, HOXA9, ITGA3, CD90, CD49F, EPCR) were measured in HPs derived from iPSCs containing the B2M-EF1a-RACR KI and FKBP12 KO edits using Version 5 protocol described in Example 13 and were harvested on Day 15 for RNA isolation. The cord blood CD34+ cells were obtained from commercial vendors and cultured in HP medium without rapamycin for 3 days prior to harvest for RNA isolation. RNAseq was also carried out on HP and cord blood CD34+ cells (cbCD34+). The heatmap data in FIG. 28A depicts the average expression (log 2TPM+1) of the specific markers. Genes were ranked based on their expression intensity.

As shown in FIG. FIG. 28A, the RACR-HP share some markers found on HSCs from cord blood. Expression of CD133, HLF and HOXA9 was lower in RACR-HP at Day 15 of the differentiation process (maturity) compared to expression of the markers in CD34+ cord blood cells (FIG. 28A). By Day 9 of differentiation, >90% of RACR-HP were EPCR+/CD90+ (FIG. 28B).

Myeloid and erythroid potency was also determined in RACR-HP. RACR-HP cells were differentiated in StemLine II (Sigma-Aldrich; medium 1) or StemSpan FEM II (Stem Cell Technologies; medium 2) and treated with or without 20 nM rapamycin Colony Forming or Burst Forming Units were measured on Days 9, 12 and 15 of the differentiation process (FIGS. 28C-28E). In a colony formation assay, function and potency of progenitor cells are determined by the presence of myeloid or crythroid cells. The more types of colonies that form, the broader the potency of the original population of progenitors. The presence of GEMM colonies is the most highly prized, because these colonies have formed from a single hematopoietic progenitor that has broad multipotency and is able to make all types of myeloid and erythroid cells, like hematopoietic stem cells.

The results show that RACR-HP have crythroid and myeloid potency, and have the most potency early in the differentiation process at Day 9 compared to Day 12 or Day 15.

These data indicate that beyond just making iCILs, RACR-HP can make many more blood cell types, all of which have potential in clinical applications.

### Example 16: Gene Expression in CIL Cells Derived from RACR-Engineered iPSC

Gene expression of CIL cells derived from RACR-engineered iPSC (iCIL) was examined (RACR-iCIL).

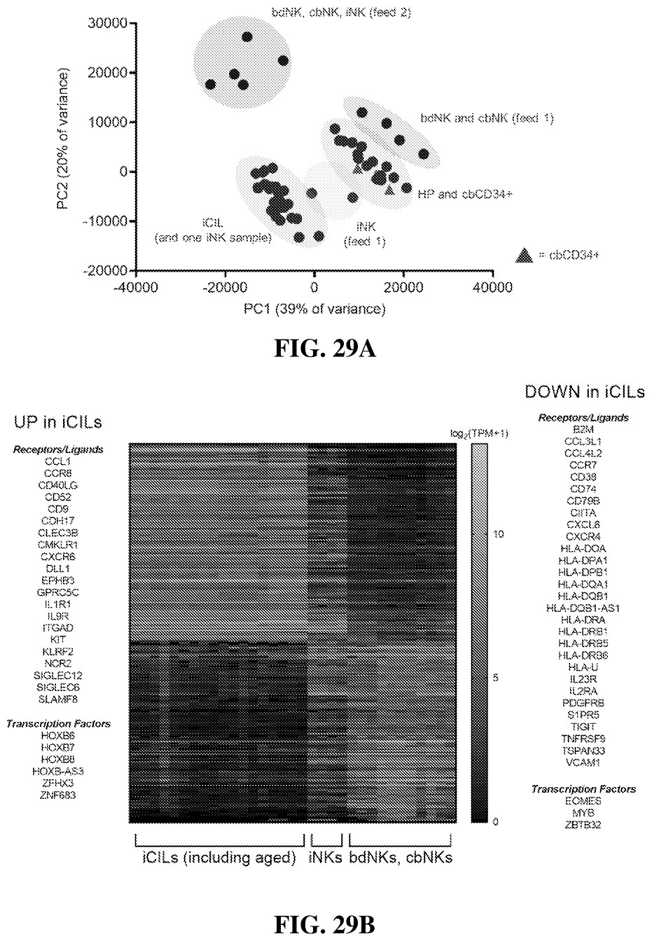

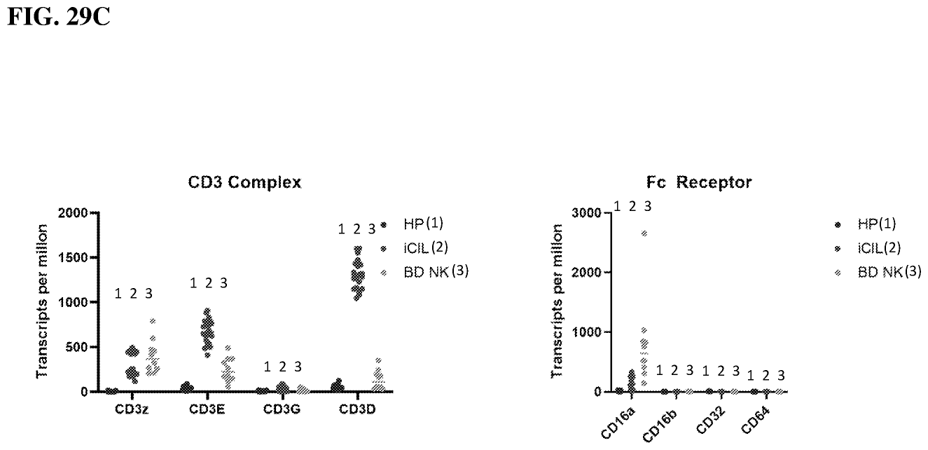

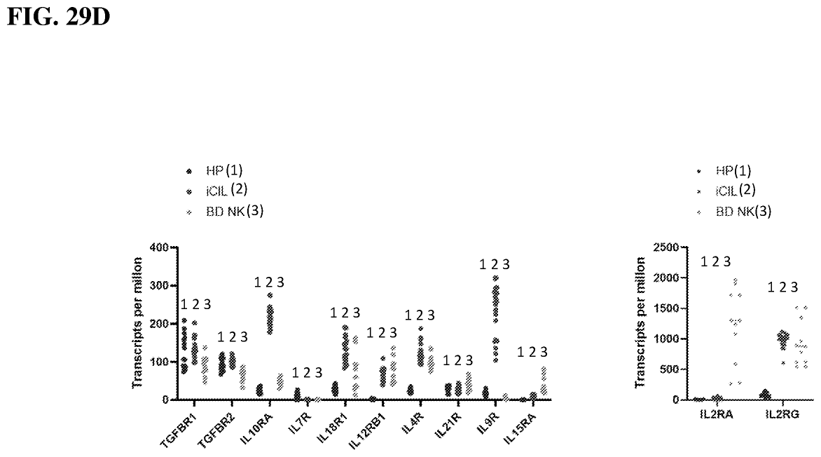

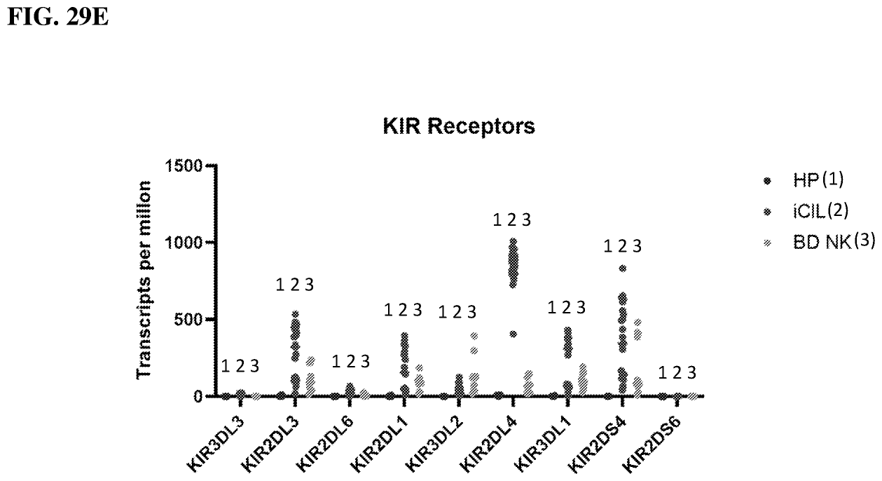

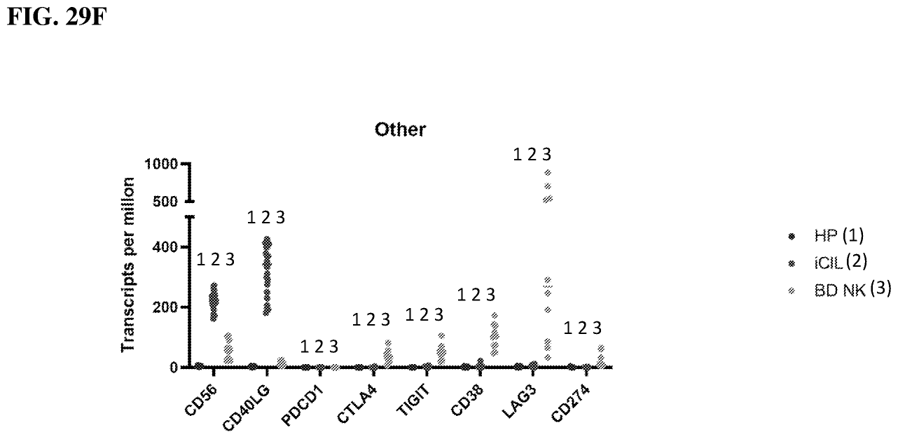

The iCILs were derived from iPSCs containing the B2M-EF1a-RACR KI and FKBP12 KO edits using Versions 3-5 described in Example 13 above and were harvested on days 40-80 for RNA isolation. For the bdNKs and chNKs, cells were obtained from commercial sources and then cocultured with feeder cells (irradiated K562 cells modified to express 41BBL and membrane-bound IL21, i.e., 41BB/mbIL21-K562 cells). Feeder cells are traditionally used in most NK cultures (including NK cells derived from iPSCs) and work by stimulating the NK cells to survive and proliferate via their innate receptors and modifications on the K562 cells. In FIG. 29A, feed 1 and feed 2 refer to the number of times the NK cells were co-cultured with the feeder cells. The iNKs were derived from iPSCs containing no genetic modifications using the STEMdiff NK kit (Stem Cell Technologies) and differentiating up to Day 26. The iNKs were then purified and expanded using irradiated 41BB/mbIL21-K562 cells as feeder cells.

RNAseq was carried out for gene expression on the complementary DNA (cDNA) samples prepared from RNA isolated from RACR-iCILs and on RNA isolated from blood-derived NK cells (bdNK), NK cells from cord blood (cbNK), and iNK differentiated from iPSCs

Evaluation of RNA-seq data by prinicipal component analysis (PCA) revealed a unique gene expression profile of RACR-iCILs (FIG. 29A). FIG. 29B provides a heatmap of gene expression of genes upregulated in iCILs and down regulated in iCILs and their differential expression compared to gene expression in iNKs, bdNKs and cbNKs. FIGS. 29C-F shows gene expression of specific receptors and inhibitory checkpoints in RACR-iCILS compared to HP or bdNK cells. These results are consistent with a finding that RACR-iCILs display a gene signature that was distinct from NK cells.

In a separate study examining the phenotypic differences between RACR-iCIL cells and iNK, bdNK or cbNK cells, RACR-iCIL were generated according to Example 2, bdNK cells were generated according to Example 1, and iNK cells were generated using the differentiation kit from Stem Cell Technologies up to Day 26, and then were purified and expanded using the irradiated feeder cells as described above.

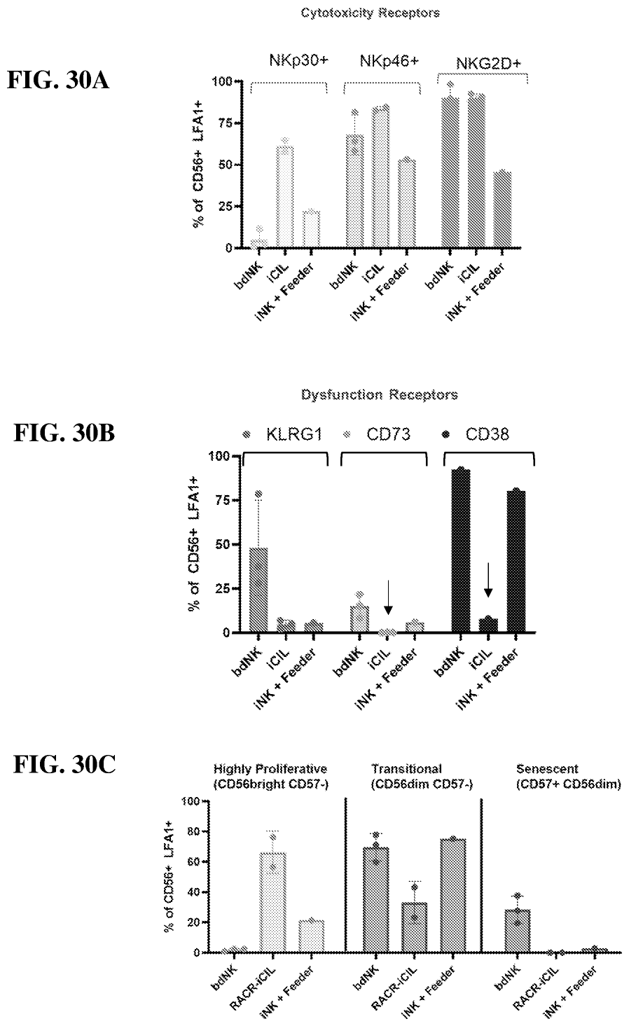

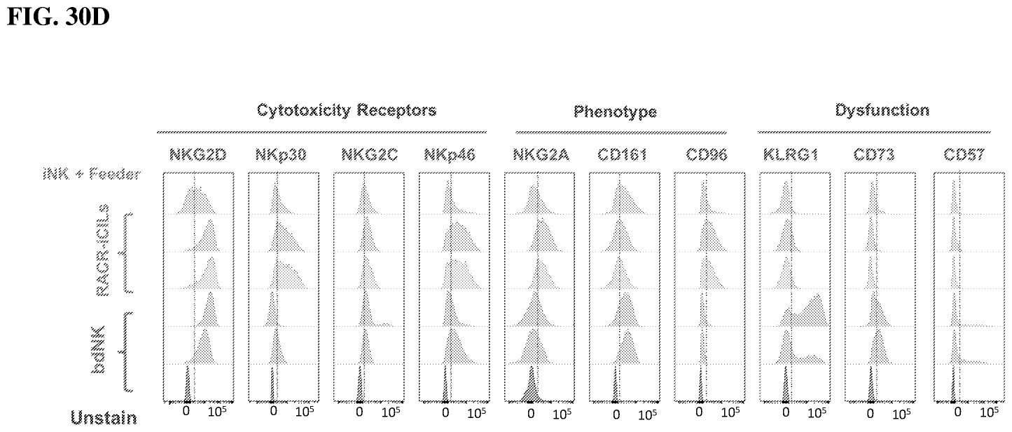

FIG. 30A depicts cytotoxicity receptor markers, FIG. 30B depicts dysfunction markers, FIG. 30C depicts proliferative, transitional and senescent markers, and FIG. 30D depicts cytotoxicity receptor markers, phenotype and dysfunction markers.

FIG. 30A shows that a greater percentage of RACR-iCIL cells express cytotoxicity receptors NKp30+ and NKp46 compared to bdNK and iNK (+feeder). A greater percentage of RACR-iCIL cells also express NKG2D compared to iNK (+feeder). FIG. 30B shows that dysfunction receptor KLRG1 was substantially lower in RACR-iCIL cells and iNK (+feeder) compared to bdNK. Dysfuntion receptor CD73 was lower in RACR-iCIL cells compared to bdNK and iNK (+feeder). Dysfunction receptor CD38 was substantially lower in RACR-iCIL cells compared to bdNK and iNK (+feeder). The absence of CD38 expression highlights an opportunity to combine iCILs with CD38 targeting mAbs. Additionally, the absence of CD38 and CD73 indicate that iCILs exhibit higher metabolic fitness based on phenotype.

FIG. 30C shows that RACR-iCIL cells are highly proliferative, less transitional and non-senescent compared to bdNK and iNK+feeder. FIG. 30D shows that iCIL cells exhibit a highly cytotoxic phenotype in comparison to feeder-expanded iNK and bdNK cells.

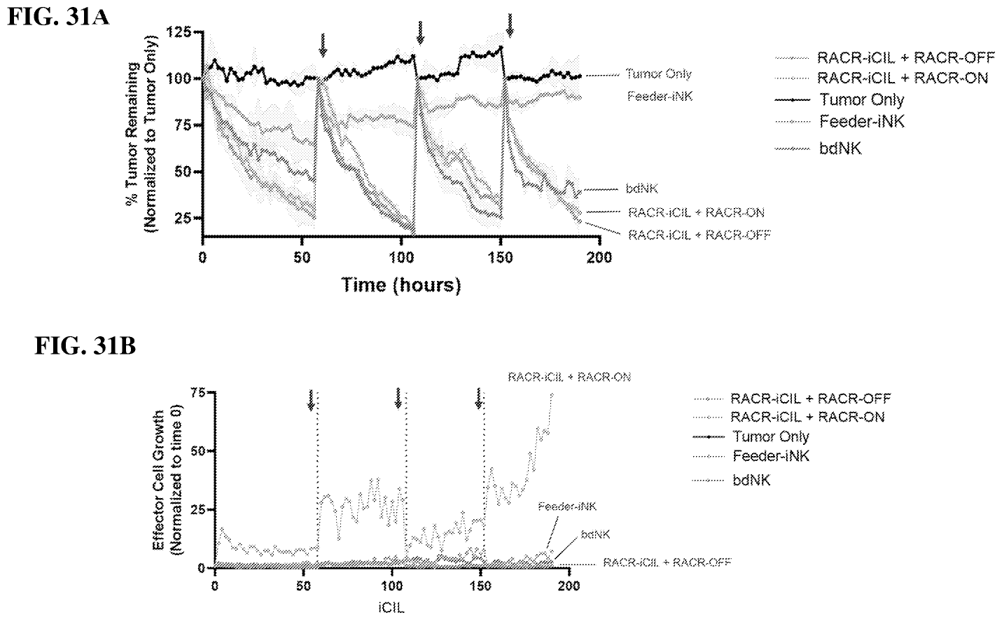

A separate study was conducted to determine whether the above differences in cytotoxicity and proliferation markers resulted in improved RACR-iCIL proliferation and tumor cell killing. Effector cells (RACR-iCIL, bdNK, iNK expanded with feeder cells) were cocultured with MDA-MB-231 cells at a ratio of 2.5:1 and stimulated with cytokines. Cytotoxicity was determined by imaging cells across 200 hours and set to 15% for total tumor clearance in images. The increase in cytotoxicity markers led to superior tumor cell killing compared to feeder expanded iNK cells and bdNK cells within the first 50 hours of a serial killing assay (FIG. 31A). The increase in proliferation markers also led to superior iCIL proliferation compared to feeder expanded iNK cells and bdNK cells up to 200 hours (FIG. 31B).

These data indicate that the harvested iCILs are a highly pure population of cells that express receptors associated with NK cytotoxicity. Functionally, RACR-iCILs demonstrated potent cytotoxicity against solid tumor cells, and activation of RACR enhanced the cells' ability to clear tumor cells, much like potency-enhancing cytokines. In fact, in serial killing assays, the RACR-iCILs not only strongly outperformed conventional NKs derived from iPSCs, but rivaled blood-derived NKs, a “gold standard” for NK function. Table E3 summarizes the unique attributes of RACR-iCILs.

TABLE E3

| Characteristic | iPSC-NK | Blood NK | iCIL |
|---|---|---|---|
| Cytotoxicity | + | ++ | +++ |
| Receptor | | | |
| Expression | | | |
| (e.g., NKG2D, | | | |
| NKp46, NKp30, | | | |
| DNAM1, NKG2c) | | | |
| Memory Potential | − | − | ++ |
| (e.g., NKp46, | | | |
| NKG2A CD57) | | | |
| Metabolic Fitness | + | − | +++ |
| (e.g., CD38, CD73) | | | |
| Tumor Cytotoxicity | + | ++ | ++++ |
| (in vitro Tumor | | | |
| Clearance) | | | |
| Persistence | − | ++ | +++ |
| Potential (Serial | | | |
| killing and | | | |
| Proliferation) | | | |
| Low Inhibitory | − | − | +++ |
| Receptor | | | |
| Expression (e.g., | | | |
| TIGIT, LAG3, | | | |
| CTLA4, PDL1) | | | |
| Controlled in vivo | − | − | +++ |
| Fitness | | | |
| (Rapamycin | | | |
| Enhanced Potency) | | | |

### Example 17: Assessment of Conditions for Generation of Hematopoietic Cells Derived from RACR-Engineered Hematopoietic Stem Cells

The protocol for hematopoietic cells derived from RACR-engineered hematopoietic stem cell generation was further investigated by changing cell culture media components and protocol timing, as an extension of methods outlined in Example 13. As described in Example 13, various aspects of the iCIL differentiation protocol were found to increase iCIL yields, purity and cytotoxicity.

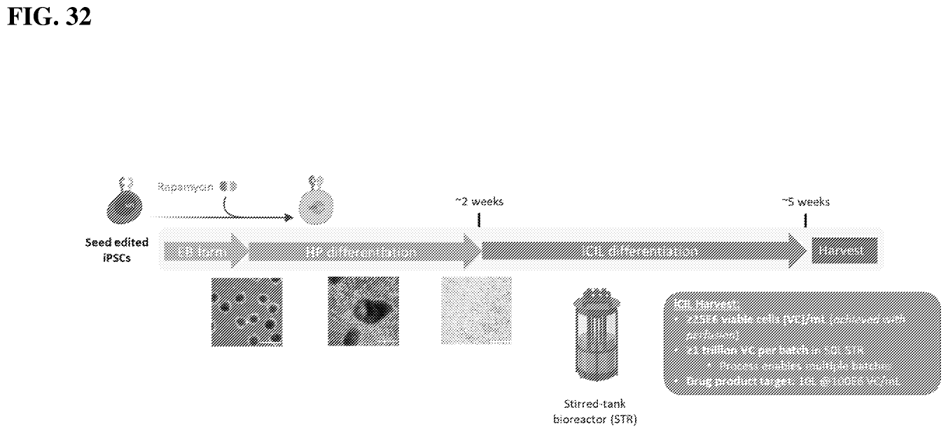

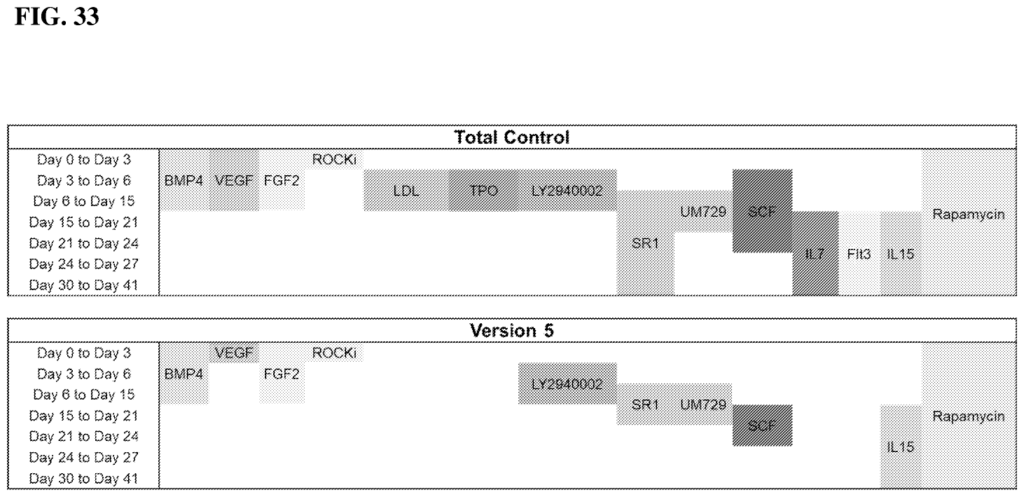

Based on experiments designed below, an exemplary protocol designated “version 5” was developed as shown in FIG. 32 and FIG. 33. In FIG. 33, factors used in the “version 5” process are depicted as compared to a Total control “version 3” protocol described in Example 13.

### EB/iPSC Formation (Days 0-3)

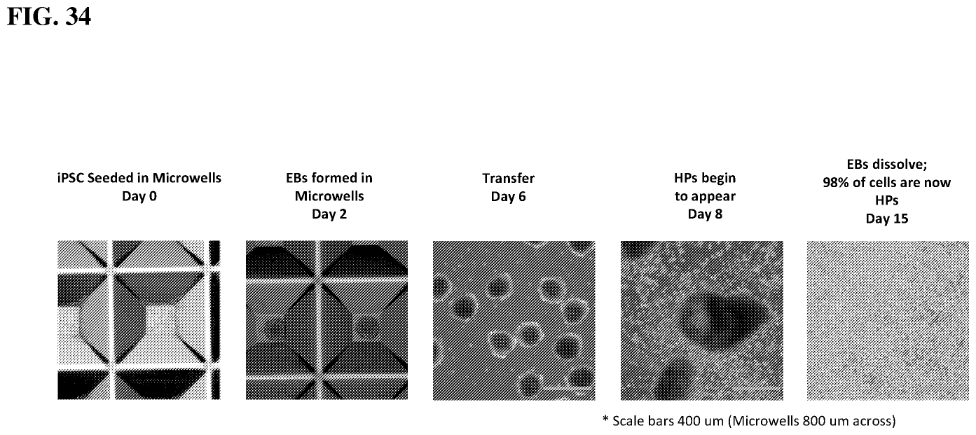

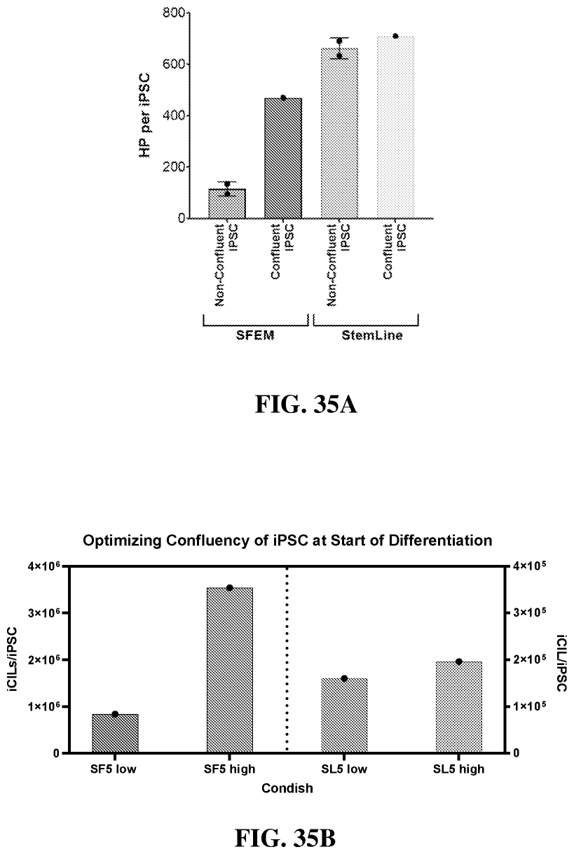

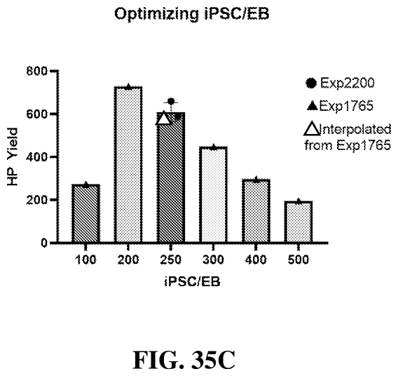

RACR iPSCs (edited B2M-EF1a-RACR and knocked out for FKBP12) were cultured in media, as described in Example 9. At day 0-2, embryoid bodies (EBs) were generated from a single cell suspension of iPSCs in STEMdiff APEL 2 Medium (STEMCELL Technologies) supplemented with BMP4, FGF2, VEGF, Y27632, and +/− rapalog (RL; AP21967) or +/− rapamycin (RM). EBs were formed using plates comprising aggregates of microwells (e.g. Aggrewell™ 800 plates) at 500 iPSC/EB or 1000 iPSC/EB, as described in Example 9 and shown in FIG. 34. As shown in FIG. 35A-B, higher iPSC confluency at the start of differentiation contributes to a higher HP yield. Further, a range of 200-250 iPSC/EBs seeded in microwells is optimal to achieve the highest HP yield on day 15 (FIG. 35C).

### HP Differentiation (Days 3-15)

### Media Components

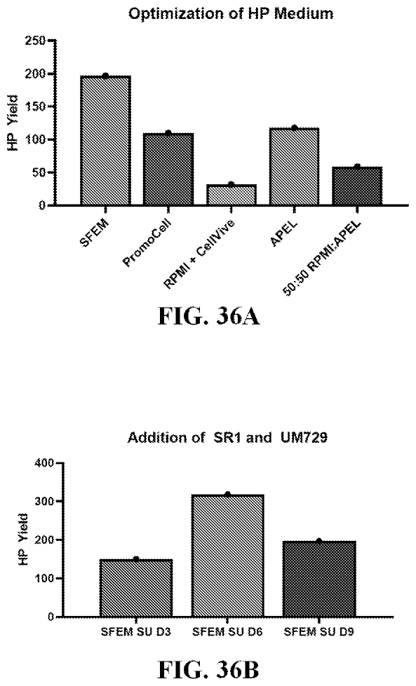

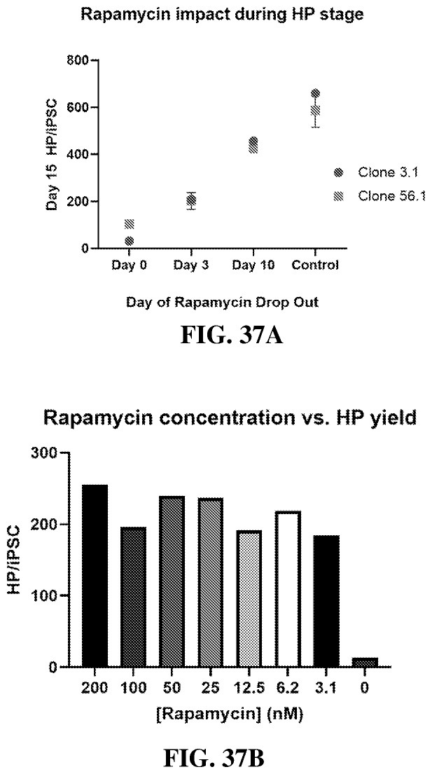

The differentiating EBs were moved to non-tissue culture treated 6-well plates (Corning) at day 6 and fresh HP Medium was added. Every 3 days from days 3-15, half of the media was replaced with HP Medium. As shown in FIG. 36A, culturing in SFEM medium (StemSPAN SFEM II Medium (STEMCELL Technologies) on days 3-12 provided the highest HP yield. HPs were defined as CD34+/CD43+/CD45+ cells as determined by flow cytometry. Further, addition of small molecules SR1 and UM729 on day 6, as opposed to day 9, drastically improved HP yield (FIG. 36B). Continuous addition of rapanycim or rapalog during the differentiation phase was necessary for high HP yields (FIG. 37A), as dropping rapamycin on day 0, 3, or 10 drastically reduced HP yield compared to the control. However, concentrations as low as 3.1 nM rapamycin were sufficient to produce comparable HP yields, suggesting the concentration of rapamycin can be drastically reduced lower than 100 nM rapamycin (FIG. 37B).

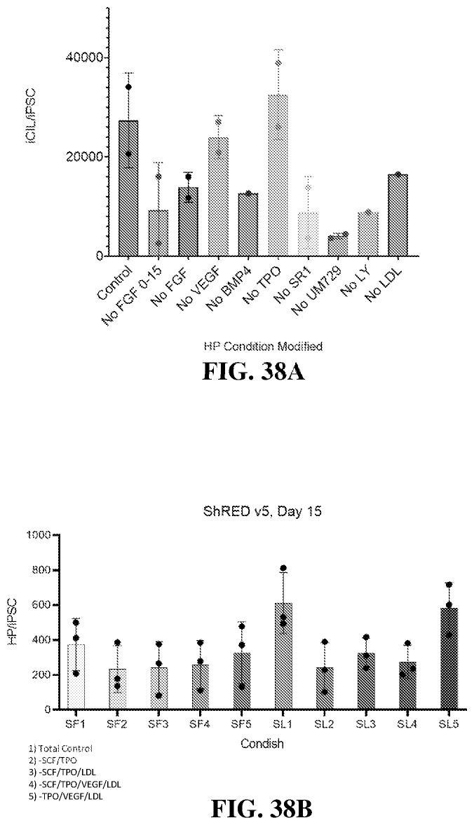

In one experiment, optimization of cell culture components and supplements during the HP differentiation stage was tested. The removal of TPO and VEGF during the HP differentiation stage increased iCIL yield at day 33, while removal of UM729, SR1, BMP4 and FGF during the differentiation stage greatly decreased iCIL purity and total cells (FIG. 38A) at day 33. Media components were then tested in combination. Removal of TPO, VEGF, and LDL together did not significantly affect HP yield at Day 15, as compared to the total control (FIG. 38B, also see FIG. 17A in Example 9). Thus, these results support that the SFEM medium used during the HP differentiation be supplemented with BMP4, FGF2. LY2940002 (PI3K inhibitor), with the addition of UM729 and SR1 beginning at Day 6 (FIG. 33).

iCIL Progenitors, Differentiation, and Maturation (Days 15-40)

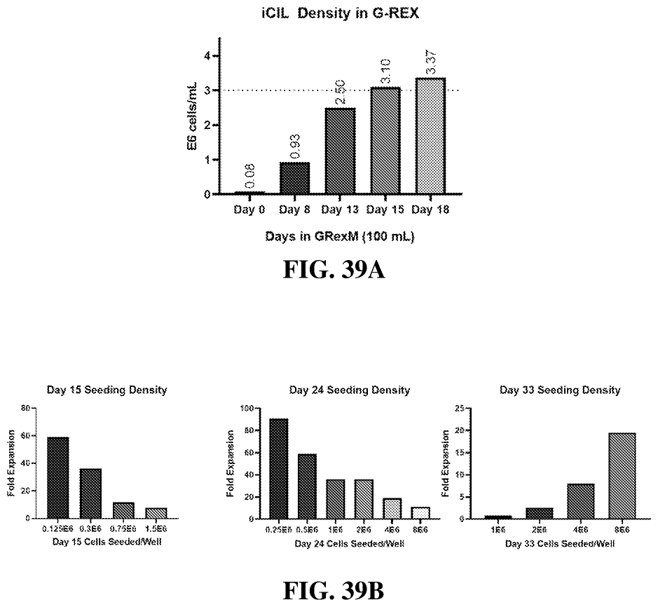

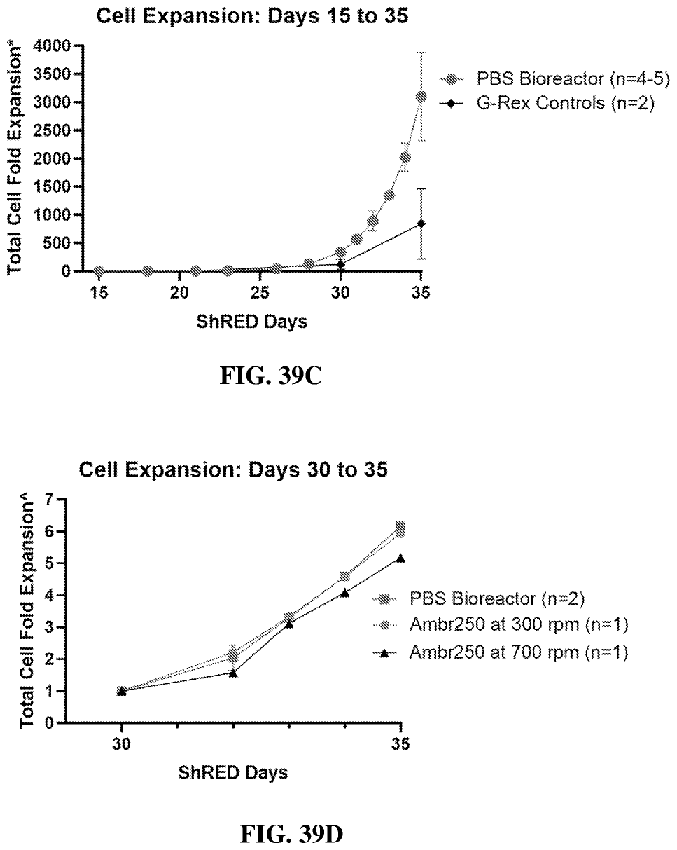

At Day 15 of culture, the differentiated cells were continued in culture for up to 40 days for iCIL cell generation. At Day 21, the GRex bioreactor was seeded with the cells, as described in Example 13. As shown in FIG. 39A, by Day 40 of differentiation, 3.37E6 cells/mL were generated utilizing the GRex bioreactor beginning at Day 15. Lower seeding densities on days 15 and 24 contributed to increased iCIL yields, while higher seeding densities contributed to higher iCIL yields on Days 30-33 (FIG. 39B). In another experiment, this step was repeated with two different stirred-tank bioreactors at various stages in the protocol. The use of the stirred-tank bioreactors dramatically increased iCIL yield, and allowed for the harvest of cells on Day 35, as opposed to Day 40 (FIG. 39C-D).

### Media Components

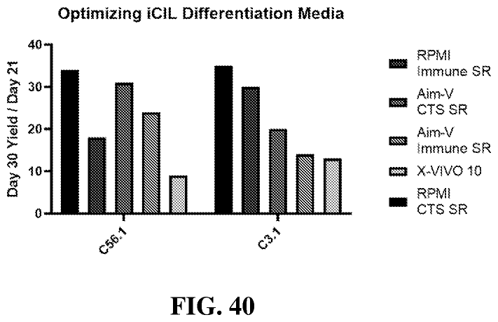

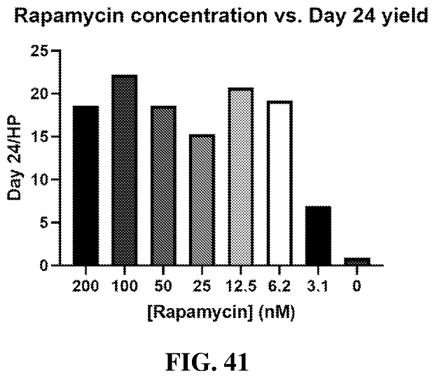

In two different clones tested for the iCIL differentiation phase, utilization of Aim V medium, as compared to RPMI medium did not affect iCIL yield (FIG. 40). Thus, Aim V was used as the base medium for the iCIL differentiation phase. Components and supplements in the Aim V medium used during the iCIL differentiation phase were tested for their effect on iCIL yield and cytotoxicity. Concentrations as low as 6.2 nM rapamycin were sufficient to expand iCIL cells, suggesting the concentration of rapamycin can be drastically reduced from the previous concentration of 100 nM rapamycin (FIG. 41).

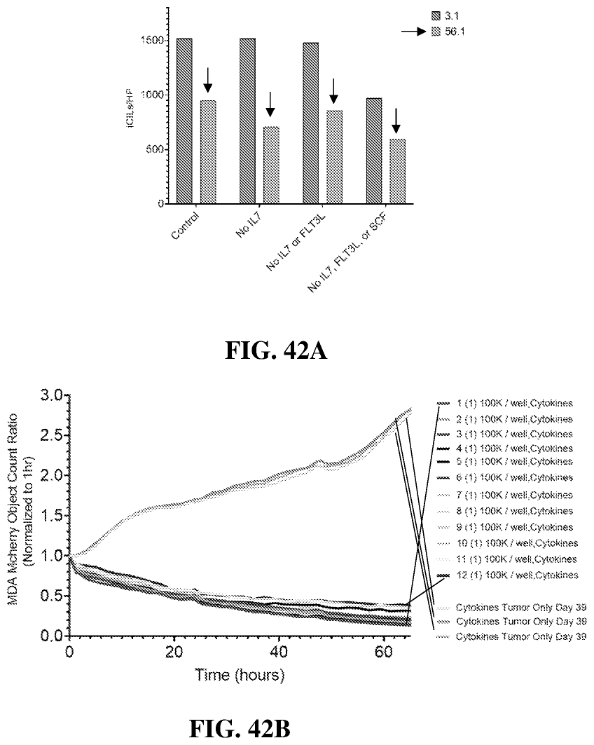

Similar to results shown in Example 13, removal of cytokines IL7 and FLT3L at iCIL differentiation phase did not significantly affect iCIL yield at Day 39, while cytotoxicity of the mature iCIL cells was improved with the removal of both cytokines (FIG. 42A-B).

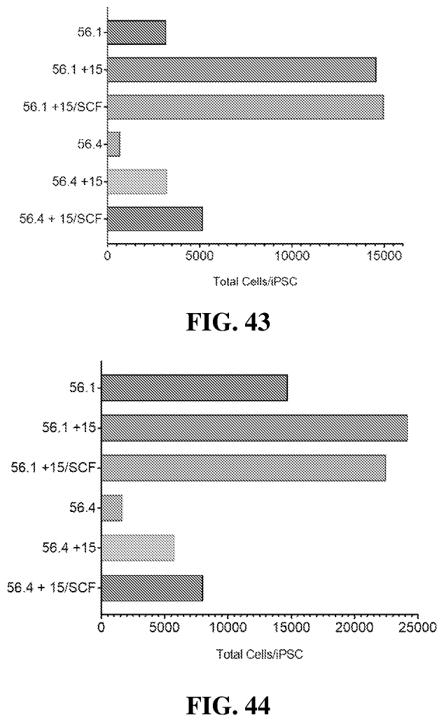

In another experiment, addition of SCF and IL-15 into the medium at Day 15 improved iCIL yield (FIG. 43. However, IL-15 alone was sufficient to continue iCIL generation and maturation post-Day 24, allowing for the removal of SCF past Day 24 (FIG. 44.

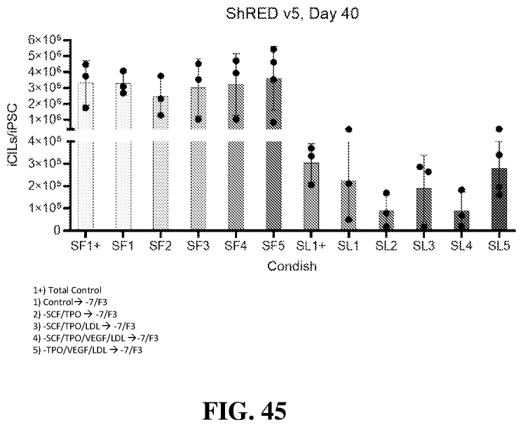

To test the effect of the cytokine removals from the HP differentiation phase with the iCIL generation/maturation phase on final iCIL yield, conditions of both differentiation phases were tested in various combinations. As shown in FIG. 45, analysis at Day 40 shows that removal of VEGF/TPO/LDL/SCF (condition/condish 4) or the same conditions with the inclusion of SCF (condish 5) at the HP phase plus IL7/FL3 removal at the iCIL phase maintained iCIL/iPSC yields that are equal to the controls.

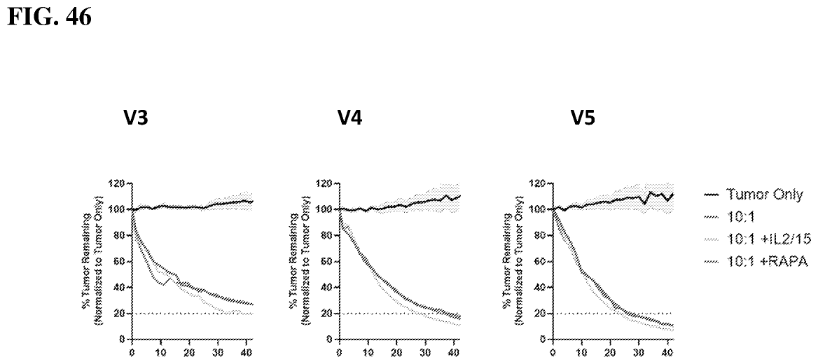

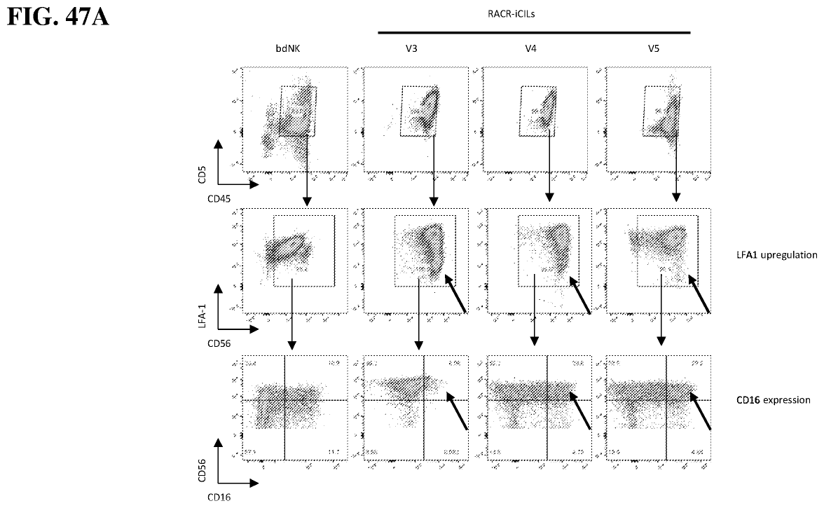

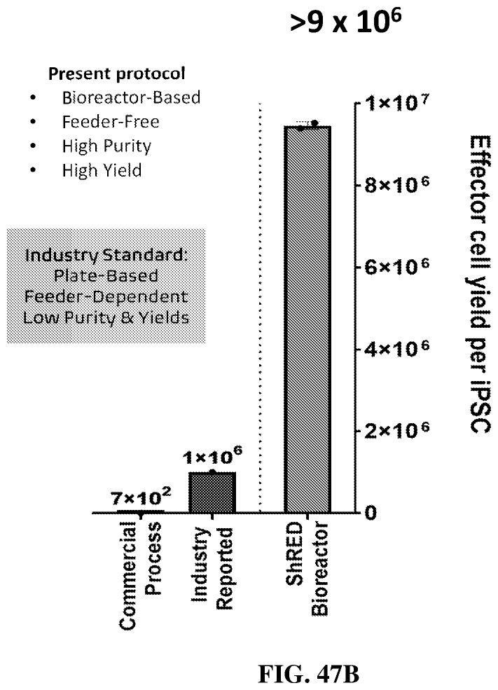

iCIL cells generated using the protocol described above were analyzed for functionality utilizing an incucyte cytotoxicity assay against triple-negative breast cancer line MDA MB 231, as described in Example 10 (FIG. 46). iCIL cells generated using the protocol described above showed at least a 15% decrease in percent tumor remaining as compared to an alternative differentiation protocol that includes addition of LDL/TPO/IL7/Flt3, demonstrating the protocol progression improved cytotoxicity of RACR-iCILs. Further, RACR-iCIL cells generated using the protocol described above demonstrated an upregulation in LFA-1 and CD16 expression (FIG. 47A) Use of the protocol described above allowed for the removal of seven different growth factors and cytokines as compared to the conventional differentiation/expansion protocol (FIG. 33). Further, use of the protocol described above resulted in greater than a 9 million-fold expansion of highly-pure iCILs that were differentiated entirely in dynamic bioreactors as a feeder-free process (FIG. 47B).

### Example 18: Assessment of RACR and CAR-Engineered CILs Cytotoxic Killing Activity in Hemetologic and Solid Tumor Cells

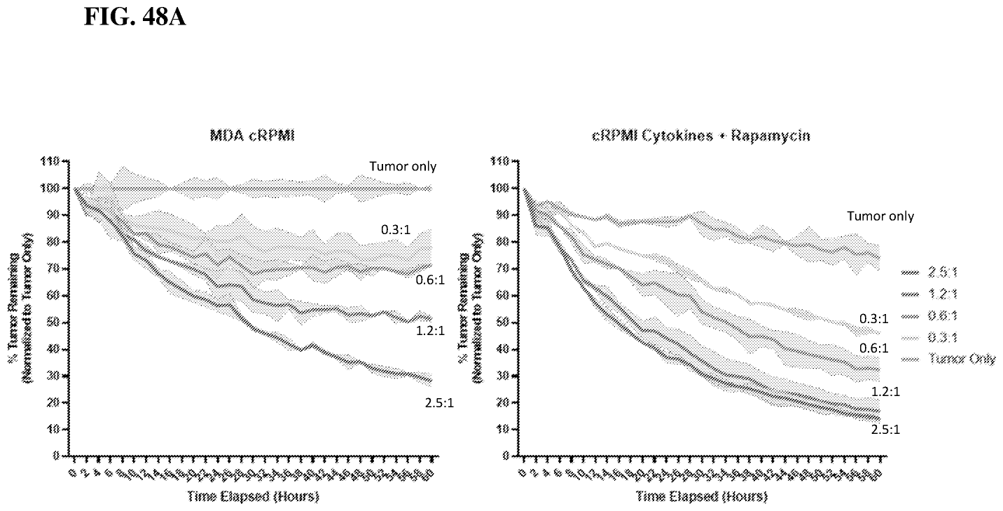

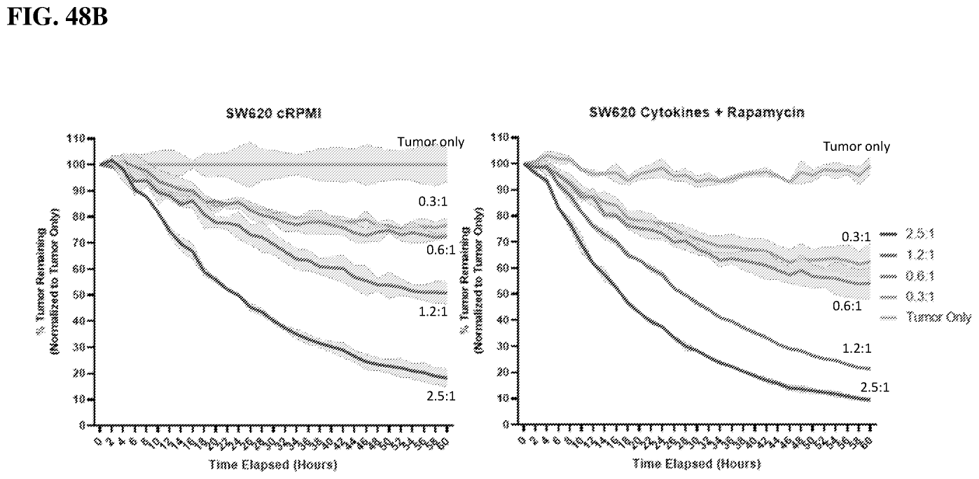

A study was conducted to determine the effect of RACR-iCIL cells on various tumor cell lines. RACR-iCIL cells were generated as described in Example 17. RACR-iCIL were then cocultured with MDA-MB-231 (FIG. 48A) or SW620 cells (FIG. 48B) at a ratio of 2.5:1. 1.2:1, 0.6:1, 0.3:1, or 0:1, and stimulated with and without rapamycin and/or cytokines. Cytotoxicity was determined by imaging cells across 60-70 hours, as described in Example 6.

RACR-iCILs demonstrated improved tumor killing with rapamycin and cytokine stimulation (FIG. 48A-B).

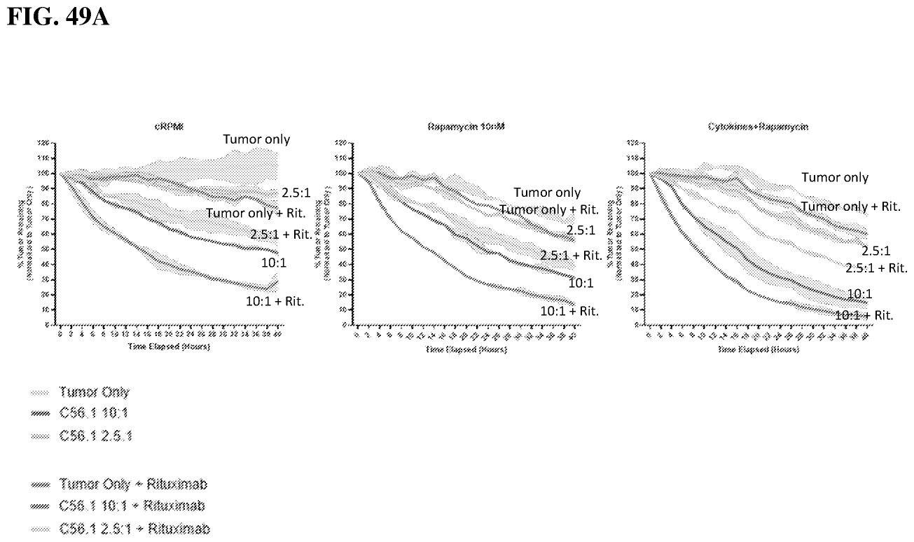

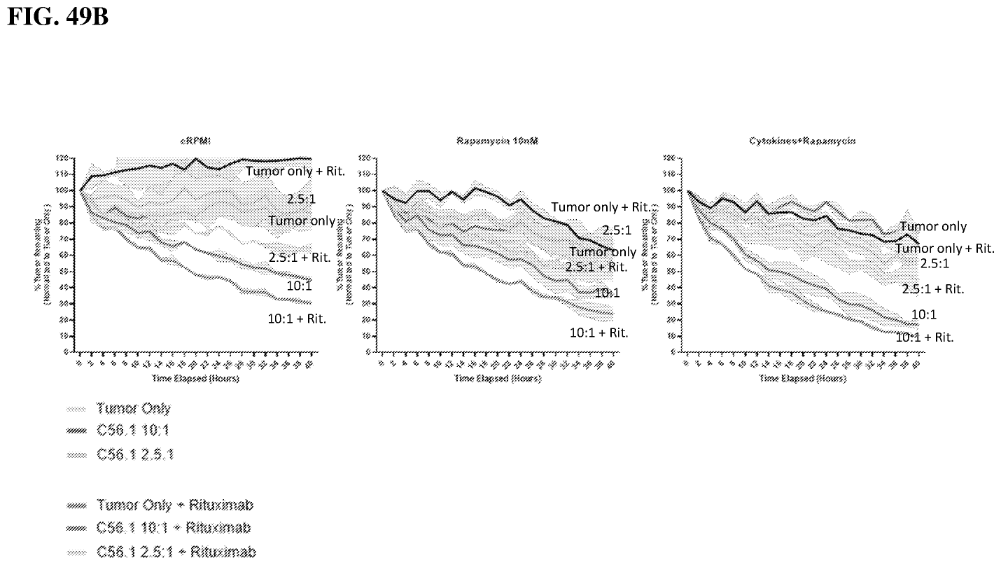

In a similar experiment, RACR-iCILs were cocultured with Burkitt's lymphoma tumor cells or non-Hodgkin's lymphoma tumor cells at a ratio of 2.5:1, 10:1, or 0:1, and stimulated with and without rapamycin and/or cytokines. Additional groups included rituximab. Cytotoxicity was determined by imaging cells across 40 hours, as described in Example 6. RACR-iCILs demonstrated improved tumor killing with rapamycin and cytokine stimulation, while addition of rituximab further increased tumor killing by 10% (FIG. 49A-B).

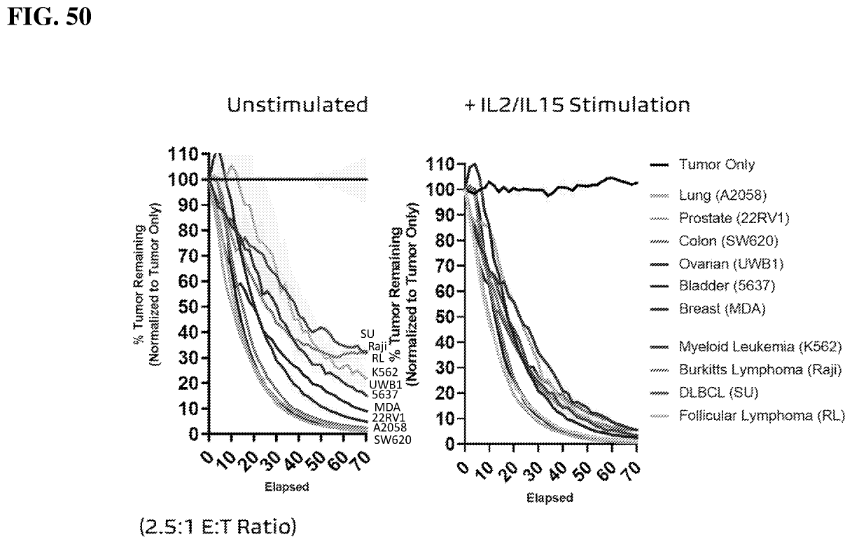

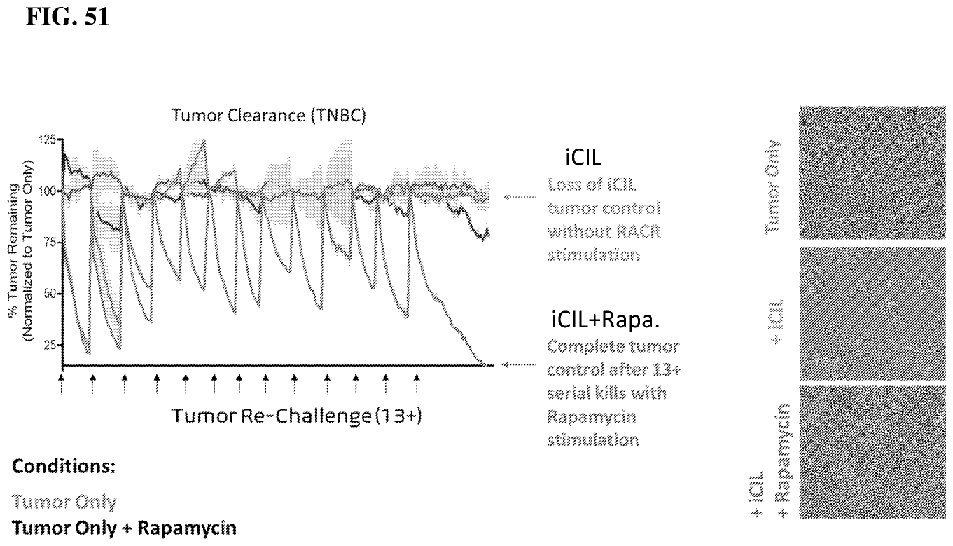

In a similar experiment, RACR-iCILs were cocultured with tumor cells from 10 tumor cell lines at a 2.5:1 ratio and stimulated with rapamycin and cytokines. RACR-iCILs demonstrated improved tumor killing with rapamycin and cytokine stimulation compared to the tumor only control (FIG. 50).Further, addition of RACR enhanced iCIL potency in an experiment related to serial kills, as described in Example 10 (FIG. 51).

This data demonstrates the superior ability of RACR-iCIL stimulation to support tumor killing, while supporting the potential for improved cytotoxicity in combination therapy.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

TABLE OF
SEQUENCES

| SEQ ID NO: | Sequence | Description |
|---|---|---|
| 1 | ERTMPRIPTLKNLEDLVTEYHGNFSAWSGVSKGLAE | IL2RG |
| | SLQPDYSERLCLVSEIPPKGGALG | Common |
| | EGPGASPCNQHSPYWAPPCYTLKPET | Gamma |
| | | Chain |
| | | Intracellular |
| | | domain |
| 2 | NCRNTGPWLKKVLKCNTPDPSKFFSQLSSEHGGDVQ | IL2RB |
| | KWLSSPFPSSSFSPGGLAPEISPL | intracellular |
| | EVLERDKVTQLLLQQDKVPEPASLSSNHSLTSCFTN | domain |
| | QGYFFFHLPDALEIEACQVYFTYD | |
| | PYSEEDPDEGVAGAPTGSSPQPLQPLSGEDDAYCTFP | |
| | SRDDLLLFSPSLLGGPSPPSTAP | |
| | GGSGAGEERMPPSLQERVPRDWDPQPLGPPTPGVPD | |
| | LVDFQPPPELVLREAGEEVPDAGP | |
| | REGVSFPWSRPPGQGEFRALNARLPLNTDAYLSLQE | |
| | LQGQDPTHLV | |
| 3 | KKRIKPIVWPSLPDHKKTLEHLCKKPRKNLNVSFNPE | IL7RB |
| | SFLDCQIHRVDDIQARDEVEGFL | intracellular |
| | QDTFPQQLEESEKQRLGGDVQSPNCPSEDVVITPESF | domain |
| | GRDSSLTCLAGNVSACDAPILSS | |
| | SRSLDCRESGKNGPHVYQDLLLSLGTTNSTLPPPFSL | |
| | QSGILTLNPVAQGQPILTSLGSN | |
| | QEEAYVTMSSFYQNQ | |
| 4 | SLKTHPLWRLWKKIWAVPSPERFFMPLYKGCSGDF | IL21RB |
| | KKWVGAPFTGSSLELGPWSPEVPST | intracellular |
| | LEVYSCHPPRSPAKRLQLTELQEPAELVESDGVPKPS | domain |
| | FWPTAQNSGGSAYSEERDRPYGL | |
| | VSIDTVTVLDAEGPCTWPCSCEDDGYPALDLDAGLE | |
| | PSPGLEDPLLDAGTTVLSCGCVSA | |
| | GSPGLGGPLGSLLDRLKPPLADGEDWAGGLPWGGR | |
| | SPGGVSESEAGSPLAGLDMDTFDSG | |
| | FVGSDCSSPVECDFTSPGDEGPPRSYLRQWVVIPPPL | |
| | SSPGPQAS | |
| 5 | GVQVETISPGDGRTFPKRGQTCVVHYTGMLEDGKK | FKBP |
| | FDSSRDRNKPFKFMLGKQEVIRGWE | domain |
| | EGVAQMSVGQRAKLTISPDYAYGATGHPGIIPPHAT | (FKBP12) |
| | LVFDVELLKLE | |
| 6 | ILWHEMWHEGLEEASRLYFGERNVKGMFEVLEPLH | FRB domain |
| | AMMERGPQTLKETSFNQAYGRDLME | |
| | AQEWCRKYMKSGNVKDLTQAWDLYYHVFRRISK | |
| 7 | ILWHEMWHEGLEEASRLYFGERNVKGMFEVLEPLH | variant FRB |
| | AMMERGPQTLKETSFNQAYGRDLME | domain (FRB |
| | AQEWCRKYMKSGNVKDLLQAWDLYYHVFRRISK | mutant |
| | | domain) |
| 8 | VVISVGSMGLIISLLCVYFWL | transmembrane |
| | | (TM) |
| | | domain |
| 9 | VAVAGCVFLLISVLLLSGL | TM domain |
| 10 | PILLTISILSFFSVALLVILACVLW | TM domain |
| 11 | GWNPHLLLLLLLVIVFIPAFW | TM domain |
| 12 | MALPVTALLLPLALLLHAARP | CD8a signal |
| | | sequence |
| 13 | ATGGCCTTACCAGTGACCGCCTTGCTCCTGCCGCT | nucleotide |
| | GGCCTTGCTGCTCCACGCCGCCAGGCCGGATGTCG | sequence |
| | TGATGACCCAGACCCCCCTCAGCCTCCCAGTGTCC | encoding a |
| | CTCGGTGACCAGGCTTCTATTAGTTGCAGATCCAG | CAR |
| | CCAGTCCCTCGTGCACTCTAACGGTAATACCTACC | |
| | TGAGATGGTATCTCCAGAAGCCCGGACAGAGCCC | |
| | TAAGGTGCTGATCTACAAAGTCTCCAACCGGGTGT | |
| | CTGGAGTCCCTGACCGCTTCTCAGGGAGCGGTTCC | |
| | GGCACCGACTTCACCCTGAAGATCAACCGGGTGG | |
| | AGGCCGAAGACCTCGGCGTCTATTTCTGCTCTCAG | |
| | AGTACACATGTGCCCTGGACCTTCGGCGGAGGGA | |
| | CCAAGCTGGAGATCAAAAGCTCCGCAGACGATGC | |
| | CAAGAAAGATGCCGCTAAGAAAGACGATGCTAAG | |
| | AAAGACGATGCAAAGAAAGACGGTGGCGTGAAG | |
| | CTGGATGAAACCGGAGGAGGTCTCGTCCAGCCAG | |
| | GAGGAGCCATGAAGCTGAGTTGCGTGACCAGCGG | |
| | ATTCACCTTTGGGCACTACTGGATGAACTGGGTGC | |
| | GACAGTCCCCAGAGAAGGGGCTCGAATGGGTCGC | |
| | TCAGTTCAGGAACAAACCCTACAATTATGAGACA | |
| | TACTATTCAGACAGCGTGAAGGGCAGGTTTACTAT | |
| | CAGTAGAGACGATTCCAAATCTAGCGTGTACCTGC | |
| | AGATGAACAATCTCAGGGTCGAAGATACAGGCAT | |
| | CTACTATTGCACAGGGGCATCCTATGGTATGGAGT | |
| | ATCTCGGTCAGGGGACAAGCGTCACAGTCAGTTTC | |
| | GTGCCGGTCTTCCTGCCAGCGAAGCCCACCACGAC | |
| | GCCAGCGCCGCGACCACCAACACCGGCGCCCACC | |
| | ATCGCGTCGCAGCCCCTGTCCCTGCGCCCAGAGGC | |
| | GTGCCGGCCAGCGGCGGGGGGCGCAGTGCACACG | |
| | AGGGGGCTGGACTTCGCCTGTGATATCTACATCTG | |
| | GGCGCCCTTGGCCGGGACTTGTGGGGTCCTTCTCC | |
| | TGTCACTGGTTATCACCCTTTACTGCAACCACAGG | |
| | AACCGTTTCTCTGTTGTTAAACGGGGCAGAAAGA | |
| | AACTCCTGTATATATTCAAACAACCATTTATGAGA | |
| | CCAGTACAAACTACTCAAGAGGAAGATGGCTGTA | |
| | GCTGCCGATTTCCAGAAGAAGAAGAAGGAGGATG | |
| | TGAACTGAGAGTGAAGTTCAGCAGGAGCGCAGAC | |
| | GCCCCCGCGTACCAGCAGGGCCAGAACCAGCTCT | |
| | ATAACGAGCTCAATCTAGGACGAAGAGAGGAGTA | |
| | CGATGTTTTGGACAAGAGACGTGGCCGGGACCCT | |
| | GAGATGGGGGGAAAGCCGAGAAGGAAGAACCCT | |
| | CAGGAAGGCCTGTACAATGAACTGCAGAAAGATA | |
| | AGATGGCGGAGGCCTACAGTGAGATTGGGATGAA | |
| | AGGCGAGCGCCGGAGGGGCAAGGGGCACGATGG | |
| | CCTTTACCAGGGTCTCAGTACAGCCACCAAGGAC | |
| | ACCTACGACGCCCTTCACATGCAGGCCCTGCCCCC | |
| | TCGCTAA | |
| 14 | MALPVTALLLPLALLLHAARPDVVMTQTPLSLPVSL | CAR amino |
| | GDQASISCRSSQSLVHSNGNTYLR | acid sequence |
| | WYLQKPGQSPKVLIYKVSNRVSGVPDRFSGSGSGTD | |
| | FTLKINRVEAEDLGVYFCSQSTHV | |
| | PWTFGGGTKLEIKSSADDAKKDAAKKDDAKKDDA | |
| | KKDGGVKLDETGGGLVQPGGAMKLSC | |
| | VTSGFTFGHYWMNWVRQSPEKGLEWVAQFRNKPY | |
| | NYETYYSDSVKGRFTISRDDSKSSVY | |
| | LQMNNLRVEDTGIYYCTGASYGMEYLGQGTSVTVS | |
| | FVPVFLPAKPTTTPAPRPPTPAPTI | |
| | ASQPLSLRPEACRPAAGGAVHTRGLDFACDIYIWAP | |
| | LAGTCGVLLLSLVITLYCNHRNRF | |
| | SVVKRGRKKLLYIFKQPFMRPVQTTQEEDGCSCRFP | |
| | EEEEGGCELRVKFSRSADAPAYQQ | |
| | GQNQLYNELNLGRREEYDVLDKRRGRDPEMGGKPR | |
| | RKNPQEGLYNELQKDKMAEAYSEIG | |
| | MKGERRRGKGHDGLYQGLSTATKDTYDALHMQAL | |
| | PPR | |
| 15 | GCCACCATGGCCTTACCAGTGACCGCCTTGCTCCT | nucleotide |
| | GCCGCTGGCCTTGCTGCTCCACGCCGCCAGGCCGG | insert |
| | ATGTCGTGATGACCCAGACCCCCCTCAGCCTCCCA | |
| | GTGTCCCTCGGTGACCAGGCTTCTATTAGTTGCAG | |
| | ATCCAGCCAGTCCCTCGTGCACTCTAACGGTAATA | |
| | CCTACCTGAGATGGTATCTCCAGAAGCCCGGACA | |
| | GAGCCCTAAGGTGCTGATCTACAAAGTCTCCAACC | |
| | GGGTGTCTGGAGTCCCTGACCGCTTCTCAGGGAGC | |
| | GGTTCCGGCACCGACTTCACCCTGAAGATCAACCG | |
| | GGTGGAGGCCGAAGACCTCGGCGTCTATTTCTGCT | |
| | CTCAGAGTACACATGTGCCCTGGACCTTCGGCGGA | |
| | GGGACCAAGCTGGAGATCAAAAGCTCCGCAGACG | |
| | ATGCCAAGAAAGATGCCGCTAAGAAAGACGATGC | |
| | TAAGAAAGACGATGCAAAGAAAGACGGTGGCGTG | |
| | AAGCTGGATGAAACCGGAGGAGGTCTCGTCCAGC | |
| | CAGGAGGAGCCATGAAGCTGAGTTGCGTGACCAG | |
| | CGGATTCACCTTTGGGCACTACTGGATGAACTGGG | |
| | TGCGACAGTCCCCAGAGAAGGGGCTCGAATGGGT | |
| | CGCTCAGTTCAGGAACAAACCCTACAATTATGAG | |
| | ACATACTATTCAGACAGCGTGAAGGGCAGGTTTA | |
| | CTATCAGTAGAGACGATTCCAAATCTAGCGTGTAC | |
| | CTGCAGATGAACAATCTCAGGGTCGAAGATACAG | |
| | GCATCTACTATTGCACAGGGGCATCCTATGGTATG | |
| | GAGTATCTCGGTCAGGGGACAAGCGTCACAGTCA | |
| | GTTTCGTGCCGGTCTTCCTGCCAGCGAAGCCCACC | |
| | ACGACGCCAGCGCCGCGACCACCAACACCGGCGC | |
| | CCACCATCGCGTCGCAGCCCCTGTCCCTGCGCCCA | |
| | GAGGCGTGCCGGCCAGCGGCGGGGGGCGCAGTGC | |
| | ACACGAGGGGGCTGGACTTCGCCTGTGATATCTAC | |
| | ATCTGGGCGCCCTTGGCCGGGACTTGTGGGGTCCT | |
| | TCTCCTGTCACTGGTTATCACCCTTTACTGCAACC | |
| | ACAGGAACCGTTTCTCTGTTGTTAAACGGGGCAGA | |
| | AAGAAACTCCTGTATATATTCAAACAACCATTTAT | |
| | GAGACCAGTACAAACTACTCAAGAGGAAGATGGC | |
| | TGTAGCTGCCGATTTCCAGAAGAAGAAGAAGGAG | |
| | GATGTGAACTGAGAGTGAAGTTCAGCAGGAGCGC | |
| | AGACGCCCCCGCGTACCAGCAGGGCCAGAACCAG | |
| | CTCTATAACGAGCTCAATCTAGGACGAAGAGAGG | |
| | AGTACGATGTTTTGGACAAGAGACGTGGCCGGGA | |
| | CCCTGAGATGGGGGGAAAGCCGAGAAGGAAGAA | |
| | CCCTCAGGAAGGCCTGTACAATGAACTGCAGAAA | |
| | GATAAGATGGCGGAGGCCTACAGTGAGATTGGGA | |
| | TGAAAGGCGAGCGCCGGAGGGGCAAGGGGCACG | |
| | ATGGCCTTTACCAGGGTCTCAGTACAGCCACCAAG | |
| | GACACCTACGACGCCCTTCACATGCAGGCCCTGCC | |
| | CCCTCGCTAA | |
| 16 | DVVMTQTPLSLPVSLGDQASISCRSSQSLVHSNGNT | CAR amino |
| | YLRWYLQKPGQSPKVLIYKVSNRVSGVPDRESGSGS | acid sequence |
| | GTDFTLKINRVEAEDLGVYFCSQSTHV | without the |
| | PWTFGGGTKLEIKSSADDAKKDAAKKDDAKKDDA | signal peptide |
| | KKDGGVKLDETGGGLVQPGGAMKLSC | |
| | VTSGFTFGHYWMNWVRQSPEKGLEWVAQFRNKPY | |
| | NYETYYSDSVKGRFTISRDDSKSSVY | |
| | LQMNNLRVEDTGIYYCTGASYGMEYLGQGTSVTVS | |
| | FVPVFLPAKPTTTPAPRPPTPAPTI | |
| | ASQPLSLRPEACRPAAGGAVHTRGLDFACDIYIWAP | |
| | LAGTCGVLLLSLVITLYCNHRNRF | |
| | SVVKRGRKKLLYIFKQPFMRPVQTTQEEDGCSCRFP | |
| | EEEEGGCELRVKFSRSADAPAYQQ | |
| | GQNQLYNELNLGRREEYDVLDKRRGRDPEMGGKPR | |
| | RKNPQEGLYNELQKDKMAEAYSEIG | |
| | MKGERRRGKGHDGLYQGLSTATKDTYDALHMQAL | |
| | PPR | |
| 17 | MALPVTALLLPLALLLHAARP | CAR amino |
| | | acid sequence |
| | | signal peptide |
| 18 | GGCCGAGATGTCTCGCTCCG | B2M gRNA |
| 19 | CACGCAGGTCTGGCCGCGCT | FKBP12 |
| | | gRNA |
| 20 | TGCTGAGCCGATGCGCGCGG | FKBP12 |
| | | gRNA |
| 21 | CTCACCGTCTCCTGGGGAGA | FKBP12 |
| | | gRNA |
| 22 | TACCCAGAGAATGGAGAAACCCTGCAGGGAATTC | B2M 5′ |
| | CCAAGCTGTAGTTATAAACAGAAGTTCTCCTTCTG | homology |
| | CTAGGTAGCATTCAAAGATCTTAATCTTCTGGGTT | arm |
| | TCCGTTTTCTCGAATGAAAAATGCAGGTCCGAGCA | |
| | GTTAACTGGCTGGGGCACCATTAGCAAGTCACTTA | |
| | GCATCTCTGGGGCCAGTCTGCAAAGCGAGGGGGC | |
| | AGCCTTAATGTGCCTCCAGCCTGAAGTCCTAGAAT | |
| | GAGCGCCCGGTGTCCCAAGCTGGGGCGCGCACCC | |
| | CAGATCGGAGGGCGCCGATGTACAGACAGCAAAC | |
| | TCACCCAGTCTAGTGCATGCCTTCTTAAACATCAC | |
| | GAGACTCTAAGAAAAGGAAACTGAAAACGGGAA | |
| | AGTCCCTCTCTCTAACCTGGCACTGCGTCGCTGGC | |
| | TTGGAGACAGGTGACGGTCCCTGCGGGCCTTGTCC | |
| | TGATTGGCTGGGCACGCGTTTAATATAAGTGGAG | |
| | GCGTCGCGCTGGCGGGC | |
| 23 | TGGCCTTAGCTGTGCTCGCGCTACTCTCTCTTTCTG | B2M 3′ |
| | GCCTGGAGGCTATCCAGCGTGAGTCTCTCCTACCC | homology |
| | TCCCGCTCTGGTCCTTCCTCTCCCGCTCTGCACCCT | arm |
| | CTGTGGCCCTCGCTGTGCTCTCTCGCTCCGTGACTT | |
| | CCCTTCTCCAAGTTCTCCTTGGTGGCCCGCCGTGG | |
| | GGCTAGTCCAGGGCTGGATCTCGGGGAAGCGGCG | |
| | GGGTGGCCTGGGAGTGGGGAAGGGGGTGCGCACC | |
| | CGGGACGCGCGCTACTTGCCCCTTTCGGCGGGGA | |
| | GCAGGGGAGACCTTTGGCCTACGGCGACGGGAGG | |
| | GTCGGGACAAAGTTTAGGGCGTCGATAAGCGTCA | |
| | GAGCGCCGAGGTTGGGGGAGGGTTTCTCTTCCGCT | |
| | CTTTCGCGGGGCCTCTGGCTCCCCCAGCGCAGCTG | |
| | GAGTGGGGGACGGGTAGGCTCGTCCCAAAGGCGC | |
| | GGCGCTGAGGTTTGTGAACGCGTGGAGGGGCGCT | |
| | TGGGGTCTGGGGGA | |
| 24 | cgtgaggctccggtgcccgtcagtgggcagagcgcacatcgcccacagtccccg | EF1alpha |
| | agaagttggggggaggggtcggcaattgaaccggtgcctagagaaggtggcgc | promoter |
| | ggggtaaactgggaaagtgatgtcgtgtactggctccgcctttttcccgagggtgg | |
| | gggagaaccgtatataagtgcagtagtcgccgtgaacgttctttttcgcaacgggttt | |
| | gccgccagaacacaggtaagtgccgtgtgtggttcccgcgggcctggcctctttac | |
| | gggttatggcccttgcgtgccttgaattacttccacgcccctggctgcagtacgtgat | |
| | tcttgatcccgagcttcgggttggaagtgggtgggagagttcgaggccttgcgctta | |
| | aggagccccttcgcctcgtgcttgagttgaggcctggcctgggcgctggggccgc | |
| | cgcgtgcgaatctggtggcaccttcgcgcctgtctcgctgctttcgataagtctctag | |
| | ccatttaaaatttttgatgacctgctgcgacgctttttttctggcaagatagtcttgtaaa | |
| | tgcgggccaagatctgcacactggtatttcggtttttggggccgcgggcggcgac | |
| | ggggcccgtgcgtcccagcgcacatgttcggcgaggcggggcctgcgagcgcg | |
| | gccaccgagaatcggacgggggtagtctcaagctggccggcctgctctggtgcct | |
| | ggcctcgcgccgccgtgtatcgccccgccctgggcggcaaggctggcccggtc | |
| | ggcaccagttgcgtgagcggaaagatggccgcttcccggccctgctgcagggag | |
| | ctcaaaatggaggacgcggcgctcgggagagcgggcgggtgagtcacccacac | |
| | aaaggaaaagggcctttccgtcctcagccgtcgcttcatgtgactccacggagtac | |
| | cgggcgccgtccaggcacctcgattagttctcgagcttttggagtacgtcgtctttag | |
| | gttggggggaggggttttatgcgatggagtttccccacactgagtgggtggagact | |
| | gaagttaggccagcttggcacttgatgtaattctccttggaatttgccctttttgagttt | |
| | ggatcttggttcattctcaagcctcagacagtggttcaaagtttttttcttccatttcagg | |
| | tgtcgtgaa | |
| 25 | CGTGAGGCTCCGGTGCCCGTCAGTGGGCAGAGCG | EF1alpha |
| | CACATCGCCCACAGTCCCCGAGAAGTTGGGGGGA | promoter |
| | GGGGTCGGCAATTGAACCGGTGCCTAGAGAAGGT | |
| | GGCGCGGGGTAAACTGGGAAAGTGATGTCGTGTA | |
| | CTGGCTCCGCCTTTTTCCCGAGGGTGGGGGAGAAC | |
| | CGTATATAAGTGCAGTAGTCGCCGTGAACGTTCTT | |
| | TTTCGCAACGGGTTTGCCGCCAGAACACAGGTAA | |
| | GTGCCGTGTGTGGTTCCCGCGGGCCTGGCCTCTTT | |
| | ACGGGTTATGGCCCTTGCGTGCCTTGAATTACTTC | |
| | CACGCCCCTGGCTGCAGTACGTGATTCTTGATCCC | |
| | GAGCTTCGGGTTGGAAGTGGGTGGGAGAGTTCGA | |
| | GGCCTTGCGCTTAAGGAGCCCCTTCGCCTCGTGCT | |
| | TGAGTTGAGGCCTGGCTTGGGCGCTGGGGCCGCC | |
| | GCGTGCGAATCTGGTGGCACCTTCGCGCCTGTCTC | |
| | GCTGCTTTCGATAAGTCTCTAGCCATTTAAAATTT | |
| | TTGATGACCTGCTGCGACGCTTTTTTTCTGGCAAG | |
| | ATAGTCTTGTAAATGCGGGCCAAGATCTGCACACT | |
| | GGTATTTCGGTTTTTGGGGCCGCGGGCGGCGACGG | |
| | GGCCCGTGCGTCCCAGCGCACATGTTCGGCGAGG | |
| | CGGGGCCTGCGAGCGCGGCCACCGAGAATCGGAC | |
| | GGGGGTAGTCTCAAGCTGGCCGGCCTGCTCTGGTG | |
| | CCTGGCCTCGCGCCGCCGTGTATCGCCCCGCCCTG | |
| | GGCGGCAAGGCTGGCCCGGTCGGCACCAGTTGCG | |
| | TGAGCGGAAAGATGGCCGCTTCCCGGCCCTGCTG | |
| | CAGGGAGCTCAAAATGGAGGACGCGGCGCTCGGG | |
| | AGAGCGGGCGGGTGAGTCACCCACACAAAGGAAA | |
| | AGGGCCTTTCCGTCCTCAGCCGTCGCTTCATGTGA | |
| | CTCCACGGAGTACCGGGCGCCGTCCAGGCACCTC | |
| | GATTAGTTCTCGAGCTTTTGGAGTACGTCGTCTTT | |
| | AGGTTGGGGGGAGGGGTTTTATGCGATGGAGTTT | |
| | CCCCACACTGAGTGGGTGGAGACTGAAGTTAGGC | |
| | CAGCTTGGCACTTGATGTAATTCTCCTTGGAATTT | |
| | GCCCTTTTTGAGTTTGGATCTTGGTTCATTCTCAAG | |
| | CCTCAGACAGTGGTTCAAAGTTTTTTTCTTCCATTT | |
| | CAGGTGTCGTGA | |
| 26 | CGTGAGGCTCCGGTGCCCGTCAGTGGGCAGAGCG | EF1alpha |
| | CACATCGCCCACAGTCCCCGAGAAGTTGGGGGGA | promoter |
| | GGGGTCGGCAATTGAACCGGTGCCTAGAGAAGGT | |
| | GGCGCGGGGTAAACTGGGAAAGTGATGTCGTGTA | |
| | CTGGCTCCGCCTTTTTCCCGAGGGTGGGGGAGAAC | |
| | CGTATATAAGTGCACTAGTCGCCGTGAACGTTCTT | |
| | TTTCGCAACGGGTTTGCCGCCAGAACACAGGTAA | |
| | GTGCCGTGTGTGGTTCCCGCGGGCCTGGCCTCTTT | |
| | ACGGGTTATGGCCCTTGCGTGCCTTGAATTACTTC | |
| | CACCTGGCTGCAGTACGTGATTCTTGATCCCGAGC | |
| | TTCGGGTTGGAAGTGGGTGGGAGAGTTCGTGGCC | |
| | TTGCGCTTAAGGAGCCCCTTCGCCTCGTGCTTGAG | |
| | TTGTGGCCTGGCCTGGGCGCTGGGGCCGCCGCGTG | |
| | CGAATCTGGTGGCACCTTCGCGCCTGTCTCGCTGC | |
| | TTTCGATAAGTCTCTAGCCATTTAAAATTTTTGAT | |
| | GACCTGCTGCGACGCTTTTTTTCTGGCAAGATAGT | |
| | CTTGTAAATGCGGGCCAAGATCAGCACACTGGTA | |
| | TTTCGGTTTTTGGGGCCGCGGGCGGCGACGGGGCC | |
| | CGTGCGTCCCAGCGCACATGTTCGGCGAGGCGGG | |
| | GCCTGCGAGCGCGGCCACCGAGAATCGGACGGGG | |
| | GTAGTCTCAAGCTGCCCGGCCTGCTCTGGTGCCTG | |
| | GCCTCGCGCCGCCGTGTATCGCCCCGCCCTGGGCG | |
| | GCAAGGCTGGCCCGGTCGGCACCAGTTGCGTGAG | |
| | CGGAAAGATGGCCGCTTCCCGGCCCTGCTGCAGG | |
| | GAGCACAAAATGGAGGACGCGGCGCTCGGGAGA | |
| | GCGGGCGGGTGAGTCACCCACACAAAGGAAAAGG | |
| | GCCTTTCCGTCCTCAGCCGTCGCTTCATGTGACTC | |
| | CACGGAGTACCGGGCGCCGTCCAGGCACCTCGAT | |
| | TAGTTCTCCAGCTTTTGGAGTACGTCGTCTTTAGG | |
| | TTGGGGGGAGGGGTTTTATGCGATGGAGTTTCCCC | |
| | ACACTGAGTGGGTGGAGACTGAAGTTAGGCCAGC | |
| | TTGGCACTTGATGTAATTCTCCTTGGAATTTGCCCT | |
| | TTTTGAGTTTGGATCTTGGTTCATTCTCAAGCCTCA | |
| | GACAGTGGTTCAAAGTTTTTTTCTTCCATTTCAGGT | |
| | GTCGTGAAAACTACCCCTAAAAGCCAAA | |
| 27 | gaacagagaaacaggagaatatgggccaaacaggatatctgtggtaagcagttcc | MND |
| | tgccccggctcagggccaagaacagttggaacagcagaatatgggccaaacag | promoter |
| | gatatctgtggtaagcagttcctgccccggctcagggccaagaacagatggtccc | |
| | cagatgcggtcccgccctcagcagtttctagagaaccatcagatgtttccagggtg | |
| | ccccaaggacctgaaatgaccctgtgccttatttgaactaaccaatcagttcgcttct | |
| | cgcttctgttcgcgcgcttctgctccccgagctctatataagcagagctcgtttagtg | |
| | aaccgtcagatc | |
| 28 | GVQVETISPGDGRTFPKRGQTCVVHYTGMLEDGKK | RACR (g) |
| | FDSSRDRNKPFKFMLGKQEVIRGWEEGVAQMSVGQ | |
| | RAKLTISPDYAYGATGHPGIIPPHATLVFDVELLKLG | |
| | EGSNTSKENPFLFALEAVVISVGSMGLIISLLCVYFW | |
| | LERTMPRIPTLKNLEDLVTEYHGNFSAWSGVSKGLA | |
| | ESLQPDYSERLCLVSEIPPKGGALGEGPGASPCNQHS | |
| | PYWAPPCYTLKPET | |
| 29 | MPLGLLWLGLALLGALHAQA | Signal |
| | | peptide |
| 30 | GVQVETISPGDGRTFPKRGQTCVVHYTGMLEDGKK | FKBP |
| | FDSSRDRNKPFKFMLGKQEVIRGWEEGVAQMSVGQ | domain |
| | RAKLTISPDYAYGATGHPGIIPPHATLVEDVELLKLG | (FKBP12) |
| | E | |
| 31 | GSNTSKENPFLFALEAVVISVGSMGLIISLLCVYFWL | IL2RG |
| | | transmembra |
| | | ne domain |
| | | and |
| | | ectodomain |
| | | portion |
| 32 | CGTGAGGCTCCGGTGCCCGTCAGTGGGCAGAGCG | RACR |
| | CACATCGCCCACAGTCCCCGAGAAGTTGGGGGGA | |
| | GGGGTCGGCAATTGAACCGGTGCCTAGAGAAGGT | |
| | GGCGCGGGGTAAACTGGGAAAGTGATGTCGTGTA | |
| | CTGGCTCCGCCTTTTTCCCGAGGGTGGGGGAGAAC | |
| | CGTATATAAGTGCAGTAGTCGCCGTGAACGTTCTT | |
| | TTTCGCAACGGGTTTGCCGCCAGAACACAGGTAA | |
| | GTGCCGTGTGTGGTTCCCGCGGGCCTGGCCTCTTT | |
| | ACGGGTTATGGCCCTTGCGTGCCTTGAATTACTTC | |
| | CACGCCCCTGGCTGCAGTACGTGATTCTTGATCCC | |
| | GAGCTTCGGGTTGGAAGTGGGTGGGAGAGTTCGA | |
| | GGCCTTGCGCTTAAGGAGCCCCTTCGCCTCGTGCT | |
| | TGAGTTGAGGCCTGGCTTGGGCGCTGGGGCCGCC | |
| | GCGTGCGAATCTGGTGGCACCTTCGCGCCTGTCTC | |
| | GCTGCTTTCGATAAGTCTCTAGCCATTTAAAATTT | |
| | TTGATGACCTGCTGCGACGCTTTTTTTCTGGCAAG | |
| | ATAGTCTTGTAAATGCGGGCCAAGATCTGCACACT | |
| | GGTATTTCGGTTTTTGGGGCCGCGGGCGGCGACGG | |
| | GGCCCGTGCGTCCCAGCGCACATGTTCGGCGAGG | |
| | CGGGGCCTGCGAGCGCGGCCACCGAGAATCGGAC | |
| | GGGGGTAGTCTCAAGCTGGCCGGCCTGCTCTGGTG | |
| | CCTGGCCTCGCGCCGCCGTGTATCGCCCCGCCCTG | |
| | GGCGGCAAGGCTGGCCCGGTCGGCACCAGTTGCG | |
| | TGAGCGGAAAGATGGCCGCTTCCCGGCCCTGCTG | |
| | CAGGGAGCTCAAAATGGAGGACGCGGCGCTCGGG | |
| | AGAGCGGGCGGGTGAGTCACCCACACAAAGGAAA | |
| | AGGGCCTTTCCGTCCTCAGCCGTCGCTTCATGTGA | |
| | CTCCACGGAGTACCGGGCGCCGTCCAGGCACCTC | |
| | GATTAGTTCTCGAGCTTTTGGAGTACGTCGTCTTT | |
| | AGGTTGGGGGGAGGGGTTTTATGCGATGGAGTTT | |
| | CCCCACACTGAGTGGGTGGAGACTGAAGTTAGGC | |
| | CAGCTTGGCACTTGATGTAATTCTCCTTGGAATTT | |
| | GCCCTTTTTGAGTTTGGATCTTGGTTCATTCTCAAG | |
| | CCTCAGACAGTGGTTCAAAGTTTTTTTCTTCCATTT | |
| | CAGGTGTCGTGA | |
| | GCCACC | |
| | ATGCCACTGGGACTGCTGTGGCTGGGACTGGCCCT | |
| | GCTGGGCGCCCTGCACGCCCAGGCCGGCGTGCAG | |
| | GTGGAGACAATCAGCCCAGGCGACGGACGCACCT | |
| | TTCCTAAGCGGGGCCAGACATGCGTGGTGCACTA | |
| | CACCGGCATGCTGGAGGATGGCAAGAAGTTCGAC | |
| | TCTAGCAGAGATAGGAACAAGCCCTTTAAGTTCAT | |
| | GCTGGGCAAGCAGGAAGTGATCAGAGGCTGGGAG | |
| | GAGGGCGTGGCCCAGATGTCTGTGGGCCAGAGGG | |
| | CCAAGCTGACAATCAGCCCAGACTACGCATATGG | |
| | AGCAACCGGACACCCAGGAATCATCCCACCTCAC | |
| | GCCACACTGGTGTTCGATGTGGAGCTGCTGAAGCT | |
| | GGGCGAGGGCTCTAACACCAGCAAGGAGAATCCA | |
| | TTTCTGTTCGCACTGGAGGCAGTGGTCATCTCCGT | |
| | GGGCTCTATGGGCCTGATCATCTCCCTGCTGTGCG | |
| | TGTACTTTTGGCTGGAGAGAACAATGCCCAGGATC | |
| | CCTACCCTGAAGAACCTGGAGGACCTGGTGACCG | |
| | AGTATCACGGCAATTTCAGCGCCTGGTCCGGCGTG | |
| | TCTAAGGGACTGGCAGAGTCCCTGCAGCCAGATT | |
| | ACTCTGAGCGGCTGTGCCTGGTGAGCGAGATCCC | |
| | ACCCAAGGGAGGCGCCCTGGGAGAGGGACCTGGA | |
| | GCAAGCCCATGCAACCAGCACTCCCCATATTGGG | |
| | CCCCTCCATGTTACACCCTGAAGCCTGAGACA | |
| | GGAAGCGGAGCGACGAATTTTAGTCTACTGAAAC | |
| | AAGCGGGAGACGTGGAGGAAAACCCTGGACCT | |
| | ATGGCACTGCCAGTGACCGCCCTGCTGCTGCCACT | |
| | GGCCCTGCTGCTGCACGCAGCACGCCCCATCCTGT | |
| | GGCACGAAATGTGGCATGAAGGCCTGGAGGAGGC | |
| | AAGCAGACTGTATTTTGGCGAGAGAAATGTGAAA | |
| | GGAATGTTTGAGGTGCTGGAGCCACTGCACGCAA | |
| | TGATGGAGAGGGGACCTCAGACCCTGAAGGAGAC | |
| | ATCCTTTAACCAGGCCTATGGCAGAGACCTGATGG | |
| | AGGCCCAGGAGTGGTGCAGGAAGTACATGAAGAG | |
| | CGGAAATGTGAAAGACCTGCTGCAGGCCTGGGAT | |
| | CTGTATTATCACGTGTTCAGAAGGATCAGCAAGG | |
| | GCAAGGATACCATCCCTTGGCTGGGACACCTGCTG | |
| | GTGGGACTGTCCGGAGCCTTTGGCTTCATCATCCT | |
| | GGTGTACCTGCTGATCAACTGCCGCAATACAGGCC | |
| | CATGGCTGAAGAAGGTGCTGAAGTGTAACACCCC | |
| | AGACCCCTCCAAGTTCTTTTCTCAGCTGTCCTCTG | |
| | AGCACGGCGGCGATGTGCAGAAGTGGCTGAGCTC | |
| | CCCTTTTCCATCTAGCTCCTTCAGCCCTGGAGGAC | |
| | TGGCACCAGAGATCTCCCCTCTGGAGGTGCTGGA | |
| | GCGGGACAAGGTGACCCAGCTGCTGCTGCAGCAG | |
| | GATAAGGTGCCCGAGCCTGCCTCCCTGTCTAGCAA | |
| | CCACAGCCTGACCTCCTGCTTTACAAATCAGGGCT | |
| | ATTTCTTTTTCCACCTGCCAGACGCACTGGAGATC | |
| | GAGGCATGTCAGGTGTACTTCACATATGATCCATA | |
| | CAGCGAGGAGGACCCAGATGAGGGAGTGGCCGGC | |
| | GCCCCTACCGGATCCTCTCCACAGCCACTGCAGCC | |
| | ACTGTCTGGCGAGGACGATGCCTACTGTACATTTC | |
| | CTAGCAGAGACGATCTGCTGCTGTTCTCCCCTTCT | |
| | CTGCTGGGAGGACCATCTCCACCTAGCACCGCACC | |
| | TGGAGGATCCGGGGCAGGGGAGGAGAGGATGCC | |
| | ACCATCTCTGCAGGAGAGGGTGCCAAGGGACTGG | |
| | GATCCTCAGCCACTGGGACCTCCAACCCCAGGAG | |
| | TGCCCGACCTGGTGGATTTCCAGCCCCCTCCAGAG | |
| | CTGGTGCTGAGAGAGGCAGGAGAGGAGGTGCCAG | |
| | ACGCAGGACCAAGAGAGGGCGTGAGCTTTCCATG | |
| | GTCCAGGCCACCTGGACAGGGAGAGTTCAGAGCC | |
| | CTGAACGCCAGGCTGCCACTGAATACAGACGCCT | |
| | ACCTGTCTCTGCAGGAGCTGCAGGGCCAGGATCC | |
| | CACCCACCTGGTG | |
| | TAAAC | |
| | AATAAAAGATCTTTATTTTCATTAGATCTGTGTGT | |
| | TGGTTTTTTGTGTG | |
| 33 | MALPVTALLLPLALLLHAARPILWHEMWHEGLEEA | RACR (b) |
| | SRLYFGERNVKGMFEVLEPLHAMMERGPQTLKETS | |
| | FNQAYGRDLMEAQEWCRKYMKSGNVKDLLQAWD | |
| | LYYHVFRRISKGKDTIPWLGHLLVGLSGAFGFIILVY | |
| | LLINCRNTGPWLKKVLKCNTPDPSKFFSQLSSEHGG | |
| | DVQKWLSSPFPSSSFSPGGLAPEISPLEVLERDKVTQL | |
| | LLQQDKVPEPASLSSNHSLTSCFTNQGYFFFHLPDAL | |
| | EIEACQVYFTYDPYSEEDPDEGVAGAPTGSSPQPLQP | |
| | LSGEDDAYCTFPSRDDLLLFSPSLLGGPSPPSTAPGGS | |
| | GAGEERMPPSLQERVPRDWDPQPLGPPTPGVPDLVD | |
| | FQPPPELVLREAGEEVPDAGPREGVSFPWSRPPGQGE | |
| | FRALNARLPLNTDAYLSLQELQGQDPTHLV | |
| 34 | MALPVTALLLPLALLLHAARP | CD8a Signal |
| | | peptide |
| 35 | GKDTIPWLGHLLVGLSGAFGFIILVYLLI | IL2RB |
| | | transmembrane |
| | | domain and |
| | | ectodomain |
| | | portion |
| 36 | IPWLGHLLVGLSGAFGFIILVYLLI | IL2RB |
| | | transmembrane |
| | | domain |
| 37 | ATGCCACTGGGACTGCTGTGGCTGGGACTGGCCCT | RACR (g) |
| | GCTGGGCGCCCTGCACGCCCAGGCCGGCGTGCAG | Nucleotides |
| | GTGGAGACAATCAGCCCAGGCGACGGACGCACCT | |
| | TTCCTAAGCGGGGCCAGACATGCGTGGTGCACTA | |
| | CACCGGCATGCTGGAGGATGGCAAGAAGTTCGAC | |
| | TCTAGCAGAGATAGGAACAAGCCCTTTAAGTTCAT | |
| | GCTGGGCAAGCAGGAAGTGATCAGAGGCTGGGAG | |
| | GAGGGCGTGGCCCAGATGTCTGTGGGCCAGAGGG | |
| | CCAAGCTGACAATCAGCCCAGACTACGCATATGG | |
| | AGCAACCGGACACCCAGGAATCATCCCACCTCAC | |
| | GCCACACTGGTGTTCGATGTGGAGCTGCTGAAGCT | |
| | GGGCGAGGGCTCTAACACCAGCAAGGAGAATCCA | |
| | TTTCTGTTCGCACTGGAGGCAGTGGTCATCTCCGT | |
| | GGGCTCTATGGGCCTGATCATCTCCCTGCTGTGCG | |
| | TGTACTTTTGGCTGGAGAGAACAATGCCCAGGATC | |
| | CCTACCCTGAAGAACCTGGAGGACCTGGTGACCG | |
| | AGTATCACGGCAATTTCAGCGCCTGGTCCGGCGTG | |
| | TCTAAGGGACTGGCAGAGTCCCTGCAGCCAGATT | |
| | ACTCTGAGCGGCTGTGCCTGGTGAGCGAGATCCC | |
| | ACCCAAGGGAGGCGCCCTGGGAGAGGGACCTGGA | |
| | GCAAGCCCATGCAACCAGCACTCCCCATATTGGG | |
| | CCCCTCCATGTTACACCCTGAAGCCTGAGACA | |
| 38 | ATGGCACTGCCAGTGACCGCCCTGCTGCTGCCACT | RACR (b) |
| | GGCCCTGCTGCTGCACGCAGCACGCCCCATCCTGT | nucleotides |
| | GGCACGAAATGTGGCATGAAGGCCTGGAGGAGGC | |
| | AAGCAGACTGTATTTTGGCGAGAGAAATGTGAAA | |
| | GGAATGTTTGAGGTGCTGGAGCCACTGCACGCAA | |
| | TGATGGAGAGGGGACCTCAGACCCTGAAGGAGAC | |
| | ATCCTTTAACCAGGCCTATGGCAGAGACCTGATGG | |
| | AGGCCCAGGAGTGGTGCAGGAAGTACATGAAGAG | |
| | CGGAAATGTGAAAGACCTGCTGCAGGCCTGGGAT | |
| | CTGTATTATCACGTGTTCAGAAGGATCAGCAAGG | |
| | GCAAGGATACCATCCCTTGGCTGGGACACCTGCTG | |
| | GTGGGACTGTCCGGAGCCTTTGGCTTCATCATCCT | |
| | GGTGTACCTGCTGATCAACTGCCGCAATACAGGCC | |
| | CATGGCTGAAGAAGGTGCTGAAGTGTAACACCCC | |
| | AGACCCCTCCAAGTTCTTTTCTCAGCTGTCCTCTG | |
| | AGCACGGCGGCGATGTGCAGAAGTGGCTGAGCTC | |
| | CCCTTTTCCATCTAGCTCCTTCAGCCCTGGAGGAC | |
| | TGGCACCAGAGATCTCCCCTCTGGAGGTGCTGGA | |
| | GCGGGACAAGGTGACCCAGCTGCTGCTGCAGCAG | |
| | GATAAGGTGCCCGAGCCTGCCTCCCTGTCTAGCAA | |
| | CCACAGCCTGACCTCCTGCTTTACAAATCAGGGCT | |
| | ATTTCTTTTTCCACCTGCCAGACGCACTGGAGATC | |
| | GAGGCATGTCAGGTGTACTTCACATATGATCCATA | |
| | CAGCGAGGAGGACCCAGATGAGGGAGTGGCCGGC | |
| | GCCCCTACCGGATCCTCTCCACAGCCACTGCAGCC | |
| | ACTGTCTGGCGAGGACGATGCCTACTGTACATTTC | |
| | CTAGCAGAGACGATCTGCTGCTGTTCTCCCCTTCT | |
| | CTGCTGGGAGGACCATCTCCACCTAGCACCGCACC | |
| | TGGAGGATCCGGGGCAGGGGAGGAGAGGATGCC | |
| | ACCATCTCTGCAGGAGAGGGTGCCAAGGGACTGG | |
| | GATCCTCAGCCACTGGGACCTCCAACCCCAGGAG | |
| | TGCCCGACCTGGTGGATTTCCAGCCCCCTCCAGAG | |
| | CTGGTGCTGAGAGAGGCAGGAGAGGAGGTGCCAG | |
| | ACGCAGGACCAAGAGAGGGCGTGAGCTTTCCATG | |
| | GTCCAGGCCACCTGGACAGGGAGAGTTCAGAGCC | |
| | CTGAACGCCAGGCTGCCACTGAATACAGACGCCT | |
| | ACCTGTCTCTGCAGGAGCTGCAGGGCCAGGATCC | |
| | CACCCACCTGGTG | |
| 39 | AATAAAAGATCTTTATTTTCATTAGATCTGTGTGT | Synthetic |
| | TGGTTTTTTGTGTG | polyA |
| 40 | TACCCAGAGAATGGAGAAACCCTGCAGGGAATTC | RACR_B2M |
| | CCAAGCTGTAGTTATAAACAGAAGTTCTCCTTCTG | SSODN |
| | CTAGGTAGCATTCAAAGATCTTAATCTTCTGGGTT | |
| | TCCGTTTTCTCGAATGAAAAATGCAGGTCCGAGCA | |
| | GTTAACTGGCTGGGGCACCATTAGCAAGTCACTTA | |
| | GCATCTCTGGGGCCAGTCTGCAAAGCGAGGGGGC | |
| | AGCCTTAATGTGCCTCCAGCCTGAAGTCCTAGAAT | |
| | GAGCGCCCGGTGTCCCAAGCTGGGGCGCGCACCC | |
| | CAGATCGGAGGGCGCCGATGTACAGACAGCAAAC | |
| | TCACCCAGTCTAGTGCATGCCTTCTTAAACATCAC | |
| | GAGACTCTAAGAAAAGGAAACTGAAAACGGGAA | |
| | AGTCCCTCTCTCTAACCTGGCACTGCGTCGCTGGC | |
| | TTGGAGACAGGTGACGGTCCCTGCGGGCCTTGTCC | |
| | TGATTGGCTGGGCACGCGTTTAATATAAGTGGAG | |
| | GCGTCGCGCTGGCGGGC | |
| | CGTGAGGCTCCGGTGCCCGTCAGTGGGCAGAGCG | |
| | CACATCGCCCACAGTCCCCGAGAAGTTGGGGGGA | |
| | GGGGTCGGCAATTGAACCGGTGCCTAGAGAAGGT | |
| | GGCGCGGGGTAAACTGGGAAAGTGATGTCGTGTA | |
| | CTGGCTCCGCCTTTTTCCCGAGGGTGGGGGAGAAC | |
| | CGTATATAAGTGCAGTAGTCGCCGTGAACGTTCTT | |
| | TTTCGCAACGGGTTTGCCGCCAGAACACAGGTAA | |
| | GTGCCGTGTGTGGTTCCCGCGGGCCTGGCCTCTTT | |
| | ACGGGTTATGGCCCTTGCGTGCCTTGAATTACTTC | |
| | CACGCCCCTGGCTGCAGTACGTGATTCTTGATCCC | |
| | GAGCTTCGGGTTGGAAGTGGGTGGGAGAGTTCGA | |
| | GGCCTTGCGCTTAAGGAGCCCCTTCGCCTCGTGCT | |
| | TGAGTTGAGGCCTGGCTTGGGCGCTGGGGCCGCC | |
| | GCGTGCGAATCTGGTGGCACCTTCGCGCCTGTCTC | |
| | GCTGCTTTCGATAAGTCTCTAGCCATTTAAAATTT | |
| | TTGATGACCTGCTGCGACGCTTTTTTTCTGGCAAG | |
| | ATAGTCTTGTAAATGCGGGCCAAGATCTGCACACT | |
| | GGTATTTCGGTTTTTGGGGCCGCGGGCGGCGACGG | |
| | GGCCCGTGCGTCCCAGCGCACATGTTCGGCGAGG | |
| | CGGGGCCTGCGAGCGCGGCCACCGAGAATCGGAC | |
| | GGGGGTAGTCTCAAGCTGGCCGGCCTGCTCTGGTG | |
| | CCTGGCCTCGCGCCGCCGTGTATCGCCCCGCCCTG | |
| | GGCGGCAAGGCTGGCCCGGTCGGCACCAGTTGCG | |
| | TGAGCGGAAAGATGGCCGCTTCCCGGCCCTGCTG | |
| | CAGGGAGCTCAAAATGGAGGACGCGGCGCTCGGG | |
| | AGAGCGGGCGGGTGAGTCACCCACACAAAGGAAA | |
| | AGGGCCTTTCCGTCCTCAGCCGTCGCTTCATGTGA | |
| | CTCCACGGAGTACCGGGCGCCGTCCAGGCACCTC | |
| | GATTAGTTCTCGAGCTTTTGGAGTACGTCGTCTTT | |
| | AGGTTGGGGGGAGGGGTTTTATGCGATGGAGTTT | |
| | CCCCACACTGAGTGGGTGGAGACTGAAGTTAGGC | |
| | CAGCTTGGCACTTGATGTAATTCTCCTTGGAATTT | |
| | GCCCTTTTTGAGTTTGGATCTTGGTTCATTCTCAAG | |
| | CCTCAGACAGTGGTTCAAAGTTTTTTTCTTCCATTT | |
| | CAGGTGTCGTGA | |
| | GCCACC | |
| | ATGCCACTGGGACTGCTGTGGCTGGGACTGGCCCT | |
| | GCTGGGCGCCCTGCACGCCCAGGCCGGCGTGCAG | |
| | GTGGAGACAATCAGCCCAGGCGACGGACGCACCT | |
| | TTCCTAAGCGGGGCCAGACATGCGTGGTGCACTA | |
| | CACCGGCATGCTGGAGGATGGCAAGAAGTTCGAC | |
| | TCTAGCAGAGATAGGAACAAGCCCTTTAAGTTCAT | |
| | GCTGGGCAAGCAGGAAGTGATCAGAGGCTGGGAG | |
| | GAGGGCGTGGCCCAGATGTCTGTGGGCCAGAGGG | |
| | CCAAGCTGACAATCAGCCCAGACTACGCATATGG | |
| | AGCAACCGGACACCCAGGAATCATCCCACCTCAC | |
| | GCCACACTGGTGTTCGATGTGGAGCTGCTGAAGCT | |
| | GGGCGAGGGCTCTAACACCAGCAAGGAGAATCCA | |
| | TTTCTGTTCGCACTGGAGGCAGTGGTCATCTCCGT | |
| | GGGCTCTATGGGCCTGATCATCTCCCTGCTGTGCG | |
| | TGTACTTTTGGCTGGAGAGAACAATGCCCAGGATC | |
| | CCTACCCTGAAGAACCTGGAGGACCTGGTGACCG | |
| | AGTATCACGGCAATTTCAGCGCCTGGTCCGGCGTG | |
| | TCTAAGGGACTGGCAGAGTCCCTGCAGCCAGATT | |
| | ACTCTGAGCGGCTGTGCCTGGTGAGCGAGATCCC | |
| | ACCCAAGGGAGGCGCCCTGGGAGAGGGACCTGGA | |
| | GCAAGCCCATGCAACCAGCACTCCCCATATTGGG | |
| | CCCCTCCATGTTACACCCTGAAGCCTGAGACA | |
| | GGAAGCGGAGCGACGAATTTTAGTCTACTGAAAC | |
| | AAGCGGGAGACGTGGAGGAAAACCCTGGACCT | |
| | ATGGCACTGCCAGTGACCGCCCTGCTGCTGCCACT | |
| | GGCCCTGCTGCTGCACGCAGCACGCCCCATCCTGT | |
| | GGCACGAAATGTGGCATGAAGGCCTGGAGGAGGC | |
| | AAGCAGACTGTATTTTGGCGAGAGAAATGTGAAA | |
| | GGAATGTTTGAGGTGCTGGAGCCACTGCACGCAA | |
| | TGATGGAGAGGGGACCTCAGACCCTGAAGGAGAC | |
| | ATCCTTTAACCAGGCCTATGGCAGAGACCTGATGG | |
| | AGGCCCAGGAGTGGTGCAGGAAGTACATGAAGAG | |
| | CGGAAATGTGAAAGACCTGCTGCAGGCCTGGGAT | |
| | CTGTATTATCACGTGTTCAGAAGGATCAGCAAGG | |
| | GCAAGGATACCATCCCTTGGCTGGGACACCTGCTG | |
| | GTGGGACTGTCCGGAGCCTTTGGCTTCATCATCCT | |
| | GGTGTACCTGCTGATCAACTGCCGCAATACAGGCC | |
| | CATGGCTGAAGAAGGTGCTGAAGTGTAACACCCC | |
| | AGACCCCTCCAAGTTCTTTTCTCAGCTGTCCTCTG | |
| | AGCACGGCGGCGATGTGCAGAAGTGGCTGAGCTC | |
| | CCCTTTTCCATCTAGCTCCTTCAGCCCTGGAGGAC | |
| | TGGCACCAGAGATCTCCCCTCTGGAGGTGCTGGA | |
| | GCGGGACAAGGTGACCCAGCTGCTGCTGCAGCAG | |
| | GATAAGGTGCCCGAGCCTGCCTCCCTGTCTAGCAA | |
| | CCACAGCCTGACCTCCTGCTTTACAAATCAGGGCT | |
| | ATTTCTTTTTCCACCTGCCAGACGCACTGGAGATC | |
| | GAGGCATGTCAGGTGTACTTCACATATGATCCATA | |
| | CAGCGAGGAGGACCCAGATGAGGGAGTGGCCGGC | |
| | GCCCCTACCGGATCCTCTCCACAGCCACTGCAGCC | |
| | ACTGTCTGGCGAGGACGATGCCTACTGTACATTTC | |
| | CTAGCAGAGACGATCTGCTGCTGTTCTCCCCTTCT | |
| | CTGCTGGGAGGACCATCTCCACCTAGCACCGCACC | |
| | TGGAGGATCCGGGGCAGGGGAGGAGAGGATGCC | |
| | ACCATCTCTGCAGGAGAGGGTGCCAAGGGACTGG | |
| | GATCCTCAGCCACTGGGACCTCCAACCCCAGGAG | |
| | TGCCCGACCTGGTGGATTTCCAGCCCCCTCCAGAG | |
| | CTGGTGCTGAGAGAGGCAGGAGAGGAGGTGCCAG | |
| | ACGCAGGACCAAGAGAGGGCGTGAGCTTTCCATG | |
| | GTCCAGGCCACCTGGACAGGGAGAGTTCAGAGCC | |
| | CTGAACGCCAGGCTGCCACTGAATACAGACGCCT | |
| | ACCTGTCTCTGCAGGAGCTGCAGGGCCAGGATCC | |
| | CACCCACCTGGTG | |
| | TAAAC | |
| | AATAAAAGATCTTTATTTTCATTAGATCTGTGTGT | |
| | TGGTTTTTTGTGTG | |
| | TGGCCTTAGCTGTGCTCGCGCTACTCTCTCTTTCTG | |
| | GCCTGGAGGCTATCCAGCGTGAGTCTCTCCTACCC | |
| | TCCCGCTCTGGTCCTTCCTCTCCCGCTCTGCACCCT | |
| | CTGTGGCCCTCGCTGTGCTCTCTCGCTCCGTGACTT | |
| | CCCTTCTCCAAGTTCTCCTTGGTGGCCCGCCGTGG | |
| | GGCTAGTCCAGGGCTGGATCTCGGGGAAGCGGCG | |
| | GGGTGGCCTGGGAGTGGGGAAGGGGGTGCGCACC | |
| | CGGGACGCGCGCTACTTGCCCCTTTCGGCGGGGA | |
| | GCAGGGGAGACCTTTGGCCTACGGCGACGGGAGG | |
| | GTCGGGACAAAGTTTAGGGCGTCGATAAGCGTCA | |
| | GAGCGCCGAGGTTGGGGGAGGGTTTCTCTTCCGCT | |
| | CTTTCGCGGGGCCTCTGGCTCCCCCAGCGCAGCTG | |
| | GAGTGGGGGACGGGTAGGCTCGTCCCAAAGGCGC | |
| | GGCGCTGAGGTTTGTGAACGCGTGGAGGGGCGCT | |
| | TGGGGTCTGGGGGA | |
| 41 | GGAAGCGGAGCGACGAATTTTAGTCTACTGAAAC | PQR linker |
| | AAGCGGGAGACGTGGAGGAAAACCCTGGACCT | nucleotide |
| 42 | GSGATNFSLLKQAGDVEENPGP | PQR linker |
| 43 | VKQTLNFDLLKLAGDVESNPGP | F2A |
| 44 | QCTNYALLKLAGDVESNPGP | E2A |
| 45 | LEGGGEGRGSLLTCGDVEENPGPR | T2A |
| 46 | EGRGSLLTCGDVEENPGP | T2A |
| 47 | GSGATNFSLLKQAGDVEENPGP | P2A |
| 48 | ATNFSLLKQAGDVEENPGP | P2A |
| 49 | GVQVETISPGDGRTFPKRGQTCVVHYTGMLEDGKK | FKBP |
| | FDSSRDRNKPFKFMLGKQEVIRGWE | domain |
| | EGVAQMSVGQRAKLTISPDYAYGATGHPGIIPPHAT | (FKBP12) |
| | LVFDVELLKL | |

1. An engineered stem cell comprising a synthetic cytokine receptor for a non-physiological ligand,
wherein the cytokine receptor comprises:
a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

2. The engineered stem cell of claim 1, wherein the first dimerization domain and the second dimerization domain are extracellular domains.

3. The engineered stem cell of claim 1 or claim 2, wherein the synthetic gamma chain polypeptide comprises, in N- to C-terminal order, the first dimerization domain, the first transmembrane domain, and the interleukin-2 receptor subunit gamma (IL-2RG) intracellular domain, and
the synthetic beta chain polypeptide comprises, in N- to C-terminal order, the second dimerization domain, the second transmembrane domain, and the intracellular domain.

4. The engineered stem cell of any of claims 1-3, wherein the IL-2RG intracellular domain comprises a polypeptide sequence at least 95% identical to SEQ ID NO: 1, or a polypeptide sequence as set forth in SEQ ID NO: 1.

5. The engineered stem cell of any of claims 1-4, wherein the first transmembrane domain comprises the IL-2RG transmembrane domain.

6. The engineered stem cell of claim 5, wherein the IL-2RG transmembrane domain comprises a polypeptide sequence at least 95% identical to SEQ ID NO: 8 or 31, or a polypeptide sequence as set forth in SEQ ID NO: 8 or 31.

7. The engineered stem cell of any of claims 1-6, wherein the beta chain intracellular domain comprises the IL-2RB intracellular domain.

8. The engineered stem cell of claim 7, wherein the IL-2RB intracellular domain comprises a polypeptide sequence at least 95% identical to SEQ ID NO: 2, or a polypeptide sequence as set forth in SEQ ID NO: 2.

9. The engineered stem cell of any of claims 1-6, wherein the beta chain intracellular domain comprises the IL-7RB intracellular domain.

10. The engineered stem cell of claim 9, wherein the IL-7RB intracellular domain comprises a polypeptide sequence at least 95% identical to SEQ ID NO: 3, or a polypeptide sequence as set forth in SEQ ID NO: 3.

11. The engineered stem cell of any of claims 1-6, wherein the beta chain intracellular domain comprises the IL-21RB intracellular domain.

12. The engineered stem cell of claim 11, wherein the IL-21RB intracellular domain comprises a polypeptide sequence at least 95% identical to SEQ ID NO: 4, or a polypeptide sequence as set forth in SEQ ID NO: 4.

13. The engineered stem cell of any of claims 1-12, wherein the second transmembrane domain comprises a transmembrane domain from the same beta chain intracellular domain.

14. The engineered stem cell of claims 1-8 and 13, wherein the second transmembrane domain is a transmembrane domain of IL-2RB comprising a polypeptide sequence at least 95% identical to SEQ ID NO: 35 or 36, or a polypeptide sequence as set forth in SEQ ID NO: 35 or 36.

15. The engineered stem cell of any of claims 1-8, 13 and 14, wherein:
the synthetic gamma chain polypeptide contains an IL-2RG TM domain comprising the sequence set forth in SEQ ID NO: 8 or 31 and a IL-2RG intracellular domain comprising the sequence set forth in SEQ ID NO:1; and
the synthetic beta chain polypeptide contains an IL-2RB TM domain comprising the sequence set forth in SEQ ID NO: 35 or 36 and a IL-2RB intracellular domain comprising the sequence set forth in SEQ ID NO:2.

16. The engineered stem cell of any one of claims 1 to 15, wherein the first dimerization domain and the second dimerization domain are heterodimerization domains selected from selected from FK506-Binding Protein of size 12 kD (FKBP) and a FKBP12-rapamycin binding (FRB) domain; and/or
wherein the non-physiological ligand is rapamycin or a rapalog.

17. The engineered stem cell of claim 16, wherein the FRB domain comprises a polypeptide sequence at least 95% identical to SEQ ID NO: 6 or SEQ ID NO: 7.

18. The engineered stem cell of claim 16, wherein the FRB domain comprises the polypeptide sequence set forth in SEQ ID NO: 6 or SEQ ID NO: 7.

19. The engineered stem cell of any one of claims 1 to 15, wherein the first dimerization domain and the second dimerization domain are heterodimerization domains selected from selected from FK506-Binding Protein of size 12 kD (FKBP) and a calcineurin domain; and/or
wherein the non-physiological ligand is FK506 or an analogue thereof.

20. The engineered stem cell of any one of claims 1 to 16, wherein the FKBP domain comprises a polypeptide sequence at least 95% identical to SEQ ID NO: 5 or SEQ ID NO: 30.

21. The engineered stem cell of any one of claims 1 to 16, wherein the FKBP domain comprises the polypeptide sequence set forth in SEQ ID NO: 5 or SEQ ID NO:30.

22. The engineered stem cell of any of claims 1-8 and 13-21, wherein the synthetic gamma chain polypeptide has the sequence of amino acids set forth in SEQ ID NO:28 or a sequence of amino acids that is at least 90%, at least 95%, at least 96%, at least 97%, at least 98%, at least 99%, or 100% identical to SEQ ID NO: 28, and the synthetic beta chain polypeptide has the sequence of amino acids set forth in SEQ ID NO:33 or a sequence of amino acids that is at least 90%, at least 95%, at least 96%, at least 97%, at least 98%, at least 99%, or 100% identical to SEQ ID NO: 33.

23. The engineered stem cell of any of claims 1-8 and 13-22, wherein the synthetic gamma chain polypeptide has the sequence of amino acids set forth in SEQ ID O: 28 and the synthetic beta chain polypeptide has the sequence of amino acids set forth in SEQ ID NO:33.

24. The engineered stem cell of any one of claims 1 to 15, wherein the first dimerization domain and the second dimerization domain are homodimerization domains selected from:
i) FK506-Binding Protein of size 12 kD (FKBP);
ii) cyclophiliA (CypA); or
iii) gyrase B (CyrB);
and the non-physiological ligand is, respectively:
i) FK1012, AP1510, AP1903, or AP20187 or an analog thereof;
ii) cyclosporin-A (CsA) or an analog thereof; or
iii) coumermycin or an analog thereof.

25. The engineered stem cell of any of claims 1-24, wherein the stem cell is a pluripotent stem cell.

26. The engineered stem cell of any one of claims 1 to 25, wherein the stem cells are induced pluripotent stem cells (iPSCs).

27. The engineered stem cell of any of claims 1-26, wherein the stem cell is resistant to rapamycin-mediated mTOR inhibition.

28. The engineered stem cell of any one of claims 1 to 27, wherein the stem cells express a cytosolic polypeptide that binds to the non-physiological ligand.

29. The engineered stem cell of any one of claims 1 to 28, wherein the non-physiological ligand is rapamycin or a rapalog, and the stem cells express a cytosolic FRB domain or variant thereof.

30. The engineered stem cell of claim 29, wherein the cytosolic FRB domain comprises a polypeptide sequence at least 95% identical to SEQ ID NO: 6 or SEQ ID NO: 7.

31. The engineered stem cell of claim 29, wherein the cytosolic FRB domain comprises a polypeptide sequence at least 98% identical to SEQ ID NO: 6 or SEQ ID NO: 7.

32. The engineered stem cell of any of claims 1-31, wherein the stem cell comprises a disrupted FKBP12 gene that reduces expression of FKBP12.

33. The engineered stem cell of any of claims 1-32, wherein the stem cell comprises knock out of the FKBP12 gene.

34. The engineered stem cell of any one of claims 1 to 33, wherein the stem cells comprise a nucleotide sequence encoding the synthetic cytokine receptor inserted into the genome of the stem cell.

35. The engineered stem cell of claim 34, wherein the nucleotide sequence encoding the synthetic cytokine receptor is inserted into a non-target locus in the genome of the stem cell.

36. The engineered stem cell of claim 34, wherein the nucleotide sequence encoding the synthetic cytokine receptor is inserted into an endogenous gene of the stem cell.

37. The engineered stem cell of claim 36, wherein the insertion reduces expression of the endogenous gene in the locus.

38. The engineered stem cell of claim 36 or claim 37, wherein the insertion knocks out the endogenous gene in the locus.

39. The engineered stem cell of any of claims 36-38 wherein the insertion is by homology-directed repair.

40. The engineered stem cell of any of claims 36-39, wherein the endogenous gene is a housekeeping gene, a blood-lineage specific loci or an immune-related gene.

41. The engineered stem cell of claim 40, wherein endogenous gene is a housekeeping gene and the housekeeping gene is selected from eukaryotic translation elongation factor 1 alpha (EEF1A), glylceraldehyde-3-phosphate dehydrogenase (GAPDH), ubiquitin C (UBC), and actin beta (ACTB).

42. The engineered stem cell of claim 40, wherein the endogenous gene is a blood-lineage specific loci and the blood-lineage specific loci is selected from protein tyrosine phosphatase receptor type C (PTPRC), IL2RG, and IL2RB.

43. The engineered stem cell of claim 40, wherein the immune-related gene is selected from a beta-2-microglobulin (B2M) gene, a T cell receptor alpha constant (TRAC) gene, and a signal regulatory protein alpha (SIRPA) gene.

44. The engineered stem cell of any of claims 36-40 and 43, wherein the endogenous gene is B2M.

45. The engineered stem cell of any one of claims 1-43, wherein the stem cell comprises a B2M knockout.

46. The engineered stem cell of any of claims 1-44, wherein the cell has a disruption of a gene encoding FKBP12.

47. The engineered stem cell of claim 46, wherein the disruption is a FKBP12 knockout that inactivates the gene encoding FKBP12.

48. The engineered stem cell of any of claims 1-44, wherein the stem cell comprises a B2M knockout and a FKBP12 knockout.

49. The engineered stem cell of any one of claims 1-48, comprising a chimeric antigen receptor (CAR).

50. The engineered stem cell of claim 49, wherein the CAR is an anti-FITC CAR.

51. The engineered stem cell of any of claims 1-50, wherein binding of the non-physiological ligand to the synthetic cytokine receptor activates the synthetic cytokine receptor in the stem cells to induce differentiation of the engineered stem cells in the cell population.

52. A cell population comprising engineered stem cells of any of claims 1-51.

53. A method of genetically engineering stem cells to express a synthetic cytokine receptor, comprising:
contacting a population of stem cells with (i) a guide RNA (gRNA) targeting a target site in an endogenous gene, (ii) an RNA-guided endonuclease, and (iii) a recombinant vector comprising a nucleotide sequence encoding a synthetic cytokine receptor for a non-physiological ligand,
thereby inserting the nucleotide sequence into the endogenous gene;
wherein the cytokine receptor comprises:
a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

54. The method of claim 53, wherein the nucleotide sequence is inserted via homology directed repair (HDR).

55. The method of claim 54, wherein the vector comprises a nucleic acid comprising from 5′ to 3′ (a) a nucleotide sequence homologous with a region located upstream of the target site, (b) the nucleotide sequence encoding a synthetic cytokine receptor for a non-physiological ligand, and (c) a nucleotide sequence homologous with a region located downstream.

56. The method of claim 53, wherein the nucleotide sequence is inserted via non-homologous end joining (NHEJ).

57. The method of any one of claims 53-56, wherein the RNA-guided endonuclease is selected from a Cas endonuclease, a Mad endonuclease, and a Cpf1 endonuclease.

58. The method of any one of claims 53-57, wherein the RNA-guided endonuclease is Cas9.

59. The method of any one of claims 53-57, wherein the RNA-guided endonuclease is Mad7.

60. The method of any of claims 53-59, wherein the endogenous gene is selected from B2M, TRAC and SIRPA.

61. The method of any of claims 53-60, wherein the endogenous gene is B2M.

62. The method of any of claims 53-61, wherein the gRNA comprises the sequence set forth in SEQ ID NO:18.

63. The method of any of claims 55-62, wherein the nucleotide sequence homologous with a region located upstream of the target site comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 22; and the nucleotide sequence homologous with a region located downstream comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 23.

64. The method of any of claims 53-63, wherein the nucleotide sequence encoding the synthetic cytokine receptor comprises a first nucleic acid sequence encoding a gamma chain that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 37, and a second nucleic acid sequence encoding a beta chain that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 38.

65. The method of claim 64, wherein the first nucleic acid sequence and second nucleic acid sequence are separated by a cleavable linker or an IRES.

66. The method of claim 65, wherein the cleavable linker is a protein quantitation reporter linker (PQR), optionally set forth in SEQ ID NO:42.

67. The method of any of claims 53-66, wherein the nucleotide sequence encoding a synthetic cytokine receptor for a non-physiological ligand is under the operable control of a heterologous promoter.

68. The method of claim 67, wherein the heterologous promoter is the EF1α promoter or the MND promoter.

69. The method of any of claims 53-68, wherein the nucleotide sequence encoding the synthetic cytokine receptor comprises a polyadenylation sequence.

70. The method of any of claims 53-69, wherein the recombinant vector comprises the sequence set forth in SEQ ID NO:40 or a sequence that has at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 40.

71. The method of any one of claims 53-70, further comprising engineering the population of stem cells to be resistant to rapamycin-mediated mTOR inhibition.

72. The method of claim 71, wherein engineering the population of stem cells to be resistant to rapamycin comprises knocking out a FKBP12 gene.

73. The method of claim 72, wherein the method comprises further contacting the population of stem cells with an RNA-guided endonuclease and a guide RNA (gRNA) targeting a target site in the FKBP12 gene.

74. The method of claim 73, wherein the RNA-guided endonuclease is selected from a Cas endonuclease, a Mad endonuclease, and a Cpf1 endonuclease.

75. The method of claim 73 or claim 74, wherein the RNA-guided endonuclease is Cas9.

76. The method of claim 73 or claim 74, wherein the RNA-guided endonuclease is Mad7.

77. The method of any of claims 73-76, wherein the further contacting is carried out simultaneously with the contacting in (i) with a guide RNA (gRNA) targeting a target site in an endogenous gene, optionally in combination with the same RNA-guided endonuclease.

78. The method of any one of claims 73-77, wherein the gRNA comprises one or more gRNA selected from a gRNA comprising the sequence set forth in SEQ ID NO: 19, SEQ ID NO:20 or SEQ ID NO:21.

79. The method of claim 78, wherein the one or more gRNA is a pool of gRNA comprising 2 or 3 gRNA.

80. The method of any of claims 53-79, further comprising introducing into the population of stem cells a chimeric antigen receptor (CAR).

81. The method of claim 80, wherein the CAR is an anti-FITC CAR.

82. The method of any of claims 53-81, wherein the stem cells are pluripotent stem cells.

83. The method of any one of claims 53-82, wherein the stem cells are iPSCs.

84. A cell population produced by the method of any one of claims 53-83.

85. A pharmaceutical composition comprising the cell population of claim 52 or claim 84.

86. A method for generating hematopoietic progenitor (HP) cells, the method comprising:
a) culturing a cell population comprising engineered iPSCs of any of claims 1-51 or claim 84 under conditions to form an aggregate;
b) culturing the cells produced in a) under conditions to induce mesoderm formation in a plurality of the cells, wherein the initiation of the culturing in b) is day 0; and
c) culturing the cells produced in b) under conditions to differentiate cells into a population of hematopoietic progenitors (HP),
wherein at least a portion of one or more of steps a)-c) are carried out in the presence of a non-physiological ligand of the synthetic cytokine receptor.

87. A method for generating cytotoxic innate lymphoid (iCIL) cells, the method comprising culturing a cell population comprising engineered iPSCs of any of claims 1-51 under conditions to differentiate the iPSCs to cytotoxic innate lymphoid (iCILs), wherein a non-physiological ligand of the synthetic cytokine receptor is added during at least a portion of the culturing.

88. The method of claim 87, wherein the culturing comprises:
a) culturing the cell population comprising engineered iPSCs under conditions to form an aggregate;
b) culturing the cells produced in a) under conditions to induce mesoderm formation in a plurality of the cells, wherein the initiation of the culturing in b) is on day 0;
c) culturing the cells produced in b) under conditions to differentiate cells into a population of hematopoietic progenitors (HP); and
d) culturing the cells produced in c) under conditions to generate iCIL cells,
wherein at least a portion of one or more of steps a)-d) are carried out in the presence of the non-physiological ligand of the synthetic cytokine receptor.

89. A method for generating cytotoxic innate lymphoid (iCIL) cells, the method comprising:
a) culturing a cell population comprising engineered iPSCs of any of claims 1-51 under conditions to form an aggregate;
b) culturing the cells produced in a) under conditions to induce mesoderm formation in a plurality of the cells, wherein the initiation of the culturing in b) is day 0;
c) culturing the cells produced in b) under conditions to differentiate cells into a population of hematopoietic progenitors (HP); and
d) culturing the cells produced in c) under conditions to generate iCIL cells,
wherein at least a portion of one or more of steps a)-d) are carried out in the presence of a non-physiological ligand of the synthetic cytokine receptor.

90. The method of any of claims 86-89, wherein the culturing is carried out in a vessel treated to promote cell adhesion and growth.

91. The method of claim 90, wherein the vessel is a Matrigel.

92. The method of any of claims 86-89, wherein the culturing is carried out in a non-adherent culture vessel.

93. The method of claim 92, wherein the non-adherent culture vessel is Aggrewell™ plate.

94. The method of any of claims 86 and 88-93, wherein the aggregate in a) is an Embryoid body (EB).

95. The method of any of claims 86-89, wherein the culturing is carried out in suspension.

96. The method of any of claims 86-89 and 94, wherein the culturing is carried out in culture vessel that is not treated to promote cell adhesion and proliferation.

97. The method of claim 94 or claim 95, wherein step a) comprises:
(i) performing a first incubation comprising culturing the cell population of engineered stem cells under conditions to form a first aggregate;
(ii) contacting the aggregate with a dissociating agent to form a population of dissociated cells; and
(iii) performing a second incubation comprising culturing the population of dissociated cells under conditions to form the second aggregate.

98. The method of any of claims 86-97, wherein the culturing in b) is in a media comprising one or more of BMP4, FGF2, VEGF and a Rock Inhibitor, optionally wherein the Rock Inhibitor is Y27632.

99. The method of any of claims 86-98, wherein the culturing in b) is in a media comprising BMP4, FGF2, VEGF and Y27632.

100. The method of any of claims 86-98, wherein the culturing in b) is in a media comprising BMP4, FGF2 and VEGF.

101. The method of any of claims 86-100, wherein the culturing in b) is in a media comprising the non-physiological ligand.

102. The method of any of claims 86-96 and 101, wherein the culturing in b) is in a media comprising the non-physiological ligand without any additional growth factors.

103. The method of any of claims 86-102, wherein the culturing in b) is for 2 to 4 days, optionally for at or about 3 days

104. The method of any of claims 86-103, wherein the culturing in c) is in a media comprising one or more of BMP4, FGF2, VEGF, TPO, SCF, and LDL.

105. The method of any of claims 86-103, wherein the culturing in c) is in a media comprising one or more of BMP4, FGF2, VEGF and LDL.

106. The method of any of claims 86-103, wherein the culturing in c) is in a media comprising BMP4 and FGF2.

107. The method of claim 106, wherein the culturing in c) with BMP4 and FGF2 is for days 3 to 15.

108. The method of any of claims 86-106, wherein the culturing in c) comprises a PI3K inhibitor.

109. The method of claim 108, wherein the PI3K inhibitor is LY2940002.

110. The method of claims 108 and 109, wherein the PI3K inhibitor is added during a portion of the culturing in c).

111. The method of any of claims 108-110, wherein the PI3K inhibitor is added from about day 6 to day 15.

112. The method of any of claims 86-109, wherein the culturing in c) is in a media without SCF and TPO.

113. The method of any of claims 86-112, wherein the culturing in c) is in a media comprising the non-physiological ligand.

114. The method of any of claims 86-102 and 112, wherein the culturing in c) is in a media comprising the non-physiological ligand without any additional growth factors, cytokines or both.

115. The method of any of claims 86-112, wherein the culturing in c) is on days 3 to 15 days.

116. The method of any of claims 86-115, wherein during at least a portion of the culturing in c) the media comprises an aryl hydrocarbon receptor (AHR) antagonist e.g., a pyrimido-[4,5-b]-indole derivative e.g. or both.

117. The method of claim 116, wherein the AHR antagonist is StemRegenin 1 (SR1).

118. The method of claim 116 or claim 117, wherein the pyrimido-[4,5-b]-indole derivative is UM729.

119. The method of any of claims 116-118, wherein SR1 and UM729 are added to the culturing in c) beginning at a day from day 6 to day 9.

120. The method of claim 116 or claim 119, wherein SR1 and UM729 are added to the culturing in c) beginning at about day 6.

121. The method of any of claims 88-119, wherein the culturing in d) is in a media comprising one or more of stem cell factor (SCF), FLT3L, IL-7, IL-12, IL-15, SR-1 and UM729.

122. The method of any of claims 88-121, wherein the culturing in d) is in a media comprising the non-physiological ligand.

123. The method of any of claims 88-120 and 122, wherein the culturing in d) is in a media comprising the non-physiological ligand without any additional growth factors, cytokines or both.

124. The method of any of claims 88-123, wherein the culturing in d) is for a time between days 15 and 40.

125. The method of any of claims 88-123, wherein the culturing in d) is for days 15 and 30.

126. A method for generating cytotoxic innate lymphoid (iCIL) cells, the method comprising:
a) culturing a cell population comprising engineered iPSCs of any of claims 1-48 under conditions to form an aggregate;
b) culturing the cells produced in a) in a media comprising one or more selected from the group of BMP4, VEGF, FGF2, and ROCKi to induce mesoderm formation in a plurality of the cells, wherein the initiation of the culturing in b) is day 0;
c) culturing the cells produced in b) in a media comprising BMP4, FGF2, and LY2940002 to differentiate cells into a population of hematopoietic progenitors (HP), wherein the initiation of the culturing in c) is day 3; and
d) culturing the cells produced in d) in a media comprising SCF and IL-15 to generate iCIL cells, wherein the initiation of the culturing in b) is day 15,
wherein at least a portion of one or more of steps a)-d) are carried out in the presence of a non-physiological ligand of the synthetic cytokine receptor.

127. The method of claim 126, wherein SR1 and UM729 are added to the culturing in c) beginning at a day from day 6 to day 9.

128. The method of claim 126 or claim 127, wherein SR1 and UM729 are added to the culturing in c) beginning at about day 6.

129. A method for generating cytotoxic innate lymphoid (iCIL) cells, comprising contacting a cell population comprising an engineered stem cell of any one of claims 1-48 with the non-physiological ligand for a first period of time sufficient to generate CLPs, and contacting the CLPs with a differentiation media for a second period of time sufficient to generate iCILs.

130. The method of claim 129, wherein the differentiation media comprises stem cell factor (SCF), FLT3L, IL-7, IL-12, IL-15, SR-1 and UM729.

131. The method of claim 130, wherein the differentiation media comprises the non-physiological ligand.

132. The method of any one of claims 129-131, wherein the first period of time is 1-15 days, and wherein the second period of time is 1-15 days.

133. The method of any one of claims 129-132, comprising contacting the iCILs with a pre-activation media comprising IL-7, IL-12, IL-15, IL-18 and IL-21 for a third period of time sufficient to generate mature iCILs.

134. The method of claim 133, wherein the pre-activation media comprises the non-physiological ligand.

135. The method of claim 133 or 134, wherein the third period of time is 1-10 days.

136. The method of claim 133 or claim 134, wherein mature iCILs express NKp46, NKG2D, LFA1, DNAM1, CD16 and CD56.

137. The method of any of claims 86-136, wherein the non-physiological ligand is rapamycin or a rapamycin analog.

138. The method of claim 137, wherein the rapamycin analog is rapalog.

139. The method of any of claims 86-138, wherein the non-physiological ligand is added to the media at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM and 150 nM and 200 nM.

140. The method of any of claims 86-139, wherein the non-physiological ligand is added to the media at a concentration of at or about 10 nM.

141. The method of any of claims 86-139, wherein the non-physiological ligand is added to the media at a concentration of at or about 100 nM.

142. The method of any of claims 86-138, wherein the non-physiological ligand is added to the media at a concentration from 2.5 nM to 10 nM.

143. The method of any of claims 86-138 and 142, wherein the non-physiological ligand is added to the media at a concentration from 3 nM to 7 nM.

144. A hematopoietic progenitor (HP) cell produced by the method of any of claims 86 and 90-119.

145. The HP cell of claim 144, wherein the HP cells comprise lower expression of HLF, HOXA9, and/or CD133 compared to a CD34+ cord blood cell.

146. The HP cell of claim 145, wherein the expression of HLF, HOXA9, and/or CD133 in HP cells is 8-fold, 7-fold, 6-fold, 5-fold, 4-fold, 3-fold, 2-fold, or 1-fold lower compared to a CD34+ cord blood cell.

147. The HP cell of claim 145 or claim 146, wherein the CD34+ cord blood cell comprises a hematopoietic stem cell (HSC).

148. A cytotoxic innate lymphoid (iCIL) cell produced by the method of any of claims 87-136.

149. A hematopoietic progenitor (HP) cell that has been differentiated from a pluripotent stem cell of any of claims 1-51, wherein the HP comprises a synthetic cytokine receptor.

150. A cytotoxic innate lymphoid (iCIL) that has been differentiated from a pluripotent stem cell of any of claims 1-51, wherein the iCIL comprises a synthetic cytokine receptor.

151. A population of hematopoietic progenitor (HP) cells produced by the method of any of claims 86 and 74-95.

152. The population of claim 151, wherein the population of HP cells comprise lower expression of HLF, HOXA9, and/or CD133 compared to a population of CD34+ cord blood cells.

153. The population of claim 153, wherein the expression of HLF, HOXA9, and/or CD133 in HP cells is 8-fold, 7-fold, 6-fold, 5-fold, 4-fold, 3-fold, 2-fold, or 1-fold lower compared to a population of CD34+ cord blood cells.

154. The population of claim 152 or claim 153, wherein the population of CD34+ cord blood cell comprises a hematopoietic stem cell (HSC).

155. A population of cytotoxic innate lymphoid (iCIL) cells produced by the method of any of claims 87-141.

156. A population of cells comprising the iCILs of claim 148 or claim 150.

157. The iCIL of claim 148 or claim 150 or the population of iCIL of claim 155 or claim 156, wherein the iCIL comprise a B2M knockout.

158. The iCIL of claim 148 or claim 150 or the population of iCIL of claim 155 or claim 156, wherein the iCIL comprise a B2M knockout and a FKBP12 knockout.

159. A pharmaceutical composition comprising the iCIL or population of iCILs of any of claims 148-158.

160. A method of expanding a cytotoxic innate lymphoid cell (iCIL), the method comprising contacting an iCIL or population of iCILs of any of claims 148-158 or the pharmaceutical composition of claim 159 with the non-physiological ligand of the synthetic cytokine receptor.

161. A method of killing or inhibiting the proliferation of cancer cells, comprising contacting cancer cells with the iCIL or population of iCILs of any of claims 148-158, or the pharmaceutical composition of claim 159 with the non-physiological ligand of the synthetic cytokine receptor.

162. The method of claim 160 or claim 161 that is performed in vitro or ex vivo.

163. The method of any of claims 160-162, wherein the non-physiological ligand is rapamycin or a rapamycin analog.

164. The method of claim 163, wherein the rapamycin analog is rapalog.

165. The method of any of claims 160-164, wherein the non-physiological ligand is contacted at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM and 150 nM and 200 nM.

166. The method of any of claims 160-165, wherein the non-physiological ligand is contacted at a concentration of at or about 10 nM.

167. The method of any of claims 160-165, wherein the non-physiological ligand is contacted at a concentration of at or about 100 nM.

168. The method of any of claims 160-164, wherein the non-physiological ligand is added to the media at a concentration from 2.5 nM to 10 nM.

169. The method of any of claims 160-164 and 168, wherein the non-physiological ligand is added to the media at a concentration from 3 nM to 7 nM.

170. The method of any of claims 160-164 and 168-169, wherein the non-physiological ligand is added to the media at a concentration of at or about 3.1 nM.

171. The method of any of claims 160, 161 and 163-166, wherein the method is performed in vivo in a subject and the non-physiological ligand is administered to the subject.

172. A method of treating a cancer in a subject, comprising administering to the subject an effective amount of the cell population of any one of claims 1-51 and 84, or the pharmaceutical composition of claim 85.

173. A method of treating a cancer in a subject, comprising administering to the subject an effective amount of the iCIL or population of iCILs of any of claims 148-158, or the pharmaceutical composition of claim 159.

174. The method of any of claims 171-173, wherein the subject has not been administered a lymphodepleting therapy prior to the administering the iCIL, population of iCILs or the pharmaceutical composition.

175. The method of any of claims 160-174, wherein the iCIL express a CAR targeting cancer cells in the subject.

176. The method of claim 175, wherein the CAR is an anti-FITC CAR and the subject has been administered a FITC-ligand to tag a cancer cell in the subject, wherein the ligand specifically binds a molecule expressed on a tumor.

177. The method of claim 176, wherein the FITC-ligand is FITC-folate.

178. The method of any of claims 171-177, comprising administering to the subject the non-physiological ligand of the synthetic cytokine receptor.

179. The method of any of claims 171-178, wherein the non-physiological ligand is rapamycin or a rapamycin analog.

180. The method of claim 179, wherein the rapamycin analog is rapalog.

181. The method of any of claims 171-180, wherein the non-physiological ligand is administered at a dose of 1 mg to 100 mg, optionally between 10-100 mg, optionally at or about 10 mg, 20 mg, 25 mg, 30 mg, 40 mg, 50 mg or any value between any of the foregoing.

182. The method of any of claims 171-181, wherein multiple doses of the non-physiological ligand are administered to the subject.

183. The method of claim 182, wherein the multiple doses are administered intermittently or at regular intervals after administration of the iCIL population or composition thereof to the subject, optionally for a predetermined period of time.

184. The method of claim 171-183, wherein 2 to 8 doses of the non-physiological ligand are administered to the subject.

185. The method of any of claims 171-181, wherein a single dose of the non-physiological ligand is administered to the subject.

186. The method of any of claims 172-185, wherein the iCIL population or composition thereof is administered at a dose that is from at or about 1×10⁸ iCIL cells to at or about 100×10⁹ iCIL cells.

187. The method of any of claims 172-186, wherein the iCIL population or composition thereof is administered at a dose that is greater than at or about 5×10⁹ iCIL cells, optionally wherein the dose is from at or about 5×10⁹ iCIL cells to at or about 100×10⁹ iCIL cells.

188. The method of any of claims 171-187, wherein multiple doses of the iCIL cells are administered to the subject.

189. The method of claim 188, wherein the multiple doses of iCIL cells are administered intermittently or at regular intervals, optionally for a predetermined period of time.

190. The method of claim 171-189, wherein 2 to 8 doses of the iCIL cells are administered to the subject.

191. The method of any of claims 171-187, wherein a single dose of the iCIL cells is administered to the subject.

192. A kit comprising the engineered stem cells of any one of claims 1-51 and 84 and instructions for differentiating the cell population to cytotoxic innate lymphoid cells.

193. A kit comprising the iCIL or the population of iCIL of 148-158 or the pharmaceutical composition of claim 159 and instructions for administering to a subject in need thereof.

194. The kit of claim 192 or claim 193, further comprising a container comprising the non-physiological ligand and instructions for administering the non-physiological ligand to the subject after administration of the cell population.

195. The kit of any of claims 192-194, wherein the subject has a cancer.

196. A population of induced cytotoxic innate lymphoid (iCIL) cells, wherein the iCILs are mature iCILs expressing CD56 and LFA1, and wherein:
at least 25% of the iCILs express a cytotoxicity receptor;
no more than 75% of the iCILs express a dysfunction receptor; and/or
at least 25% of the iCILs are proliferative.

197. The population of claim 196, wherein at least 25% of the iCILs express a cytotoxicity receptor.

198. The population of claim 196 or claim 197, wherein the cytotoxicity receptor is one or more of NKp30, NKp46, and NKG2D.

199. The population of any one of claims 196-198, wherein at least 25%, at least 30%, at least 35%, at least 40%, at least 45%, at least 50%, at least 55%, or at least 60% of the iCILs express NKp30+.

200. The population of any one of claims 196-199, wherein at least 55%, at least 60%, at least 65%, at least 70%, at least 75%, or at least 80% of the iCILs express NKp46.

201. The population of any one of claims 196-200, wherein at least 50%, at least 55%, at least 60%, at least 65%, at least 70%, at least 75%, at least 80%, at least 85%, or at least 90% of the iCILs express NKG2D.

202. The population of any one of claims 196-201, wherein no more than 75% of the iCILs express a dysfunction receptor.

203. The population of any one of claims 196-202, wherein the dysfunction receptor is one or more of KLRG1, CD73, and CD38.

204. The population of any one of claims 196-203, wherein no more than 45%, no more than 40%, no more than 35%, no more than 30%, no more than 25%, no more than 20%, no more than 15%, no more than 10%, or no more than 5% of the iCILs express KLRG1.

205. The population of any one of claims 196-204, wherein no more than 10%, no more than 9%, no more than 8%, no more than 7%, no more than 6%, no more than 5%, no more than 4%, no more than 3%, no more than 2%, or no more than 1% of the iCILs express CD73.

206. The population of any one of claims 196-205, wherein no more than 75%, no more than 65%, no more than 55%, no more than 45%, no more than 35%, no more than 25%, no more than 15%, or no more than 5% of the iCILs express CD38.

207. The population of any one of claims 196-206, wherein at least 25%, at least 30%, at least 35%, at least 40%, at least 45%, at least 50%, at least 55%, or at least 60% of the iCILs are proliferative.

208. The population of any one of claims 196-207, wherein the iCILs that are proliferative are CD56bright CD57−.

209. The population of any one of claims 196-208, wherein the iCILs further comprise a synthetic cytokine receptor for a non-physiological ligand,
wherein the cytokine receptor comprises:
a synthetic gamma chain polypeptide comprising a first dimerization domain, a first transmembrane domain, and an interleukin-2 receptor subunit gamma (IL-2RG) intracellular domain, and
a synthetic beta chain polypeptide comprising a second dimerization domain, a second transmembrane domain, and an intracellular domain selected from an interleukin-2 receptor subunit beta (IL-2RB) intracellular domain, an interleukin-7 receptor subunit beta (IL-7RB) intracellular domain, and/or an interleukin-21 receptor subunit beta (IL-21RB) intracellular domain.

210. The population of claim 209, wherein the first dimerization domain and the second dimerization domain are extracellular domains.

211. The population of claim 209 or claim 210, wherein the synthetic gamma chain polypeptide comprises, in N- to C-terminal order, the first dimerization domain, the first transmembrane domain, and the interleukin-2 receptor subunit gamma (IL-2RG) intracellular domain, and
the synthetic beta chain polypeptide comprises, in N- to C-terminal order, the second dimerization domain, the second transmembrane domain, and the intracellular domain.

212. The population of any one of claims 209-211, wherein the IL-2RG intracellular domain comprises a polypeptide sequence at least 95% identical to SEQ ID NO: 1, or a polypeptide sequence as set forth in SEQ ID NO: 1.

213. The population of any one of claims 209-212, wherein the first transmembrane domain comprises the IL-2RG transmembrane domain.

214. The population of claim 213, wherein the IL-2RG transmembrane domain comprises a polypeptide sequence at least 95% identical to SEQ ID NO: 8 or 31, or a polypeptide sequence as set forth in SEQ ID NO: 8 or 31.

215. The population of any one of claims 209-214, wherein the beta chain intracellular domain comprises the IL-2RB intracellular domain.

216. The population of claim 215, wherein the IL-2RB intracellular domain comprises a polypeptide sequence at least 95% identical to SEQ ID NO: 2, or a polypeptide sequence as set forth in SEQ ID NO: 2.

217. The population of any one of claims 209-214, wherein the beta chain intracellular domain comprises the IL-7RB intracellular domain.

218. The population of claim 217, wherein the IL-7RB intracellular domain comprises a polypeptide sequence at least 95% identical to SEQ ID NO: 3, or a polypeptide sequence as set forth in SEQ ID NO: 3.

219. The population of any one of claims 209-214, wherein the beta chain intracellular domain comprises the IL-21RB intracellular domain.

220. The population of claim 219, wherein the IL-21RB intracellular domain comprises a polypeptide sequence at least 95% identical to SEQ ID NO: 4, or a polypeptide sequence as set forth in SEQ ID NO: 4.

221. The population of any one of claims 209-220, wherein the second transmembrane domain comprises a transmembrane domain from the same beta chain intracellular domain.

222. The population of any one of claims 209-216 and 221, wherein the second transmembrane domain is a transmembrane domain of IL-2RB comprising a polypeptide sequence at least 95% identical to SEQ ID NO: 35 or 36, or a polypeptide sequence as set forth in SEQ ID NO: 35 or 36.

223. The population of any one of claims 209-216, 221, and 222, wherein:
the synthetic gamma chain polypeptide contains an IL-2RG TM domain comprising the sequence set forth in SEQ ID NO: 8 or 31 and a IL-2RG intracellular domain comprising the sequence set forth in SEQ ID NO:1; and
the synthetic beta chain polypeptide contains an IL-2RB TM domain comprising the sequence set forth in SEQ ID NO: 35 or 36 and a IL-2RB intracellular domain comprising the sequence set forth in SEQ ID NO:2.

224. The population of any one of claims 209-223, wherein the first dimerization domain and the second dimerization domain are heterodimerization domains selected from FK506-Binding Protein of size 12 kD (FKBP) and a FKBP12-rapamycin binding (FRB) domain; and/or
wherein the non-physiological ligand is rapamycin or a rapalog.

225. The population of claim 224, wherein the FRB domain comprises a polypeptide sequence at least 95% identical to SEQ ID NO: 6 or SEQ ID NO: 7.

226. The population of claim 224, wherein the FRB domain comprises the polypeptide sequence set forth in SEQ ID NO: 6 or SEQ ID NO: 7.

227. The population of any one of claims 209-223, wherein the first dimerization domain and the second dimerization domain are heterodimerization domains selected from FK506-Binding Protein of size 12 kD (FKBP) and a calcineurin domain; and/or
wherein the non-physiological ligand is FK506 or an analogue thereof.

228. The population of any one of claims 209-224, wherein the FKBP domain comprises a polypeptide sequence at least 95% identical to SEQ ID NO: 5 or SEQ ID NO:30.

229. The population of any one of claims 209-224, wherein the FKBP domain comprises the polypeptide sequence set forth in SEQ ID NO: 5 or SEQ ID NO:30.

230. The population of any one of claims 209-216 and 221-229, wherein the synthetic gamma chain polypeptide has the sequence of amino acids set forth in SEQ ID NO:28 or a sequence of amino acids that is at least 90%, at least 95%, at least 96%, at least 97%, at least 98%, at least 99%, or 100% identical to SEQ ID NO: 28, and the synthetic beta chain polypeptide has the sequence of amino acids set forth in SEQ ID NO:33 or a sequence of amino acids that is at least 90%, at least 95%, at least 96%, at least 97%, at least 98%, at least 99%, or 100% identical to SEQ ID NO: 33.

231. The population of any one of claims 209-216 and 221-230, wherein the synthetic gamma chain polypeptide has the sequence of amino acids set forth in SEQ ID O: 28 and the synthetic beta chain polypeptide has the sequence of amino acids set forth in SEQ ID NO: 33.

232. The population of any one of claims 209-223, wherein the first dimerization domain and the second dimerization domain are homodimerization domains selected from:
i) FK506-Binding Protein of size 12 kD (FKBP);
ii) cyclophiliA (CypA); or
iii) gyrase B (CyrB);
and the non-physiological ligand is, respectively:
i) FK1012, AP1510, AP1903, or AP20187 or an analog thereof;
ii) cyclosporin-A (CsA) or an analog thereof; or
iii) coumermycin or an analog thereof.

233. The population of any one of claims 196-232, wherein the population comprises from about 1×10⁶ to about 1×10¹² iCILs, from about 1×10⁶ to about 1×10¹⁰ iCILs, from about 1×10⁶ to about 1×10⁸ iCILs, from about 1×10⁸ to about 1×10¹² iCILs, from about 1×10⁸ to about 1×10¹⁰ iCILs, or from about 1×10¹⁰ to about 1×10¹² iCILs.

234. The population of any one of claims 196-233, wherein the volume of the population is from about 1 mL to about 100 mL, from about 1 mL to about 80 mL, from about 1 mL to about 60 mL, from about 1 mL to about 40 mL, from about 1 mL to about 20 mL, from about 1 mL to about 10 mL, from about 10 mL to about 100 mL, from about 10 mL to about 80 mL, from about 10 mL to about 60 mL, from about 10 mL to about 40 mL, from about 10 mL to about 20 mL, from about 20 mL to about 100 mL, from about 20 mL to about 80 mL, from about 20 mL to about 60 mL, from about 20 mL to about 40 mL, from about 40 mL to about 100 mL, from about 40 mL to about 80 mL, from about 40 mL to about 60 mL, from about 60 mL to about 100 mL, from about 60 mL to about 80 mL, or from about 80 mL to about 100 mL.

235. A pharmaceutical composition comprising the population of iCILs of any one of claims 196-234.

236. The pharmaceutical composition of claim 235, further comprising a cryoprotectant.

237. A cryopreserved composition comprising the population of iCILs of any one of claims 196-234.

238. The composition of any one of claims 235-237, wherein the composition comprises from about 1×10⁶ to about 1×10¹² iCILs, from about 1×10⁶ to about 1×10¹⁰ iCILs, from about 1×10⁶ to about 1×10⁸ iCILs, from about 1×10⁸ to about 1×10¹² iCILs, from about 1×10⁸ to about 1×10¹⁰ iCILs, or from about 1×10¹⁰ to about 1×10¹² iCILs.

239. The composition of any one of claims 235-238, wherein the volume of the composition is from about 1 mL to about 100 mL, from about 1 mL to about 80 mL, from about 1 mL to about 60 mL, from about 1 mL to about 40 mL, from about 1 mL to about 20 mL, from about 1 mL to about 10 mL, from about 10 mL to about 100 mL, from about 10 mL to about 80 mL, from about 10 mL to about 60 mL, from about 10 mL to about 40 mL, from about 10 mL to about 20 mL, from about 20 mL to about 100 mL, from about 20 mL to about 80 mL, from about 20 mL to about 60 mL, from about 20 mL to about 40 mL, from about 40 mL to about 100 mL, from about 40 mL to about 80 mL, from about 40 mL to about 60 mL, from about 60 mL to about 100 mL, from about 60 mL to about 80 mL, or from about 80 mL to about 100 mL.

240. A method of killing or inhibiting the proliferation of target cells, comprising contacting target cells with the population of iCILs of any one of claims 196-234 or the composition of any one of claims 235-239.

241. The method of claim 240, wherein the target cells are cancer cells.

242. The method of claim 240 or claim 241, wherein the iCILs further comprise the synthetic cytokine receptor for the non-physiological ligand, and the method comprises contacting the target cells with the non-physiological ligand of the synthetic cytokine receptor.

243. The method of any one of claims 240-242 that is performed in vitro or ex vivo.

244. The method of claim 242 or claim 243, wherein the non-physiological ligand is rapamycin or a rapamycin analog.

245. The method of claim 244, wherein the rapamycin analog is rapalog.

246. The method of any one of claims 242-245, wherein the non-physiological ligand is contacted at a concentration of between 5 nM and 200 nM, 5 nM and 150 nM, 5 nM and 100 nM, 5 nM and 50 nM, 5 nM and 20 nM, 5 nM and 10 nM, 10 nM and 200 nM, 10 nM and 150 nM, 10 nM and 100 nM, 10 nM and 50 nM, 10 nM and 20 nM, 20 nM and 200 nM, 20 nM and 150 nM 20 nM and 100 nM, 20 nM and 50 nM, 50 nM and 200 nM, 50 nM and 150 nM, 50 nM and 100 nM, 100 nM and 200 nM, 100 nM and 150 nM, or 150 nM and 200 nM.

247. The method of any one of claims 242-246, wherein the non-physiological ligand is contacted at a concentration of at or about 10 nM.

248. The method of any one of claims 242-246, wherein the non-physiological ligand is contacted at a concentration of at or about 100 nM.

249. The method of any one of claims 242-245, wherein the non-physiological ligand is added to the media at a concentration from 2.5 nM to 10 nM.

250. The method of any one of claims 242-245 and 249, wherein the non-physiological ligand is added to the media at a concentration from 3 nM to 7 nM.

251. The method of any one of claims 240-242 and 244-248, wherein the method is performed in vivo in a subject, and the population of iCILs or composition thereof is administered to the subject.

252. The method of claim 251, wherein the iCILs further comprise the synthetic cytokine receptor for the non-physiological ligand, and the method comprises administering the non-physiological ligand to the subject.

253. A method of inducing natural killer (NK) cell-mediated cell killing in a subject, comprising administering to the subject an effective amount of the population of iCILs of any one of claims 196-234 or the composition of any one of claims 235-239.

254. A method of treating a cancer in a subject, comprising administering to the subject an effective amount of the population of iCILs of any one of claims 196-234 or the composition of any one of claims 235-239.

255. The method of any one of claims 251-254, wherein the subject has not been administered a lymphodepleting therapy prior to the administering of the population of iCILs or composition thereof.

256. The method of any one of claims 251-255, wherein the iCILs express a CAR targeting cancer cells in the subject.

257. The method of claim 256, wherein the CAR is an anti-FITC CAR, and the subject has been administered a FITC-ligand to tag a cancer cell in the subject, wherein the ligand specifically binds a molecule expressed on a tumor.

258. The method of claim 257, wherein the FITC-ligand is FITC-folate.

259. The method of any one of claims 253-258, wherein the iCILs further comprise the synthetic cytokine receptor for the non-physiological ligand, and the method comprises administering to the subject the non-physiological ligand of the synthetic cytokine receptor.

260. The method of any one of claims 252 and 255-259, wherein the non-physiological ligand is rapamycin or a rapamycin analog.

261. The method of claim 260, wherein the rapamycin analog is rapalog.

262. The method of any one of claims 252 and 255-261, wherein the non-physiological ligand is administered at a dose of 1 mg to 100 mg, optionally between 10-100 mg, optionally at or about 10 mg, 20 mg, 25 mg, 30 mg, 40 mg, 50 mg or any value between any of the foregoing.

263. The method of any one of claims 252 and 255-262, wherein multiple doses of the non-physiological ligand are administered to the subject.

264. The method of claim 263, wherein the multiple doses are administered intermittently or at regular intervals after the administration of the population of iCILs or composition thereof to the subject, optionally for a predetermined period of time.

265. The method of any one of claims 252 and 255-264, wherein 2 to 8 doses of the non-physiological ligand are administered to the subject.

266. The method of any one of claims 252 and 255-262, wherein a single dose of the non-physiological ligand is administered to the subject.

267. The method of any one of claims 251-266, wherein the population of iCILs or composition thereof is administered at a dose that is from at or about 1×10⁸ iCIL cells to at or about 100×10⁹ iCIL cells.

268. The method of any one of claims 251-267, wherein the population of iCILs or composition thereof is administered at a dose that is greater than at or about 5×10⁹ iCIL cells, optionally wherein the dose is from at or about 5×10⁹ iCIL cells to at or about 100×10⁹ iCIL cells.

269. The method of any one of claims 251-268, wherein multiple doses of the iCIL cells are administered to the subject.

270. The method of claim 269, wherein the multiple doses of iCIL cells are administered intermittently or at regular intervals, optionally for a predetermined period of time.

271. The method of any one of claims 251-270, wherein 2 to 8 doses of the iCIL cells are administered to the subject.

272. The method of any one of claims 251-268, wherein a single dose of the iCIL cells is administered to the subject.

273. A kit comprising the population of iCILs of any one of claims 196-234 or the composition of any one of claims 235-239 and instructions for administering the population of iCILs or composition thereof to a subject in need thereof.

274. The kit of claim 273, wherein the iCILs further comprise the synthetic cytokine receptor for the non-physiological ligand, and the kit further comprises a container comprising the non-physiological ligand and instructions for administering the non-physiological ligand to the subject after administration of the population of iCILs or composition thereof.

275. The kit of claim 273 or claim 274, wherein the subject has a cancer.
